(19) United States
(12) Patent Application Publication
(10) Pub. No.: US 2023/0279120 A1
(43) Pub. Date: Sep. 7, 2023

(54) METHODS OF TREATING OR INHIBITING CARDIOVASCULAR DISEASES

(71) Applicant: TrueBinding, Inc., Foster City, CA (US)

(72) Inventors: Dongxu Sun, Palo Alto, CA (US); Suhail Rasool, Foster City, CA (US); Yew Ann Leong, San Fransisco, CA (US); George M. Haig, Melbourne, FL (US); Jenny Ulrika Johansson, Mountainview, CA (US); Ludmila Anatolievna Volobouevna, Walnut Creek, CA (US)

(21) Appl. No.: 18/053,191

(22) Filed: Nov. 7, 2022

Related U.S. Application Data
(60) Provisional application No. 63/263,773, filed on Nov. 9, 2021.

(51) Int. Cl. C07K 16/28; A61P 9/10; A61P 25/28

(52) U.S. Cl. CPC C07K 16/2851; A61P 9/10; A61P 25/28; A61K 2039/545

(57) ABSTRACT

Disclosed herein are methods and compositions for inhibiting, preventing, ameliorating, reducing, or treating cardiovascular diseases, for example, stroke, traumatic brain injury, cerebral amyloid angiopathy, atherosclerosis, myocardial infarction, and/or diseases associated with fibrin activity or dysfunction. These methods and compositions involve antibodies that can bind to Galectin-3 and inhibit, prevent, ameliorate, reduce, or treat the cardiovascular diseases in a patient by reducing inflammation and/or inhibiting oligomerization of proteins associated with pathology such as amyloid beta or fibrin.

### CROSS-REFERENCE TO RELATED APPLICATIONS

This application claims the benefit of priority of U.S. Provisional Pat. Application No. 63/263,773, filed Nov. 09, 2021, which is hereby expressly incorporated by reference in its entirety, including any appendices filed therewith.

### REFERENCE TO SEQUENCE LISTING

The present application is being filed along with a Sequence Listing in electronic format. The Sequence Listing is provided as a file entitled ReplacementIMMUT032ASeqListing. XML, which was created and last modified on May 15, 2023, which is 1,425,609 bytes in size. The information in the electronic Sequence Listing is hereby incorporated by reference in its entirety.

### FIELD

Aspects of the present disclosure relate generally to methods and compositions for inhibiting, preventing, ameliorating, reducing, or treating cardiovascular diseases. These methods and compositions involve the use of antibodies that bind to Galectin-3 (Gal3).

### BACKGROUND

Galectin-3 (Gal3, GAL3) is a lectin, or a carbohydrate-binding protein, with specificity towards beta-galactosides. In human cells, Gal3 is expressed and can be found in the nucleus, cytoplasm, cell surface, and in the extracellular space. Gal3 recognizes and interacts with beta-galactose conjugates on various proteins.

### SUMMARY

Galectin-3 (Gal3) has been implicated to have immunomodulatory activity. An example of this is the interaction between Gal3 and T-cell immunoglobulin and mucin-domain containing-3 (TIM-3), which causes suppression of immune responses such as T cell activation and may enable cancer cells to evade immune clearance. This phenomenon and methods to inhibit the same are exemplified in WO 2019/023247 and WO 2020/160156, each of which is hereby expressly incorporated by reference in its entirety. Disclosed herein are methods for inhibiting, reducing, preventing, and/or treating stroke in a subject in need thereof. In some embodiments, the methods comprise administering an anti-Gal3 antibody or binding fragment thereof to the subject, thereby inhibiting, reducing, preventing, and/or treating the stroke. In some embodiments, the stroke is an ischemic stroke, thrombotic stroke, embolic stroke, transient ischemic attack, hemorrhagic stroke, intracerebral hemorrhage, or subarachnoid hemorrhage.

Also disclosed herein are methods for inhibiting, reducing, preventing, and/or treating traumatic brain injury (TBI) in a subject in need thereof. In some embodiments, the methods comprise administering an anti-Gal3 antibody or binding fragment thereof to the subject, thereby inhibiting, reducing, preventing, and/or treating the TBI. In some embodiments, the TBI is associated with a concussion, edema, diffuse axonal injury, spinal cord injury, coma, neuroinflammation, microhemorrhage, astrocytosis, activated microglia, and/or hematoma.

Also disclosed herein are methods for inhibiting, reducing, preventing, and/or treating a disorder associated with fibrin activity or dysfunction in a subject in need thereof. In some embodiments, the methods comprise administering an anti-Gal3 antibody or binding fragment thereof to the subject, thereby inhibiting, reducing, preventing, and/or treating the disorder associated with fibrin activity or dysfunction. In some embodiments, the disorder associated with fibrin activity or dysfunction comprises atherosclerosis, thrombosis, thromboembolism, carotid artery disease, coronary artery disease, peripheral artery disease, myocardial infarction, heart failure, heart attack, hypertension, chronic kidney disease, coagulopathy, or thrombocytopathy.

Also disclosed herein are methods for inhibiting, reducing, preventing, and/or treating a disorder associated with inflammation and elevation of proinflammatory cytokines in a subject in need thereof. The methods comprise administering an anti-Gal3 antibody or binding fragment thereof to the subject, thereby treating the disorder associated with inflammation. In some embodiments, the administration reduces proinflammatory cytokines, such as circulating cytokines, in the subject.

Also disclosed herein are methods comprising administering 5 unit doses of an anti-Gal3 antibody or binding fragment thereof, where each unit dose comprises 1000 mg or about 1000 mg of the anti-Gal3 antibody or binding fragment thereof, where the unit doses are administered every 7 days or about 7 days, and where each unit dose is administered over the course of 1 hour or about 1 hour.

Also disclosed herein are single-use sealed injectable glass vials comprising 8 mL of 20 mg/mL of an anti-Gal3 antibody or binding fragment thereof.

Also disclosed herein are IV infusion bags comprising 1000 mg or about 1000 mg of an anti-Gal3 antibody or binding fragment thereof dissolved in 250 mL or about 250 mL of saline.

In some embodiments, the anti-Gal3 antibody or binding fragment is any one or more of the anti-Gal3 antibodies or binding fragments thereof, or any portion or component of any one or more of the anti-Gal3 antibodies or binding fragments thereof disclosed herein, including but not limited to 1, 2, 3, 4, 5, or 6 CDRs, heavy chain variable regions, light chain variable regions, heavy chains, or light chains.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts exemplary protein sequences of Gal3.

FIG. 2 depicts exemplary peptide sequences of Gal3 used to generate and analyze antibodies.

FIG. 3A depicts exemplary variable heavy chain complementarity-determining region (CDR) 1 for anti-Gal3 antibodies disclosed herein. In some embodiments, any of the compositions or methods provided herein can include one or more of the variable heavy chain CDR1 provided herein.

FIG. 3B depicts exemplary variable heavy chain CDR2 for anti-Gal3 antibodies disclosed herein. In some embodiments, any of the compositions or methods provided herein can include one or more of the variable heavy chain CDR2 provided herein.

FIG. 3C depicts exemplary variable heavy chain CDR3 for anti-Gal3 antibodies disclosed herein. In some embodiments, any of the compositions or methods provided herein can include one or more of the variable heavy chain CDR3 provided herein.

FIG. 4A depicts exemplary variable light chain CDR1 for anti-Gal3 antibodies disclosed herein. In some embodiments, any of the compositions or methods provided herein can include one or more of the variable light chain CDR1 provided herein.

FIG. 4B depicts exemplary variable light chain CDR2 for anti-Gal3 antibodies disclosed herein. In some embodiments, any of the compositions or methods provided herein can include one or more of the variable light chain CDR2 provided herein.

FIG. 4C depicts exemplary variable light chain CDR3 for anti-Gal3 antibodies disclosed herein. In some embodiments, any of the compositions or methods provided herein can include one or more of the variable light chain CDR3 provided herein.

FIG. 5 depicts exemplary heavy chain variable region (VH) sequences for anti-Gal3 antibodies disclosed herein. In some embodiments, any of the compositions or methods provided herein can include one or more of the VH sequences provided herein.

FIG. 6 depicts exemplary light chain variable region (VL) sequences for anti-Gal3 antibodies disclosed herein. In some embodiments, any of the compositions or methods provided herein can include one or more of the VL sequences provided herein.

FIG. 7 depicts exemplary combinations of heavy and light chain CDRs (CDR1, CDR2, and CDR3) of exemplary anti-Gal3 antibodies disclosed herein. In some embodiments, any of the compositions or methods provided herein can include one or more of the heavy and light chain CDR combinations provided herein.

FIG. 8 depicts exemplary combinations of heavy and light chain variable regions of exemplary anti-Gal3 antibodies disclosed herein. In some embodiments, any of the compositions or methods provided herein can include one or more of the heavy and light chain variable region combinations provided herein.

FIG. 9 depicts exemplary heavy chain (HC) sequences and light chain (LC) sequences, and possible pairings for exemplary anti-Gal3 antibodies disclosed herein. In some embodiments, any of the compositions or methods provided herein can include one or more of the HC or LC, or pairs of HC and LC sequences provided herein.

FIG. 10 depicts peptides that were found to bind to exemplary anti-Gal3 antibodies disclosed herein (according to the peptide nomenclature depicted in FIG. 2 as discussed herein) and binning of these exemplary antibodies.

FIG. 11A depicts K_{D} (M) values of Gal3 binding for exemplary anti-Gal3 antibodies disclosed herein.

FIG. 11B depicts antibodies affinities (K_{D}) of anti-Gal3 humanized antibodies TB001 (IMT001) and TB006 (IMT006) for human, cynomolgus, rat, and mouse Gal3.

FIG. 12 depicts antibody names used throughout the present disclosure refer to the same antibody (with exemplary peptide and nucleic acid sequences provided elsewhere in the disclosure and appropriately attributed to at least one of the depicted names) and may be used interchangeably. The names shown in a column correspond to the same antibody.

FIG. 13 depicts an alignment of hinge and constant heavy chain domain 2 (C_{H}2) domain amino acid sequences of wild-type human immunoglobulin G1 (IgG1), IgG2 and IgG4 as well as their sigma variants. The alignment above uses EU numbering. Residues identical to wild-type IgG1 are indicated as dots; gaps are indicated with hyphens. Sequence is given explicitly if it differs from wild-type IgG1 or from the parental subtype for σ variants. Open boxes beneath the alignment correspond to International Immunogenetics Information System (IMGT) strand definitions. Boxes beneath the alignment correspond to the strand and helix secondary structure assignment for wild-type IgG1. Residues 267-273 form the BC loop and 322-332 form the FG loop. Also provided are exemplary constant regions for human IgG4 heavy (S228P mutant) and light (kappa) chains (SEQ ID NOs: 931-932) and murine IgG2A (LALAPG and LALA mutants) (SEQ ID NOs: 933-934). In some embodiments, any one or more of the VH/VL and/or CDRs provided in the other figures or otherwise disclosed herein can be paired with any one or more of the exemplary constant regions provided herein.

FIG. 14 depicts nucleic acid sequences that encode for exemplary heavy chain variable regions of anti-Gal3 antibodies disclosed herein. In some embodiments, any of the compositions or methods provided herein can include one or more of the heavy chain variable regions encoded by the nucleic acids provided herein.

FIG. 15 depicts nucleic acid sequences that encode for exemplary light chain variable regions of anti-Gal3 antibodies disclosed herein. In some embodiments, any of the compositions or methods provided herein can include one or more of the light chain variable regions encoded by the nucleic acids provided herein.

FIG. 16 depicts nucleic acid sequences that encode for exemplary heavy chains of anti-Gal3 antibodies disclosed herein. In some embodiments, any of the compositions or methods provided herein can include one or more of the heavy chains encoded by the nucleic acids provided herein.

FIG. 17 depicts nucleic acid sequences that encode for exemplary light chains of anti-Gal3 antibodies disclosed herein. In some embodiments, any of the compositions or methods provided herein can include one or more of the light chains encoded by the nucleic acids provided herein.

FIGS. 18A-B depicts an exemplary alignment for the heavy chain CDRs (FIG. 18A) and light chain CDRs (FIG. 18B) for the exemplary anti-Gal3 antibodies disclosed herein.

FIGS. 19A-D each depict a table with a subset of anti-Gal3 antibodies disclosed herein. In some embodiments, sequences, including heavy chain variable region CDRs, light chain variable region CDRs, heavy chain variable regions, light chain variable regions, heavy chains, and light chains, or combinations thereof, can be selected from any one or more of the anti-Gal3 antibodies provided as subsets in each table.

FIGS. 20A-D each depict a table with a subset of anti-Gal3 antibodies disclosed herein. In some embodiments, sequences, including heavy chain variable region CDRs, light chain variable region CDRs, heavy chain variable regions, light chain variable regions, heavy chains, and light chains, or combinations thereof, can be selected from any one or more of the anti-Gal3 antibodies provided as subsets in each table.

FIGS. 21A-B depict quantification of locomotor activity in an intracerebral hemorrhage (ICH) stroke mouse model showing significantly shorter latency to fall (FIG. 21A) and less distance travelled (FIG. 21B) as compared to age matched healthy control mice.

FIG. 22 depicts an exemplary dose regimen after inducing stroke (ICH). Mice were dosed with 8 doses of mTB001 or MOPC21 for 4 weeks (2 doses per week).

FIGS. 23A-D depict quantification of locomotor activity in an ICH mouse model before and after treatment with mTB001 or MOPC21 and compared to control wild-type. Treated mice were assessed after 4 (FIG. 23A), 6 (FIG. 23B), and 8 doses. After 8 doses of treatment, stroke-induced mice treated with mTB001 showed significant improvement in terms of latency to fall (FIG. 23C) and distance travelled (FIG. 23D), with locomotor ability similar to age matched healthy control mice.

FIGS. 24A-C depict reduction in microhemorrhage in ICH model mice treated with mTB001 as quantified by Prussian Blue staining. Representative Prussian Blue-stained brain slices of mTB001-treated mice showed significantly reduced microhemorrhage compared to MOPC21-treated mice (FIG. 24A). Healthy control mice showed no microhemorrhage (FIG. 24B). FIG. 24C is the quantification of Prussian Blue-positive surface area of the representative brain slices of FIGS. 24A-B.

FIGS. 25A-B depict a reduction of activated microglia in ICH model mice treated with mTB001. FIG. 25A shows brain slices of mTB001 or MOPC21 isotype control treated ICH mice and wild type control mice stained with the microglia marker Iba1. FIG. 25B is the quantification of Iba1 positive staining of the representative brain slices of FIG. 24A.

FIGS. 26A-B depict a reduction of activated astrocytes in ICH model mice treated with mTB001. FIG. 26A shows brain slices of mTB001 or MOPC21 isotype control treated ICH mice and wild type control mice stained with the astrocyte marker GFAP. FIG. 26B is the quantification of GFAP positive staining of the representative brain slices of FIG. 26A.

FIGS. 27A-F depict a mouse model of TBI treated with exemplary anti-Gal3 antibody mTB001 exhibiting reduced levels of activated microglia and astrocytes, and levels of myeloid immune cells in the affected brain. The brains of TBI model and wild type mice were probed with antibodies against the activated microglia marker Iba1 (FIG. 27A), the activated astrocyte marker GFAP (FIG. 27C), and the myeloid cell marker CD68 (FIG. 27E). FIG. 27B shows quantification of FIG. 27A showing that mTB001 significantly reduces activated microglia compared to isotype control. FIG. 27D shows quantification of FIG. 27C showing that mTB001 significantly reduces activated astrocytes compared to isotype control. FIG. 27F shows quantification of FIG. 27E showing that mTB001 significantly reduces levels of myeloid cells, such as macrophages and microglia, compared to isotype control.

FIGS. 28A-D depict a mouse model of TBI treated with exemplary anti-Gal3 antibody mTB001 exhibiting reduced levels of hyperphosphorylated tau and microhemorrhage in the affected brain. The brains of TBI model and wild type mice were probed with antibody AT8, which binds to phosphorylated tau (FIG. 28A), or stained with Prussian Blue (FIG. 28C). FIG. 28B shows quantification of FIG. 28A showing that mTB001 significantly reduces levels of hyperphosphorylated tau compared to isotype control. FIG. 28D shows quantification of FIG. 28C showing that mTB001 significantly reduces microhemorrhage compared to isotype control.

FIG. 28E depicts a mouse model of TBI treated with exemplary antibody mTB001 exhibiting reduced levels of neurofilament-light (NF-L) in plasma after 48 hours of treatment.

FIG. 29 depicts exemplary anti-Gal3 antibodies assessed for activity against fibrin oligomerization of Example 3.

FIGS. 30A-B depict a time course of Gal3-mediated fibrin oligomerization over a course of 0-5 hours. FIG. 30A shows a dot blot of fibrin incubated with or without Gal3 (100 ng/mL each) for 0-5 hours and probed with oligomer specific conformational antibody A11. FIG. 30B shows quantification of FIG. 30A.

FIGS. 31A-B depict a time course of Gal3-mediated fibrin oligomerization over a course of 0-24 hours. FIG. 31A shows a dot blot of fibrin incubated with or without Gal3 (100 µg/mL each) for 0-24 hours and probed with oligomer specific conformational antibody A11. FIG. 31B shows quantification of FIG. 31A.

FIG. 32 depicts the ability for exemplary anti-Gal3 antibodies TB001 and TB006 to degrade fibrin oligomers. 100 µg/mL each of fibrin and Gal3 were incubated for 24 hours prior to the addition of Gal3 antibodies at a concentration of 0, 3, 10, or 100 µg/mL and incubated for 3 hours. Dot blot of samples were probed with A11 antibody. Degradation of fibrin oligomers were observed at all tested concentrations of antibody.

FIGS. 33A-C depict the ability for additional exemplary anti-Gal3 antibodies to degrade fibrin oligomers. FIG. 33A shows a time course of fibrin aggregation in a dot blot with samples of 100 µg/mL each of fibrin and Gal3 incubated for 0, 2, 4, 6, or 24 hours and probed with A11 antibody. FIG. 33B shows a dot blot showing degradation of fibrin oligomers by exemplary anti-Gal3 antibodies as listed according to assigned numbers shown in FIG. 29. 100 µg/mL each of fibrin and Gal3 were incubated for 24 hours prior to the addition of 100 µg/mL of the listed anti-Gal3 antibody clones. Antibody incubation was done for 24 hours, and samples were probed on a dot blot with A11 antibody. Dot blot was done in duplicate for each condition. FIG. 33C shows quantification of FIGS. 33A-B (24 hour time point for fibrin or fibrin/Gal3 only; and 24 hour fibrin/Gal3 incubation + 24 hour antibody incubation [one of the two duplicates of FIG. 33B was quantified]).

FIG. 34 depicts Gal3 induction of cytokine release by activated neutrophils (using an HL60 neutrophil-like cell line model) and inhibition of this effect by anti-Gal3 antibodies. Expression of TNF-α, IL-6, and IL-8 was measured by ELISA.

FIGS. 35A-C depict Gal3 induction of cytokine release by activated monocytes (using a THP-1 monocyte-like cell line model) and inhibition of this effect by anti-Gal3 antibodies. After 6 or 24 hours of cell incubation, expression of TNF-α (FIG. 35A), IL-6 (FIG. 35B), and IL-8 (FIG. 35C) was measured by ELISA. No expression of IL-6 was observed at the 6 hour time point.

FIG. 36 depicts an exemplary administration and follow up schema for the acute ischemic stroke (AIS) treatment using TB006 as described in Example 12.

### DETAILED DESCRIPTION OF THE DISCLOSURE

Galectin-3 (Gal3, GAL3) plays a role in cell proliferation, adhesion, differentiation, angiogenesis, and apoptosis. This activity is, at least in part, due to immunomodulatory properties and binding affinity towards other immune regulatory proteins, signaling proteins, and other cell surface markers.

Gal3 functions by distinct N-terminal and C-terminal domains. The N-terminal domain (isoform 1: amino acids 1-111, isoform 3: amino acids 1-125) comprise a tandem repeat domain (TRD, isoform 1: amino acids 36-109, isoform 3: amino acids 50-123) and is largely responsible for oligomerization of Gal3. The C-terminal domain (isoform 1: amino acids 112-250, isoform 3: amino acids 126-264) comprise a carbohydrate-recognition-binding domain (CRD), which binds to β-galactosides.

An exemplary sequence for isoform 1 of human Gal3 (NCBI Reference No. NP_002297.2) is shown in SEQ ID NO: 1. An exemplary sequence for isoform 3 of human Gal3 (NCBI Reference No. NP_001344607.1) is shown in SEQ ID NO: 2.

Disclosed herein are methods for inhibiting, reducing, preventing, and/or treating stroke in a subject in need thereof. The methods comprise administering an anti-Gal3 antibody or binding fragment thereof to the subject, thereby inhibiting, reducing, preventing, and/or treating the stroke. In some embodiments, the stroke is an ischemic stroke, thrombotic stroke, embolic stroke, transient ischemic attack, hemorrhagic stroke, intracerebral hemorrhage, or subarachnoid hemorrhage.

Also disclosed herein are methods for inhibiting, reducing, preventing, and/or treating traumatic brain injury (TBI) in a subject in need thereof. The methods comprise administering an anti-Gal3 antibody or binding fragment thereof to the subject, thereby inhibiting, reducing, preventing, and/or treating the TBI. In some embodiments, the TBI is associated with a concussion, edema, diffuse axonal injury, spinal cord injury, coma, neuroinflammation, microhemorrhage, astrocytosis, activated microglia, and/or hematoma.

Also disclosed herein are methods for inhibiting, reducing, preventing, and/or treating a disorder associated with fibrin activity or dysfunction in a subject in need thereof. The methods comprise administering an anti-Gal3 antibody or binding fragment thereof to the subject, thereby inhibiting, reducing, preventing, and/or treating the disorder associated with fibrin activity or dysfunction. In some embodiments, the disorder associated with fibrin activity or dysfunction comprises atherosclerosis, thrombosis, thromboembolism, carotid artery disease, coronary artery disease, peripheral artery disease, myocardial infarction, heart failure, heart attack, hypertension, chronic kidney disease, coagulopathy, or thrombocytopathy.

Any and all of the methods provide herein are also contemplated for use in the preparation of a medicament for the treatment of the noted disorder and/or as the corresponding composition for use in the treatment of the noted disorder.

In some embodiments, the anti-Gal3 antibody or binding fragment is any one or more of the anti-Gal3 antibodies or binding fragments thereof, or any portion or component of any one or more of the anti-Gal3 antibodies or binding fragments thereof disclosed herein, including but not limited to 1, 2, 3, 4, 5, or 6 CDRs, heavy chain variable regions, light chain variable regions, heavy chains, or light chains.

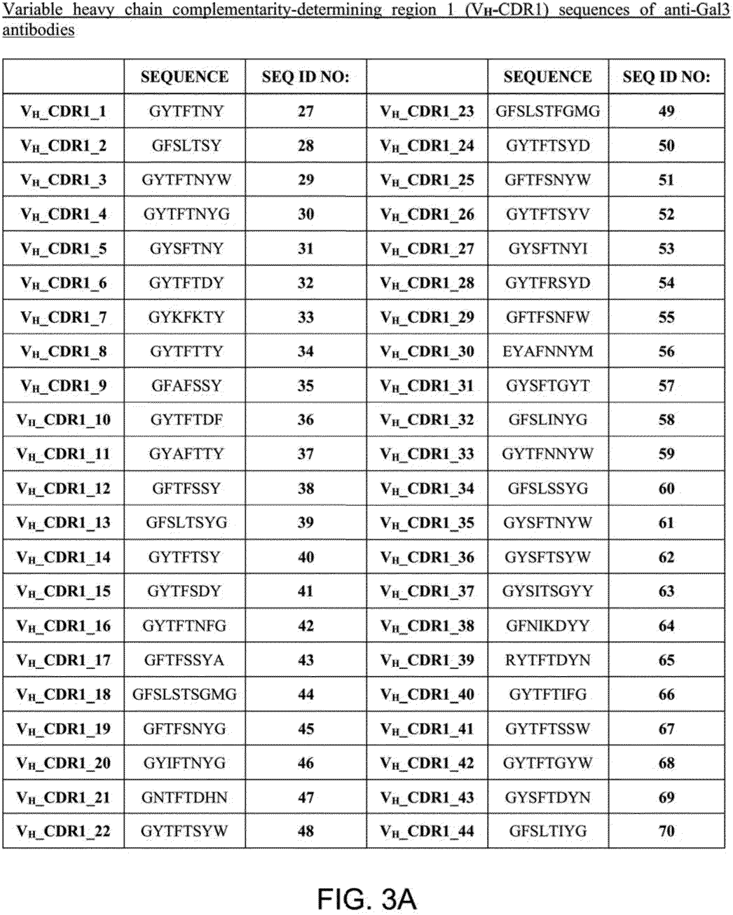

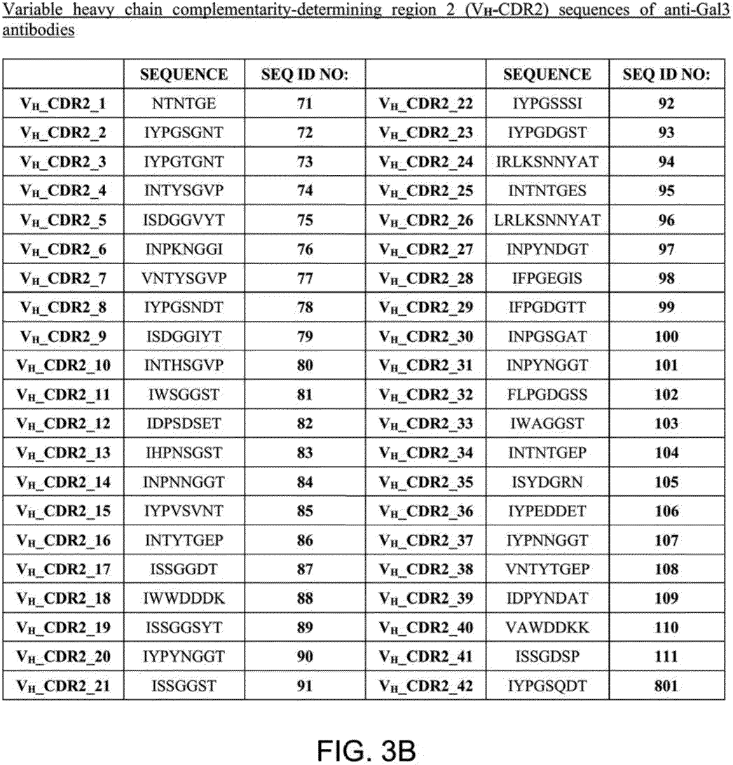

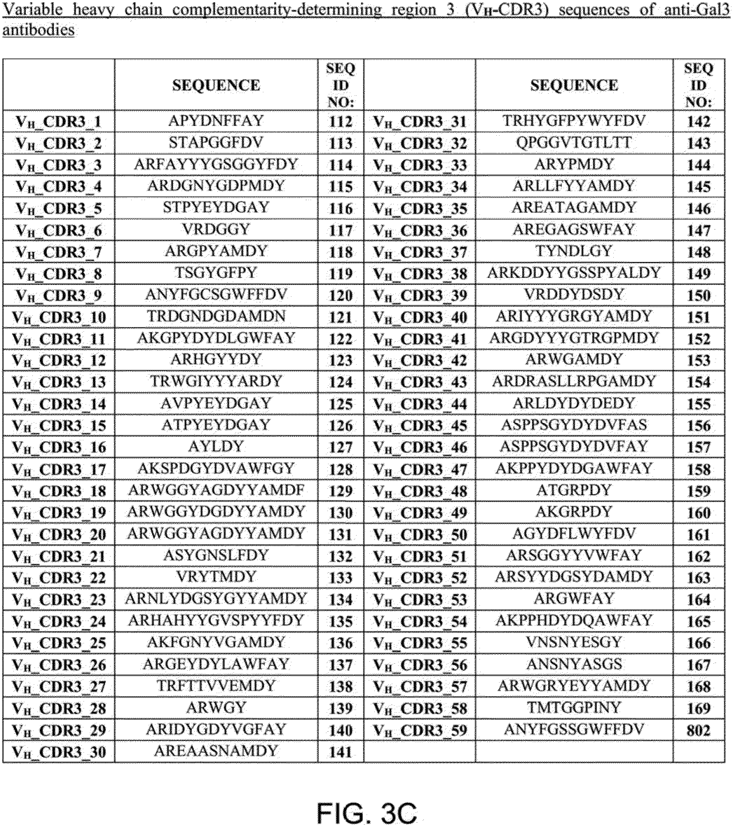

In some embodiments, the anti-Gal3 antibody or binding fragment thereof is or comprises any one or more of the sequences illustrated in FIGS. 3A-C, 4A-C, 5, 6, 7, 8, or 9, includes any 1, 2, 3, 4, 5, or 6 of the CDRs in SEQ ID NOs: 27-538, 801-865, and/or includes one or both of the VH/VL in SEQ ID NOs: 297-447, 803-865, 926-929. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 27, 71, 112, 170, 221, 248. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 31, 72, 113, 171, 222, 249. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 30, 86, 130, 189, 230, 263. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 116, 174, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 32, 84, 119, 186, 228, 255. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 37, 77, 118, 178, 229, 256. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 126, 187, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 66, 108, 164, 215, 225, 291.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The section headings used herein are for organizational purposes only and are not to be construed as limiting the subject matter described.

In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician’s assistant, an orderly or a hospice worker).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies may be polyclonal antibodies, although monoclonal antibodies may be preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)₂, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (e.g. U.S. Pat. No. 5,985,660, hereby expressly incorporated by reference in its entirety).

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, either alone or in combination.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In some embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003, Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM.TM., A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, IMGT, Paratome, AbM, and/or conformational definitions, or a combination of any of the foregoing.

As disclosed herein, sequences having a % identity to any of the sequences disclosed herein are envisioned and may be used. The terms “% identity” refer to the percentage of units (i.e. amino acids or nucleotides) that are the same between two or more sequences relative to the length of the sequence. When the two or more sequences being compared are the same length, the % identity will be respective that length. When two or more sequences being compared are different lengths, deletions and/or insertions may be introduced to obtain the best alignment. In some embodiments, these sequences may include peptide sequences, nucleic acid sequences, CDR sequences, variable region sequences, or heavy or light chain sequences. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein may be used. The changes in sequences may apply to, for example, single amino acids, single nucleic acid bases, or nucleic acid codons; however, differences in longer stretches of sequences are also envisioned. As applied to antibody sequences, these differences in sequences may apply to antigen-binding regions (e.g., CDRs) or regions that do not bind to antigens or are only secondary to antigen binding (e.g., framework regions).

As disclosed herein, sequences having a % homology to any of the sequences disclosed herein are envisioned and may be used. The term “% homology” refers to the degree of conservation between two sequences when considering their three-dimensional structure. For example, homology between two protein sequences may be dependent on structural motifs, such as beta strands, alpha helices, and other folds, as well as their distribution throughout the sequence. Homology may be determined through structural determination, either empirically or in silico. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein, which may or may not affect the overall % homology, may be used.

As applied herein, sequences having a certain % similarity to any of the sequence disclosed herein are envisioned and may be used. In some embodiments, these sequences may include peptide sequences, nucleic acid sequences, CDR sequences, variable region sequences, or heavy or light chain sequences. As understood in the art with respect to peptide sequences, “similarity” refers to the comparison of amino acids based on their properties, including but not limited to size, polarity, charge, pK, aromaticity, hydrogen bonding properties, or presence of functional groups (e.g. hydroxyl, thiol, amine, carboxyl, and the like). The term “% similarity” refers to the percentage of units (i.e. amino acids) that are the same between two or more sequences relative to the length of the sequence. When the two or more sequences being compared are the same length, the % similarity will be respective that length. When two or more sequences being compared are different lengths, deletions and/or insertions may be introduced to obtain the best alignment. The similarity of two amino acids may dictate whether a certain substitution is conservative or non-conservative. Methods of determining the conservativeness of an amino acid substitution are generally known in the art and may involve substitution matrices. Commonly used substitution matrices include BLOSUM45, BLOSUM62, BLOSUM80, PAM100, PAM120, PAM160, PAM200, PAM250, but other substitution matrices or approaches may be used as considered appropriate by the skilled person. A certain substitution matrix may be preferential over the others when considering aspects such as stringency, conservation and/or divergence of related sequences (e.g. within the same species or broader), and length of the sequences in question. As used herein, a peptide sequence having a certain % similarity to another sequence will have up to that % of amino acids that are either identical or an acceptable substitution as governed by the method of similarity determination used. In some embodiments, a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 similar substitutions relative to any of the sequences disclosed herein may be used. As applied to antibody sequences, these similar substitutions may apply to antigen-binding regions (i.e. CDRs) or regions that do not bind to antigens or are only secondary to antigen binding (i.e. framework regions).

The term “consensus sequence” as used herein with regard to sequences refers to the generalized sequence representing all of the different combinations of permissible amino acids at each location of a group of sequences. A consensus sequence may provide insight into the conserved regions of related sequences where the unit (e.g. amino acid or nucleotide) is the same in most or all of the sequences, and regions that exhibit divergence between sequences. In the case of antibodies, the consensus sequence of a CDR may indicate amino acids that are important or dispensable for antigen binding. It is envisioned that consensus sequences may be prepared with any of the sequences provided herein, and the resultant various sequences derived from the consensus sequence can be validated to have similar effects as the template sequences.

The term “compete,” as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody “specifically binds” or “preferentially binds” to a target if it binds with greater affinity, and/or avidity, and/or more readily, and/or with greater duration than it binds to other substances. For example, an antibody that specifically or preferentially binds to a target epitope is an antibody that binds this epitope with greater affinity, and/or avidity, and/or more readily, and/or with greater duration than it binds to other target epitopes or non-target epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

The term “block” or “disrupt” as used herein with regard to an antibody refers to the ability of an antibody to interfere with a biological process, including but not limited to activity of an enzyme, binding of two or more biological molecules (e.g. two or more proteins, peptides, nucleic acids, lipids, and the like), or advancement of a signaling cascade. Generally, interference with a biological process will involve the antibody binding to its target or an epitope thereof, thereby interfering with the normal function of said target, such as occluding an active site of the target, occluding another region of the target important for its function, or altering the localization and/or transport of the target. The blocking or disruption activity of an antibody may be quantified in terms of the reduction of the biological process in question relative to a control condition where the biological process is not disrupted. In other cases, the blocking or disruption activity of an antibody may be quantified in terms of a modulation in another biological process known to be associated with the target biological process, whether it be directly related or inversely related. In some embodiments, the blocking or disruption activity may cause a change of at least 5%, 10%, 20%, 30%, 40%, 50%, 60%, 70%, 80%, 90%, or 100%, or any percentage within a range defined by any two of the aforementioned values, relative to a control condition. In some embodiments provided herein, an interaction between Gal3 and fibrin is a biological process that can be disrupted by an anti-Gal3 antibody. It is envisioned that the interaction between Gal3 and fibrin may or may not be a direct interaction between Gal3 and fibrin, and the anti-Gal3 antibody may interfere with some other aspect of the activity of Gal3 and/or fibrin.

As used herein, the term “antigen binding molecule” refers to a molecule that comprises an antigen binding portion that binds to an antigen and, optionally, a scaffold or framework portion that allows the antigen binding portion to adopt a conformation that promotes binding of the antigen binding portion or provides some additional properties to the antigen binding molecule. In some embodiments, the antigen is Gal3. In some embodiments, the antigen binding portion comprises at least one CDR from an antibody that binds to the antigen. In some embodiments, the antigen binding portion comprises all three CDRs from a heavy chain of an antibody that binds to the antigen or from a light chain of an antibody that binds to the antigen. In some embodiments, the antigen binding portion comprises all six CDRs from an antibody that binds to the antigen (three from the heavy chain and three from the light chain). In some embodiments, the antigen binding portion is an antibody fragment.

Nonlimiting examples of antigen binding molecules include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, pig, dog, cat, horse, donkey, guinea pig, goat, or camelid. Antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis. The antigen binding molecule can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold.

An antigen binding molecule can also include a protein comprising one or more antibody fragments incorporated into a single polypeptide chain or into multiple polypeptide chains. For instance, antigen binding molecule can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol.17:315-23, 2004); a peptibody (one or more peptides attached to an Fc region, see WO 00/24782); a linear antibody (a pair of tandem Fd segments, VH-CH1-VH-CH1) which, together with complementary light chain polypeptides, form a pair of antigen binding regions, see Zapata et al., Protein Eng., Vol. 8:1057-1062, 1995); a small modular immunopharmaceutical (see U.S. Pat. Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc).

In certain embodiments, an antigen binding molecule can have, for example, the structure of an immunoglobulin. An “immunoglobulin” is a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Unless otherwise specified, the complementarity defining regions disclosed herein follow the IMGT definition. In some embodiments, any of the CDRs disclosed herein can instead be interpreted by Kabat, Chothia, or other definitions accepted by those of skill in the art.

The term “humanized” as applies to a non-human (e.g. rodent or primate) antibodies are hybrid immunoglobulins, immunoglobulin chains or fragments thereof which contain minimal sequence derived from non-human immunoglobulin.

As used herein, the term “cytokine” refers to small proteins, polypeptides, or peptides that are involved in inflammatory signaling or proteins released by one cell population that act on another cell as intercellular mediators or have an autocrine effect on the cells producing the proteins. Cytokines include but are not limited to chemokines, interferons, interleukins, lymphokines, monokines, tumor necrosis factors, CCL1, CCl2, CCL3, CCL4, CCL5, CCL6, CCL7, CCL8, CCL9, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CX3CL1, XCL1, XCL2, INFα, INFβ, INFγ, IL-1, IL-1α, IL-1β, IL-1Ra, IL-2, IL-3, IL-4, IL-5, IL-6, IL-7, IL-8, IL-9, IL-10, IL-11, IL-12, IL-13, IL-14, IL-15, IL-16, IL-17, IL-17A-F, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-36Ra, IL-36α, IL-36β, IL-36γ, IL-37, IL-38, adesleukin, GM-CSF, TNFα, TNFβ, TNFγ, TGF-I-3 TNFSF4, TNFSF5, TNFSF6, TNFSF7, TNFSF8, TNFSF9, TNFSF10, TNFSF11, TNFSF12, TNFSF13, TNFSF13B, TNFSF14, TNFSF15, TNFSF18, or TNFSF19, leukemia inhibitor factor (LIF), ciliary neurotrophic factor (CNTF), CNTF-like cytokine (CLC), cardiotrophin (CT), Kit ligand (KL), or any combination thereof.

As used herein, the terms “treating” or “treatment”, and related terms such as “inhibiting”, “reducing”, and “preventing”, (and as well understood in the art) means an approach for obtaining beneficial or desired results in a subject’s condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease’s transmission or spread, delaying or slowing of disease progression, amelioration or palliation of the disease state, diminishment of the reoccurrence of disease, and remission, whether partial or total and whether detectable or undetectable. As provided herein, “preventing” may include one or more of “inhibiting” or “reducing”. “Treating” and “treatment” as used herein may include a therapeutic treatment (e.g., in response to a current disease state) and/or a prophylactic treatment (e.g. to prevent the occurrence or severity of an anticipated disease state). Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The terms “effective amount” or “effective dose” as used herein have their plain and ordinary meaning as understood in light of the specification, and refer to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

In some non-limiting embodiments, an effective amount or effective dose of a composition or compound may relate to the amount or dose that provides a significant, measurable, or sufficient therapeutic effect towards the treatment of any one or more of the diseases provided herein, such as cardiovascular diseases, for example, stroke, traumatic brain injury, cerebral amyloid angiopathy, atherosclerosis, myocardial infarction, and/or diseases associated with fibrin activity or dysfunction, or cancers. In some embodiments, the effective amount or effective dose of a composition or compound may treat, ameliorate, or prevent the progression of symptoms of any one or more of the diseases provided herein.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

As used herein, a “carrier” refers to a compound, particle, solid, semi-solid, liquid, or diluent that facilitates the passage, delivery and/or incorporation of a compound to cells, tissues and/or bodily organs. For example, without limitation, a lipid nanoparticle (LNP) is a type of carrier that can encapsulate an oligonucleotide to thereby protect the oligonucleotide from degradation during passage through the bloodstream and/or to facilitate delivery to a desired organ, such as to the lungs.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “excipient” has its ordinary meaning as understood in light of the specification, and refers to inert substances, compounds, or materials added to a pharmaceutical composition to provide, without limitation, bulk, consistency, stability, binding ability, lubrication, disintegrating ability etc., to the composition. Excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, dextran, fructose, mannose, lactose, galactose, sucrose, sorbitol, cellulose, methyl cellulose, hydroxypropyl methyl cellulose (hypromellose), glycerin, polyvinyl alcohol, povidone, propylene glycol, serum, amino acids, polyethylene glycol, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), tris(hydroxymethyl)aminomethane (Tris), citric acid, ascorbic acid, acetic acid, salts, phosphates, citrates, acetates, succinates, chlorides, bicarbonates, borates, sulfates, sodium chloride, sodium bicarbonate, sodium phosphate, sodium borate, sodium citrate, potassium chloride, potassium phosphate, magnesium sulfate sugars, dextrose, dextran 40, fructose, mannose, lactose, trehalose, galactose, sucrose, sorbitol, mannitol, cellulose, serum, amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, polysorbate 20, polysorbate 40, polysorbate, 60, polysorbate 80, poloxamer, poloxamer 188, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the formulation at a percentage that is at least 0%, 0. 1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy.

As used herein, the term “inhibit” refers to the reduction or decrease in an expected activity, such as a cellular activity. The reduction or decrease may be by at least 5%, 10%, 20%, 30%, 40%, 50%, 60%, 70%, 80%, 90%, or 100%, or any percentage that is within a range defined by any two of the aforementioned values, where a reduction or decrease of 100% indicates a complete inhibition and any lower percentage indicates a partial inhibition. The reduction or decrease of the expected activity may be observed in a direct or indirect way.

As used herein, the term “stroke” refers to a medical disorder involving disruption of normal blood flow supplying the brain. There are generally two categories of stroke: ischemic stroke (including thrombotic stroke, embolic stroke, or transient ischemic attack) where blood supply is cut off from the brain, such as from blockage due to a blood clot, blood vessel compression, or atherosclerotic plaques; and hemorrhagic stroke (including intracerebral hemorrhage or subarachnoid hemorrhage) where disruption of blood vessels leads to bleeding in the brain, which may be caused by a ruptured aneurism or physical trauma. Symptoms of stroke involve neurological issues such as paralysis, loss of feeling, loss of vision, dizziness, difficulty speaking, or issues with cognitive ability. Patients with a stroke or at risk of developing a stroke may be selected by assessing causative factors, such as high blood pressure, smoking, obesity, high blood cholesterol, diabetes, kidney disease, or heart dysfunction. As disclosed herein, the compositions disclosed herein may be used for the treatment or prophylaxis for strokes or symptoms thereof, including those provided herein or otherwise known in the art.

As used herein, the term “traumatic brain injury (TBI)” refers to a medical condition involving damage to the brain caused by an external force, such as blunt trauma, falls, collisions, sudden changes in acceleration, or explosions. The external force exerted on the brain can result in damage to brain tissue and resulting loss of function of the affected region. Symptoms, which may differ in severity based on the severity of the TBI, may include dizziness, headache, vomiting, lethargy, increased intracranial pressure, edema, unconsciousness, and issues with cognitive ability. Patients with traumatic brain injury may be assessed by medical examination following the external insult. Individuals who are at risk of developing traumatic brain injury may be assessed based on their lifestyle, such as occupation, hobbies, age, or tendencies in behavior. As disclosed herein, the compositions disclosed herein may be used for the treatment or prophylaxis for TBI or symptoms thereof, including those provided herein or otherwise known in the art.

As used herein, the term “fibrin” refers to the protein that is involved in blood clotting, and is also known as “Factor Ia”. Upon blood vessel disruption, circulating fibrinogen is proteolytically processed by thrombin to form fibrin, which polymerizes and forms an insoluble network that, among other functions, captures platelets in order to block the blood vessel injury, preventing additional blood from escaping as well as preventing external substances, such as pathogens, from entering. The activity of fibrin and other components of the coagulation cascade may lead to harmful blood clots and thrombosis. As disclosed herein, the compositions disclosed herein may be used for the treatment or prophylaxis for disorders associated with fibrin activity or dysfunction, or symptoms thereof, including those provided herein or otherwise known in the art.

The term “% w/w” or “% wt/wt” means a percentage expressed in terms of the weight of the ingredient or agent over the total weight of the composition multiplied by 100.

It is understood that an antibody with an antibody name described herein can be referred using a shortened version of the antibody name, as long as there are no conflicts with another antibody described herein. For example, F846C.1B2 can also be referred to as 846C.1B2, or 846.1B2. This can also refer to fragments of the antibody (e.g., with the same 1, 3, or 6 CDRs).

### Exemplary Methods of Treatment and/or Use

Any of the anti-Gal3 antibodies or binding fragments thereof, or antigen binding molecules, and formulations thereof, provided herein may be used in methods as provided herein.

Disclosed herein in some embodiments are methods for inhibiting, reducing, preventing, and/or treating stroke in a subject in need thereof. The methods comprise administering an anti-Gal3 antibody or binding fragment thereof to the subject, thereby inhibiting, reducing, preventing, and/or treating the stroke. In some embodiments, any of the methods disclosed herein involving an anti-Gal3 antibody or binding fragment can be performed with an antigen binding molecule that binds to Gal3.

In some embodiments, a method for inhibiting, reducing, preventing, and/or treating traumatic brain injury (TBI) in a subject in need thereof is disclosed. In some embodiments, the method comprises administering an anti-Gal3 antibody or binding fragment thereof to the subject, thereby inhibiting, reducing, preventing, and/or treating the TBI.

In some embodiments, a method for inhibiting, reducing, preventing, and/or treating a disorder associated with fibrin activity or dysfunction in a subject in need thereof is disclosed. In some embodiments, the method comprises administering an anti-Gal3 antibody or binding fragment thereof to the subject, thereby inhibiting, reducing, preventing, and/or treating the disorder associated with fibrin activity or dysfunction.

In some embodiments, a method for administering an anti-Gal3 antibody is disclosed. In some embodiments, the method comprises administering 5 unit doses of an anti-Gal3 antibody or binding fragment thereof, wherein each unit dose comprise 1000 mg or about 1000 mg of the anti-Gal3 antibody or binding fragment thereof, wherein the unit doses are administered every 7 days or about 7 days, and wherein each unit dose is administered over the course of 1 hour or about 1 hour.

In some embodiments, a single-use sealed injectable glass vial is disclosed. In some embodiments, the single-use sealed injectable glass vial comprises 8 mL of 20 mg/mL of an anti-Gal antibody or binding fragment thereof.

In some embodiments, an IV infusion bag is disclosed. In some embodiments, the IV infusion bag comprises 1000 mg or about 1000 mg of an anti-Gal3 antibody or binding fragment thereof dissolved in 250 mL or about 250 mL of saline.

In some embodiments, the methods further comprise selecting the subject as having the stroke or at risk of contracting the stroke prior to the administering step. In some embodiments, selecting the subject as having the stroke or at risk of contracting the stroke comprises assessing the subject according to the National Institutes of Health Stroke Scale (NIHSS), modified Rankin Scale (mRS), Fugl-Meyer Assessment (FMA), Montreal Cognitive Assessment (MoCA), neuroimaging (such as CT or MRI), and/or detecting stroke biomarkers (including but not limited to S100 calcium binding protein B (S100B), glial fibrillary acidic protein (GFAP), neuron-specific enolase (NSE), and/or matrix metalloproteinase-9 (MMP9)). In some embodiments, the methods further comprise detecting an amelioration of symptoms associated with the stroke after the administering step. In some embodiments, the stroke is an ischemic stroke, thrombotic stroke, embolic stroke, transient ischemic attack, hemorrhagic stroke, intracerebral hemorrhage, or subarachnoid hemorrhage. In some embodiments, the stroke is intracerebral hemorrhage (ICH). In some embodiments, the ICH is associated with deposition of amyloid aggregates and/or cerebral amyloid angiopathy (CAA).

In some embodiments, the method for inhibiting, reducing, preventing, and/or treating stroke in a subject comprises detecting decreased loss of locomotor dysfunction, decreased incidence of microhemorrhage, decreased levels of activated microglia, decreased levels of activated astrocytes, decreased circulating proinflammatory immune cells, decreased circulating proinflammatory cytokines, or any combination thereof, in the subject.

In some embodiments, the reduction in locomotor dysfunction is assessed by the Fugl-Meyer Assessment (FMA), Motor Assessment Scale (MAS), and/or the Chedoke-McMaster Stroke Assessment (CMSA).

In some embodiments, the reduction in locomotor dysfunction in the subject is by at least 50%. In some embodiments, the reduction in locomotor dysfunction in the subject, is by at least 5%, 10%, 20%, 30%, 40%, 50%, 60%, 70%, 80%, 90%, or 95%, or any percentage within a range defined by any two of the aforementioned reductions. For example, in some embodiments, the reduction in locomotor dysfunction in the subject, is by at least 5%-95%, 5%-90%, 5%-75% 5%-50%,5%-25%, 5%-10%, 5%-7%, 7%-95%, 7%-90%, 7%-75%, 7%-50%, 7%-25%,7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, the reduction in microhemorrhage is assessed by neuroimaging, MRI, and/or Prussian blue staining.

In some embodiments, the reduction in microhemorrhage in the subject, is by at least 50%. In some embodiments, the reduction in microhemorrhage in the subject, is by at least 5%, 10%, 20%, 30%, 40%, 50%, 60%, 70%, 80%, 90%, or 95%, or any percentage within a range defined by any two of the aforementioned reductions. For example, in some embodiments, reduction in microhemorrhage is by at least 5%-95%, 5%-90%, 5%-75% 5%-50%, 5%-25%, 5%-10%, 5%-7%, 7%-95%, 7%-90%, 7%-75%, 7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, the reduction in levels of activated microglia is assessed by detection of microglia activation markers, optionally ionized calcium-binding adaptor molecule 1 (Iba1), monocyte chemoattractant protein-1 (MCP-1), chitinase-3-like protein 1 (YKL-40), and/or soluble CD14 (sCD14), optionally from the cerebrospinal fluid of the subject.

In some embodiments, the reduction in levels of activated microglia in the subject is by at least 20%. In some embodiments, the reduction in levels of activated microglia, reduction in levels of activated astrocytes, reduction in levels of circulating proinflammatory cytokines, or any combination thereof, in the subject, is by at least 5%, 10%, 20%, 30%, 40%, 50%, 60%, 70%, 80%, 90%, or 95%, or any percentage within a range defined by any two of the aforementioned reductions. For example, in some embodiments, the reduction in levels of activated microglia in the subject, is by at least 5%-95%, 5%-90%, 5%-75% 5%-50%, 5%-25%, 5%-10%, 5%-7%, 7%-95%, 7%-90%, 7%-75%, 7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, the reduction in levels of activated astrocytes is assessed by detection of astrocyte activation markers, optionally GFAP and/or S100B, optionally from the cerebrospinal fluid of the subject.

In some embodiments, the reduction in levels of activated astrocytes in the subject is by at least 30%. In some embodiments, the reduction in levels of activated astrocytes in the subject, is by at least 5%, 10%, 20%, 30%, 40%, 50%, 60%, 70%, 80%, 90%, or 95%, or any percentage within a range defined by any two of the aforementioned reductions. For example, in some embodiments, the reduction in levels of activated astrocytes in the subject, is by at least 5%-95%, 5%-90%, 5%-75% 5%-50%, 5%-25%, 5%-10%, 5%-7%, 7%-95%, 7%-90%, 7%-75%,7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, the reduction in levels of circulating proinflammatory cytokines in the subject, is by at least 5%, 10%, 20%, 30%, 40%, 50%, 60%, 70%, 80%, 90%, or 95%, or any percentage within a range defined by any two of the aforementioned reductions. For example, in some embodiments, the reduction in levels of circulating proinflammatory cytokines in the subject, is by at least 5%-95%, 5%-90%, 5%-75% 5%-50%, 5%-25%, 5%-10%, 5%-7%, 7%-95%, 7%-90%, 7%-75%, 7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, the reduction in levels circulating proinflammatory immune cells in the subject, is by at least 5%, 10%, 20%, 30%, 40%, 50%, 60%, 70%, 80%, 90%, or 95%, or any percentage within a range defined by any two of the aforementioned reductions. For example, in some embodiments, the reduction in locomotor dysfunction, reduction in microhemorrhage, reduction in circulating proinflammatory immune cells in the subject is by at least 5%-95%, 5%-90%, 5%-75% 5%-50%, 5%-25%, 5%-10%, 5%-7%, 7%-95%7%-90%, 7%-75%, 7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, a method for inhibiting, reducing, preventing, and/or treating traumatic brain injury (TBI) in a subject in need thereof is disclosed. In some embodiments, the TBI is associated with a concussion, edema, diffuse axonal injury, spinal cord injury, coma, neuroinflammation, microhemorrhage, astrocytosis, activated microglia, and/or hematoma.

In some embodiments, administration of the anti-Gal3 antibody or binding fragment thereof prevents elevation of levels of activated microglia, prevents elevation of levels of activated astrocytes, prevents elevation of levels of macrophages, prevents elevation of levels of hyperphosphorylated Tau, prevents incidence of microhemorrhage, prevents neuroinflammation, prevents elevation of levels of circulating proinflammatory cytokines, or any combination thereof, in the subject.

In some embodiments, wherein administration of the anti-Gal3 antibody or binding fragment thereof improves the level of consciousness, memory loss, and/or the Glasgow Coma Scale of the patient; and/or reduces levels of activated microglia, reduces levels of activated astrocytes, reduces levels of macrophages, reduces levels of hyperphosphorylated Tau, reduces microhemorrhage, reduces neuroinflammation, reduces levels of circulating proinflammatory cytokines or any combination thereof, in the subject.

In some embodiments, administration of the anit-Gal3 antibody results in a reduction in levels of activated microglia, reduction in levels of activated astrocytes, reduction in levels of macrophages, reduction in levels of hyperphosphorylated Tau, reduction in microhemorrhage, reduction in neuroinflammation, reduction in levels of circulating proinflammatory cytokines, or any combination thereof, in the subject.

In some embodiments, the reduction in levels of activated microglia in the subject is by at least 70%. In some embodiments, the reduction in levels of activated microglia in the subject is by at least 5%, 10%, 20%, 30%, 40%, 50%, 60%, 70%, 80%, 90%, or 95%, or any percentage within a range defined by any two of the aforementioned reductions. For example, in some embodiments, the reduction in levels of activated microglia in the subject, is by at least 5%-95%, 5%-90%, 5%-75% 5%-50%, 5%-25%, 5%-10%, 5%-7%, 7%-95%, 7%-90%, 7%-75%, 7%-50%, 7%-25%,7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, the reduction in levels of activated astrocytes is assessed by detection of astrocyte activation markers, optionally GFAP, optionally from the cerebrospinal fluid of the subject.

In some embodiments, the reduction in levels of activated astrocytes in the subject is by at least 90%. In some embodiments, the reduction in levels of activated astrocytes in the subject is by at least 5%, 10%, 20%, 30%, 40%, 50%, 60%, 70%, 80%, 90%, or 95%, or any percentage within a range defined by any two of the aforementioned reductions. For example, in some embodiments, the reduction in levels of activated astrocytes in the subject, is by at least 5%-95%, 5%-90%, 5%-75% 5%-50%, 5%-25%, 5%-10%, 5%-7%, 7%-95%, 7%-90%, 7%-75%, 7%-50%, 7%-25%, 7%-10%, 10%-95%, 10%-90%, 10%-75%, 10%-50%, 10%-25%, 25%-95%, 25%-90%,25%-75%, 25%-50%, 50%-95%, 50%-90%, 50%-75%, 75%-95%, 75%-90%, and 90%-95%.

In some embodiments, the reduction in levels of macrophages is assessed by detection of macrophage activation markers, optionally CD68, optionally from the cerebrospinal fluid of the subject.

In some embodiments, the reduction in levels of macrophages in the subject is by at least 70%. In some embodiments, the reduction in levels of macrophages in the subject is by at least 5%, 10%, 20%, 30%, 40%, 50%, 60%, 70%, 80%, 90%, or 95%, or any percentage within a range defined by any two of the aforementioned reductions. For example, in some embodiments, the reduction in levels of macrophages in the subject, is by at least 5%-95%, 5%-90%, 5%-75% 5%-50%, 5%-25%, 5%-10%, 5%-7%, 7%-95%, 7%-90%, 7%-75%, 7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, the reduction in levels of hyperphosphorylated Tau in the subject is by at least 80%. In some embodiments, the reduction in levels of hyperphosphorylated Tau in the subject is by at least 5%, 10%, 20%, 30%, 40%, 50%, 60%, 70%, 80%, 90%, or 95%, or any percentage within a range defined by any two of the aforementioned reductions. For example, in some embodiments, the reduction in levels of hyperphosphorylated Tau in the subject, is by at least 5%-95%, 5%-90%, 5%-75% 5%-50%, 5%-25%, 5%-10%, 5%-7%, 7%-95%, 7%-90%, 7%-75%, 7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, the reduction in levels of microhemorrhage is assessed by neuroimaging, MRI, and/or Prussian blue staining.

In some embodiments, the reduction in microhemorrhage in the subject is by at least 80%. In some embodiments, the reduction in microhemorrhage in the subject is by at least 5%, 10%, 20%, 30%, 40%, 50%, 60%, 70%, 80%, 90%, or 95%, or any percentage within a range defined by any two of the aforementioned reductions. For example, in some embodiments, the reduction in microhemorrhage in the subject, is by at least 5%-95%, 5%-90%, 5%-75% 5%-50%, 5%-25%, 5%-10%, 5%-7%, 7%-95%, 7%-90%, 7%-75%, 7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, the reduction in neuroinflammation in the subject is by at least 70%. In some embodiments, the reduction in neuroinflammation in the subject is by at least 5%, 10%, 20%, 30%, 40%, 50%, 60%, 70%, 80%, 90%, or 95%, or any percentage within a range defined by any two of the aforementioned reductions. For example, in some embodiments, the reduction in neuroinflammation in the subject, is by at least 5%-95%, 5%-90%, 5%-75% 5%-50%, 5%-25%, 5%-10%, 5%-7%, 7%-95%, 7%-90%, 7%-75%, 7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

In some embodiments, a method for inhibiting, reducing, preventing, and/or treating a disorder associated with fibrin activity or dysfunction in a subject in need thereof is disclosed. In some embodiments, amelioration of one or more symptoms associated with the disorder associated with fibrin activity or dysfunction is detected after the administering step. In some embodiments, the one or more symptoms comprise neuroinflammation and/or fibrin oligomerization. In some embodiments, administration of the anti-Gal3 antibody or binding fragment thereof to the subject reduces neuroinflammation and/or fibrin oligomerization in the subject.

In some embodiments, the reduction in fibrin oligomerization in the subject is by at least 5%, 10%, 20%, 30%, 40%, 50%, 60%, 70%, 80%, 90%, or 95%, or any percentage within a range defined by any two of the aforementioned reductions. For example, in some embodiments, the reduction in fibrin oligomerization in the subject, is by at least 5%-95%, 5%-90%, 5%-75% 5%-50%, 5%-25%,5%-10%, 5%-7%, 7%-95%, 7%-90%, 7%-75%, 7%-50%, 7%-25%, 7%-10%, 10%-95%, 10%-90%, 10%-75%, 10%-50%, 10%-25%, 25%-95%, 25%-90%, 25%-75%, 25%-50%, 50%-95%, 50%-90%, 50%-75%, 75%-95%, 75%-90%, and 90%-95%.

Exemplary standardized approaches for assessing severity of neurological issues such as stroke and TBI are described herein. It should be understood that these and other approaches have been recognized in the art for their utility in quantifying neurological capabilities in patients. Other approaches used in the common knowledge are also envisioned as alternatives to those provided herein.

National Institutes of Health Stroke Scale (NIHSS): The NIHSS provides an objective quantification of the impairment caused by a stroke. The NIHSS was designed to be a standardized and repeatable assessment of stroke patients utilized by large multi-center clinical trials. It has good interrater and test-retest reliability. The NIHSS has also been used to predict 3-month outcomes (e.g. hospital stay, mortality) of patients following a stroke.

The NIHSS is composed of 11 items, each of which rates a specific parameter of ability or function. Each item is rated from 0 to 4. A score of 0 indicates normal function, with higher scores indicating greater degree of impairment. The individual item scores are added to calculate the NIHSS total score. The maximum possible score is 42 (death), with the minimum score being a 0 (no impairment). The items scored are level of consciousness, horizontal eye movement, visual field, facial palsy, motor arm, motor leg, limb ataxia, sensory, language, speech, and extinction and inattention.

In some embodiments, evaluation by the NIHSS can be assessed 7, 35, and/or 75 days after the step of administering the anti-Gal3 antibody or binding fragment thereof.

Modified Rankin Scale: The mRS is another scale commonly used in stroke clinical trials for measuring the degree of disability or dependence in the daily activities of patients who have suffered a stroke. It consists of well-defined and easily understood grades that describe the range of global disability. The mRS exhibits a strong relationship with clinical measurements of stroke severity in addition to other disability and outcomes endpoints.

Scoring of the mRS involves a semi-structured interview between the patient and a trained facilitator. The scores range from “0” (no symptoms) to “6” (dead). It also has good interrater and test-retest reliability.

Fugl-Meyer Assessment: The FMA scale is an assessment of the sensorimotor impairment in patients who have had a stroke. It is widely used in clinical trials of stroke for the clinical assessment of motor function. The FMA score is found to have excellent consistency, responsivity and good accuracy.

Each item on the FMA is scored on a 3-point scale: 0 = cannot perform, 1 = task performed partially, 2 = task performed fully. The maximum possible score on the FMA scale is 226, which corresponds to full recovery. The 5 domains on the FMA are: motor function, sensory function, balance, range of motion, and joint pain.

Montreal Cognitive Assessment: The MoCA is a brief screening instrument designed to assess mild cognitive impairment in a variety of patient conditions. Scores on the MoCA range from 0 to 30, with lower scores indicating greater impairment. It takes approximately 10 minutes to administer the MoCA. The MoCA assesses eight cognitive domains: visuospatial ability, executive function, short-term memory recall, attention, concentration, working memory, language, and orientation to time and space. The MoCA is a particularly useful assessment for patients with impairment due to vascular conditions primarily because of the assessment of executive functions and the presence of more demanding visual construction tasks. The MoCA is one of the more commonly used cognitive assessments used in the research stroke setting.

A validation study indicated the sensitivity and specificity of the MoCA in detecting mild cognitive impairment were 90% and 97%, respectively, which was superior to that of the Mini Mental Status Examination.

The exemplary assessments described herein, as well as other standardized assessments known in the art, can be used to determine an improvement and/or amelioration of stroke or symptoms associated with the stroke in a subject. In some embodiments, detecting an amelioration of symptoms associated with the stroke after the administering step comprises detecting an improvement by the subject according to the NIHSS, mRS, FMA, or MoCA, which may be at endpoints determined by a practitioner. In some embodiments, a) the improvement by the subject according to the NIHSS comprises at least a 4 point decrease on the NIHSS; b) the improvement by the subject according to the mRS comprises at least a 1 point decrease on the mRS; c) the improvement by the subject according to the FMA comprises at least a 1 point increase on the FMA; and/or d) the improvement by the subject according to the MoCA comprises at least a 1 point increase on the MoCA, after the administering step. In some embodiments, the improvement by the subject is assessed 7, 35, and/or 75 days after the administering step.

In some embodiments, any of the endpoints depicted in Table 1 can be achieved or is the goal of any one of the appropriate methods provided herein. In some embodiments, the methods are performed to achieve any 1, 2, 3, or 4 of the objectives and/or are determined by any 1, 2, 3, or 4 of the endpoints depicted in Table 1.

In some embodiments, any one of the appropriate methods provided herein are performed to comprise assessing the subject by the NIHSS at Baseline (day 1 at the initial administration) and further assessed at Days 15, 36, 64, and 104 following the initial intervention. In some embodiments, any one of the appropriate methods provided herein comprise assessing the subject by the NIHSS at Baseline (day 1 at the initial administration) and further assessed at about Days 10-20, 31-41, 59-69, and 99-109 following the initial intervention. In some embodiments, the subject’s NIHSS is assessed at about month 1, 2, 3, 4, 5, 6, 7, 8, 9, 10, 11, or 12, following the initial administration, or at a timepoint that in a range that is defined by any two of the preceding values. For example, in some embodiments, the subject’s NIHSS is assessed at between about month 1 and 12, 1 and 8, 1 and 6, 1 and 4, 1 and 3, 3 and 12, 3 and 8, 3 and 6, 3 and 4, 4 and 12, 4 and 8, 4 and 6, 6 and 12, 6 and 8, or 8 and 12, following the initial intervention. In some embodiments, any one of the appropriate methods provided herein are performed to comprise assessing the subject by the NIHSS at Baseline (day 1 at the initial administration) and further assessed at one or more time points following the initial intervention. For example, the Subject’s NIHSS may be assessed at Baseline (day 1 at the initial administration) and further assessed at 1, 2, 3, 4, 5, 6, 7, 8, 9, 10, or more timepoints following the initial intervention, or assessed at a number of timepoints that is defined by any two of the preceding values. For example, in some embodiments, the Subject’s NIHSS is assessed at between about 1 and 10, 1 and 7, 1 and 5, 1 and 4, 1 and 3, 3 and 10, 3 and 7, 3 and 5, 5 and 10, or 5 and 7 timepoints following the initial intervention. In some embodiments, the Subject’s NIHSS is assessed more than 1 year following the initial intervention.

In some embodiments, the Subject’s NIHSS is decreased from Baseline (day 1 at the initial administration by day 36 following the initial intervention. In some embodiments, the Subject’s NIHSS is decreased from Baseline (day 1 at the initial administration by about day 31, 32, 33, 34, 35, 36, 37, 38, 39, 40, or 41, following the initial intervention, or at a time point that is in a range that is defined by any two of the preceding values. For example, in some embodiments, the Subject’s NIHSS is decreased from Baseline (day 1 at the initial administration by about days 31-41, 31 to 38, 31 to 36, 31 to 33, 33 to 41, 33 to 38, 33 to 36, 34 to 41, 34 to 37, or 37 to 41. In some embodiments, the subject’s decreased NIHSS is sustained from day 36 through day 64 following the initial intervention. In some embodiments, the subject’s decreased NIHSS is sustained from day 36 through day 104 following the initial intervention. In some embodiments, the subject’s decreased NIHSS is sustained from about day 31, 32, 33, 34, 35, 36, 37, 38, 39, 40, or 41 through about day 99, 100, 101, 102, 103, 104, 105, 105, 107, 108, or 109 following the initial intervention. In some embodiments, the subject’s decreased NIHSS is sustained from about month 1 to about month 2, 3, 4, 5, 6, 7, 8, 9, 10, 11, or 12, following the initial intervention, or for a period that is defined by any two of the preceding values. In some embodiments, the Subject’s NIHSS decreases within one month of the initial intervention. In some embodiments, the Subject’s decreased NIHSS is sustained for greater than about 3, 4, 5, 6, 7, 8, 9 10, 11, or 12 months following the initial intervention. In some embodiments, the Subject’s decreased NIHSS is sustained for greater than 12 months. For example, in some embodiments, the Subject’s decreased NIHSS is sustained for greater than about 1, 2, 3, 4, 5, 6, 7, 8, 9, 10, 11, 12, 13, 14, 15, 16, 17, 18, 19, or 20, years, or by a range that is defined by any two of the preceding values. For example, in some embodiments, the Subject’s decreased NIHSS is sustained for greater than about 1-20, 1-15, 1-10, 1-7, 1-5, 1-3, 3-20, 3-15, 3-10, 3-7, 3-5, 5-20, 5-15, 5-10, 5-7, 7-20, 7-15, 7-10, 10-20, 10-15, or 15-20 years.

In some embodiments, administration of the anti-Gal3 antibody or binding fragment thereof to the subject decreases the subject’s NIHSS by 1, 2, 3, and/or 4 points. In some embodiments, administration of the anti-Gal3 antibody or binding fragment thereof to the subject decreases the subject’s NIHSS by about 1, 2, 3, 4, 5, 6, 7, 8, 9, 10, 11, or 12, points, or by a range that is defined by any two of the preceding values. For example, in some embodiments, administration of the anti-Gal3 antibody or binding fragment thereof to the subject decreases the subject’s NIHSS by between about 1 and 12, 1 and 10, 1 and 8, 1 and 6, 1 and 4, 2 and 12, 2 and 10, 2 and 8, 2 and 6, 2 and 4, 4 and 12, 4 and 10, 4 and 8, 8 and 12, or 8 and 10 points. In some embodiments, administration of the anti-Gal3 antibody or binding fragment thereof to the subject decreases the subject’s NIHSS by about 1, 2, 3, 4, 5, 6, 7, 8, 9, 10, 15, 20, 25, 30, 40, 50, 60, 70, 75, 80, 90, 100, 200, 250, 300, 400, 500, 600, 700, 750, 800, 900, or 1000%, or by a range that is defined by any two of the preceding values. For example, in some embodiments, administration of the anti-Gal3 antibody or binding fragment thereof to the subject decreases the subject’s NIHSS by between about 1 and 1000, 1 and 750, 1 and 500, 1 and 250, 1 and 100, 1 and 75, 1 and 50, 1 and 25, 1 and 10, 1 and 5, 5 and 1000, 5 and 750, 5 and 500, 5 and 250, 5 and 100, 5 and 75, 5 and 50, 5 and 10, 10 and 1000, 10 and 750, 10 and 500, 10 and 250, 10 and 100, 10 and 75, 10 and 50, 10 and 25, 25 and 1000, 25 and 750, 25 and 500, 25 and 250, 25 and 100, 25 and 75, 25 and 50, 50 and 1000, 50 and 750, 50 and 500, 50 and 250, 50 and 100, 100 and 1000, 100 and 750, 100 and 500, 100 and 250, 250 and 1000, 250 and 750, 250 and 500, 500 and 1000, 500 and 750, or 750 and 1000%.

In some embodiments, administration of the anti-Gal3 antibody or binding fragment thereof to the subject decreases the subject’s level of consciousness score on the NIHSS by about 1, 2, 3, 4, 5, 6, or 7, points, or by a range that is defined by any two of the preceding values. For example, in some embodiments, administration of the anti-Gal3 antibody or binding fragment thereof to the subject decreases the subject’s level of consciousness score on the NIHSS by between about 1 and 7, 1 and 5, 1 and 3, 3 and 7, or 5 and 7 points.

In some embodiments, administration of the anti-Gal3 antibody or binding fragment thereof to the subject decreases the subject’s horizontal extraocular movements score on the NIHSS by about 1 or 2 points.

In some embodiments, administration of the anti-Gal3 antibody or binding fragment thereof to the subject decreases the subject’s visual fields score on the NIHSS by about 1, 2, or 3 points.

In some embodiments, administration of the anti-Gal3 antibody or binding fragment thereof to the subject decreases the subject’s facial palsy score on the NIHSS by about 1, 2, or 3 points.

In some embodiments, administration of the anti-Gal3 antibody or binding fragment thereof to the subject decreases the subject’s left arm motor drift score on the NIHSS by about 1, 2, 3, or 4 points.

In some embodiments, administration of the anti-Gal3 antibody or binding fragment thereof to the subject decreases the subject’s right arm motor drift score on the NIHSS by about 1, 2, 3, or 4 points.

In some embodiments, administration of the anti-Gal3 antibody or binding fragment thereof to the subject decreases the subject’s left leg motor drift score on the NIHSS by about 1, 2, 3, or 4 points.

In some embodiments, administration of the anti-Gal3 antibody or binding fragment thereof to the subject decreases the subject’s right leg motor drift score on the NIHSS by about 1, 2, 3, or 4 points.

In some embodiments, administration of the anti-Gal3 antibody or binding fragment thereof to the subject decreases the subject’s limb ataxia score on the NIHSS by about 1 or 2 points.

In some embodiments, administration of the anti-Gal3 antibody or binding fragment thereof to the subject decreases the subject’s language/aphasia score on the NIHSS by about 1, 2, or 3 points.

In some embodiments, administration of the anti-Gal3 antibody or binding fragment thereof to the subject decreases the subject’s dysarthria score on the NIHSS by about 1 or 2 points.

In some embodiments, administration of the anti-Gal3 antibody or binding fragment thereof to the subject decreases the subject’s extinction/inattention score on the NIHSS by about 1 or 2 points.

In some embodiments, any one of the appropriate methods provided herein are performed to comprise assessing the subject by the modified Rankin Scale (mRS) at Baseline (day 1 at the initial administration) and further assessed at Days 15, 36, 64, and 104 following the initial intervention. In some embodiments, any one of the appropriate methods provided herein are performed to comprise assessing the subject by the mRS at Baseline (day 1 at the initial administration) and further assessed at about Days 10-20, 31-41, 59-69, and 99-109 following the initial intervention. In some embodiments, the subject’s mRS is assessed at about month 1, 2, 3, 4, 5, 6, 7, 8, 9, 10, 11, or 12, following the initial administration, or at a timepoint that in a range that is defined by any two of the preceding values. For example, in some embodiments, the subject’s mRS is assessed at between about month 1 and 12, 1 and 8, 1 and 6, 1 and 4, 1 and 3, 3 and 12, 3 and 8, 3 and 6, 3 and 4, 4 and 12, 4 and 8, 4 and 6, 6 and 12, 6 and 8, or 8 and 12, following the initial intervention. In some embodiments, any one of the appropriate methods provided herein are performed to comprise assessing the subject by the mRS at Baseline (day 1 at the initial administration) and further assessed at one or more time points following the initial intervention. For example, the Subject’s mRS may be assessed at Baseline (day 1 at the initial administration) and further assessed at 1, 2, 3, 4, 5, 6, 7, 8, 9, 10, or more timepoints following the initial intervention, or assessed at a number of timepoints that is defined by any two of the preceding values. For example, in some embodiments, the Subject’s mRS is assessed at between about 1 and 10, 1 and 7, 1 and 5, 1 and 4, 1 and 3, 3 and 10, 3 and 7, 3 and 5, 5 and 10, or 5 and 7 timepoints following the initial intervention. In some embodiments, the Subject’s mRS is assessed more than 1 year following the initial intervention.

In some embodiments, the Subject’s mRS is decreased from Baseline (day 1 at the initial administration by day 36 following the initial intervention. In some embodiments, the Subject’s mRS is decreased from Baseline (day 1 at the initial administration by about day 31, 32, 33, 34, 35, 36, 37, 38, 39, 40, or 41, following the initial intervention, or at a time point that is in a range that is defined by any two of the preceding values. For example, in some embodiments, the Subject’s mRS is decreased from Baseline (day 1 at the initial administration by about days 31-41, 31 to 38, 31 to 36, 31 to 33, 33 to 41, 33 to 38, 33 to 36, 34 to 41, 34 to 37, or 37 to 41. In some embodiments, the subject’s decreased mRS is sustained from day 36 through day 64 following the initial intervention. In some embodiments, the subject’s decreased mRS is sustained from day 36 through day 104 following the initial intervention. In some embodiments, the subject’s decreased mRS is sustained from about day 31, 32, 33, 34, 35, 36, 37, 38, 39, 40, or 41 through about day 99, 100, 101, 102, 103, 104, 105, 105, 107, 108, or 109 following the initial intervention. In some embodiments, the subject’s decreased mRS is sustained from about month 1 to about month 2, 3, 4, 5, 6, 7, 8, 9, 10, 11, or 12, following the initial intervention, or for a period that is defined by any two of the preceding values. In some embodiments, the Subject’s mRS decreases within one month of the initial intervention. In some embodiments, the Subject’s decreased mRS is sustained for greater than about 3, 4, 5, 6, 7, 8, 9 10, 11, or 12 months following the initial intervention.

In some embodiments, administration of the anti-Gal3 antibody or binding fragment thereof to the subject decreases the subject’s mRS by 1 point. In some embodiments, administration of the anti-Gal3 antibody or binding fragment thereof to the subject decreases the subject’s mRS by about 1, 2, 3, 4, 5, or 6 points, or by a range that is defined by any two of the preceding values. In some embodiments, administration of the anti-Gal3 antibody or binding fragment thereof to the subject decreases the subject’s mRS by about 1, 2, 3, 4, 5, 6, 7, 8, 9, 10, 15, 20, 25, 30, 40, 50, 60, 70, 75, 80, 90, 100, 200, 250, 300, 400, 500, 600, 700, 750, 800, 900, or 1000%, or by a range that is defined by any two of the preceding values. For example, in some embodiments, administration of the anti-Gal3 antibody or binding fragment thereof to the subject decreases the subject’s mRS by between about 1 and 1000, 1 and 750, 1 and 500, 1 and 250, 1 and 100, 1 and 75, 1 and 50, 1 and 25, 1 and 10, 1 and 5, 5 and 1000, 5 and 750, 5 and 500, 5 and 250, 5 and 100, 5 and 75, 5 and 50, 5 and 10, 10 and 1000, 10 and 750, 10 and 500, 10 and 250, 10 and 100, 10 and 75, 10 and 50, 10 and 25, 25 and 1000, 25 and 750, 25 and 500, 25 and 250, 25 and 100, 25 and 75, 25 and 50, 50 and 1000, 50 and 750, 50 and 500, 50 and 250, 50 and 100, 100 and 1000, 100 and 750, 100 and 500, 100 and 250, 250 and 1000, 250 and 750, 250 and 500, 500 and 1000, 500 and 750, or 750 and 1000%.

In some embodiments, any one of the appropriate methods provided herein are performed to comprise assessing the subject by the Fugl-Meyer Assessment (FMA) at Baseline (day 1 at the initial administration) and further assessed at Days 15, 36, 64, and 104 following the initial intervention. In some embodiments, any one of the appropriate methods provided herein are performed to comprise assessing the subject by the FMA at Baseline (day 1 at the initial administration) and further assessed at about Days 10-20, 31-41, 59-69, and 99-109 following the initial intervention. In some embodiments, the subject’s mRS is assessed at about month 1, 2, 3, 4, 5, 6, 7, 8, 9, 10, 11, or 12, following the initial administration, or at a timepoint that in a range that is defined by any two of the preceding values. For example, in some embodiments, the subject’s FMA is assessed at between about month 1 and 12, 1 and 8, 1 and 6, 1 and 4, 1 and 3, 3 and 12, 3 and 8, 3 and 6, 3 and 4, 4 and 12, 4 and 8, 4 and 6, 6 and 12, 6 and 8, or 8 and 12, following the initial intervention. In some embodiments, any one of the appropriate methods provided herein are performed to comprise assessing the subject by the mRS at Baseline (day 1 at the initial administration) and further assessed at one or more time points following the initial intervention. For example, the Subject’s FMA may be assessed at Baseline (day 1 at the initial administration) and further assessed at 1, 2, 3, 4, 5, 6, 7, 8, 9, 10, or more timepoints following the initial intervention, or assessed at a number of timepoints that is defined by any two of the preceding values. For example, in some embodiments, the Subject’s FMA is assessed at between about 1 and 10, 1 and 7, 1 and 5, 1 and 4, 1 and 3, 3 and 10, 3 and 7, 3 and 5, 5 and 10, or 5 and 7 timepoints following the initial intervention. In some embodiments, the Subject’s FMA is assessed more than 1 year following the initial intervention.

In some embodiments, the Subject’s FMA is increased from Baseline (day 1 at the initial administration by day 36 following the initial intervention. In some embodiments, the Subject’s FMA is increased from Baseline (day 1 at the initial administration by about day 31, 32, 33, 34, 35, 36, 37, 38, 39, 40, or 41, following the initial intervention, or at a time point that is in a range that is defined by any two of the preceding values. For example, in some embodiments, the Subject’s FMA is increased from Baseline (day 1 at the initial administration by about days 31-41, 31 to 38, 31 to 36, 31 to 33, 33 to 41, 33 to 38, 33 to 36, 34 to 41, 34 to 37, or 37 to 41. In some embodiments, the subject’s increased FMA is sustained from day 36 through day 64 following the initial intervention. In some embodiments, the subject’s decreased FMA is sustained from day 36 through day 104 following the initial intervention. In some embodiments, the subject’s increased FMA is sustained from about day 31, 32, 33, 34, 35, 36, 37, 38, 39, 40, or 41 through about day 99, 100, 101, 102, 103, 104, 105, 105, 107, 108, or 109 following the initial intervention. In some embodiments, the subject’s increased FMA is sustained from about month 1 to about month 2, 3, 4, 5, 6, 7, 8, 9, 10, 11, or 12, following the initial intervention, or for a period that is defined by any two of the preceding values. In some embodiments, the Subject’s FMA increases within one month of the initial intervention. In some embodiments, the Subject’s increased FMA is sustained for greater than about 3, 4, 5, 6, 7, 8, 9 10, 11, or 12 months following the initial intervention.

In some embodiments, administration of the anti-Gal3 antibody or binding fragment thereof to the subject increases the subject’s FMA by about 1, 2, 3, 4, 5, 6, 7, 8, 9, 10, 11, 12, 13, 14, 15, 20, 25, 30, 40, 50, 60, 70, 80, 90, 100, 110, 120, 130, 140, 150, 160, 170, 180, 190, 200, 210, 220, 225, or 226 points, or by a range that is defined any two of the preceding values. For example, in some embodiments, administration of the anti-Gal3 antibody or binding fragment thereof to the subject increases the subject’s FMA by between about 1 and 226, 1 and 200, 1 and 150, 1 and 100, 1 and 75, 1 and 50, 1 and 25, 1 and 10, 1 and 5, 5 and 226, 5 and 200, 5 and 150, 5 and 100, 5 and 75, 5 and 50, 5 and 25, 5 and 10, 10 and 226, 10 and 200, 10 and 150, 10 and 100, 10 and 75, 10 and 50, 10 and 25, 25 and 226, 25 and 200, 25 and 150, 25 and 100, 25 and 75, 25 and 50, 50 and 226, 50 and 200, 50 and 150, 50 and 100, 50 and 75, 75 and 226, 75 and 200, 75 and 150, 75 and 100, 100 and 226, 100 and 200, 100 and 150, 150 and 226, 150 and 200, or 200 and 226, points. In some embodiments, administration of the anti-Gal3 antibody or binding fragment thereof to the subject decreases the subject’s FMA by about 1, 2, 3, 4, 5, 6, 7, 8, 9, 10, 15, 20, 25, 30, 40, 50, 60, 70, 75, 80, 90, 100, 200, 250, 300, 400, 500, 600, 700, 750, 800, 900, or 1000%, or by a range that is defined by any two of the preceding values. For example, in some embodiments, administration of the anti-Gal3 antibody or binding fragment thereof to the subject increases the subject’s FMA by between about 1 and 1000, 1 and 750, 1 and 500, 1 and 250, 1 and 100, 1 and 75, 1 and 50, 1 and 25, 1 and 10, 1 and 5, 5 and 1000, 5 and 750, 5 and 500, 5 and 250, 5 and 100, 5 and 75, 5 and 50, 5 and 10, 10 and 1000, 10 and 750, 10 and 500, 10 and 250, 10 and 100, 10 and 75, 10 and 50, 10 and 25, 25 and 1000, 25 and 750, 25 and 500, 25 and 250, 25 and 100, 25 and 75, 25 and 50, 50 and 1000, 50 and 750, 50 and 500, 50 and 250, 50 and 100, 100 and 1000, 100 and 750, 100 and 500, 100 and 250, 250 and 1000, 250 and 750, 250 and 500, 500 and 1000, 500 and 750, or 750 and 1000%.

In some embodiments, administration of the anti-Gal3 antibody or binding fragment thereof to the subject increases the subject’s motor score on the FMA by about 1, 2, 3, 4, 5, 6, 7, 8, 9, 10, 11, 12, 13, 14, 15, 20, 25, 30, 40, 50, 60, 70, 80, 90, or 100, points, or by a range that is defined any two of the preceding values. For example, in some embodiments, administration of the anti-Gal3 antibody or binding fragment thereof to the subject increases the subject’s motor score on the FMA by between about 1 and 100, 1 and 75, 1 and 50, 1 and 25, 1 and 10, 1 and 5, 5 and 100, 5 and 75, 5 and 50, 5 and 25, 5 and 10, 10 and 100, 10 and 75, 10 and 50, 10 and 25, 25 and 100, 25 and 75, 25 and 50, 50 and 100, 50 and 75, or 75 and 100, points.

In some embodiments, administration of the anti-Gal3 antibody or binding fragment thereof to the subject increases the subject’s upper extremity motor score on the FMA by about 1, 2, 3, 4, 5, 6, 7, 8, 9, 10, 11, 12, 13, 14, 15, 20, 25, 30, 40, 50, 60, 65, or 66, points, or by a range that is defined any two of the preceding values. For example, in some embodiments, administration of the anti-Gal3 antibody or binding fragment thereof to the subject increases the subject’s upper extremity motor score on the FMA by between about 1 and 66, 1 and 50, 1 and 25, 1 and 10, 1 and 5, 5 and 66, 5 and 50, 5 and 25, 5 and 10, 10 and 66, 10 and 50, 10 and 25, 25 and 66, 25 and 50, or 50 and 66, points.

In some embodiments, administration of the anti-Gal3 antibody or binding fragment thereof to the subject increases the subject’s lower extremity motor score on the FMA by about 1, 2, 3, 4, 5, 6, 7, 8, 9, 10, 11, 12, 13, 14, 15, 20, 25, 30, or 34, points, or by a range that is defined any two of the preceding values. For example, in some embodiments, administration of the anti-Gal3 antibody or binding fragment thereof to the subject increases the subject’s lower extremity motor score on the FMA by between about 1 and 34, 1 and 25, 1 and 10, 1 and 5, 5 and 34, 5 and 25, 5 and 10, 10 and 34, 10 and 25, or 25 and 34, points.

In some embodiments, administration of the anti-Gal3 antibody or binding fragment thereof to the subject increases s the subject’s sensation score on the FMA by about 1, 2, 3, 4, 5, 6, 7, 8, 9, 10, 11, 12, 13, 14, 15, 20, or 24, points, or by a range that is defined any two of the preceding values. For example, in some embodiments, administration of the anti-Gal3 antibody or binding fragment thereof to the subject increases the subject’s sensation score on the FMA by between about 1 and 24, 1 and 10, 1 and 5, 5 and 24, 5 and 10, or 10 and 24, points.

In some embodiments, administration of the anti-Gal3 antibody or binding fragment thereof to the subject increases the subject’s light touch sensation score on the FMA by about 1, 2, 3, 4, 5, 6, 7, or 8, points, or by a range that is defined any two of the preceding values. For example, in some embodiments, administration of the anti-Gal3 antibody or binding fragment thereof to the subject increases the subject’s light touch sensation score on the FMA by between about 1 and 8, 1 and 6, 1 and 4, 4 and 8, or 6 and 8, points.

In some embodiments, administration of the anti-Gal3 antibody or binding fragment thereof to the subject increases the subject’s position sensation score on the FMA by about 1, 2, 3, 4, 5, 6, 7, 8, 9, 10, 11, 12, 13, 14, 15, or 16, points, or by a range that is defined any two of the preceding values. For example, in some embodiments, administration of the anti-Gal3 antibody or binding fragment thereof to the subject d increases the subject’s position sensation score on the FMA by between about 1 and 16, 1 and 10, 1 and 5, 5 and 16, 5 and 10, or 10 and 16, points.

In some embodiments, administration of the anti-Gal3 antibody or binding fragment thereof to the subject increases the subject’s balance score on the FMA by about 1, 2, 3, 4, 5, 6, 7, 8, 9, 10, 11, 12, 13, or 14, points, or by a range that is defined any two of the preceding values. For example, in some embodiments, administration of the anti-Gal3 antibody or binding fragment thereof to the subject increases the subject’s balance score on the FMA by between about 1 and 14, 1 and 10, 1 and 5, 5 and 14, 5 and 10, or 10 and 14, points.

In some embodiments, administration of the anti-Gal3 antibody or binding fragment thereof to the subject increases the subject’s sitting balance score on the FMA by about 1, 2, 3, 4, 5, or 6, points, or by a range that is defined any two of the preceding values. For example, in some embodiments, administration of the anti-Gal3 antibody or binding fragment thereof to the subject increases the subject’s balance score on the FMA by between about 1 and 6, 1 and 3, or 3 and 6, points.

In some embodiments, administration of the anti-Gal3 antibody or binding fragment thereof to the subject increases the subject’s standing balance score on the FMA by about 1, 2, 3, 4, 5, 6, 7, or 8, points, or by a range that is defined any two of the preceding values. For example, in some embodiments, administration of the anti-Gal3 antibody or binding fragment thereof to the subject increases the subject’s balance score on the FMA by between about 1 and 8, 1 and 6, 1 and 4, 4 and 8, 4 and 6, or 6 and 8, points.

In some embodiments, administration of the anti-Gal3 antibody or binding fragment thereof to the subject increases the subject’s joint range of motion score on the FMA by about 1, 2, 3, 4, 5, 6, 7, 8, 9, 10, 11, 12, 13, 14, 15, 20, 25, 30, 40, or 44, points, or by a range that is defined any two of the preceding values. For example, in some embodiments, administration of the anti-Gal3 antibody or binding fragment thereof to the subject increases the subject’s joint range of motion score on the FMA by between about 1 and 44, 1 and 30, 1 and 25, 1 and 10, 1 and 5, 5 and 44, 5 and 30, 5 and 25, 5 and 10, 10 and 44, 10 and 30, 10 and 25, 25 and 44, 25 and 30, or 30 and 44, points.

In some embodiments, administration of the anti-Gal3 antibody or binding fragment thereof to the subject increases the subject’s joint pain score on the FMA by about 1, 2, 3, 4, 5, 6, 7, 8, 9, 10, 11, 12, 13, 14, 15, 20, 25, 30, 40, or 44, points, or by a range that is defined any two of the preceding values. For example, in some embodiments, administration of the anti-Gal3 antibody or binding fragment thereof to the subject increases the subj ect’s joint pain score on the FMA by between about 1 and 44, 1 and 30, 1 and 25, 1 and 10, 1 and 5, 5 and 44, 5 and 30, 5 and 25, 5 and 10, 10 and 44, 10 and 30, 10 and 25, 25 and 44, 25 and 30, or 30 and 44, points.

In some embodiments, any one of the appropriate methods provided herein are performed to comprise assessing the subject by the Montreal Cognitive Assessment (MoCA) at Baseline (day 1 at the initial administration) and further assessed at Days 15, 36, 64, and 104 following the initial intervention. In some embodiments, any one of the appropriate methods provided herein are performed to comprise assessing the subject by the MoCA at Baseline (day 1 at the initial administration) and further assessed at about Days 10-20, 31-41, 59-69, and 99-109 following the initial intervention. In some embodiments, the subject’s MoCA is assessed at about month 1, 2, 3, 4, 5, 6, 7, 8, 9, 10, 11, or 12, following the initial administration, or at a timepoint that in a range that is defined by any two of the preceding values. For example, in some embodiments, the subject’s MoCA is assessed at between about month 1 and 12, 1 and 8, 1 and 6, 1 and 4, 1 and 3, 3 and 12, 3 and 8, 3 and 6, 3 and 4, 4 and 12, 4 and 8, 4 and 6, 6 and 12, 6 and 8, or 8 and 12, following the initial intervention. In some embodiments, any one of the appropriate methods provided herein are performed to comprise assessing the subject by the MoCA at Baseline (day 1 at the initial administration) and further assessed at one or more time points following the initial intervention. For example, the Subject’s MoCA may be assessed at Baseline (day 1 at the initial administration) and further assessed at 1, 2, 3, 4, 5, 6, 7, 8, 9, 10, or more timepoints following the initial intervention, or assessed at a number of timepoints that is defined by any two of the preceding values. For example, in some embodiments, the Subject’s MoCA is assessed at between about 1 and 10, 1 and 7, 1 and 5, 1 and 4, 1 and 3, 3 and 10, 3 and 7, 3 and 5, 5 and 10, or 5 and 7 timepoints following the initial intervention. In some embodiments, the Subject’s MoCA is assessed more than 1 year following the initial intervention.

In some embodiments, the Subject’s MoCA is increased from Baseline (day 1 at the initial administration by day 36 following the initial intervention. In some embodiments, the Subject’s MoCA is increased from Baseline (day 1 at the initial administration by about day 31, 32, 33, 34, 35, 36, 37, 38, 39, 40, or 41, following the initial intervention, or at a time point that is in a range that is defined by any two of the preceding values. For example, in some embodiments, the Subject’s MoCA is increased from Baseline (day 1 at the initial administration by about days 31-41, 31 to 38, 31 to 36, 31 to 33, 33 to 41, 33 to 38, 33 to 36, 34 to 41, 34 to 37, or 37 to 41. In some embodiments, the subject’s increased MoCA is sustained from day 36 through day 64 following the initial intervention. In some embodiments, the subject’s increased MoCA is sustained from day 36 through day 104 following the initial intervention. In some embodiments, the subject’s increased MoCA is sustained from about day 31, 32, 33, 34, 35, 36, 37, 38, 39, 40, or 41 through about day 99, 100, 101, 102, 103, 104, 105, 105, 107, 108, or 109 following the initial intervention. In some embodiments, the subject’s increased MoCA is sustained from about month 1 to about month 2, 3, 4, 5, 6, 7, 8, 9, 10, 11, or 12, following the initial intervention, or for a period that is defined by any two of the preceding values. In some embodiments, the Subject’s MoCA increases within one month of the initial intervention. In some embodiments, the Subject’s increased MoCA is sustained for greater than about 3, 4, 5, 6, 7, 8, 9 10, 11, or 12 months following the initial intervention.

In some embodiments, administration of the anti-Gal3 antibody or binding fragment thereof to the subject increases the subject’s MoCA by about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, points, or by a range that is defined by any two of the preceding values. For example, in some embodiments, administration of the anti-Gal3 antibody or binding fragment thereof to the subject increases the subject’s MoCA by between about 1 and 30, 1 and 20, 1 and 15, 1 and 10, 1 and 5, 5 and 30, 5 and 20, 5 and 15, 5 and 10, 10 and 30, 10 and 15, 15 and 30, 15 and 20, or 20 and 30 points. In some embodiments, administration of the anti-Gal3 antibody or binding fragment thereof to the subject increases the subject’s MoCA by about 1, 2, 3, 4, 5, 6, 7, 8, 9, 10, 15, 20, 25, 30, 40, 50, 60, 70, 75, 80, 90, 100, 200, 250, 300, 400, 500, 600, 700, 750, 800, 900, or 1000%, or by a range that is defined by any two of the preceding values. For example, in some embodiments, administration of the anti-Gal3 antibody or binding fragment thereof to the subject increases the subject’s MoCA by between about 1 and 1000, 1 and 750, 1 and 500, 1 and 250, 1 and 100, 1 and 75, 1 and 50, 1 and 25, 1 and 10, 1 and 5, 5 and 1000, 5 and 750, 5 and 500, 5 and 250, 5 and 100, 5 and 75, 5 and 50, 5 and 10, 10 and 1000, 10 and 750, 10 and 500, 10 and 250, 10 and 100, 10 and 75, 10 and 50, 10 and 25, 25 and 1000, 25 and 750, 25 and 500, 25 and 250, 25 and 100, 25 and 75, 25 and 50, 50 and 1000, 50 and 750, 50 and 500, 50 and 250, 50 and 100, 100 and 1000, 100 and 750, 100 and 500, 100 and 250, 250 and 1000, 250 and 750, 250 and 500, 500 and 1000, 500 and 750, or 750 and 1000%.

In some embodiments, administration of the anti-Gal3 antibody or binding fragment thereof to the subject prevents the stroke (which also may involve reducing the severity of a stroke that occurs). In some embodiments, administration of the anti-Gal3 antibody or binding fragment thereof prevents loss of locomotor dysfunction, prevents incidence of microhemorrhage, prevents elevation of levels of activated microglia, prevents elevation of levels of activated astrocytes, prevents elevation of circulating proinflammatory immune cells, prevents elevation of circulating proinflammatory cytokines, or any combination thereof, in the subject.

In some embodiments, administration of the anti-Gal3 antibody or binding fragment thereof to the subject reduces the chances of the stroke or reduces the chances of a severe stroke by at least 5%, 10%, 20%, 30%, 40%, 50%, 60%, 70%, 80%, 90%, 95%, 96%, 97%, 98%, 99%, or any percentage within a range defined by any two of the aforementioned reductions. In some embodiments, administration of the anti-Gal3 antibody or binding fragment thereof to the subject reduces the chances of the stroke or reduces the chances of a severe stroke by at least 50%, 60%, 70%, 80%, 90%, 95%, 96%, 97%, 98%, 99% or higher or any percentage within a range defined by any two of the aforementioned reductions.

In some embodiments, administration of the anti-Gal3 antibody or binding fragment thereof to the subject treats the stroke. In some embodiments, administration of the anti-Gal3 antibody or binding fragment thereof reduces locomotor dysfunction, reduces microhemorrhage, reduces levels of activated microglia, reduces levels of activated astrocytes, reduces levels of circulating proinflammatory immune cells, reduces levels of circulating proinflammatory cytokines, or any combination thereof, in the subject.

In some embodiments, the reduction in locomotor dysfunction, reduction in microhemorrhage, reduction in levels of activated microglia, reduction in levels of activated astrocytes, reduction in levels of circulating proinflammatory cytokines, or any combination thereof, in the subject, is by at least 5%, 10%, 20%, 30%, 40%, 50%, 60%, 70%, 80%, 90%, 95%, 96%, 97%, 98%, 99% or higher or any percentage within a range defined by any two of the aforementioned reductions. In some embodiments, the reduction in locomotor dysfunction in the subject is by at least 50%. In some embodiments, the reduction in locomotor dysfunction is assessed by the Fugl-Meyer Assessment (FMA), Motor Assessment Scale (MAS), and/or the Chedoke-McMaster Stroke Assessment (CMSA). In some embodiments, the reduction in microhemorrhage in the subject is by at least 50%. In some embodiments, the reduction in microhemorrhage is assessed by neuroimaging, MRI, and/or Prussian blue staining. In some embodiments, the reduction in levels of activated microglia in the subject is by at least 20%. In some embodiments, the reduction in levels of activated microglia is assessed by detection of microglia activation markers. In some embodiments, the microglia activation markers include but are not limited to ionized calcium-binding adaptor molecule 1 (Iba1), monocyte chemoattractant protein-1 (MCP-1), chitinase-3-like protein 1 (YKL-40), and/or soluble CD14 (sCD14). In some embodiments, the microglia activation markers are detected in the cerebrospinal fluid of the subject. In some embodiments, the reduction in levels of activated astrocytes in the subject is by at least 30%. In some embodiments, the reduction in levels of activated astrocytes is assessed by detection of astrocyte activation markers. In some embodiments, the astrocyte activation markers include but are not limited to GFAP. In some embodiments, the astrocyte activation markers are detected in the cerebrospinal fluid of the subject. In some embodiments, the proinflammatory immune cells comprise NK cells, monocytes, and/or lymphocytes. In some embodiments, the proinflammatory cytokines include but are not limited to IL-6 and/or IL1β.

In some embodiments, the anti-Gal3 antibody or binding fragment thereof is administered with one or more additional therapeutic compositions, such as those for the treatment of stroke. In some embodiments, the stroke is ischemic stroke, thrombotic stroke, embolic stroke, or transient ischemic attack, and the one or more additional therapeutic compositions comprise thrombolytics, tissue plasminogen activator (tPA), alteplase, reteplase, tenecteplase, desmoteplase, anticoagulants, ACE inhibitors, antihypertensives, nicardipine, or any combination thereof. In some embodiments, the stroke is hemorrhagic stroke, intracerebral hemorrhage, or subarachnoid hemorrhage, and the one or more additional therapeutic compositions comprise antihypertensives, or nicardipine, or any combination thereof. The administration of the anti-Gal3 antibody or binding fragment thereof may also be accompanied with surgery.

In addition to the methods for selecting the subject as having the stroke or at risk of contracting the stroke, and/or detecting an amelioration of symptoms associated with the stroke, alternative approaches can also be used. These approaches may include the Action Research Arm Test, Barthel Index, Berg Balance Scale, Box and Block Test, Chedoke McMaster Stroke Assessment Scale, Chedoke Arm and Hand Activity Inventory, Clinical Outcome Variables Scale, Functional Ambulation Categories, Functional Independence Measure, Frenchay Activities Index, Motor Assessment Scale, Motricity Test, Nine-hole Peg Test, Rankin Handicap Scale, Rivermead Mobility Scale, Rivermead Motor Assessment, Six Minute Walk Test, Timed Up and Go, and/or Wolf Motor Function Test. Other approaches may involve analysis of inflammatory components from blood or cerebrospinal fluid samples from the patient. These inflammatory components may include immune cells, such as leukocytes, NK cells, monocytes, lymphocytes, and the like, or pro-inflammatory cytokines. The use of any one or more of these approaches to assess the incidence or risk of stroke may be dependent on the discretion of a trained medical professional.

In some embodiments, the anti-Gal3 antibody or binding fragment thereof is or comprises any one or more of the sequences illustrated in FIGS. 3A-C, 4A-C, 5, 6, 7, 8, or 9, includes any 1, 2, 3, 4, 5, or 6 of the CDRs in SEQ ID NOs: 27-538, 801-865, and/or includes one or both of the VH/VL in SEQ ID NOs: 297-447, 803-865, 926-929. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 27, 71, 112, 170, 221, 248. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 31, 72, 113, 171, 222, 249. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 30, 86, 130, 189, 230, 263. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 116, 174, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 32, 84, 119, 186, 228, 255. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 37, 77, 118, 178, 229, 256. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 126, 187, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 66, 108, 164, 215, 225, 291.

Also disclosed herein are methods for inhibiting, reducing, preventing, and/or treating traumatic brain injury (TBI) in a subject in need thereof. The methods comprise administering an anti-Gal3 antibody or binding fragment thereof to the subject, thereby inhibiting, reducing, preventing, and/or treating the TBI.

In some embodiments, the methods further comprise selecting the subject as having the TBI or at risk of contracting the TBI prior to the administering step. In some embodiments, selecting the subject as having the TBI comprises assessing the subject for level of consciousness, memory loss, and/or the Glasgow Coma Scale, neuroimaging (such as CT or MRI) and/or detecting TBI biomarkers (including but not limited to GFAP and/or ubiquitin carboxy-terminal hydrolase L1 (UCH-L1). In some embodiments, the methods further comprise detecting an amelioration of symptoms associated with the TBI after the administering step. In some embodiments, the TBI is associated with a concussion, edema, diffuse axonal injury, spinal cord injury, coma, neuroinflammation, microhemorrhage, astrocytosis, activated microglia, and/or hematoma. In some embodiments, any of the methods disclosed herein involving an anti-Gal3 antibody or binding fragment can be performed with an antigen binding molecule that binds to Gal3.

In some embodiments, administration of the anti-Gal3 antibody or binding fragment thereof to the subject prevents the TBI, concussion, edema, diffuse axonal injury, spinal cord injury, coma, neuroinflammation, microhemorrhage, astrocytosis, activated microglia, and/or hematoma. This may also involve reducing the severity of the TBI, concussion, edema, diffuse axonal injury, spinal cord injury, coma, neuroinflammation, microhemorrhage, astrocytosis, activated microglia, and/or hematoma. In some embodiments, administration of the anti-Gal3 antibody or binding fragment thereof prevents elevation of levels of activated microglia, prevents elevation of levels of activated astrocytes, prevents elevation of levels of macrophages, prevents elevation of levels of hyperphosphorylated Tau, prevents incidence of microhemorrhage, prevents neuroinflammation, prevents elevation of levels of circulating proinflammatory cytokines, or any combination thereof, in the subject.

In some embodiments, administration of the anti-Gal3 antibody or binding fragment thereof to the subject reduces the chance of the TBI, concussion, edema, diffuse axonal injury, spinal cord injury, coma, neuroinflammation, microhemorrhage, astrocytosis, activated microglia, and/or hematoma or reduces the chances of a severe form of said conditions by at least 5%, 10%, 20%, 30%, 40%, 50%, 60%, 70%, 80%, 90%, or 95%, or any percentage within a range defined by any two of the aforementioned reductions. In some embodiments, administration of the anti-Gal3 antibody or binding fragment thereof to the subject reduces the chance of the TBI, concussion, edema, diffuse axonal injury, spinal cord injury, coma, neuroinflammation, microhemorrhage, astrocytosis, activated microglia, and/or hematoma or reduces the chances of a severe form of said conditions by at least 50%, 60%, 70%, 80%, 90%, 95%, 96%, 97%, 98%, 99% or higher, or any percentage within a range defined by any two of the aforementioned reductions.

In some embodiments, administration of the anti-Gal3 antibody or binding fragment thereof to the subject treats the TBI, concussion, edema, diffuse axonal injury, spinal cord injury, coma, neuroinflammation, microhemorrhage, astrocytosis, activated microglia, and/or hematoma. In some embodiments, administration of the anti-Gal3 antibody or binding fragment thereof improves the level of consciousness, memory loss, and/or the Glasgow Coma Scale of the patient; and/or reduces levels of activated microglia, reduces levels of activated astrocytes, reduces levels of macrophages, reduces levels of hyperphosphorylated Tau, reduces microhemorrhage, reduces neuroinflammation, reduces levels of circulating proinflammatory cytokines or any combination thereof, in the subject.

In some embodiments, the reduction in levels of activated microglia, reduction in levels of activated astrocytes, reduction in levels of macrophages, reduction in levels of hyperphosphorylated Tau, reduction in microhemorrhage, reduction in neuroinflammation, reduction in levels of circulating proinflammatory cytokines, or any combination thereof, in the subject, is by at least 5%, 10%, 20%, 30%, 40%, 50%, 60%, 70%, 80%, 90%, 95%, 96%, 97%, 98%, 99% or higher, or any percentage within a range defined by any two of the aforementioned reductions. In some embodiments, the reduction in levels of activated microglia in the subject is by at least 70%. In some embodiments, the reduction in levels of activated microglia is assessed by detection of microglia activation markers. In some embodiments, the microglia activation markers include but are not limited to ionized calcium-binding adaptor molecule 1 (Ibal), monocyte chemoattractant protein-1 (MCP-1), chitinase-3-like protein 1 (YKL-40), and/or soluble CD14 (sCD14). In some embodiments, the microglia activation markers are detected in the cerebrospinal fluid of the subject. In some embodiments, the reduction in levels of activated astrocytes in the subject is by at least 90%. In some embodiments, the reduction in levels of activated astrocytes is assessed by detection of astrocyte activation markers. In some embodiments, the astrocyte activation markers include but are not limited to GFAP. In some embodiments, the astrocyte activation markers are detected in the cerebrospinal fluid of the subject. In some embodiments, the reduction in levels of macrophages in the subject is by at least 70%. In some embodiments, the reduction in levels of macrophages is assessed by detection of macrophage activation markers. In some embodiments, the macrophage activation markers include but are not limited to CD68. In some embodiments, the macrophage activation markers are detected in the cerebrospinal fluid of the subject. In some embodiments, the reduction in levels of hyperphosphorylated Tau in the subject is by at least 80%. In some embodiments, the reduction in levels of microhemorrhage in the subject is by at least 80%. In some embodiments, the reduction in levels of microhemorrhage is assessed by neuroimaging, MRI, and/or Prussian blue staining. In some embodiments, the reduction in neuroinflammation in the subject is by at least 70%. In some embodiments, the proinflammatory cytokines comprise IL-6, IL-10, and/or TNF-α.

In some embodiments, the anti-Gal3 antibody or binding fragment thereof is administered with one or more additional therapeutic compositions, such as those for treating TBI. In some embodiments, the one or more additional therapeutic compositions comprise levodopa, bromocriptine, NMDA receptor antagonists, amantadine, memantine, cholinesterase inhibitors, tacrine, rivastigmine, galantamine, donepezil, or any combination thereof. The administration of the anti-Gal3 antibody or binding fragment thereof may also be accompanied with surgery.

In some embodiments, the anti-Gal3 antibody or binding fragment thereof is or comprises any one or more of the sequences illustrated in FIGS. 3A-C, 4A-C, 5, 6, 7, 8, or 9, includes any 1, 2, 3, 4, 5, or 6 of the CDRs in SEQ ID NOs: 27-538, 801-865, and/or includes one or both of the VH/VL in SEQ ID NOs: 297-447, 803-865, 926-929. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 27, 71, 112, 170, 221, 248. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 31, 72, 113, 171, 222, 249. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 30, 86, 130, 189, 230, 263. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 116, 174, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 32, 84, 119, 186, 228, 255. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 37, 77, 118, 178, 229, 256. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 126, 187, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 66, 108, 164, 215, 225, 291.

Also disclosed herein are methods for inhibiting, reducing, preventing, and/or treating a disorder associated with fibrin activity or dysfunction in a subject in need thereof. The methods comprise administering an anti-Gal3 antibody or binding fragment thereof to the subject, thereby inhibiting, reducing, preventing, and/or treating the disorder associated with fibrin activity or dysfunction.

In some embodiments, the disorder associated with fibrin activity or dysfunction comprises atherosclerosis, thrombosis, thromboembolism, carotid artery disease, coronary artery disease, peripheral artery disease, myocardial infarction, heart failure, heart attack, hypertension, chronic kidney disease, coagulopathy, or thrombocytopathy. In some embodiments, the disorder associated with fibrin activity or dysfunction is atherosclerosis. In some embodiments, the disorder associated with fibrin activity or dysfunction is thrombosis. In some embodiments, the disorder associated with fibrin activity or dysfunction is thromboembolism. In some embodiments, the disorder associated with fibrin activity or dysfunction is carotid artery disease. In some embodiments, the disorder associated with fibrin activity or dysfunction is coronary artery disease. In some embodiments, the disorder associated with fibrin activity or dysfunction is peripheral artery disease. In some embodiments, the disorder associated with fibrin activity or dysfunction is myocardial infarction. In some embodiments, the disorder associated with fibrin activity or dysfunction is heart failure. In some embodiments, the disorder associated with fibrin activity or dysfunction is chronic kidney disease. In some embodiments, the disorder associated with fibrin activity or dysfunction is coagulopathy. In some embodiments, the disorder associated with fibrin activity or dysfunction is thrombocytopathy. In some embodiments, the methods further comprise selecting the subject as having the disorder associated with fibrin activity or dysfunction or at risk of contracting the disorder associated with fibrin activity or dysfunction prior to the administering step. In some embodiments, selecting the subject as having the disorder associated with fibrin activity or dysfunction comprises detecting thrombotic biomarkers. In some embodiments, the thrombotic biomarkers include but are not limited to fibrinogen, C-reactive protein (CRP), and/or plasminogen activator inhibitor-1 (PAI-1). In some embodiments, the methods further comprise detecting an amelioration of symptoms associated with the disorder associated with fibrin activity or dysfunction after the administering step. In some embodiments, any of the methods disclosed herein involving an anti-Gal3 antibody or binding fragment can be performed with an antigen binding molecule that binds to Gal3.

In some embodiments, administration of the anti-Gal3 antibody or binding fragment thereof to the subject prevents neuroinflammation and/or fibrin oligomerization, in the subject. In some embodiments, administration of the anti-Gal3 antibody or binding fragment thereof to the subject reduces neuroinflammation and/or fibrin oligomerization in the subject.

In some embodiments, the reduction in neuroinflammation and/or the reduction in fibrin oligomerization, in the subject, is by at least 5%, 10%, 20%, 30%, 40%, 50%, 60%, 70%, 80%, 90%, or 95%, or any percentage within a range defined by any two of the aforementioned reductions.

In some embodiments, the anti-Gal3 antibody or binding fragment thereof is administered with one or more additional therapeutic compositions, such as those used for treating a disorder associated with fibrin activity or dysfunction, atherosclerosis, thrombosis, thromboembolism, carotid artery disease, coronary artery disease, peripheral artery disease, myocardial infarction, heart failure, heart attack, hypertension, chronic kidney disease, coagulopathy, or thrombocytopathy. In some embodiments, the one or more additional therapeutic compositions comprises anti-clotting agents, aspirin, statins, antihypertensives, diuretics, calcium channel blockers, ACE inhibitors, angiotensin II receptor antagonists, adrenergic receptor antagonists, vasodilators, renin inhibitors, aldosterone receptor antagonists, endothelium receptor blockers, or any combination thereof. The administration of the anti-Gal3 antibody or binding fragment thereof may also be accompanied with surgery.

In addition to the indications provided herein, the anti-Gal3 antibodies and binding fragments thereof are also envisioned to be effective for related indications, such as those related to inflammation where a subject would benefit from a reduction in proinflammatory cytokines. In some embodiments are provided methods for inhibiting, reducing, preventing, and/or treating a disorder associated with inflammation and elevation of proinflammatory cytokines in a subject in need thereof. The methods comprise administering an anti-Gal3 antibody or binding fragment thereof to the subject, thereby treating the disorder associated with inflammation. In some embodiments, the administration reduces proinflammatory cytokines, such as circulating cytokines, in the subject.

In some embodiments, the anti-Gal3 antibody or binding fragment thereof is or comprises any one or more of the sequences illustrated in FIGS. 3A-C, 4A-C, 5, 6, 7, 8, or 9, includes any 1, 2, 3, 4, 5, or 6 of the CDRs in SEQ ID NOs: 27-538, 801-865, and/or includes one or both of the VH/VL in SEQ ID NOs: 297-447, 803-865, 926-929. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 27, 71, 112, 170, 221, 248. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 31, 72, 113, 171, 222, 249. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 30, 86, 130, 189, 230, 263. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 116, 174, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 32, 84, 119, 186, 228, 255. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 37, 77, 118, 178, 229, 256. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 126, 187, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 66, 108, 164, 215, 225, 291.

As applied to any of the methods comprising administering an anti-Gal3 antibody or binding fragment thereof to a subject disclosed herein, in some embodiments, administering an anti-Gal3 antibody or binding fragment thereof to the subject comprises administration of one or more unit doses of the anti-Gal3 antibody or binding fragment thereof, optionally 1, 2, 3, 4, 5, 6, 7, 8, 9, or 10 unit doses. In some embodiments, 5, 6, 7, 8, 9, or 10 unit doses are administered. In some embodiments, 5 unit doses are administered. In some embodiments, 8 unit doses are administered. In some embodiments, the one or more unit doses comprise 50, 60, 70, 80, 90, 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500 or 5000 mg of the anti-Gal3 antibody or binding fragment thereof per unit dose, or any amount per unit dose within a range defined by any two of the aforementioned amounts per unit dose. In some embodiments, the one or more unit doses are between 70 to 5000 mg of the anti-Gal3 antibody or binding fragment thereof per unit dose. In some embodiments, the one or more unit doses comprise 800, 900, 1000, 1100, 1200, 1300, 1400, or 1500 mg of the anti-Gal3 antibody or binding fragment thereof per unit dose, or any amount per unit dose within a range defined by any two of the aforementioned amounts per unit dose. In some embodiments, the one or more unit doses comprise 1000 mg or about 1000 mg of the anti-Gal3 antibody or binding fragment thereof per unit dose. In some embodiments, the one or more unit doses comprise 1, 1.1, 1.2, 1.3, 1.4, 1.5, 1.6, 1.7, 1.8, 1.9, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, or 150 mg/kg weight of the subject, or any amount per unit dose within a range defined by any two of the aforementioned amounts per unit dose. In some embodiments, the one or more unit doses comprise 5, 6, 7, 8, 9, 10, 11, 12, 13, 14, or 15 mg/kg weight of the subject, or any amount per unit dose within a range defined by any two of the aforementioned amounts per unit dose. In some embodiments, the one or more unit doses comprise 10 mg/kg or about 10 mg/kg weight of the subject. In some embodiments, a combined total of 1000, 2000, 3000, 4000, 5000, 6000, 7000, 8000, 9000, or 10000 mg of anti-Gal3 antibody or binding fragment thereof is administered to the subject. In some embodiments, a combined total of 4000, 4500, 5000, 5500, or 6000 mg of anti-Gal3 antibody or binding fragment thereof is administered to the subject. In some embodiments, a combined total of 5000 mg of anti-Gal3 antibody or binding fragment thereof is administered to the subject. In some embodiments, the one or more unit doses are administered every 1, 2, 3, 4, 5, 6, or 7 days, or 1, 2, 3, or 4 weeks, or any interval within a range defined by any two of the aforementioned intervals. In some embodiments, the one or more unit doses are administered every 7 days or about 7 days. In some embodiments, the one or more unit doses comprise 1000 mg or about 1000 mg of the anti-Gal3 antibody or binding fragment thereof per unit dose and the unit doses are administered every 7 days or about 7 days. In some embodiments, 5 unit doses comprise 1000 mg or about 1000 mg of the anti-Gal3 antibody or binding fragment thereof per unit dose and the unit doses are administered every 7 days or about 7 days. In some embodiments, the one or more unit doses are administered over the course of 10, 20, 30, 40, 50, 60, 70, 80, 90, or 100 minutes, or any amount of time within a range defined by any two of the aforementioned durations. In some embodiments, the one or more unit doses are administered over the course of 60 minutes or about 60 minutes. In some embodiments, the anti-Gal3 antibody or binding fragment thereof is administered enterally, orally, intranasally, parenterally, intracranially, subcutaneously, intramuscularly, intradermally, or intravenously, or any combination thereof, optionally intravenously.

In some embodiments, the anti-Gal3 antibody or binding fragment thereof is or comprises any one or more of the sequences illustrated in FIGS. 3A-C, 4A-C, 5, 6, 7, 8, or 9, includes any 1, 2, 3, 4, 5, or 6 of the CDRs in SEQ ID NOs: 27-538, 801-865, and/or includes one or both of the VH/VL in SEQ ID NOs: 297-447, 803-865, 926-929. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 27, 71, 112, 170, 221, 248. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 31, 72, 113, 171, 222, 249. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 30, 86, 130, 189, 230, 263. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 116, 174, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 32, 84, 119, 186, 228, 255. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 37, 77, 118, 178, 229, 256. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 34, 74, 126, 187, 225, 252. In some embodiments, the anti-Gal3 antibody or binding fragment thereof includes 1, 2, 3, 4, 5, or 6 of the V_{H}-CDR1, V_{H}-CDR2, V_{H}-CDR3, V_{L}-CDR1, V_{L}-CDR2, and/or V_{L}-CDR3 of SEQ ID NOs: 66, 108, 164, 215, 225, 291.

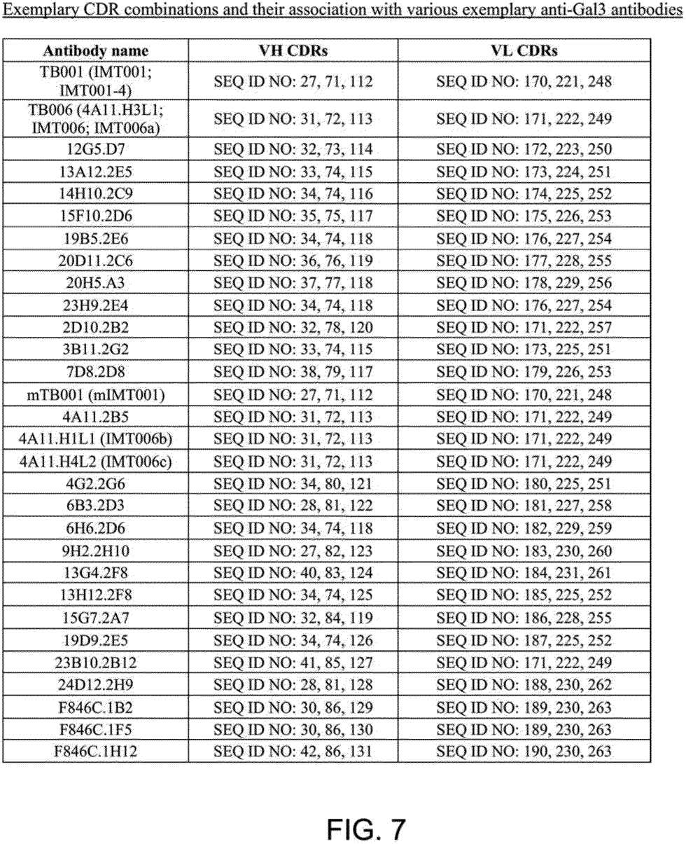

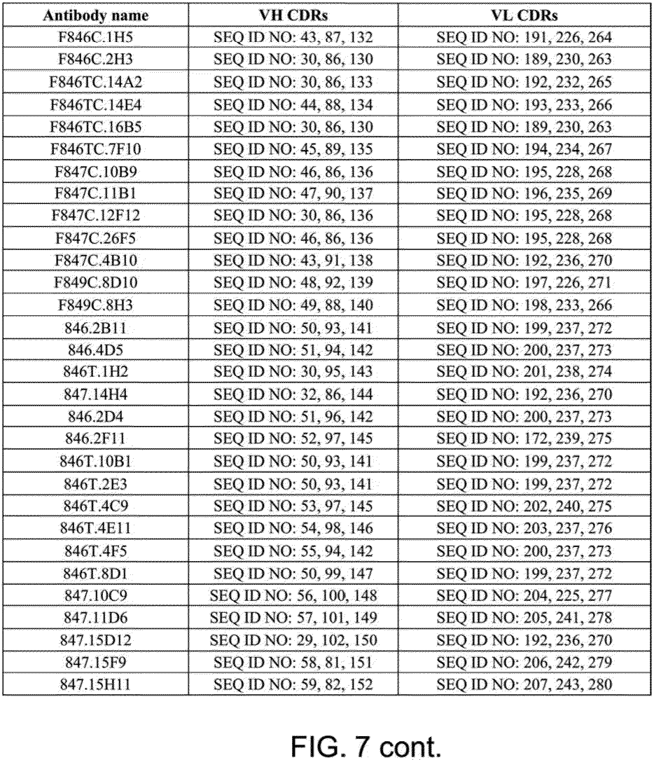

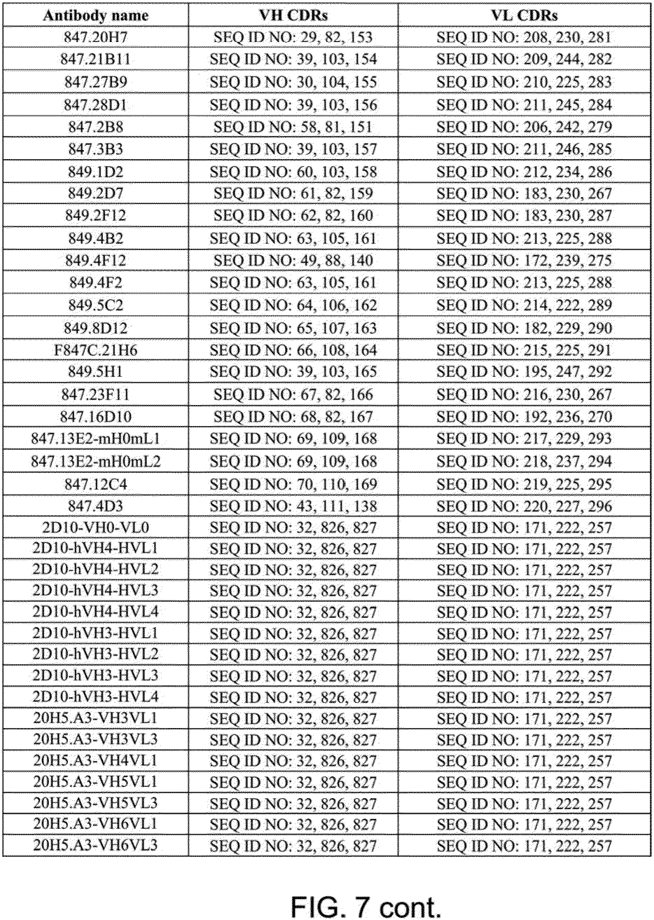

As applied to any of the methods comprising administering an anti-Gal3 antibody or binding fragment thereof, in some embodiments, the anti-Gal3 antibody or binding fragment thereof selectively binds to an N-terminal domain of Gal3. In some embodiments, the anti-Gal3 antibody or binding fragment thereof binds to Peptide 1 (SEQ ID NO: 3), Peptide 6 (SEQ ID NO: 8), or Peptide 7 (SEQ ID NO:9), or any combination thereof. In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 7. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835. In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865. In some embodiments, any of the methods disclosed herein involving an anti-Gal3 antibody or binding fragment can be performed with an antigen binding molecule that binds to Gal3.

As applied to any of the methods disclosed herein comprising administering an anti-Gal3 antibody or binding fragment thereof, in some embodiments, the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof comprises one or more of: TB001, TB006, F846C.1F5, F846TC.16B5, 14H10.2C9, 15G7.2A7, 20H5.A3, 19D9.2E5, 19B5.2E6, or F847C.21H6. In some embodiments, the anti-Gal3 antibody or binding fragment thereof comprises TB001. In some embodiments, the anti-Gal3 antibody or binding fragment thereof comprises TB006. In some embodiments, the anti-Gal3 antibody or binding fragment thereof comprises F846C.1F5. In some embodiments, the anti-Gal3 antibody or binding fragment thereof comprises F846TC.16B5. In some embodiments, the anti-Gal3 antibody or binding fragment thereof comprises 14H10.2C9. In some embodiments, the anti-Gal3 antibody or binding fragment thereof comprises 15G7.2A7. In some embodiments, the anti-Gal3 antibody or binding fragment thereof comprises 20H5.A3. In some embodiments, the anti-Gal3 antibody or binding fragment thereof comprises 19D9.2E5. In some embodiments, the anti-Gal3 antibody or binding fragment thereof comprises 19B5.2E6. In some embodiments, the anti-Gal3 antibody or binding fragment thereof comprises F847C.21H6. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof binds to one or more peptides of SEQ ID NOs: 3-26. In some embodiments, the anti-Gal3 antibody or binding fragment thereof binds to an epitope present within a region of Gal3 defined by Peptide 1 (ADNFSLHDALSGSGNPNPQG; SEQ ID NO: 3), Peptide 4 (GAGGYPGASYPGAYPGQAPP; SEQ ID NO: 6), Peptide 6 (GAYPGQAPPGAYPGAPGAYP; SEQ ID NO: 8), Peptide 7 (AYPGAPGAYPGAPAPGVYPG; SEQ ID NO: 9), or a combination thereof. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof binds to an epitope of Gal3 that includes a motif of GxYPG, where x is the amino acids alanine (A), glycine (G), or valine (V). In some embodiments, an anti-Gal3 antibody as described herein binds to an epitope of Gal3 that includes two GxYPG motifs separated by three amino acids, where x is A, G, or V. In some embodiments, any of the methods disclosed herein involving an anti-Gal3 antibody or binding fragment can be performed with an antigen binding molecule that binds to Gal3.

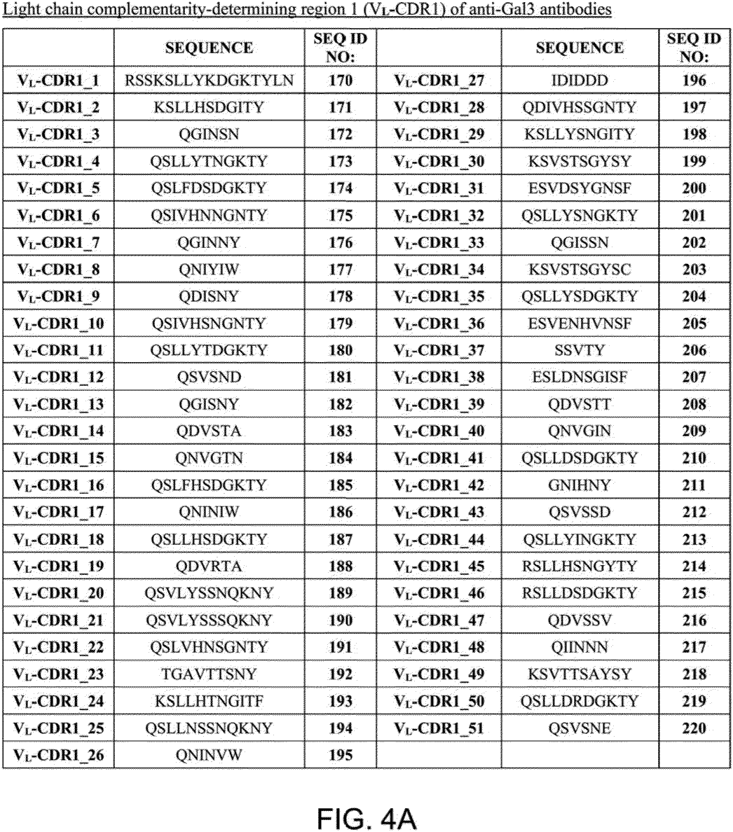

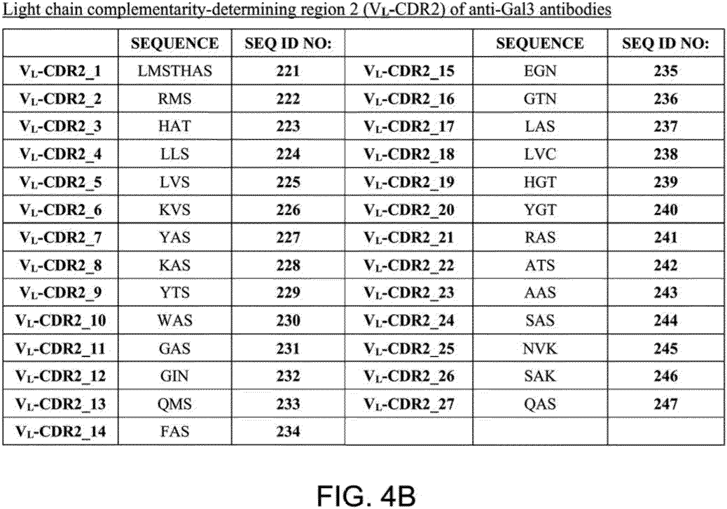

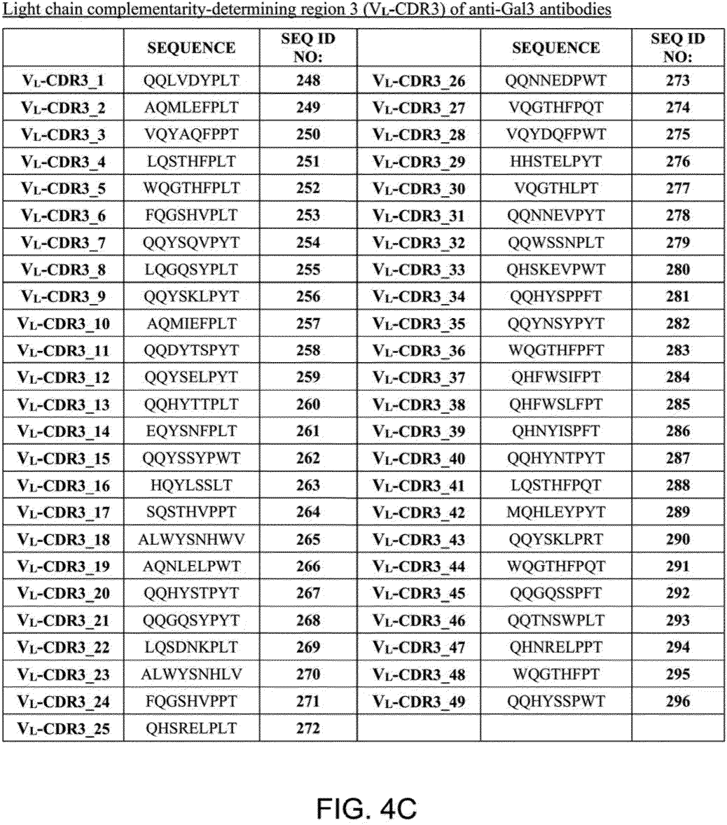

As applied to any of the methods of use disclosed herein, in some embodiments, exemplary V_{H}-CDR1 sequences are depicted in FIG. 3A. In some embodiments, exemplary V_{H}-CDR2 sequences are depicted in FIG. 3B. In some embodiments, exemplary V_{H}-CDR3 sequences are depicted in FIG. 3C. In some embodiments, exemplary V_{L}-CDR1 sequences are depicted in FIG. 4A. In some embodiments, exemplary V_{L}-CDR2 sequences are depicted in FIG. 4B. In some embodiments, exemplary V_{L}-CDR3 sequences are depicted in FIG. 4C. In some embodiments, any of the methods disclosed herein involving an anti-Gal3 antibody or binding fragment can be performed with an antigen binding molecule that binds to Gal3.

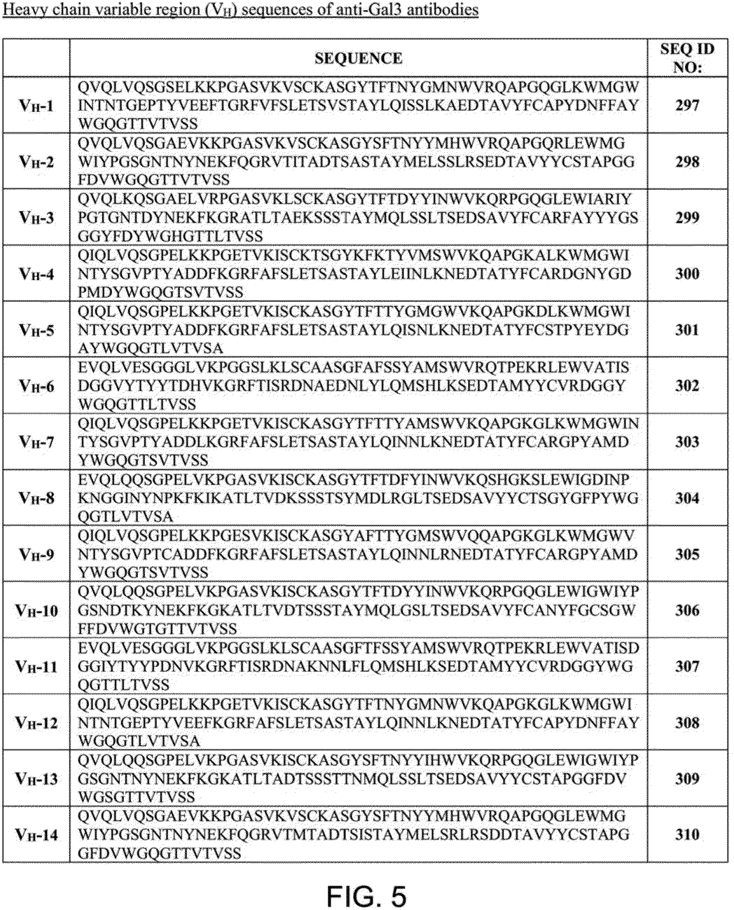

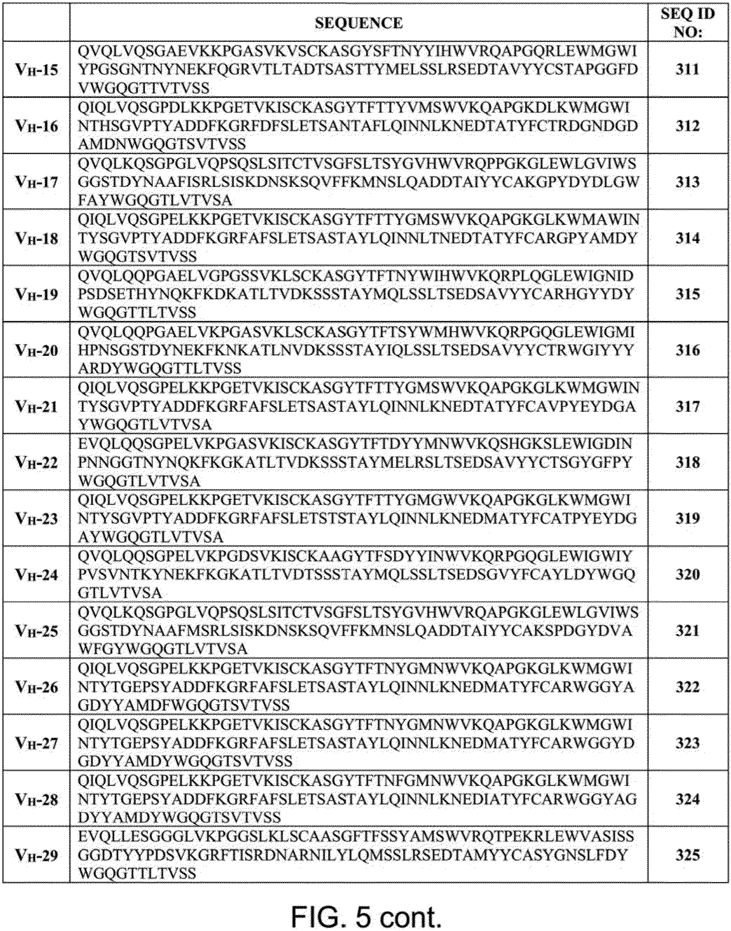

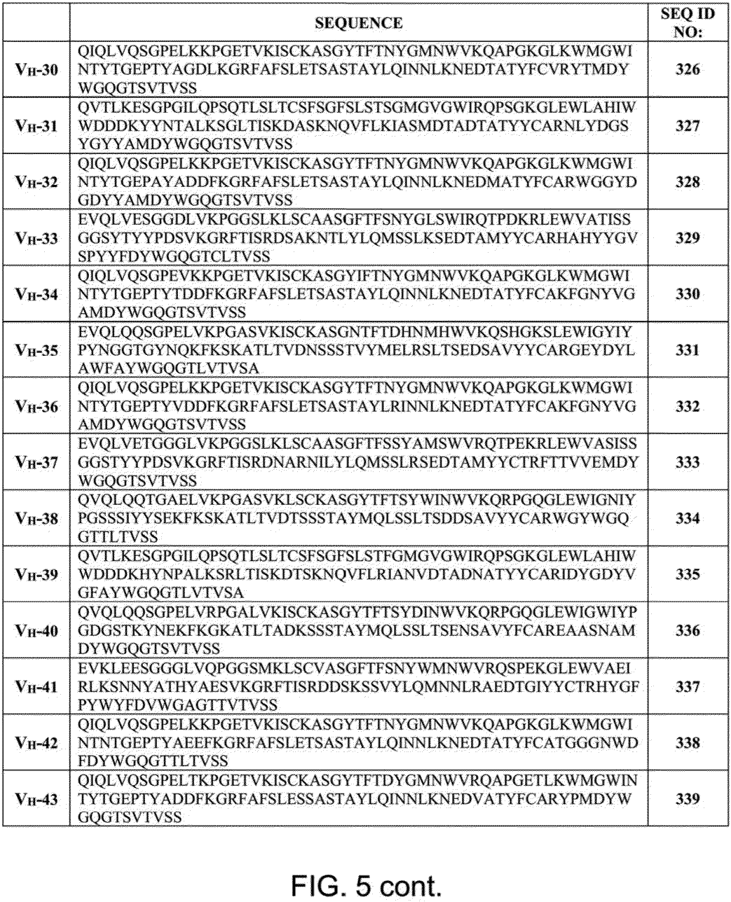

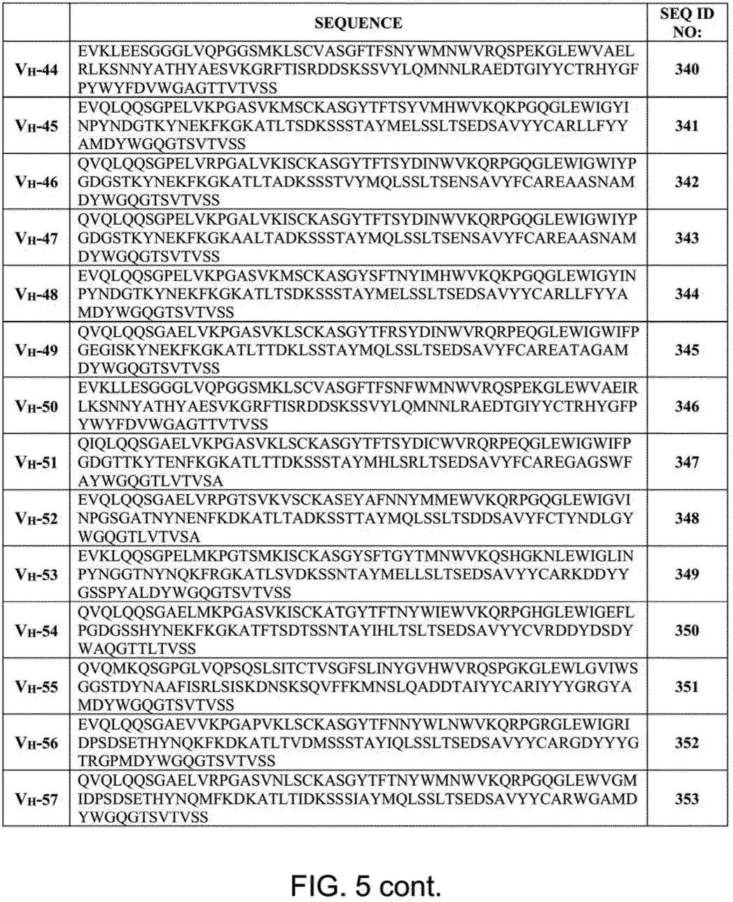

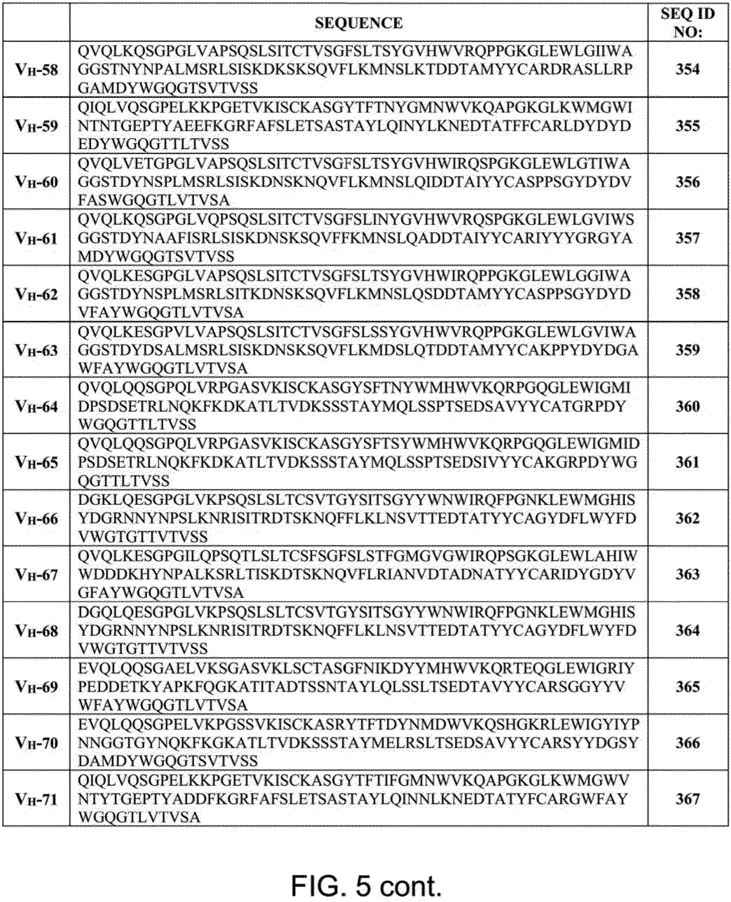

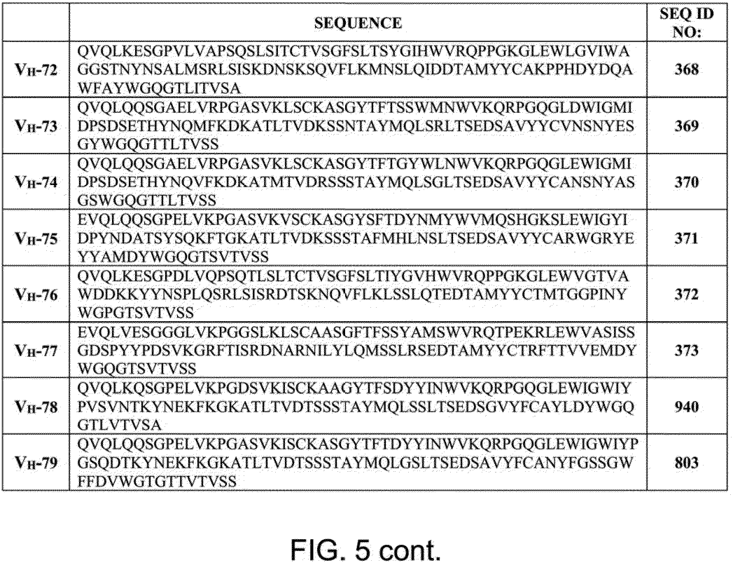

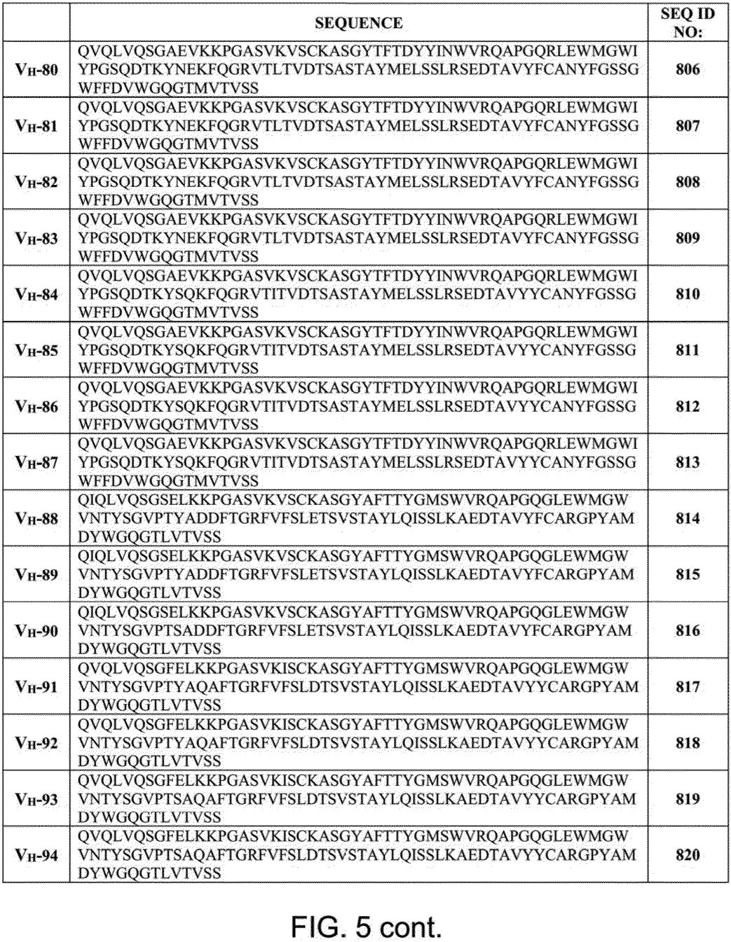

As applied to any of the methods of use disclosed herein, in some embodiments, the heavy chain variable region of any of the anti-Gal3 antibodies or binding fragments thereof disclosed herein comprises an amino acid sequence having at least 75%, 80%, 85%, 90%, 95%, or 100% sequence identity to any sequence according to SEQ ID NOs: 297-373, 803, 806-82. In some embodiments, the heavy chain variable region of any of the anti-Gal3 antibodies or binding fragments thereof disclosed herein is selected from the group consisting of SEQ ID NOs: 297-373, 803, 806-82. In some embodiments, exemplary V_{H} are depicted in FIG. 5. In some embodiments, any of the methods disclosed herein involving an anti-Gal3 antibody or binding fragment can be performed with an antigen binding molecule that binds to Gal3.

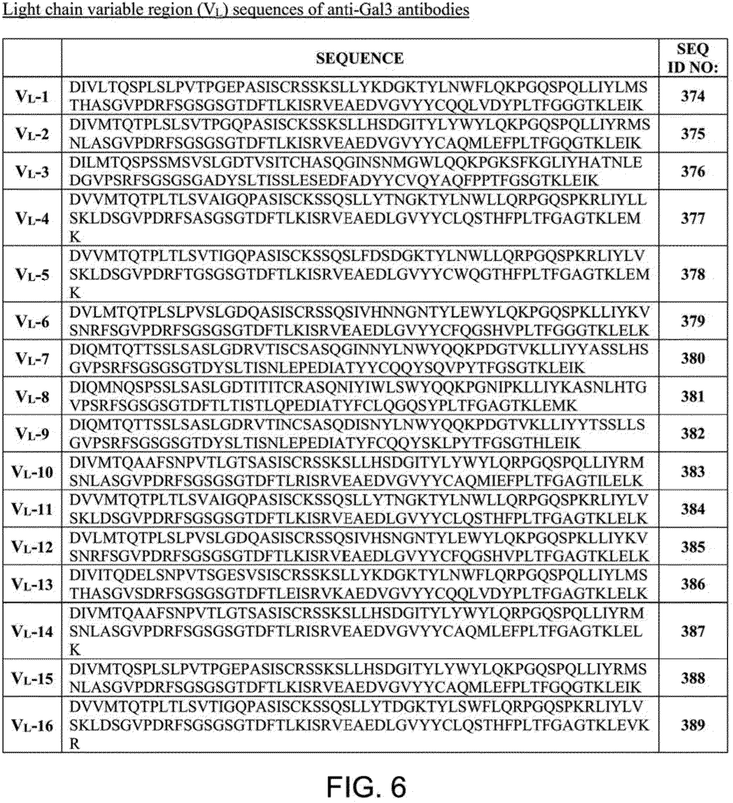

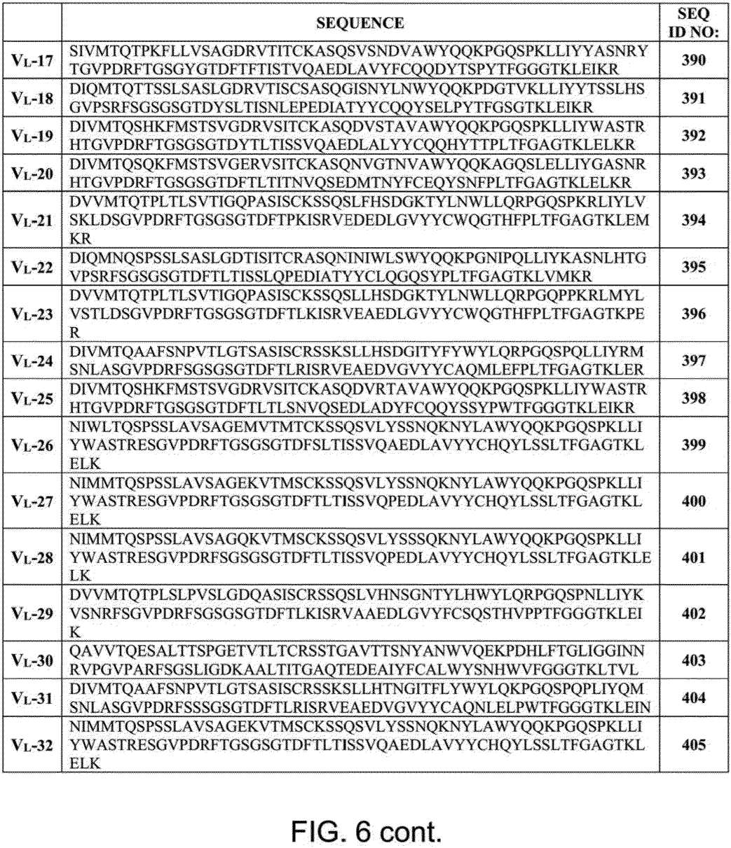

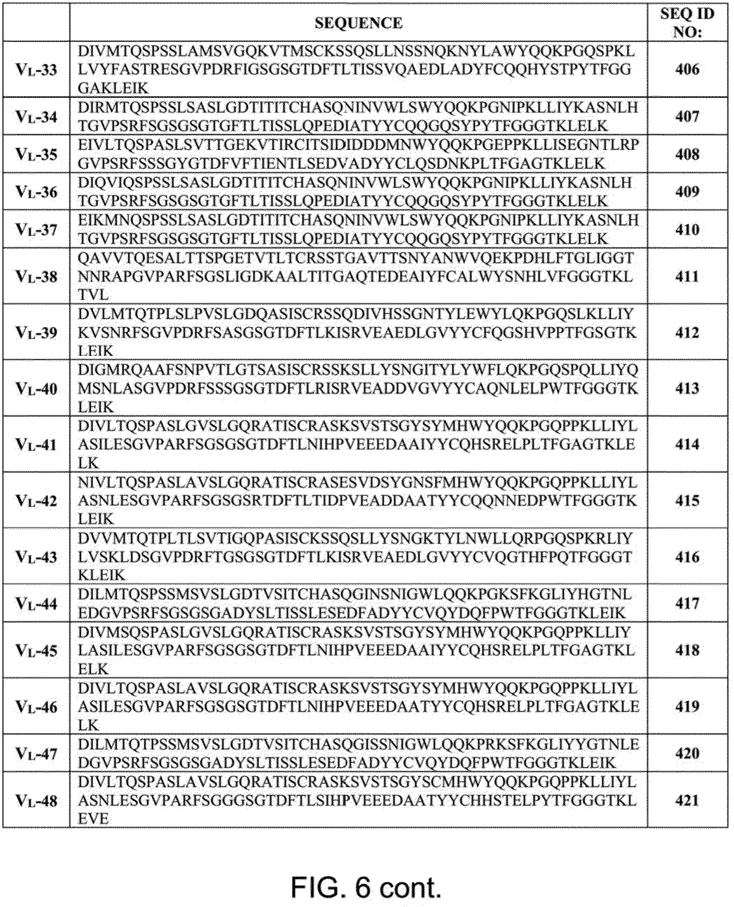

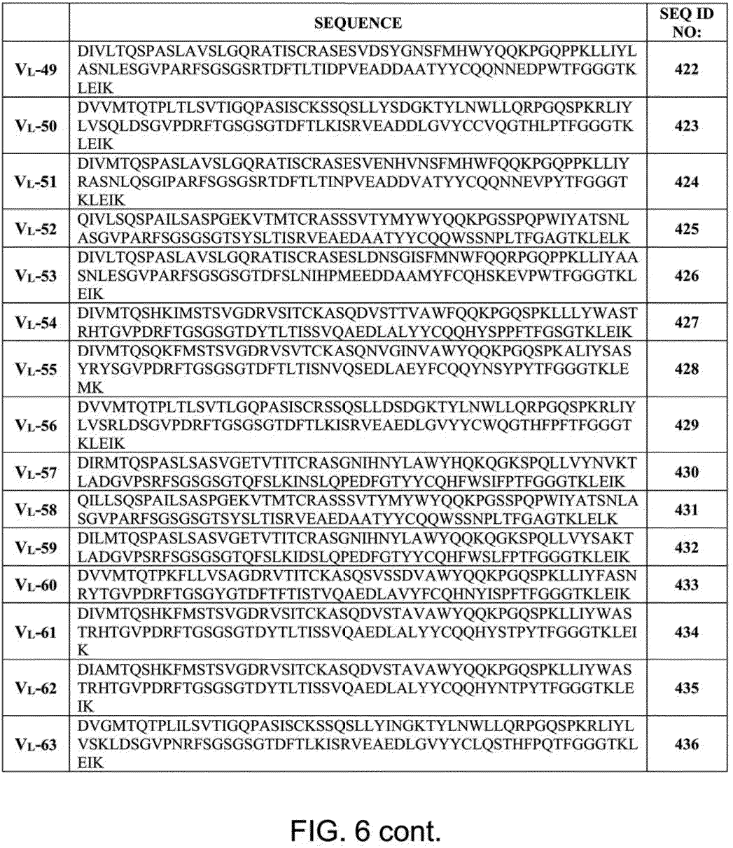

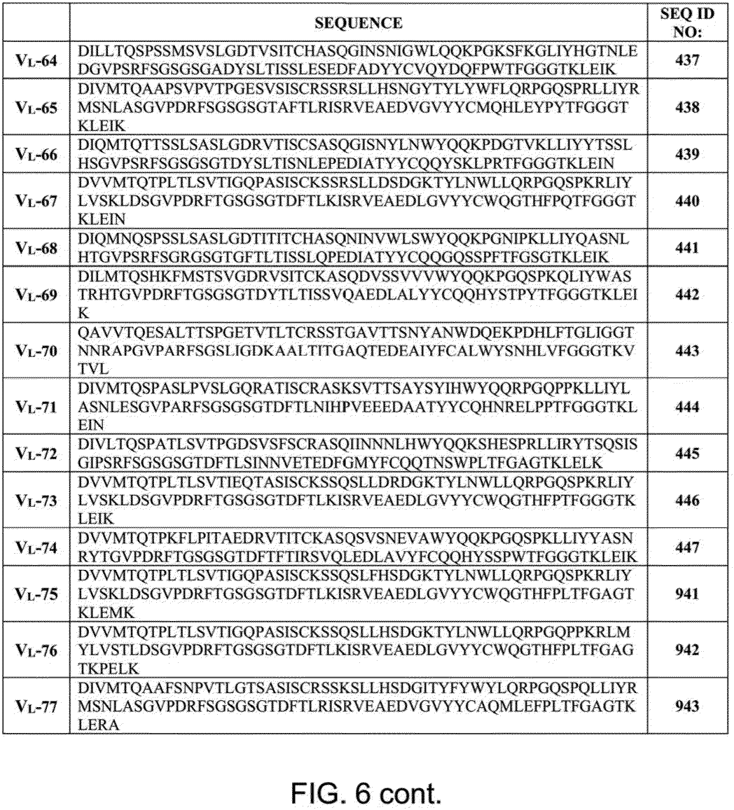

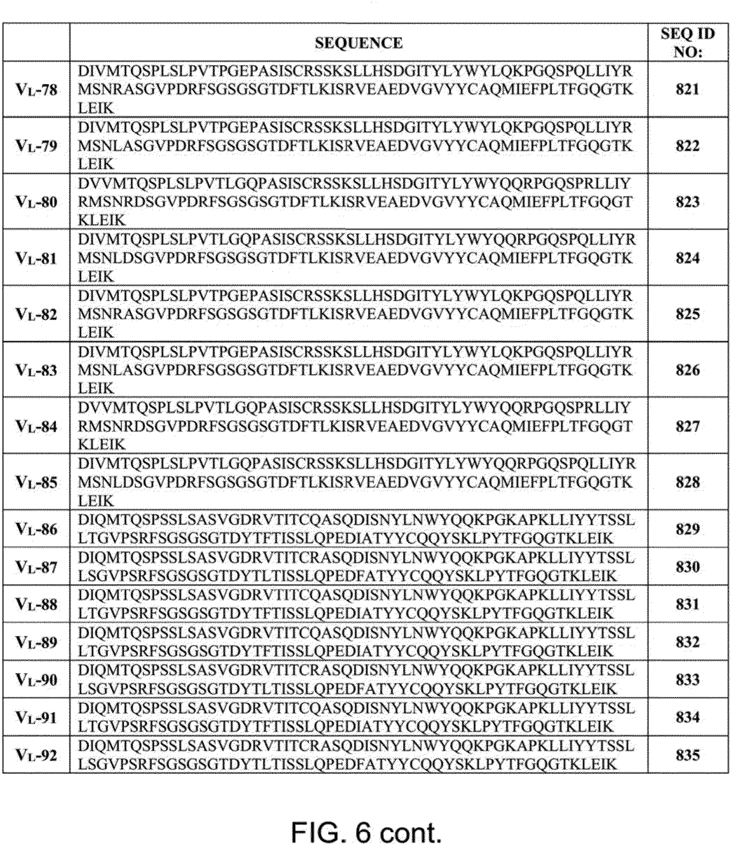

As applied to any of the methods of use disclosed herein, in some embodiments, the light chain variable region of any of the anti-Gal3 antibodies or binding fragments thereof disclosed herein comprises an amino acid sequence having at least 75%, 80%, 85%, 90%, 95%, or 100% sequence identity to any sequence according to SEQ ID NOs: 374-447, 821-835. In some embodiments, the light chain variable region of any of the anti-Gal3 antibodies or binding fragments thereof disclosed herein is selected from the group consisting of SEQ ID NOs: 374-447, 821-835. In some embodiments, exemplary V_{L} are depicted in FIG. 6. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof comprises the heavy chain sequence of any one of SEQ ID NOs: 448-494, 804, 836-850. In some embodiments, the anti-Gal3 antibody or binding fragment thereof comprises the light chain sequence of any one of SEQ ID NOs: 495-538, 805, 851-865. In some embodiments, any of the methods disclosed herein involving an anti-Gal3 antibody or binding fragment can be performed with an antigen binding molecule that binds to Gal3.

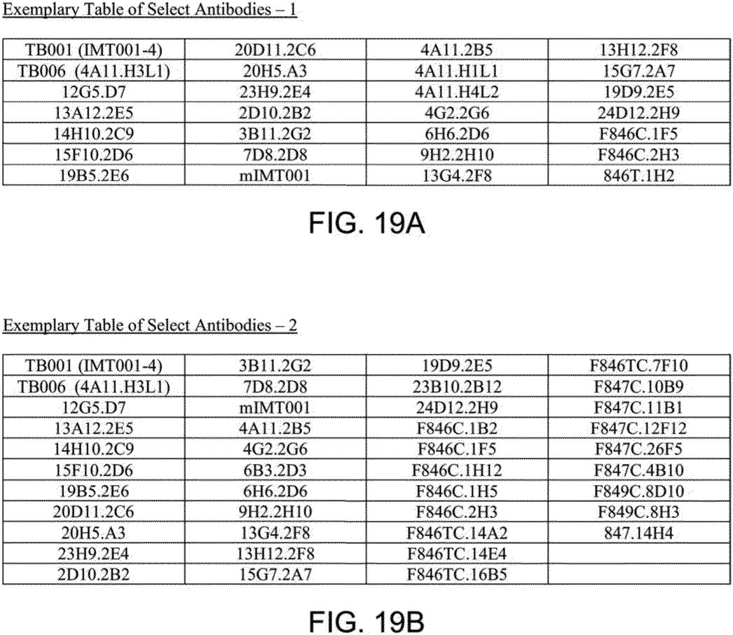

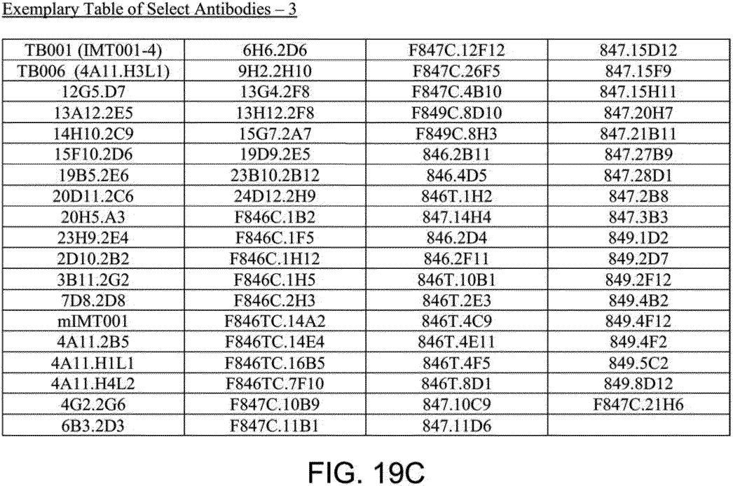

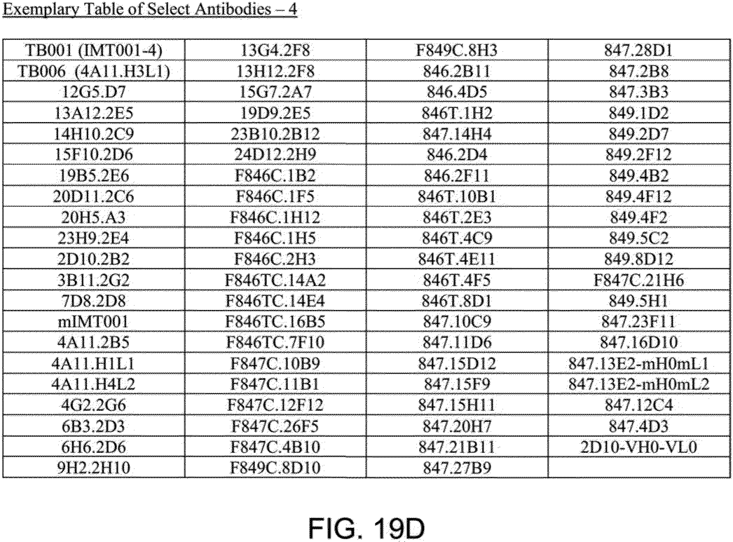

As applied to any of the methods of use disclosed herein, in some embodiments, the anti-Gal3 antibody or binding fragment thereof excludes the subset of named anti-Gal3 antibodies or binding fragments thereof depicted in any one of FIGS. 19A-D. In some embodiments, CDRs (including heavy chain and light chain CDRs), heavy chain variable regions, light chain variable regions, heavy chains, and/or light chains are excluded from the sequences associated with the subset of named anti-Gal3 antibodies or binding fragments thereof depicted in any one of FIGS. 19A-D. In some embodiments, combinations of CDRs (including heavy chain and light chain CDRs), heavy chain variable regions, light chain variable regions, heavy chains, and/or light chains are excluded from the sequences associated with the subset of named anti-Gal3 antibodies or binding fragments thereof depicted in any one of FIGS. 19A-D, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19A, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19B, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19C, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19D, or excludes any one or more of the CDRs, VH, VL, HC, and/or LC thereof.

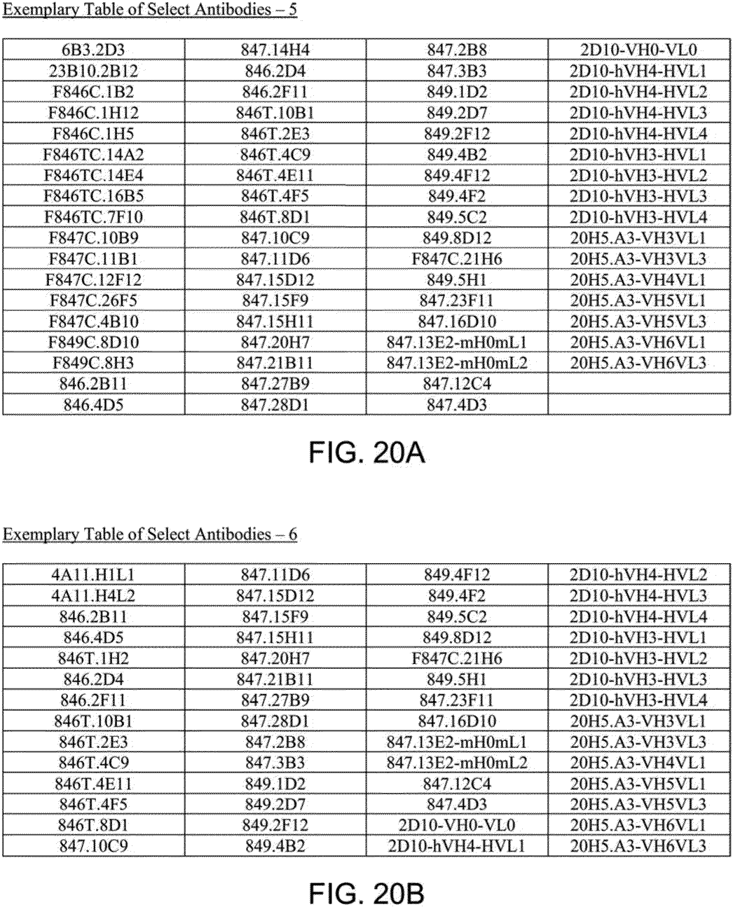

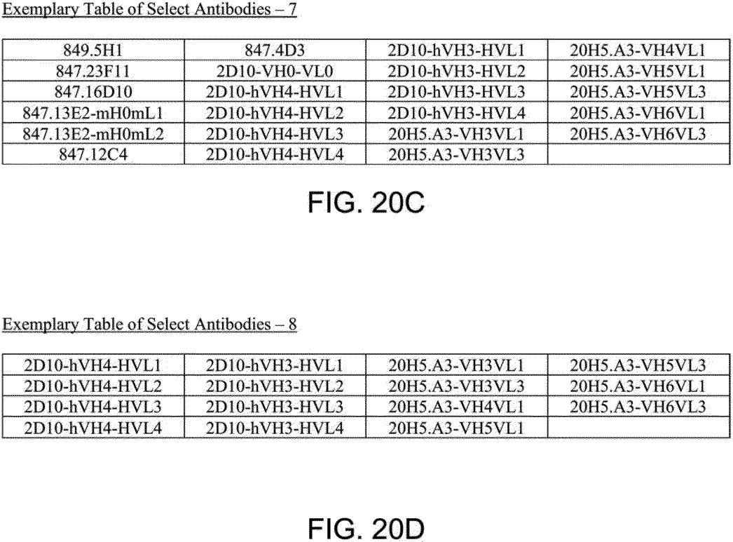

As applied to any of the methods of use disclosed herein, in some embodiments, the anti-Gal3 antibody or binding fragment thereof is selected from the subset of named anti-Gal3 antibodies or binding fragments thereof depicted in any one of FIGS. 20A-D. In some embodiments, CDRs (including heavy chain and light chain CDRs), heavy chain variable regions, light chain variable regions, heavy chains, and/or light chains are selected from the sequences associated with the subset of named anti-Gal3 antibodies or binding fragments thereof depicted in any one of FIGS. 20A-D. In some embodiments, combinations of CDRs (including heavy chain and light chain CDRs), heavy chain variable regions, light chain variable regions, heavy chains, and/or light chains are selected from the sequences associated with the subset of named anti-Gal3 antibodies or binding fragments thereof depicted in any one of FIGS. 20A-D. In some embodiments, the anti-Gal3 antibody or binding fragment thereof is selected from the group of named anti-Gal3 antibody or binding fragment thereof depicted in FIG. 20A, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 20B, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 20C, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 20D, or comprises any one or more of the CDRs, VH, VL, HC, and/or LC thereof.

As applied to any of the methods of use disclosed herein, in some embodiments, the anti-Gal3 antibody or binding fragment thereof comprises a payload. In some embodiments, the payload is conjugated to the anti-Gal3 antibody or binding fragment thereof. In some embodiments, the payload is a cytotoxic payload, microtubule disrupting agent, DNA modifying agent, Akt inhibitor, polymerase inhibitor, detectable moiety, immunomodulatory agent, immune modulator, immunotoxin, nucleic acid polymer, aptamer, peptide, or any combination thereof. In some embodiments, the payload is a detectable moiety. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof is or comprises a humanized antibody. In some embodiments, the anti-Gal3 antibody or binding fragment thereof is or comprises a full-length antibody or a binding fragment thereof. In some embodiments, the anti-Gal3 antibody or binding fragment thereof is or comprises a bispecific antibody or a binding fragment thereof. In some embodiments, the anti-Gal3-antibody or binding fragment thereof is or comprises a monovalent Fab′, a divalent Fab2, a single-chain variable fragment (scFv), a diabody, a minibody, a nanobody, a single-domain antibody (sdAb), or a camelid antibody, or binding fragment thereof. In some embodiments, the anti-Gal3 antibody or binding fragment thereof is or comprises an IgG framework. In some embodiments, the anti-Gal3 antibody or binding fragment thereof is or comprises an IgG1, IgG2, or IgG4 framework. In some embodiments, any of the methods disclosed herein involving an anti-Gal3 antibody or binding fragment can be performed with an antigen binding molecule that binds to Gal3.

As applied to any of the methods of treatment disclosed herein, in some embodiments, in some embodiments, the anti-Gal3 antibody or binding fragment thereof is administered enterally, orally, intranasally, parenterally, intracranially, subcutaneously, intramuscularly, intradermally, or intravenously, or any combination thereof.

As applied to any of the methods of treatment disclosed herein, the subject is administered TB006 sterilized drug product supplied in 8 mL glass vials, sealed with a rubber stopper and an aluminum flip top, with strength of 160 mg (20 mg/mL, 8 mL) TB006 drug product per vial.

As applied to any of the methods of treatment disclosed herein, the subject is administered TB006 sterilized drug product via IV infusion over 60 minutes, after dilution in 0.9% Sodium Chloride Injection, USP (normal saline) to a final volume of 250 mL.

As applied to any of the methods of treatment disclosed herein, in some embodiments, the anti-Gal3 antibody or binding fragment thereof is formulated for systemic administration. In some embodiments, the anti-Gal3 antibody or binding fragment thereof is formulated for parenteral administration. In some embodiments, more than one anti-Gal3 antibody or binding fragment is administered. In some embodiments, when more than one anti-Gal3 antibody or binding fragment is administered, the more than one anti-Gal3 antibodies or binding fragments thereof may be selected from the anti-Gal3 antibodies or binding fragments thereof disclosed herein. In some embodiments, any of the methods disclosed herein involving an anti-Gal3 antibody or binding fragment can be performed with an antigen binding molecule that binds to Gal3.

As applied to any of the methods of use or treatment disclosed herein, the subject is a mammal. In some embodiments, the mammal is a human, cat, dog, mouse, rat, hamster, rodent, pig, cow, horse, sheep, or goat. In some embodiments, the mammal is a human.

### Therapeutic Regimens

In some embodiments, the anti-Gal3 antibodies or binding fragments thereof disclosed herein are administered for therapeutic applications. In some embodiments, the anti-Gal3 antibody or binding fragment thereof is administered once per day, twice per day, three times per day or more. The anti-Gal3 antibody or binding fragment thereof is administered daily, every day, every alternate day, five days a week, once a week, every other week, two weeks per month, three weeks per month, once a month, twice a month, three times per month, or more. In some embodiments, the anti-Gal3 antibody or binding fragment thereof is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient’s status does improve, upon the doctor’s discretion the administration of the anti-Gal3 antibody or binding fragment thereof is given continuously; alternatively, the dose of the anti-Gal3 antibody or binding fragment thereof being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In an exemplary therapeutic regimen, an anti-Gal3 antibody or binding fragment thereof is administered in 1, 2, 3, 4, 5, 6, 7, 8, 9, or 10 unit doses. In some embodiments, 5, 6, 7, 8, 9, or 10 unit doses are administered. In some embodiments, 5 unit doses are administered. In some embodiments, 8 unit doses are administered. In some embodiments, each unit dose comprises 1000 mg or about 1000 mg of the anti-Gal3 antibody or binding fragment thereof, the unit doses are administered every 7 days or about 7 days, and where each unit dose is administered over the course of 1 hour or about 1 hour. In some embodiments, the anti-Gal3 antibody or binding fragment thereof is TB006 or binding fragment thereof. In some embodiments, the anti-Gal3 antibody or binding fragment thereof is administered enterally, orally, intranasally, parenterally, intracranially, subcutaneously. In some embodiments, the anti-Gal3 antibody or binding fragment thereof is administered intravenously.

In an exemplary therapeutic regimen, an anti-Gal3 antibody or binding fragment thereof is administered in 5 unit doses. In some embodiments, each unit dose comprises 50, 60, 70, 80, 90, 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500 or 5000 mg of the anti-Gal3 antibody or binding fragment thereof per unit dose, or any amount per unit dose within a range defined by any two of the aforementioned amounts per unit dose. In some embodiments, each unit dose comprises 800, 900, 1000, 1100, 1200, 1300, 1400, or 1500 mg of the anti-Gal3 antibody or binding fragment thereof per unit dose, or any amount per unit dose within a range defined by any two of the aforementioned amounts per unit dose. In some embodiments, each unit dose comprises 1000 mg or about 1000 mg of the anti-Gal3 antibody or binding fragment thereof per unit dose. The unit doses are administered every 7 days or about 7 days, and where each unit dose is administered over the course of 1 hour or about 1 hour. In some embodiments, the anti-Gal3 antibody or binding fragment thereof is TB006 or binding fragment thereof. In some embodiments, the anti-Gal3 antibody or binding fragment thereof is administered enterally, orally, intranasally, parenterally, intracranially, subcutaneously. In some embodiments, the anti-Gal3 antibody or binding fragment thereof is administered intravenously.

In an exemplary therapeutic regimen, an anti-Gal3 antibody or binding fragment thereof is administered in 5 unit doses, where each unit dose comprises 1000 mg or about 1000 mg of the anti-Gal3 antibody or binding fragment thereof per unit dose. In some embodiments, the unit doses are administered every 1, 2, 3, 4, 5, 6, or 7 days, or 1, 2, 3, or 4 weeks, or any interval within a range defined by any two of the aforementioned intervals. In some embodiments, the unit doses are administered every 7 days or about 7 days. Each unit dose is administered over the course of 1 hour or about 1 hour. In some embodiments, the anti-Gal3 antibody or binding fragment thereof is TB006 or binding fragment thereof. In some embodiments, the anti-Gal3 antibody or binding fragment thereof is administered enterally, orally, intranasally, parenterally, intracranially, subcutaneously. In some embodiments, the anti-Gal3 antibody or binding fragment thereof is administered intravenously.

In an exemplary therapeutic regimen, an anti-Gal3 antibody or binding fragment thereof is administered in 5 unit doses, where each unit dose comprises 1000 mg or about 1000 mg of the anti-Gal3 antibody or binding fragment thereof per unit dose, and the unit doses are administered every 7 days or about 7 days. In some embodiments, each unit dose is administered over the course of 10, 20, 30, 40, 50, 60, 70, 80, 90, or 100 minutes, or any amount of time within a range defined by any two of the aforementioned durations. In some embodiments, each unit dose is administered over the course of 1 hour or about 1 hour. In some embodiments, the anti-Gal3 antibody or binding fragment thereof is TB006 or binding fragment thereof. In some embodiments, the anti-Gal3 antibody or binding fragment thereof is administered enterally, orally, intranasally, parenterally, intracranially, subcutaneously. In some embodiments, the anti-Gal3 antibody or binding fragment thereof is administered intravenously.

In an exemplary therapeutic regimen, an anti-Gal3 antibody or binding fragment thereof is administered in 5 unit doses, where each unit dose comprises 1000 mg or about 1000 mg of the anti-Gal3 antibody or binding fragment thereof, the unit doses are administered every 7 days or about 7 days, and where each unit dose is administered over the course of 1 hour or about 1 hour. In some embodiments, the anti-Gal3 antibody or binding fragment thereof is TB006 or binding fragment thereof. In some embodiments, the anti-Gal3 antibody or binding fragment thereof is administered enterally, orally, intranasally, parenterally, intracranially, subcutaneously. In some embodiments, the anti-Gal3 antibody or binding fragment thereof is administered intravenously.

In some embodiments, an anti-Gal3 antibody or binding fragment thereof, such as TB006, is supplied in 8 mL glass vials, sealed with a rubber stopper and an aluminum flip top, with strength of 160 mg (20 mg/mL, 8 mL) anti-Gal3 antibody or binding fragment per vial.

In some embodiments, the anti-Gal3 antibody or binding fragment thereof, such as TB006, is administered via IV infusion over 60 minutes, after dilution in 0.9% Sodium Chloride Injection, USP (normal saline) to a final volume of 250 mL.

Once improvement of the patient’s condition has occurred, a maintenance dose is administered if necessary. Subsequently, the dosage or the frequency of administration, or both, can be reduced, as a function of the symptoms, to a level at which the treated disease, disorder, or condition is retained.

In some embodiments, any one of the appropriate regimens provided herein are performed to comprise administering the subject 5 unit doses of 1000 mg or about 1000 mg of the anti-Gal3 antibody or binding fragment thereof, such as TB006, every 7 days or about 7 days, and assessing the subject by the NIHSS at Baseline (day 1 at the initial administration) and further assessed at Days 15, 36, 64, and 104 following the initial intervention.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Exemplary Anti-Gal3 Antibodies and Binding Fragments Thereof

Disclosed herein and as applicable to any of the methods or uses disclosed herein are anti-Gal3 antibodies or binding fragments thereof. In some embodiments, the anti-Gal3 antibody or binding fragment thereof binds to the N-terminal domain of Gal3, N-terminus of Gal3, or the tandem repeat domain (TRD) of Gal3. In some embodiments, the anti-Gal3 antibody or binding fragment thereof does not bind to the N-terminus of Gal3, the N-terminal domain of Gal3, or the TRD of Gal3. In some embodiments, the anti-Gal3 antibody or binding fragment thereof binds to the C-terminus of Gal3, the C-terminal domain of Gal3, or the CRD of Gal3. In some embodiments, the anti-Gal3 antibody or binding fragment thereof does not bind to the C-terminus of Gal3, the C-terminal domain of Gal3, or the CRD of Gal3. In some embodiments, any of the anti-Gal3 antibodies or binding fragments thereof or any arrangement of any of the anti-Gal3 antibodies or binding fragments provided herein may be substituted with an antigen binding molecule that binds to Gal3.

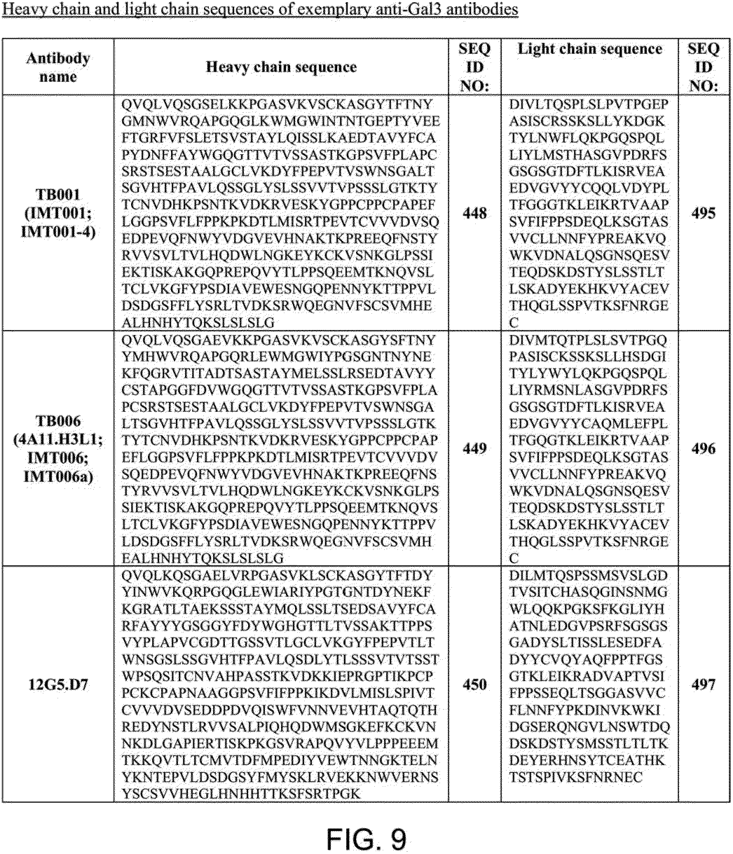

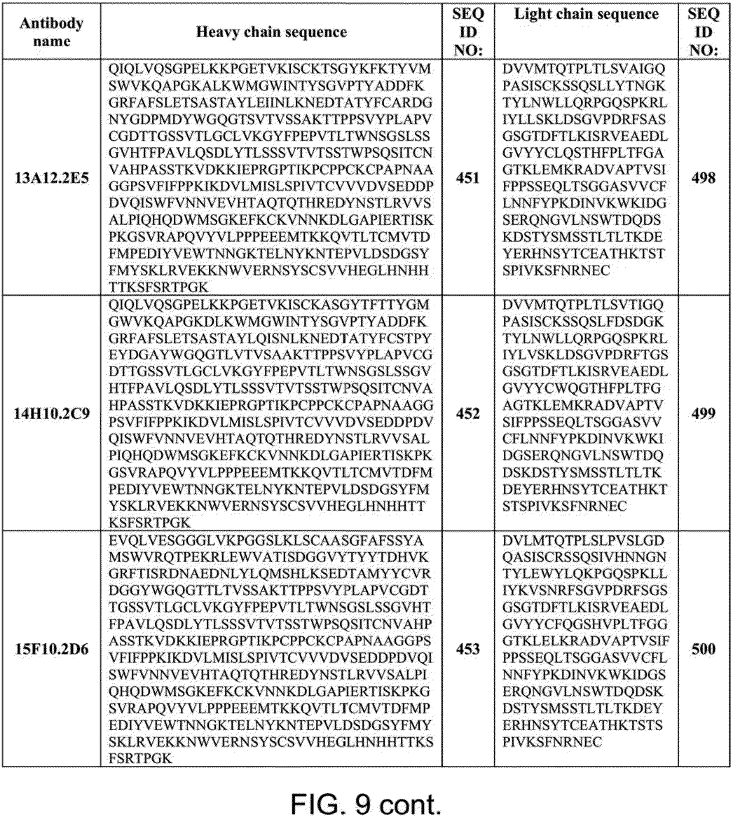

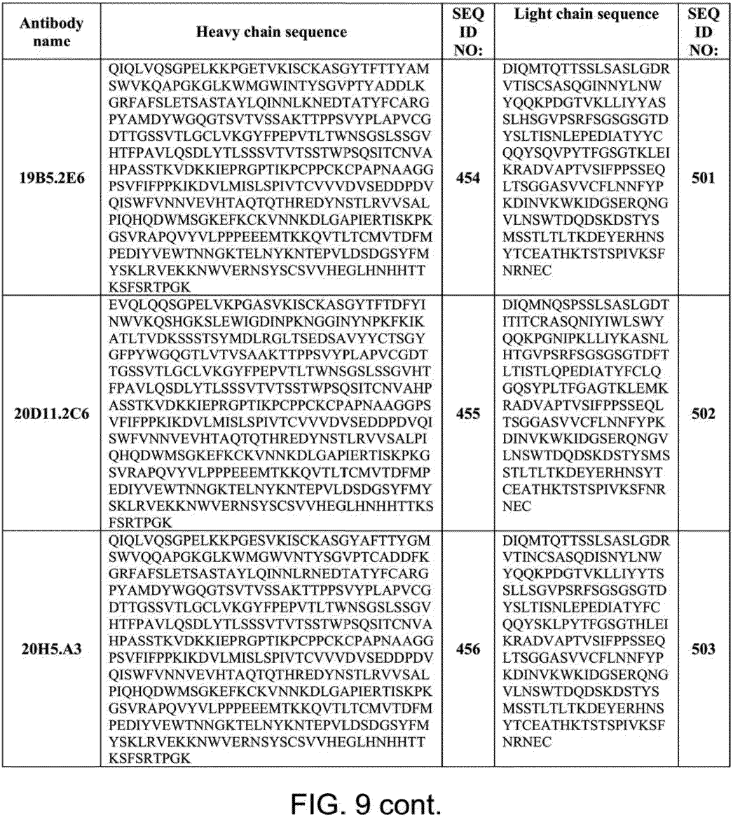

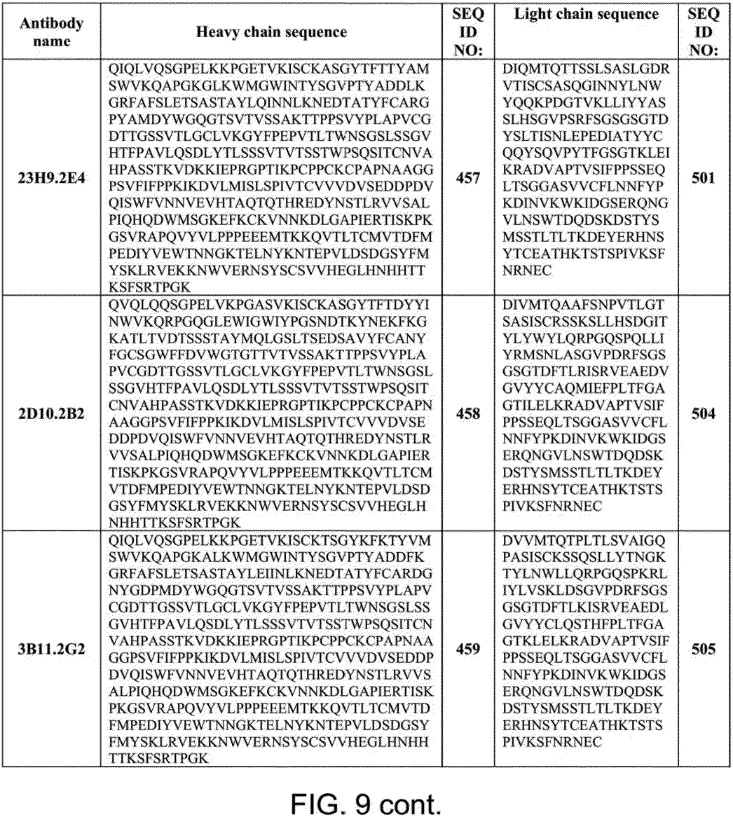

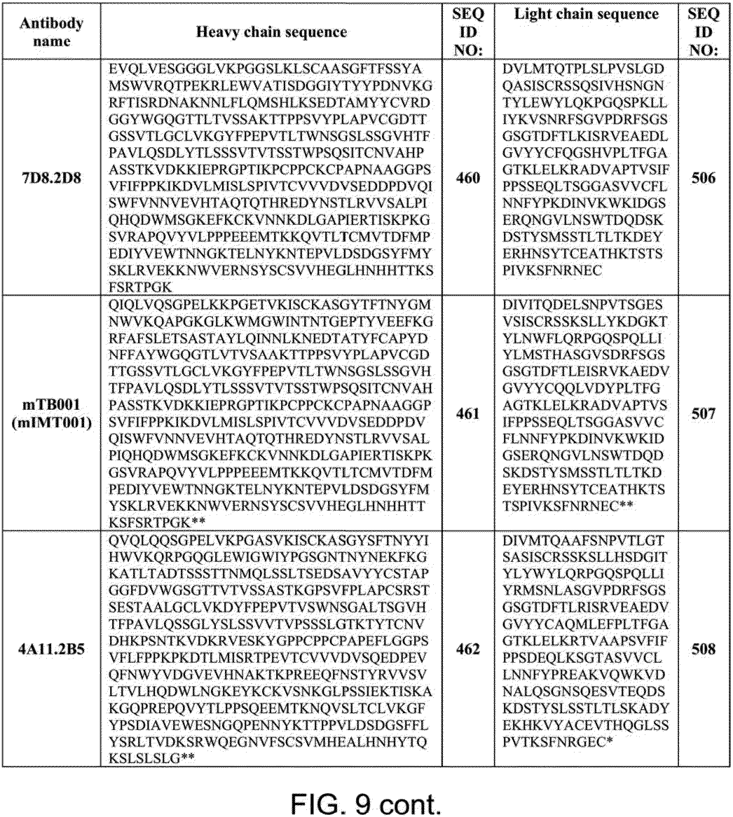

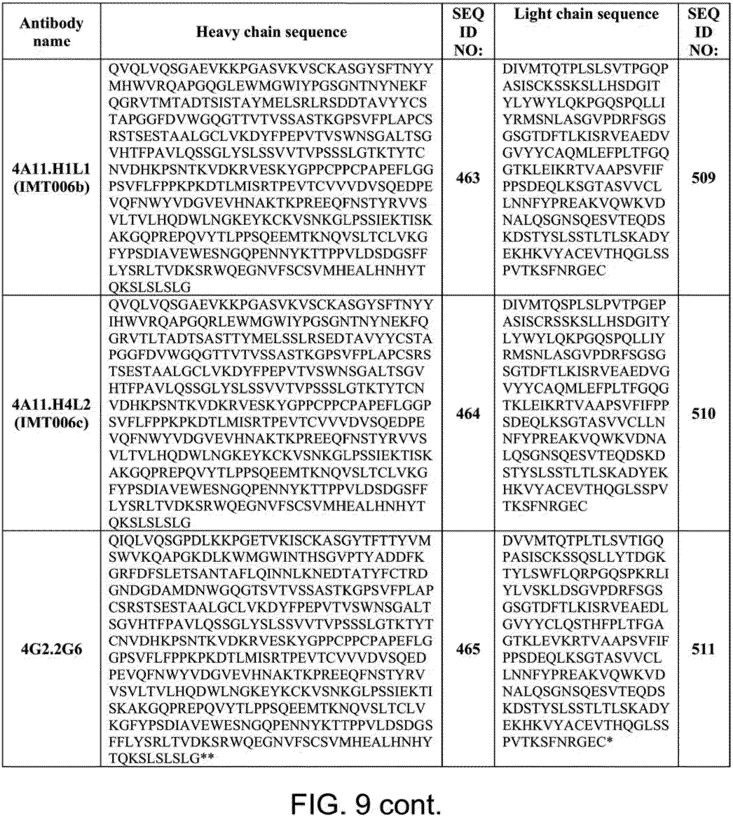

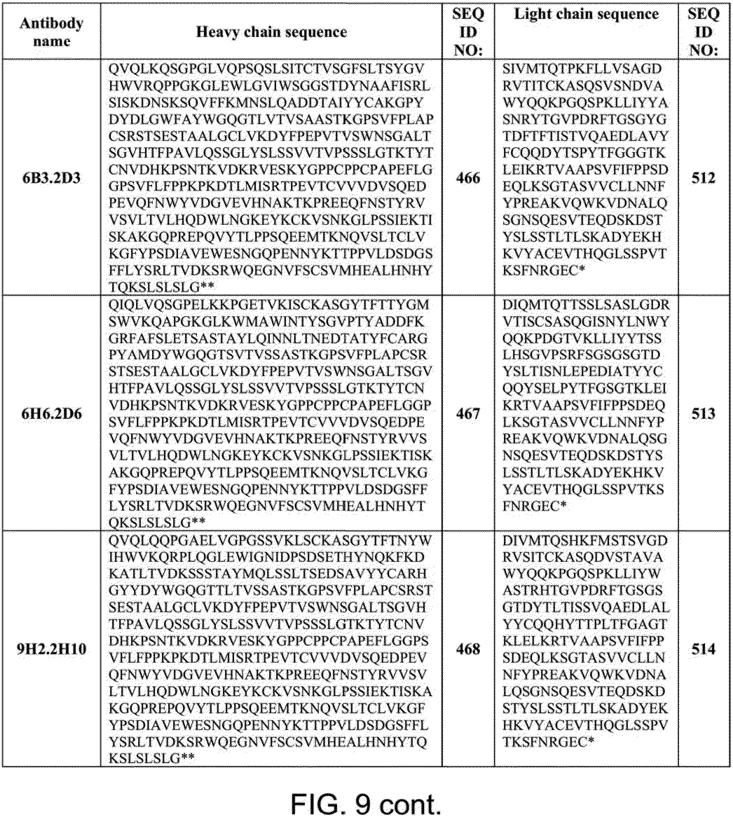

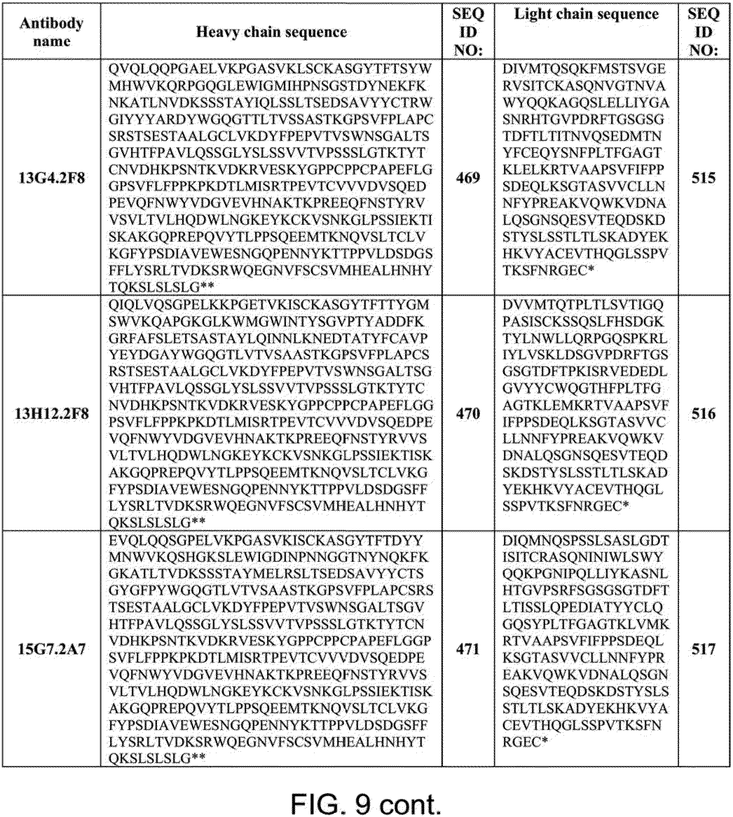

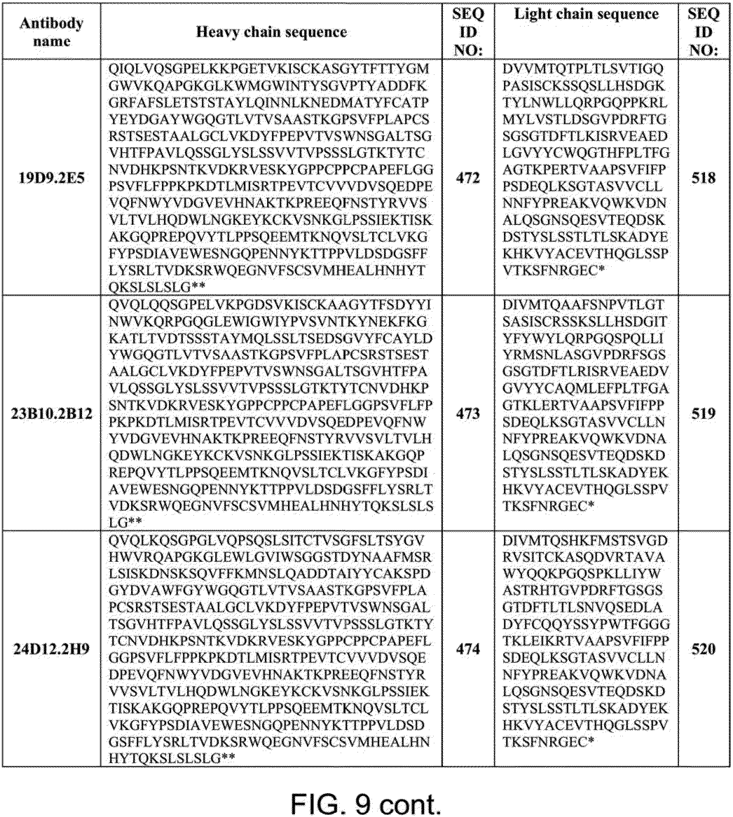

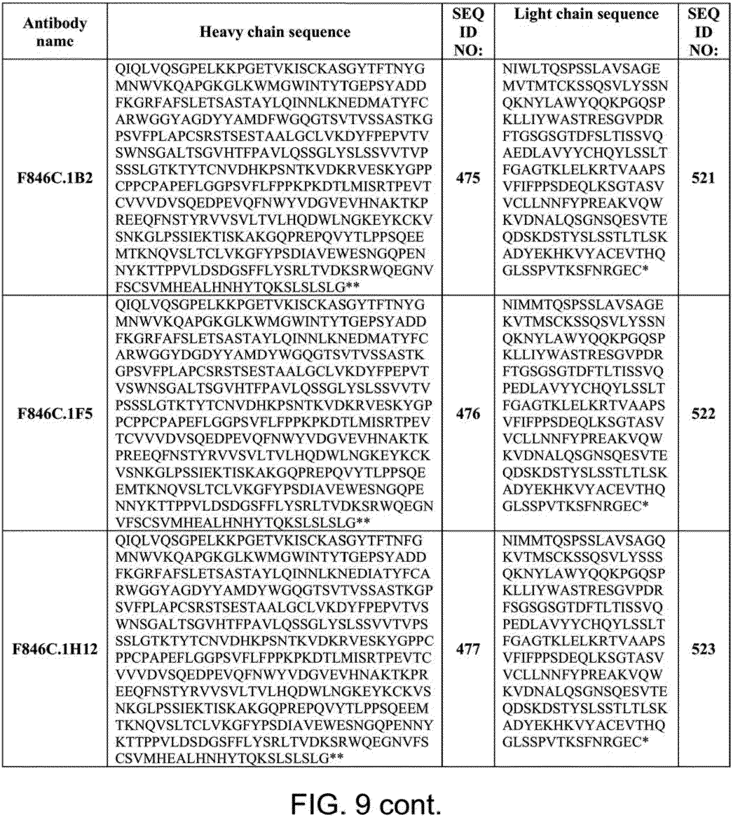

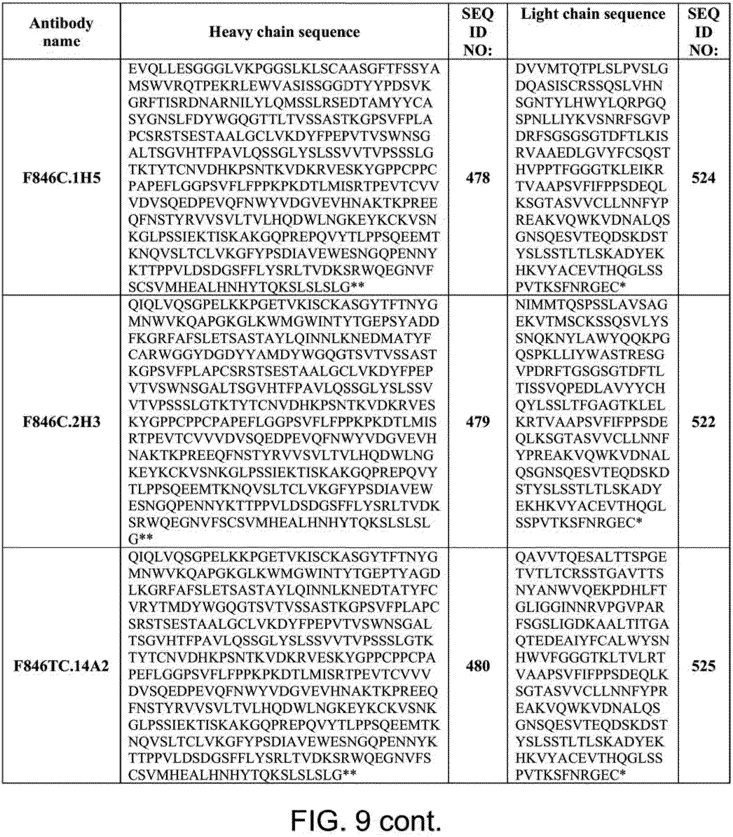

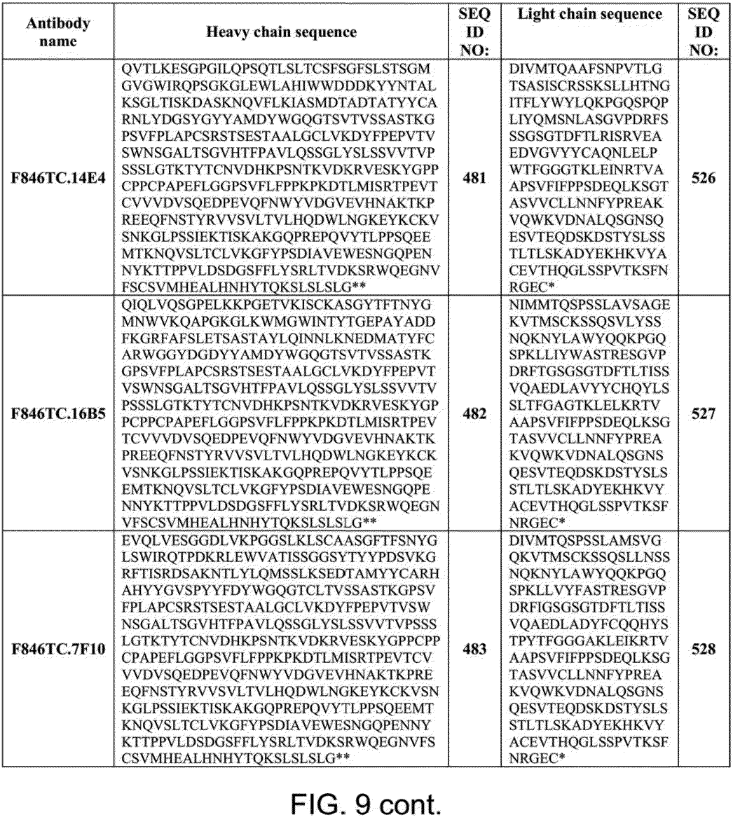

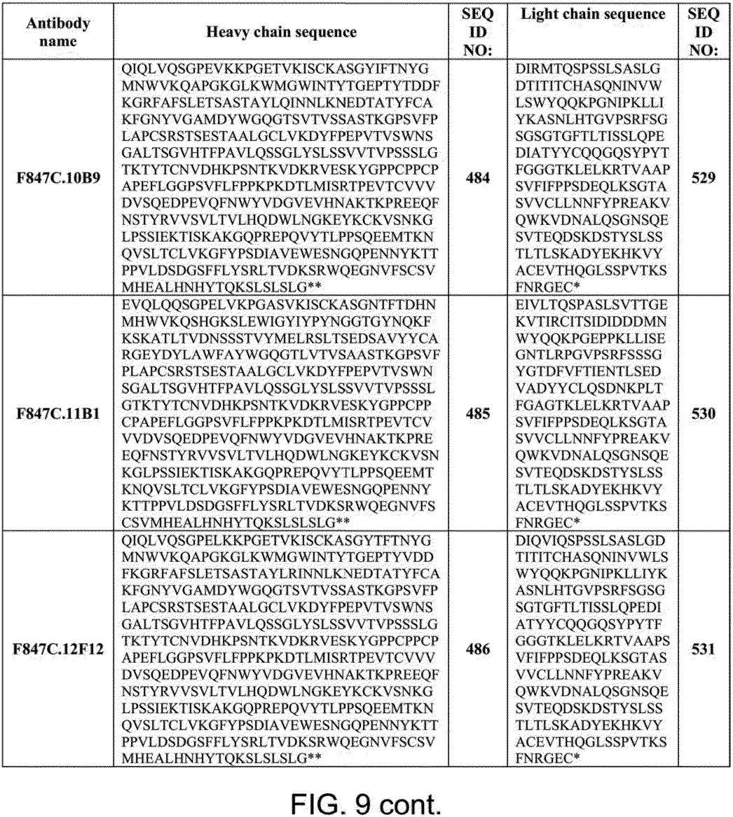

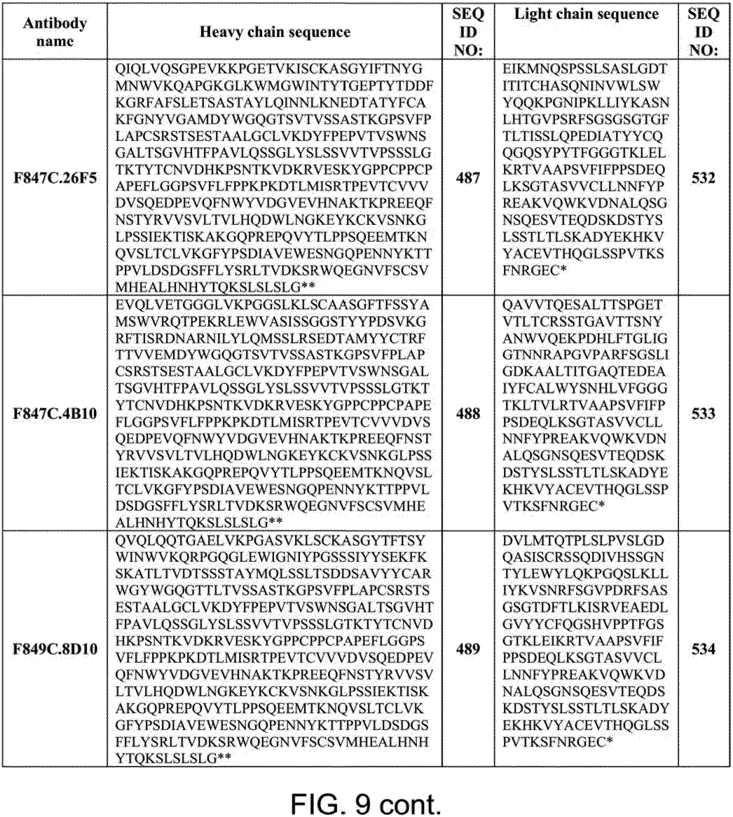

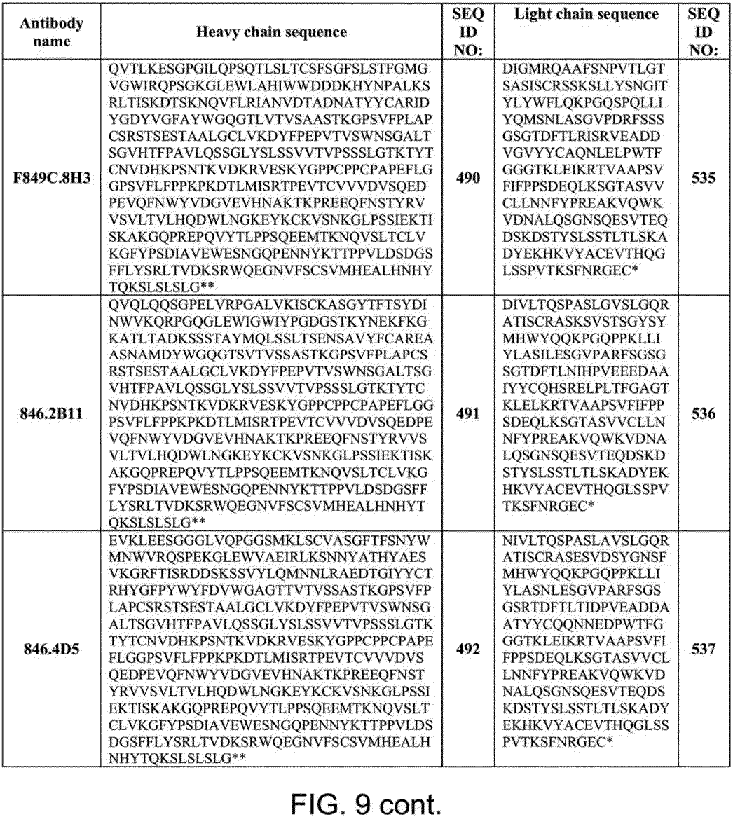

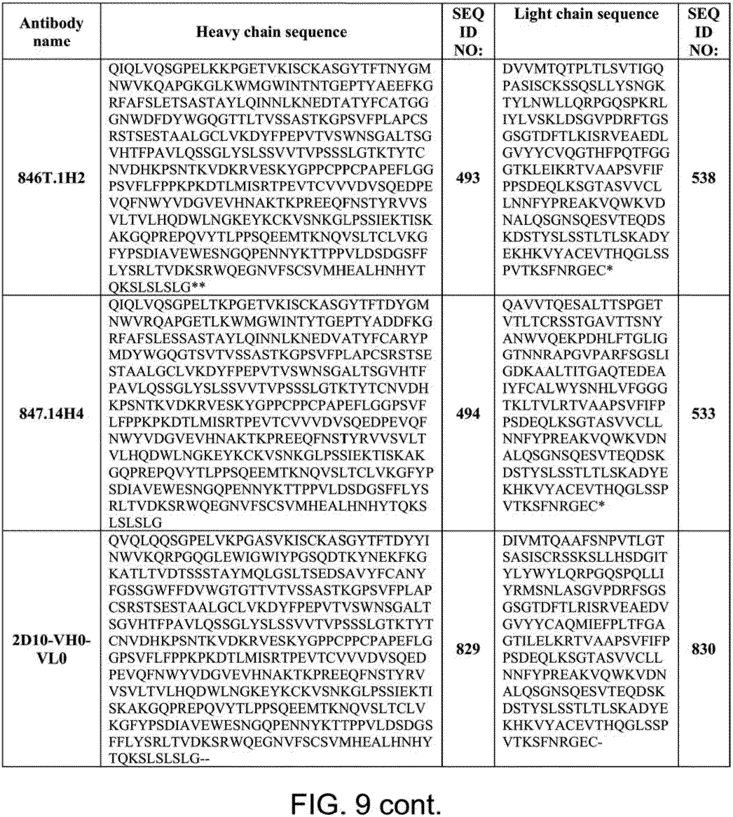

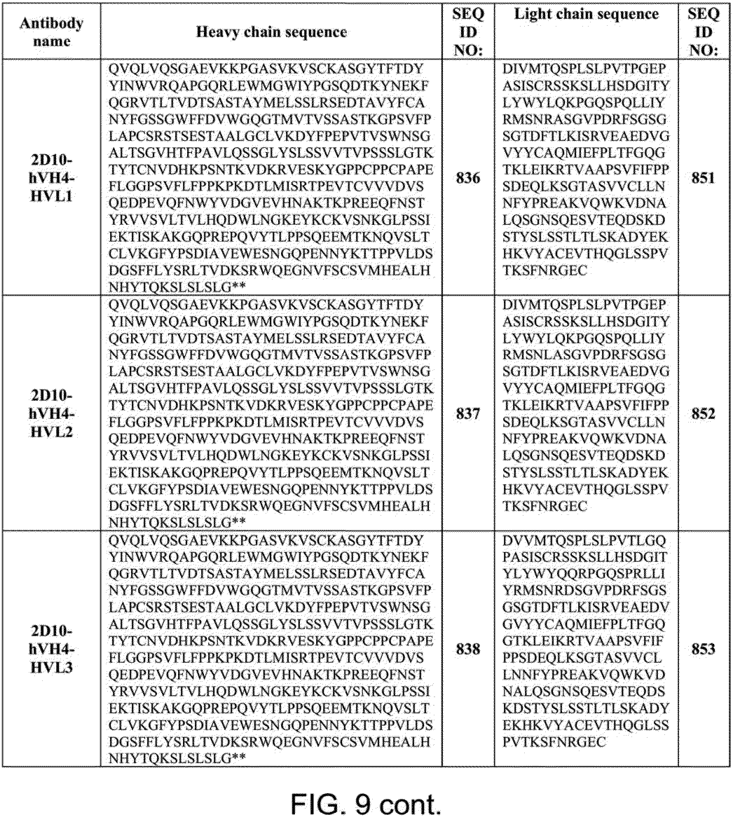

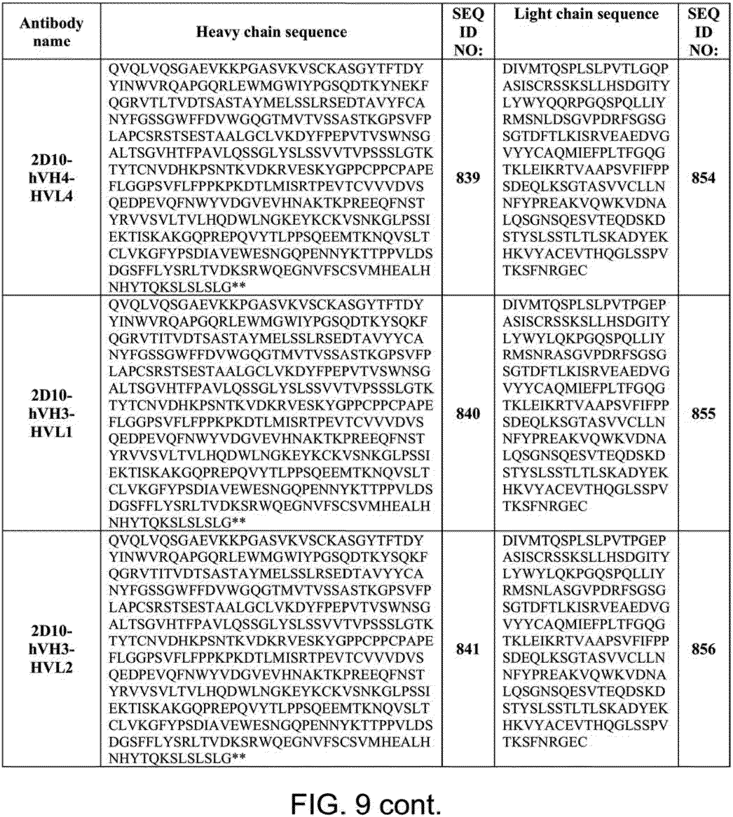

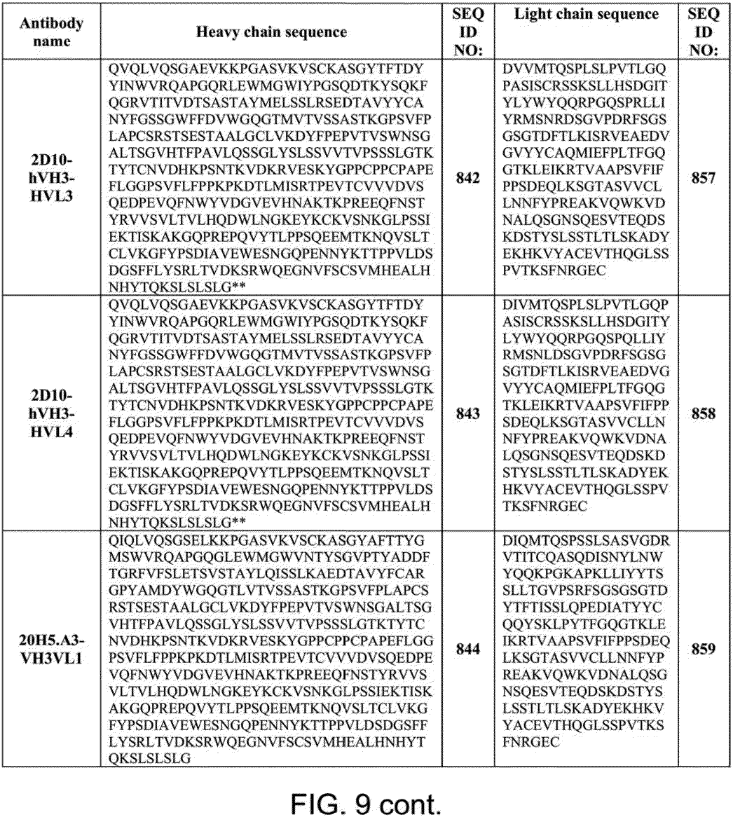

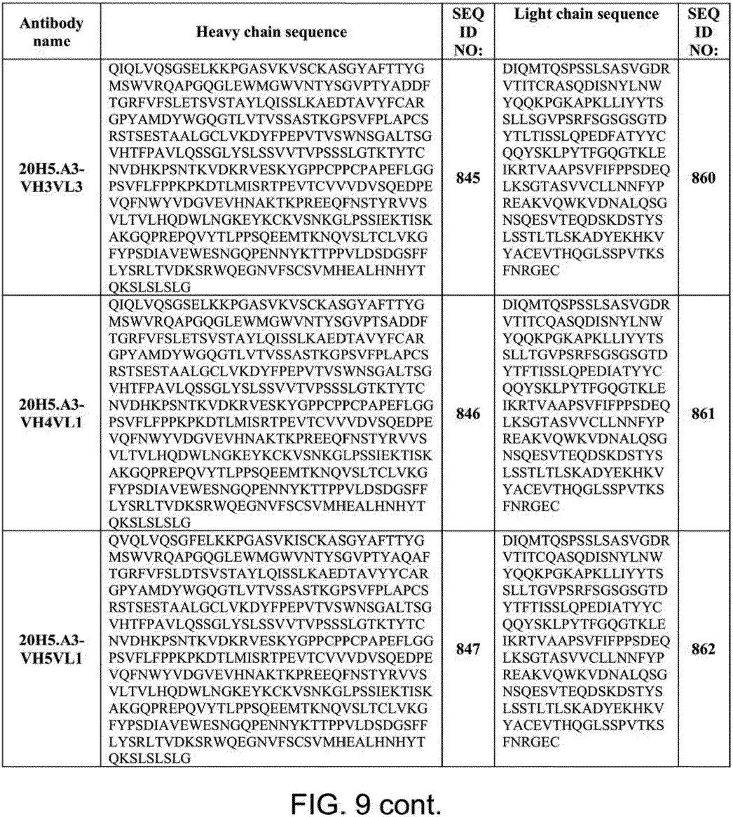

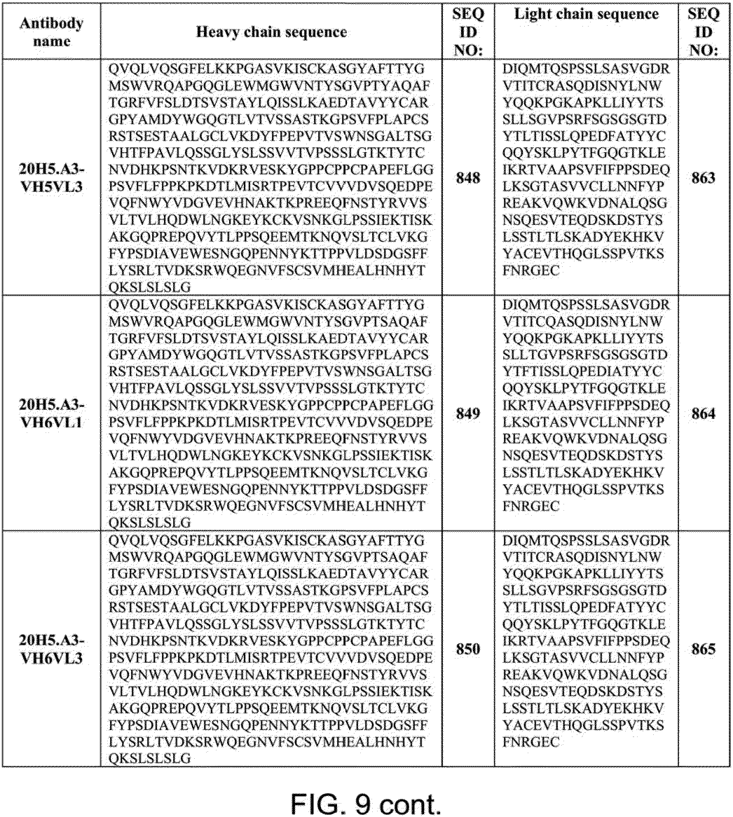

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity to any amino acid sequence according to SEQ ID NOs: 27-70. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity to any amino acid sequence according to SEQ ID NOs: 71-111, 801. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity to any amino acid sequence according to SEQ ID NOs: 112-169, 802. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity to any amino acid sequence according to SEQ ID NOs: 170-220. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity to any amino acid sequence according to SEQ ID NOs: 221-247. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity to any amino acid sequence according to SEQ ID NOs: 248-296. In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity or similarity to a VL sequence, a VH sequence, a VL/VH pairing, and/or V_{H}-CDR1, V_{H}-CDR2, V_{H}-CDR3, V_{L}-CDR1, V_{L}-CDR2, V_{L}-CDR3 (including 1, 2, 3, 4, or 5 amino acid substitutions of any one or more of these CDRs) set from the heavy chain and light chain sequences as depicted in FIG. 9.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 0, 1, 2, 3, 4, 5, or 6 substitutions relative to any amino acid sequence according to SEQ ID NOs: 27-70. In some embodiments, the V_{H}-CDR2 comprises an amino acid sequence having at least 0, 1, 2, 3, 4, 5, or 6 substitutions relative to any amino acid sequence according to SEQ ID NOs: 71-111, 801. In some embodiments, the V_{H}-CDR3 comprises an amino acid sequence having at least 0, 1, 2, 3, 4, 5, or 6 substitutions relative to any amino acid sequence according to SEQ ID NOs: 112-169, 802. In some embodiments, the V_{L}-CDR1 comprises an amino acid sequence having at least 0, 1, 2, 3, 4, 5, or 6 substitutions relative to any amino acid sequence according to SEQ ID NOs: 170-220. In some embodiments, the V_{L}-CDR2 comprises an amino acid sequence having at least 0, 1, 2, 3, 4, 5, or 6 substitutions relative to any amino acid sequence according to SEQ ID NOs: 221-247. In some embodiments, the V_{L}-CDR3 comprises an amino acid sequence having at least 0, 1, 2, 3, 4, 5, or 6 substitutions relative to any amino acid sequence according to SEQ ID NOs: 248-296.

In some embodiments, the antibody or binding fragment thereof comprises a combination of a V_{L}-CDR1, a V_{L}-CDR2, a V_{L}-CDR3, a V_{H}-CDR1, a V_{H}-CDR2, and a V_{H}-CDR3 as illustrated in FIG. 7.

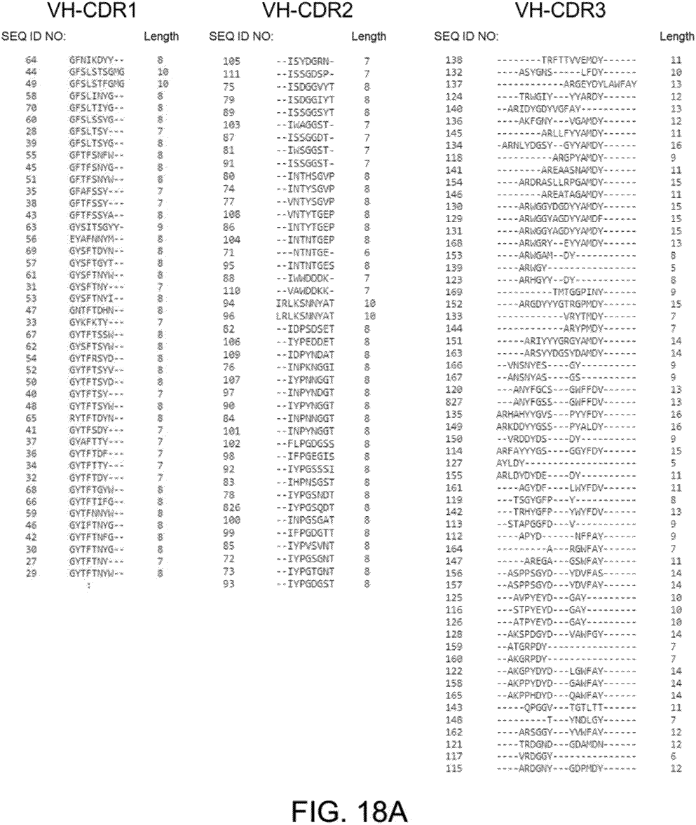

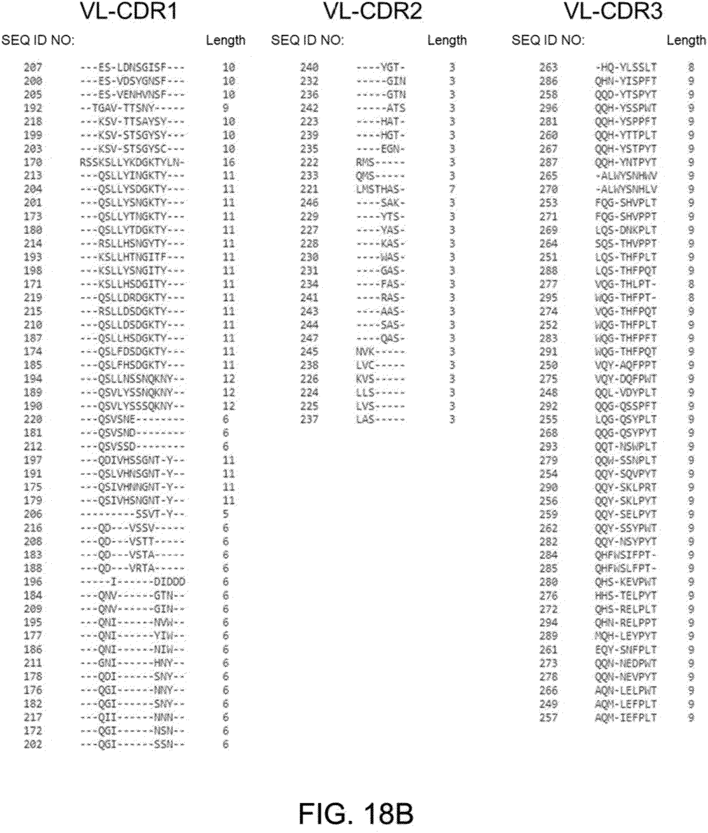

In some embodiments, the antibody or binding fragment thereof comprises a combination of a V_{H}-CDR1, a V_{H}-CDR2, a V_{H}-CDR3, V_{L}-CDR1, a V_{L}-CDR2, and a V_{L}-CDR3, where one or more of these CDRs is defined by a consensus sequence. The consensus sequences provided herein have been derived from the alignments of CDRs depicted in FIGS. 18A-B. However, it is envisioned that alternative alignments may be done (e.g. using global or local alignment, or with different algorithms, such as Hidden Markov Models, seeded guide trees, Needleman-Wunsch algorithm, or Smith-Waterman algorithm) and as such, alternative consensus sequences can be derived.

In some embodiments, the V_{H}-CDR1 is defined by the formula X₁X₂X₃X₄X_{S}X₆X₇X_{S}X₉X₁₀, (SEQ ID No. 935) where X₁ is E, G, or R; X₂ is F, N, or Y; X₃ is A, I, K, N, S, or T; X₄ is F, I, or L; X₅ is I, K, N, R, S, or T; X₆ is D, G, I, N, S, or T; X₇ is F, G, H, S, or Y; X₈ is no amino acid, A, D, G, I, M, N, T, V, W, or Y; X₉ is no amino acid, M, or Y; X₁₀ is no amino acid or G; In some embodiments, the V_{H}-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR1 comprises a sequence having 0, 1, 2, 3, 4, 5, or 6 substitutions from this consensus sequence (SEQ ID No. 935).

In some embodiments, the V_{H}-CDR2 is defined by the formula X₁X₂X₃X₄X₅X₆X₇X₈X₉X₁₀, (SEQ ID No. 936) where X₁ is no amino acid, I, or L; X₂ is no amino acid or R; X₃ is no amino acid, F, I, L, or V; X₄ is A, D, F, H, K, L, N, S, W, or Y; X₅ is A, D, P, S, T, W, or Y; X₆ is D, E, G, H, K, N, S, V, or Y; X₇ is D, E, G, N, S, or T; X₈ is D, G, I, K, N, Q, R, S, V, or Y; X₉ is A, D, E, G, I, K, N, P, S, T, V, or Y; X₁₀ is no amino acid, I, P, S, or T. In some embodiments, the V_{H}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR2 comprises a sequence having 0, 1, 2, 3, 4, 5, or 6 substitutions from this consensus sequence (SEQ ID No. 936).

In some embodiments, the V_{H}-CDR3 is defined by the formula X₁X₂X₃X₄X_{S}X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅ (SEQ ID No. 937), where X₁ is no amino acid or A; X₂ is no amino acid, A, R, or Y; X₃ is no amino acid, A, F, H, K, L, R, S, or V; X₄ is no amino acid, A, D, K, N, R, S, or T; X₅ is no amino acid, A, D, G, H, I, L, N, P, R, S, T, V, or Y; X₆ is no amino acid, A, D, G, H, K, N, P, Q, R, S, or Y; X₇ is no amino acid, D, F, G, H, P, R, S, W, or Y; X₈ is no amino acid, A, D, E, G, I, R, or S; X₉ is no amino acid, A, C, D, E, F, G, I, N, R, S, T, V, or Y; X₁₀ is no amino acid, A, D, M, P, R, S, T, V, or Y; X₁₁ is no amino acid, A, D, E, F, L, T, V, or Y; X₁₂ is no amino acid, A, G, L, M, R, or T; X₁₃ is no amino acid, A, D, E, F, G, R, S, T, or V; X₁₄ is no amino acid, A, D, G, L, P, Q, R, S, T, V, or Y; X₁₅ is no amino acid, A, D, G, N, S, V, W, or Y; X₁₆ is no amino acid, A, D, E, F, L, P, T, V, W, or Y; X₁₇ is no amino acid, F, I, L, M, R, or Y; X₁₈ is no amino acid, A, D, G, N, or T; X₁₉ is no amino acid, F, N, S, T, V, or Y; X₂₀ is no amino acid or L; X₂₁ is no amino acid or A; X₂₂ is no amino acid or W; X₂₃ is no amino acid or F; X₂₄ is no amino acid or A; X₂₅ is no amino acid or Y. In some embodiments, the V_{H}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR3 comprises a sequence having 0, 1, 2, 3, 4, 5, or 6 substitutions from this consensus sequence (SEQ ID No. 937).

In some embodiments, the V_{L}-CDR1 is defined by the formula X₁X₂X₃X₄X₅X₇X₇X₈X₉X₁₀X₁₁X₁₂X₁₃X₁₄X₁₅X₁₆X₁₇ (SEQ ID No. 938), where X₁ is no amino acid or R; X₂ is no amino acid or S; X₃ is no amino acid, S, or T; X₄ is no amino acid, E, G, K, Q, or R; X₅ is no amino acid, A, D, G, I, N, or S; X₆ is no amino acid, I, L, or V; X₇ is no amino acid, F, L, S, or V; X₈ is no amino acid, D, E, H, N, S, T, or Y; X₉ is no amino acid, D, E, I, K, N, R, S, T, or V; X₁₀ is no amino acid, D, H, N, R, S, or Y; X₁₁ is no amino acid, A, G, N, S, T, or V; X₁₂ is no amino acid, A, I, K, N, Q, T, V, or Y; X₁₃ is no amino acid, D, G, H, K, N, S, T, or Y; X₁₄ is no amino acid, C, F, I, N, S, T, V, or Y; X₁₅ is no amino acid, D, L, N, W, or Y; X₁₆ is no amino acid, N, or D; X₁₇ is no amino acid or D. In some embodiments, the V_{L}-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence (SEQ ID No. 938). In some embodiments, the V_{L}-CDR1 comprises a sequence having 0, 1, 2, 3, 4, 5, or 6 substitutions from this consensus sequence.

In some embodiments, the V_{L}-CDR2 is defined by the formula X₁X₂X₃X₄X₅X₆X₇X₈ (SEQ ID No. 939), where X₁ is no amino acid, K, L, N, Q, or R; X₂ is no amino acid, A, L, M, or V; X₃ is no amino acid, C, K, or S; X₄ is no amino acid or T; X₅ is no amino acid, A, E, F, G, H, K, Q, R, S, W, or Y; X₆ is no amino acid, A, G, or T; X₇ is no amino acid, I, K, N, S, or T; X₈ is no amino acid, N, or S. In some embodiments, the V_{L}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR2 comprises a sequence having 0, 1, 2, 3, 4, 5, or 6 substitutions from this consensus sequence (SEQ ID No. 939).

In some embodiments, the V_{L}-CDR3 is defined by the formula X₁X₂X₃X₄X₅X₆X₇X_{S}X₉X₁₀ (SEQ ID No. 940), where X₁ is no amino acid, A, E, F, H, L, M, Q, S, V, or W; X₂ is A, H, or Q; X₃ is D, F, G, H, L, M, N, Q, S, T, W, or Y; X₄ is no amino acid or W; X₅ is A, D, I, K, L, N, Q, R, S, T, V, or Y; X₆ is D, E, H, I, K, L, N, Q, S, or T; X₇ is D, F, K, L, N, P, S, T, V, W, or Y; X₈ is H, P, or S; X₉ is F, L, P, Q, R, T, W, or Y; X₁₀ is no amino acid, T, or V. In some embodiments, the V_{L}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR3 comprises a sequence having 0, 1, 2, 3, 4, 5, or 6 substitutions from this consensus sequence (SEQ ID No. 940).

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity or similarity to the sequence selected from SEQ ID NOs: 297-373, 803, 806-820, 926. In some embodiments, the light chain variable region of the antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity or similarity to the sequence selected from SEQ ID NOs: 374-447, 821-835, 927-929. In some embodiments, the antibodies or binding fragments thereof are anti-Gal3 antibodies or binding fragments thereof.

In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity or similarity to a VL sequence, a VH sequence, a VL/VH pairing, and/or V_{L}-CDR1, V_{L}-CDR2, V_{L}-CDR3, V_{H}-CDR1, V_{H}-CDR2, V_{H}-CDR3 (including 1, 2, 3, 4, or 5 amino acid substitutions of any one or more of these CDRs) set from the heavy chain and light chain sequences as depicted in FIG. 9.

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises one of the amino acid sequences of SEQ ID NOs: 27-70, the V_{H}-CDR2 comprises one of the amino acid sequences of SEQ ID NOs: 71-111, 801, the V_{H}-CDR3 comprises one of the amino acid sequences of SEQ ID NO: 112-169, 802, the V_{L}-CDR1 comprises one of the amino acid sequences of SEQ ID NOs: 170-220, the V_{L}-CDR2 comprises one of the amino acid sequences of SEQ ID NOs: 211-247, the V_{L}-CDR3 comprises one of the amino acid sequences of SEQ ID NOs: 248-296, the heavy chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID NOs: 297-373, 803, 806-820, 926, and the light chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID NOs: 374-447, 821-835, 927-929.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity or similarity to the sequence selected from SEQ ID NOs: 448-494, 804, 836-850. In some embodiments, the antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequence selected from SEQ ID NOs: 495-538, 805, 851-865. In some embodiments, the antibodies or binding fragments thereof are anti-Gal3 antibodies or binding fragments thereof.

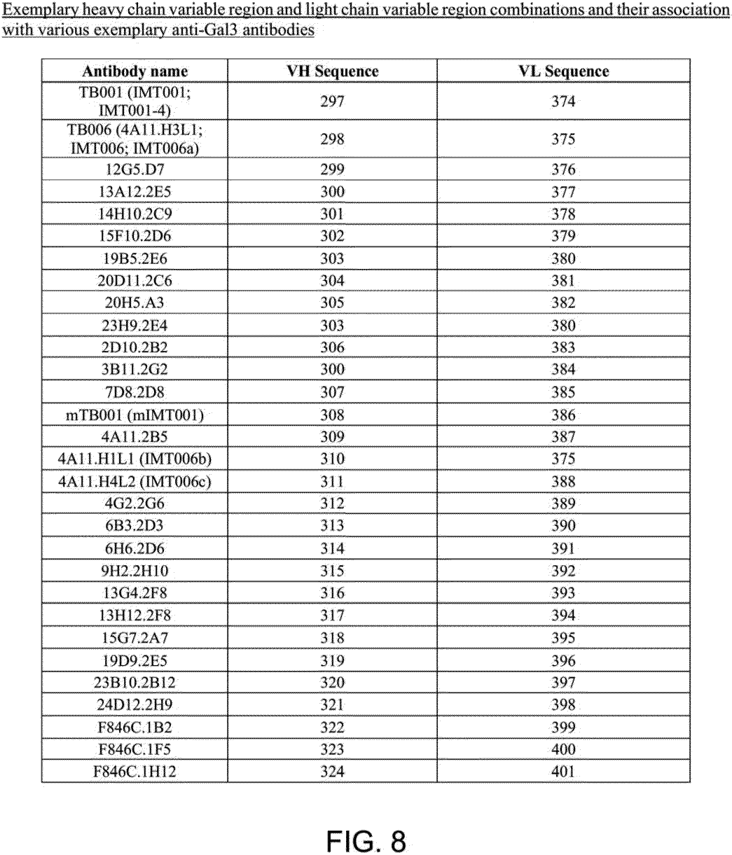

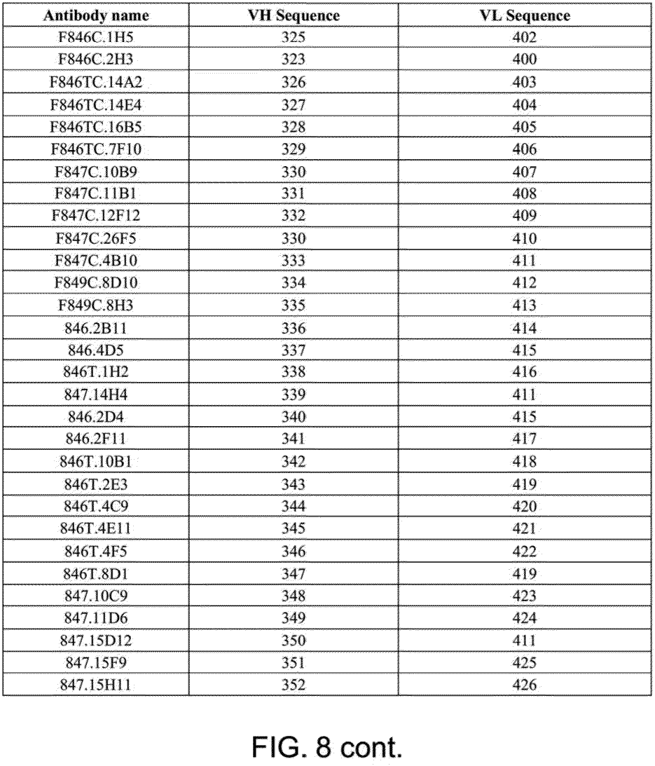

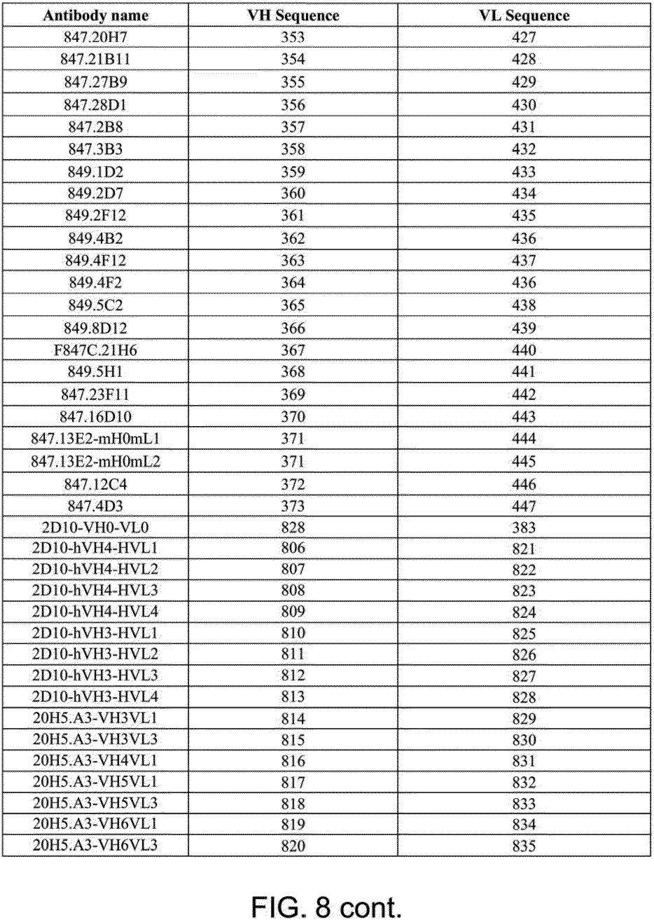

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprise a heavy chain variable region and a light chain variable region. In some embodiments, the heavy chain variable region is paired with an IgG4 heavy chain constant domain or an IgG2 heavy chain constant domain. In some embodiments, the IgG4 heavy chain constant domain or IgG2 heavy chain constant domain are human or murine. In some embodiments, the IgG4 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 931. In some embodiments, the IgG4 heavy chain constant domain is an S228P mutant. In some embodiments, the IgG2 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 933 or SEQ ID NO: 934. In some embodiments, the IgG2 heavy chain constant domain is a LALAPG or a LALA mutant. In some embodiments, the light chain variable region is paired with an IgG4 kappa chain constant domain. In some embodiments, the IgG4 kappa chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 932. In some embodiments, the light chain variable region and/or heavy chain variable region may be selected from those depicted in FIGS. 5 and 6 and/or the combinations of light chain variable region and heavy chain variable region as depicted in FIG. 8. In some embodiments, the light chain variable region and/or heavy chain variable regions comprise one or more CDRs depicted in FIGS. 3A-C, 4A-C and/or the combinations of CDRs depicted in FIG. 7.

In some embodiments, the antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

In some embodiments, the antibody or binding fragment thereof comprises a sequence (e.g. CDR, VL, VH, LC, HC) having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or similarity to a sequence of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

In some embodiments, the anti-Gal3 antibody or binding fragment thereof excludes the subset of named anti-Gal3 antibodies or binding fragments thereof depicted in any one of FIGS. 19A-D. In some embodiments, CDRs (including heavy chain and light chain CDRs), heavy chain variable regions, light chain variable regions, heavy chains, and/or light chains are excluded from the sequences associated with the subset of named anti-Gal3 antibodies or binding fragments thereof depicted in any one of FIGS. 19A-D. In some embodiments, combinations of CDRs (including heavy chain and light chain CDRs), heavy chain variable regions, light chain variable regions, heavy chains, and/or light chains are excluded from the sequences associated with the subset of named anti-Gal3 antibodies or binding fragments thereof depicted in any one of FIGS. 19A-D. In some embodiments, the anti-Gal3 antibody or binding fragment thereof excludes those selected from the group of named anti-Gal3 antibody or binding fragment thereof depicted in FIG. 19A, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19B, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19C, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19D, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19A, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19B, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19C, or excludes any one or more of the CDRs, VH, VL, HC, and/or LC thereof. In some embodiments, the anti-Gal3 antibody or binding fragment thereof excludes those selected from the group of named anti-Gal3 antibody or binding fragment thereof depicted in FIG. 19D, or excludes any one or more of the CDRs, VH, VL, HC, and/or LC thereof. In other embodiments, any of these constructs are used for any of the methods provided herein.

In some embodiments, the anti-Gal3 antibody or binding fragment thereof is selected from the subset of named anti-Gal3 antibodies or binding fragments thereof depicted in any one of FIGS. 20A-D. In some embodiments, CDRs (including heavy chain and light chain CDRs), heavy chain variable regions, light chain variable regions, heavy chains, and/or light chains are selected from the sequences associated with the subset of named anti-Gal3 antibodies or binding fragments thereof depicted in any one of FIGS. 20A-D. In some embodiments, combinations of CDRs (including heavy chain and light chain CDRs), heavy chain variable regions, light chain variable regions, heavy chains, and/or light chains are selected from the sequences associated with the subset of named anti-Gal3 antibodies or binding fragments thereof depicted in any one of FIGS. 20A-D. In some embodiments, the anti-Gal3 antibody or binding fragment thereof is selected from the group of named anti-Gal3 antibody or binding fragment thereof depicted in FIG. 20A, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 20B, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 20C, or comprises any one or more of the CDRs, VH, VL, HC, and/or LC thereof. In some embodiments, the anti-Gal3 antibody or binding fragment thereof is selected from the group of named anti-Gal3 antibody or binding fragment thereof depicted in FIG. 20D, or comprises any one or more of the CDRs, VH, VL, HC, and/or LC thereof.

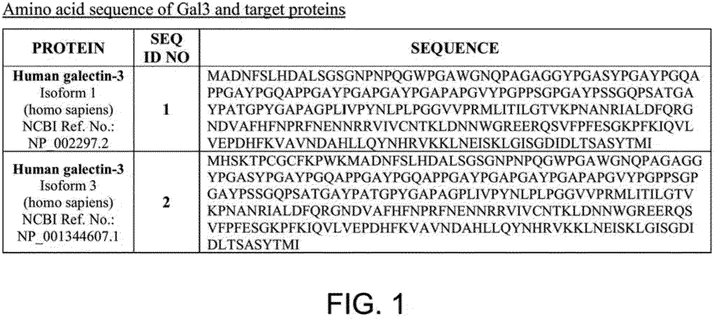

In some embodiments, the anti-Gal3 antibody or binding fragment thereof binds to specific epitopes within a Gal3 protein. In some embodiments, the anti-Gal3 antibody or binding fragment thereof binds to a specific epitope within a Gal3 protein having an amino acid sequence according to SEQ ID NO: 1-2, provided in FIG. 1.

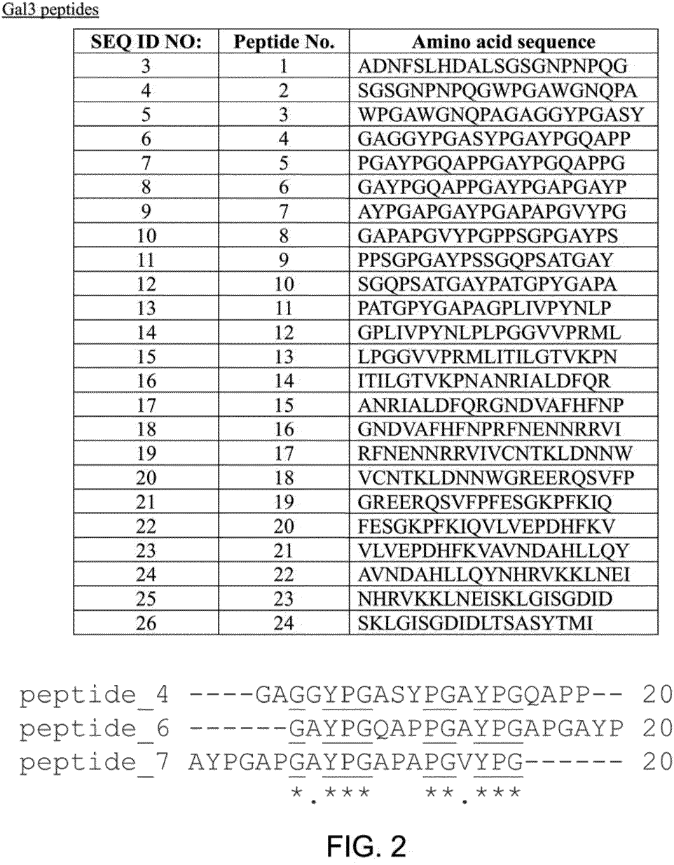

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within a peptide illustrated in FIG. 2 (SEQ ID NOs: 3-26).

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 1-20 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 31-50 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 51-70 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 61-80 of SEQ ID NO: 1-2.

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3), Peptide 4 (SEQ ID NO: 6), Peptide 6 (SEQ ID NO: 8), or Peptide 7 (SEQ ID NO: 9). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3). In some embodiments, the anti-Gal3 antibody or binding fragment thereof may bind to at least 11, 2, 3, 4, 5, 6, 7, 8, 9, 10, 11, 12, 13, 14, 15, 16, 17, 18, 19, or 20 amino acid residues within Peptide 4 (SEQ ID NO: 6). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 6 (SEQ ID NO: 8). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 7 (SEQ ID NO: 9). In some embodiments, the anti-Gal3 antibody or binding fragment thereof binds to an epitope present within a region of Gal3 defined by Peptide 1 (SEQ ID NO: 3). In some embodiments, the anti-Gal3 antibody or binding fragment thereof binds to an epitope present within a region of Gal3 defined by Peptide 4 (SEQ ID NO: 6). In some embodiments, the anti-Gal3 antibody or binding fragment thereof binds to an epitope present within a region of Gal3 defined by Peptide 6 (SEQ ID NO: 8). In some embodiments, the anti-Gal3 antibody or binding fragment thereof binds to an epitope present within a region of Gal3 defined by Peptide 7 (SEQ ID NO: 9). In some embodiments, the antibody is one that binds to 1, 2, or all 3 of peptides 1, 6, and/or 7.

In some embodiments, an anti-Gal3 antibody or binding fragment thereof as described herein may bind to the N-terminal domain of Gal3 or a portion thereof. In some embodiments, an anti-Gal3 antibody or binding fragment thereof as described herein may bind to an epitope of Gal3 that includes a motif of GxYPG, where x is the amino acids alanine (A), glycine (G), or valine (V). In some embodiments, an anti-Gal3 antibody or binding fragment thereof as described herein may bind to an epitope of Gal3 that includes two GxYPG motifs separated by three amino acids, where x is A, G, or V.

In some embodiments, the anti-Gal3 antibody or binding fragment thereof binds to Gal3. In some embodiments, the anti-Gal3 antibody or binding fragment thereof binds to the N-terminus of Gal3, the N-terminal domain of Gal3, or the TRD of Gal3. In some embodiments, the anti-Gal3 antibody or binding fragment thereof does not bind to the N-terminus of Gal3, the N-terminal domain of Gal3, or the TRD of Gal3. In some embodiments, the anti-Gal3 antibody or binding fragment thereof binds to the C-terminus of Gal3, the C-terminal domain of Gal3, or the CRD of Gal3. In some embodiments, the anti-Gal3 antibody or binding fragment thereof does not bind to the C-terminus of Gal3, the C-terminal domain of Gal3, or the CRD of Gal3. In some embodiments, the anti-Gal3 antibody or binding fragment thereof binds to Gal3 isoform 1. In some embodiments, the anti-Gal3 antibody or binding fragment thereof binds to the N-terminus of Gal3 isoform 1, the N-terminal domain of Gal3 isoform 1, amino acids 1-111 of Gal3 isoform 1, the TRD of Gal3 isoform 1, or amino acids 36-109 of Gal3 isoform 1. In some embodiments, the anti-Gal3 antibody or binding fragment thereof does not bind to the N-terminus of Gal3 isoform 1, the N-terminal domain of Gal3 isoform 1, amino acids 1-111 of Gal3, the TRD of Gal3 isoform 1, or amino acids 36-109 of Gal3 isoform 1. In some embodiments, the anti-Gal3 antibody or binding fragment thereof binds to the C-terminus of Gal3 isoform 1, the C-terminal domain of Gal3 isoform 1, amino acids 112-250 of Gal3, or the CRD of Gal3. In some embodiments, the anti-Gal3 antibody or binding fragment thereof does not bind to the C-terminus of Gal3 isoform 1, the C-terminal domain of Gal3 isoform 1, amino acids 112-250 of Gal3 isoform 1, or the CRD of Gal3. In some embodiments, the anti-Gal3 antibody or binding fragment thereof binds to the N-terminus of Gal3 isoform 3, the N-terminal domain of Gal3 isoform 3, amino acids 1-125 of Gal3, the TRD of Gal3 isoform 3, or amino acids 50-123 of Gal3 isoform 3. In some embodiments, the anti-Gal3 antibody or binding fragment thereof does not bind to the N-terminus of Gal3 isoform 3, the N-terminal domain of Gal3 isoform 3, amino acids 1-125 of Gal3 isoform 3, the TRD of Gal3, or amino acids 50-123 of Gal3 isoform 3. In some embodiments, the anti-Gal3 antibody or binding fragment thereof binds to the C-terminus of Gal3 isoform 3, the C-terminal domain of Gal3 isoform 3, amino acids 126-264 of Gal3 isoform 3, or the CRD of Gal3. In some embodiments, the anti-Gal3 antibody or binding fragment thereof does not bind to the C-terminus of Gal3 isoform 3, the C-terminal domain of Gal3 isoform 3, amino acids 126-264 of Gal3 isoform 3, or the CRD of Gal3 isoform 3.

In some embodiments, the anti-Gal3 antibody or binding fragment thereof reduces an interaction between Gal3 and a target protein to less than 80%, less than 75%, less than 70%, less than 60%, less than 59%, less than 50%, less than 40%, less than 34%, less than 30%, less than 20%, less than 14%, less than 10%, less than 7%, less than 5%, less than 4%, or less than 1%.

In some embodiments, the anti-Gal3 antibody or binding fragment thereof binds to Gal3 with a dissociation constant (K_{D}) of less than 1 nM, less than 1.2 nM, less than 2 nM, less than 5 nM, less than 10 nM, less than 13.5 nM, less than 15 nM, less than 20 nM, less than 25 nM, or less than 30 nM. In some embodiments, the anti-Gal3 antibody or binding fragment thereof binds to Gal3 with a K_{D} of less than 1 nM. In some embodiments, the anti-Gal3 antibody or binding fragment thereof binds to Gal3 with a K_{D} of less than 1.2 nM. In some embodiments, the anti-Gal3 antibody or binding fragment thereof binds to Gal3 with a K_{D} of less than 2 nM. In some embodiments, the anti-Gal3 antibody or binding fragment thereof binds to Gal3 with a K_{D} of less than 5 nM. In some embodiments, the anti-Gal3 antibody or binding fragment thereof binds to Gal3 with a K_{D} of less than 10 nM. In some embodiments, the anti-Gal3 antibody or binding fragment thereof binds to Gal3 with a K_{D} of less than 13.5 nM. In some embodiments, the anti-Gal3 antibody or binding fragment thereof binds to Gal3 with a K_{D} of less than 15 nM. In some embodiments, the anti-Gal3 antibody or binding fragment thereof binds to Gal3 with a K_{D} of less than 20 nM. In some embodiments, the anti-Gal3 antibody or binding fragment thereof binds to Gal3 with a K_{D} of less than 25 nM. In some embodiments, the anti-Gal3 antibody or binding fragment thereof binds to Gal3 with a K_{D} of less than 30 nM.

In some embodiments, the anti-Gal3 antibody or binding fragment thereof comprises any one of the variable heavy chain complementarity-determining region 1 (V_{H}-CDR1) sequences illustrated in FIG. 3A (SEQ ID NOs: 27-70). In some embodiments, the anti-Gal3 antibody comprises a V_{H}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or similarity to any one of SEQ ID NOs: 27-70.

In some embodiments, the anti-Gal3 antibody or binding fragment thereof comprises any one of the variable heavy chain complementarity-determining region 2 (V_{H}-CDR2) sequences illustrated in FIG. 3B (SEQ ID NOs: 71-111, 801). In some embodiments, the anti-Gal3 antibody or binding fragment thereof comprises a V_{H}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or similarity to any one of SEQ ID NOs: 71-111, 801.

In some embodiments, the anti-Gal3 antibody or binding fragment thereof comprises any one of the variable heavy chain complementarity-determining region 3 (V_{H}-CDR3) sequences illustrated in FIG. 3C (SEQ ID NOs: 112-169, 802). In some embodiments, the anti-Gal3 antibody or binding fragment thereof comprises a V_{H}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or similarity to any one of SEQ ID NOs: 112-169, 802.

In some embodiments, the anti-Gal3 antibody or binding fragment thereof comprises any one of the variable light chain complementarity-determining region 1 (V_{L}-CDR1) sequences illustrated in FIG. 4A (SEQ ID NOs: 170-220). In some embodiments, the anti-Gal3 antibody or binding fragment thereof comprises a V_{L}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or similarity to any one of SEQ ID NOs: 170-220.

In some embodiments, the anti-Gal3 antibody or binding fragment thereof comprises any one of the variable light chain complementarity-determining region 2 (V_{L}-CDR2) sequences illustrated in FIG. 4B (SEQ ID NOs: 221-247). In some embodiments, the anti-Gal3 antibody or binding fragment thereof comprises a V_{L}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or similarity to any one of SEQ ID NOs: 221-247.

In some embodiments, the anti-Gal3 antibody or binding fragment thereof comprises any one of the variable light chain complementarity-determining region 3 (V_{L}-CDR3) sequences illustrated in FIG. 4C (SEQ ID NOs: 248-296). In some embodiments, the anti-Gal3 antibody or binding fragment thereof comprises a V_{L}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or similarity to any one of SEQ ID NOs: 248-296.

In some embodiments, the anti-Gal3 antibody or binding fragment thereof comprises a heavy chain variable region (V_{H}) and a light chain variable region (V_{L}). In some embodiments, the V_{H} may comprise a V_{H}-CDR1, a V_{H}-CDR2, and/or a V_{H}-CDR3 selected from any of FIGS. 3A-C. In some embodiments, the V_{L} may comprise a V_{L}-CDR1, a V_{L}-CDR2, and/or a V_{L}-CDR3 selected from any of FIGS. 4A-C. In some embodiments, the anti-Gal3 antibody or binding fragment thereof comprises CDRs within the V_{H} and V_{L} sequences as illustrated in FIGS. 5 and 6.

In some embodiments, the anti-Gal3 antibody or binding fragment thereof comprises a heavy chain variable region (V_{H}) sequence selected from FIG. 5 (SEQ ID NOs: 297-373, 803, 806-820, 926). In some embodiments, the anti-Gal3 antibody or binding fragment thereof comprises a V_{H}- sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or similarity to any one of SEQ ID NOs: 297-373, 803, 806-820, 926.

In some embodiments, the anti-Gal3 antibody or binding fragment thereof comprises a light chain variable region (V_{L}) sequence selected from FIG. 6 (SEQ ID NOs: 374-447, 821-835, 927-929). In some embodiments, the anti-Gal3 antibody or binding fragment thereof comprises a V_{L} sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or similarity to any one of SEQ ID NOs: 374-447, 821-835, 927-929.

In some embodiments, the anti-Gal3 antibody or binding fragment thereof comprises a combination of heavy chain variable region and light chain variable region as illustrated in FIG. 8.

In some embodiments, the anti-Gal3 antibody or binding fragment thereof comprises heavy chain and light chain sequences as illustrated in FIG. 9 (SEQ ID NOs: 448-538, 804-805, 836-865.

In some embodiments, the anti-Gal3 antibody or binding fragment thereof is selected from the group of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or a binding fragment thereof.

In some embodiments, the anti-Gal3 antibody or binding fragment thereof comprises, consists essentially of, or consists of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or a binding fragment thereof.

In some embodiments, the anti-Gal3 antibody or binding fragment thereof comprises one or more heavy chain variable region CDRs depicted in FIGS. 3A-C. In some embodiments, the anti-Gal3 antibody or binding fragment thereof comprises one or more light chain variable region CDRs depicted in FIGS. 4A-C. In some embodiments, the anti-Gal3 antibody or binding fragment thereof comprises a heavy chain variable region depicted in FIG. 5. In some embodiments, the anti-Gal3 antibody or binding fragment thereof comprises a light chain variable region depicted in FIG. 6. In some embodiments, the anti-Gal3 antibody or binding fragment thereof comprises a combination of heavy chain variable region and light chain variable region depicted in FIG. 8. In some embodiments, the anti-Gal3 antibody or binding fragment thereof comprises a heavy chain and/or light chain depicted in FIG. 9. In some embodiments, the anti-Gal3 antibody or binding fragment thereof can comprise or include any one or more of the sequences provided in any one or more of FIGS. 3A-C, 4A-C, 5, 6, 7, 8, 9, or any one or more of a sequence that is at least 80%, 81%, 82%, 83%, 84%, 85%, 86%, 87%, 88%, 89%, 90%, 91%, 92%, 93%, 94%, 95%, 96%, 97%, 98%, 99% or greater identical thereto. In some embodiments, the anti-Gal3 antibody or binding fragment thereof can comprise or include any one or more of the sequences provided in any one or more of FIGS. 3A-C, 4A-C, 5, 6, 7, 8, 9, or any one or more of a sequence that is at least 80%, 81%, 82%, 83%, 84%, 85%, 86%, 87%, 88%, 89%, 90%, 91%, 92%, 93%, 94%, 95%, 96%, 97%, 98%, 99% or greater similar thereto.

In some embodiments, the anti-Gal3 antibody or binding fragment thereof comprises a humanized antibody or binding fragment thereof. In other instances, the anti-Gal3 antibody or binding fragment thereof comprises a chimeric antibody or binding fragment thereof. In some embodiments, the anti-Gal3 antibody comprises a full-length antibody or a binding fragment thereof. In some embodiments, the anti-Gal3 antibody or binding fragment thereof comprises a bispecific antibody or a binding fragment thereof. In some embodiments, the anti-Gal3 antibody or binding fragment thereof comprises a monovalent Fab′, a divalent Fab2, a single-chain variable fragment (scFv), a diabody, a minibody, a nanobody, a single-domain antibody (sdAb), or a camelid antibody or binding fragment thereof.

In some embodiments, the anti-Gal3 antibody or binding fragment thereof is a bispecific antibody or binding fragment thereof. Exemplary bispecific antibody formats include, but are not limited to, Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), Azymetric, Biclonics, Fab-scFv-Fc, Two-in-one/Dual Action Fab (DAF), FinomAb, scFv-Fc-(Fab)-fusion, Dock-aNd-Lock (DNL), Tandem diAbody (TandAb), Dual-affinity-ReTargeting (DART), nanobody, triplebody, tandems scFv (taFv), triple heads, tandem dAb/VHH, triple dAb/VHH, or tetravalent dAb/VHH. In some embodiments, the anti-Gal3 antibody or binding fragment thereof is a bispecific antibody or binding fragment thereof comprising a bispecific antibody format illustrated in Brinkmann and Kontermann, “The making of bispecific antibodies,” MABS 9(2): 182-212 (2017).

In some embodiments, the anti-Gal3 antibody or binding fragment thereof can comprise an IgM, IgG (e.g., IgG1, IgG2, IgG3, or IgG4), IgA, or IgE framework. The IgG framework can be IgG1, IgG2, IgG3 or IgG4. In some embodiments, the anti-Gal3 antibody or binding fragment thereof comprises an IgG1 framework. In some embodiments, the anti-Gal3 antibody or binding fragment thereof comprises an IgG2 framework. In some embodiments, the anti-Gal3 antibody or binding fragment thereof comprises an IgG4 framework. The anti-Gal3 antibody or binding fragment thereof can further comprise a Fc mutation.

In some embodiments, the Fc region comprises one or more mutations that modulate Fc receptor interactions, e.g., to enhance effector functions such as ADCC and/or CDC. In such instances, exemplary residues when mutated modulate effector functions include S239, K326, A330, I332, or E333, in which the residue position correspond to IgG1 and the residue numbering is in accordance to Kabat numbering (EU index of Kabat et al 1991 Sequences of Proteins of Immunological Interest). In some embodiments, the one or more mutations comprise S239D, K326W, A330L, I332E, E333A, E333S, or a combination thereof. In some embodiments, the one or more mutations comprise S239D, I332E, or a combination thereof. In some embodiments, the one or more mutations comprise S239D, A330L, I332E, or a combination thereof. In some embodiments, the one or more mutations comprise K326W, E333S, or a combination thereof. In some embodiments, the mutation comprises E333A.

In some embodiments, an anti-Gal3 antibody or binding fragment thereof comprises a humanization score of above 70, above 80, above 81, above 82, above 83, above 84, above 85, above 86, above 87, above 88, above 89, above 90, or above 95. In some embodiments, the anti-Gal3 antibody or binding fragment thereof comprises a humanization score of above 80. In some embodiments, the anti-Gal3 antibody or binding fragment thereof comprises a humanization score of above 83. In some embodiments, the anti-Gal3 antibody or binding fragment thereof comprises a humanization score of above 85. In some embodiments, the anti-Gal3 antibody or binding fragment thereof comprises a humanization score of above 87. In some embodiments, the anti-Gal3 antibody or binding fragment thereof comprises a humanization score of above 90. In some embodiments, the anti-Gal3 antibody or binding fragment thereof comprises a humanization score of the heavy chain of above 70, above 80, above 81, above 82, above 83, above 84, above 85, above 86, above 87, above 88, above 89, above 90, or above 95, optionally above 80, above 85, or above 87. In some embodiments, the anti-Gal3 antibody or binding fragment thereof comprises a humanization score of the light chain of above 70, above 80, above 81, above 82, above 83, above 84, above 85, above 86, above 87, above 88, above 89, above 90, or above 95, optionally above 80, above 83, or above 85.

### Pharmaceutical Formulations

A pharmaceutical formulation for treating a disease as provided herein can comprise an anti-Gal3 antibody or binding fragment thereof described herein. The anti-Gal3 antibody or binding fragment thereof can be formulated for systemic administration. Alternatively, the anti-Gal3 antibody or binding fragment thereof can be formulated for parenteral administration.

In some embodiments, an anti-Gal3 antibody or binding fragment thereof is formulated as a pharmaceutical composition for administration to a subject by, but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some instances, the pharmaceutical composition described herein is formulated for parenteral (e.g., intravenous, subcutaneous, intramuscular, intraarterial, intradermal, intraperitoneal, intravitreal, intracerebral, or intracerebroventricular) administration. In other instances, the pharmaceutical composition described herein is formulated for systemic administration. In other instances, the pharmaceutical composition described herein is formulated for oral administration. In still other instances, the pharmaceutical composition described herein is formulated for intranasal administration. In some embodiments, the antibodies or binding fragments thereof disclosed herein are provided in a subcutaneous formulation or an intravenous formulation. In some embodiments, the antibodies or binding fragments thereof disclosed herein are provided in an intravenous formulation.

In some instances, the pharmaceutical compositions further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and trishydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical compositions further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner’s sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multi-particulate formulations (e.g., nanoparticle formulations), and mixed immediate and controlled release formulations.

Provided herein in some embodiments are pharmaceutical formulations of any one or more of the anti-Gal3 antibodies or binding fragments disclosed herein. These pharmaceutical formulations may be in the form of prepared solutions that are compatible for administration, such as contained in a sterile IV infusion bag or other container. In some embodiments, the pharmaceutical formulations are prepared as unit doses. In some embodiments, the pharmaceutical formulations are unit doses formulated as 50, 60, 70, 80, 90, 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500 or 5000 mg of the anti-Gal3 antibody or binding fragment thereof per unit dose, or any amount per unit dose within a range defined by any two of the aforementioned amounts per unit dose. In some embodiments, the pharmaceutical formulations are unit doses formulated as 1000 mg or about 1000 mg of the anti-Gal3 antibody or binding fragment thereof. In some embodiments, the pharmaceutical formulations are unit doses (such as in the amounts provided herein) prepared in 100, 110, 120, 130, 140, 150, 160, 170, 180, 190, 200, 210, 220, 230, 240, 250, 260, 270, 280, 290, or 300 mL of sterile saline or other compatible excipient, carrier, or diluent known in the art. In some embodiments, the pharmaceutical formulations are unit doses (such as in the amounts provided herein) prepared in 250 mL or about 250 mL of sterile saline or other compatible excipient, carrier, or diluent known in the art.

Also provided herein are pharmaceutical formulations comprising 1000 mg or about 1000 mg of an anti-Gal3 antibody or binding fragment thereof dissolved in 250 mL or about 250 mL of saline. In some embodiments, the pharmaceutical formulation is prepared in an IV infusion bag.

Also provided herein are pharmaceutical formulations comprising 1000 mg or about 1000 mg of an anti-Gal3 antibody diluted in 0.9% Sodium Chloride Injection, USP (normal sterile saline) in a volume of 250 mL. In some embodiments, the anti-Gal3 antibody is TB006.

Also provided herein are single-use sealed injectable glass vials comprising a pharmaceutical formulation of any one or more of the anti-Gal3 antibodies or binding fragments thereof disclosed herein. In some embodiments, the vials comprise 4, 5, 6, 7, 8, 9, 10, 11, or 12 mL of a concentrated solution of the anti-Gal3 antibody or binding fragment thereof. In some embodiments, the vials comprise 8 mL of a concentrated solution of the anti-Gal3 antibody or binding fragment thereof. In some embodiments, the concentrated solution, which is meant to be diluted prior to administration, is 15, 16, 17, 18, 19, 20, 21, 22, 23, 24, or 25 mg/mL. In some embodiments, the concentrated solution, which is meant to be diluted prior to administration, is 20 mg/mL of the anti-Gal3 antibody or binding fragment thereof. In some embodiments, the pharmaceutical compositions disclosed herein for administration (e.g. those prepared in an IV infusion bag or other container) may be prepared by diluting the concentrated solution in the vials.

Also provided herein are single-use sealed injectable glass vials comprising 8 mL or about 8 mL of 20 mg/mL or about 20 mg/mL (i.e. total of 160 mg or about 160 mg) of an anti-Gal3 antibody or binding fragment thereof.

Also provided herein are pharmaceutical formulations comprising TB006 supplied in 8 mL glass vials, sealed with a rubber stopper and an aluminum flip top, with a strength of 160 mg (20 mg/mL, 8 mL) TB006 per vial. In some embodiments, the pharmaceutical formulation is stored at 2-8° C.

### Polynucleotides and Vectors

In some embodiments, the present disclosure provides isolated nucleic acids encoding any of the anti-Gal3 antibodies or binding fragments thereof disclosed herein. In another embodiment, the present disclosure provides vectors comprising a nucleic acid sequence encoding any anti-Gal3 antibody or binding fragment thereof disclosed herein. In some embodiments, this disclosure provides isolated nucleic acids that encode heavy chain variable regions, light chain variable regions, heavy chains, or light chains of an anti-Gal3 antibody or binding fragment thereof disclosed herein.

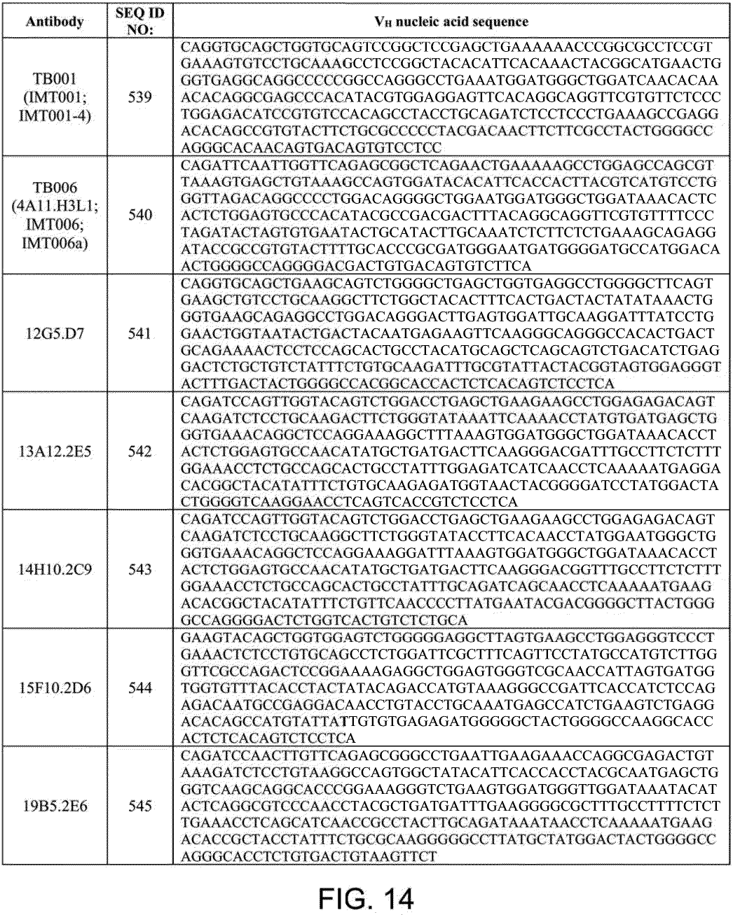

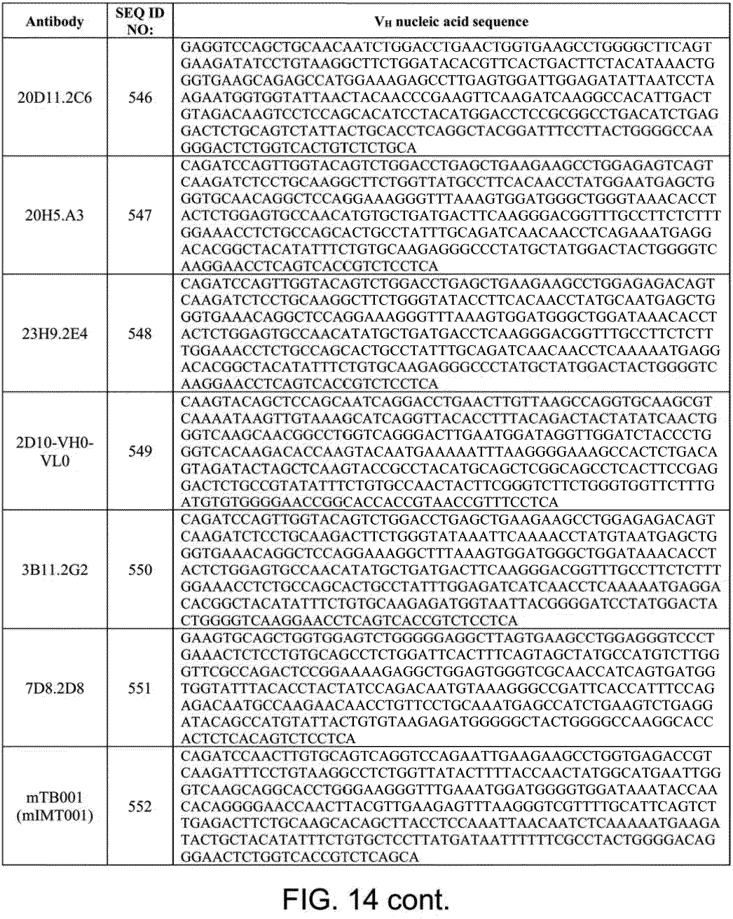

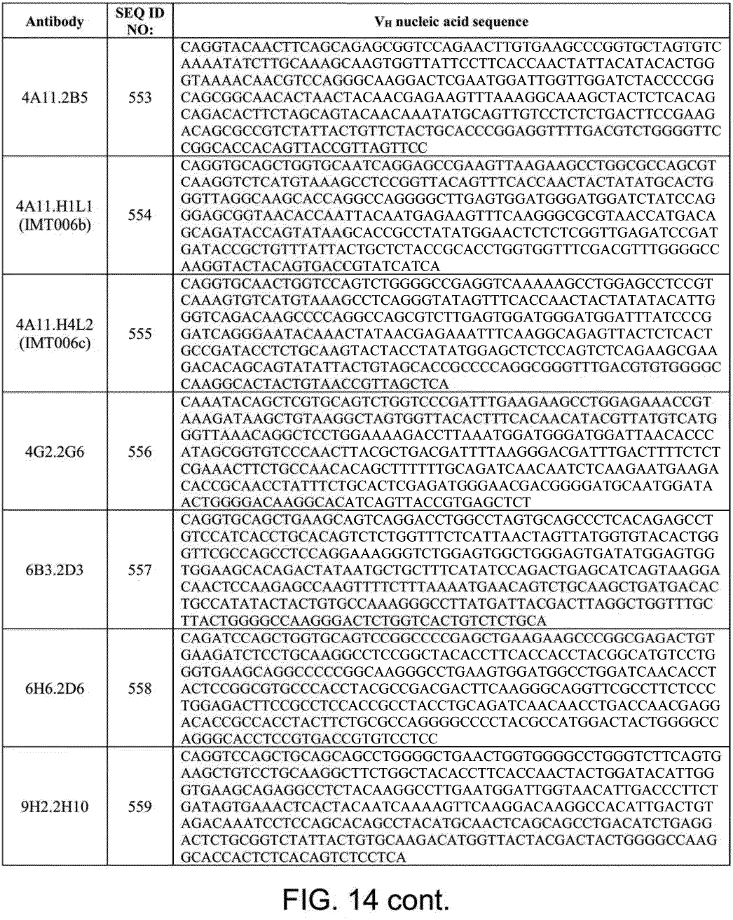

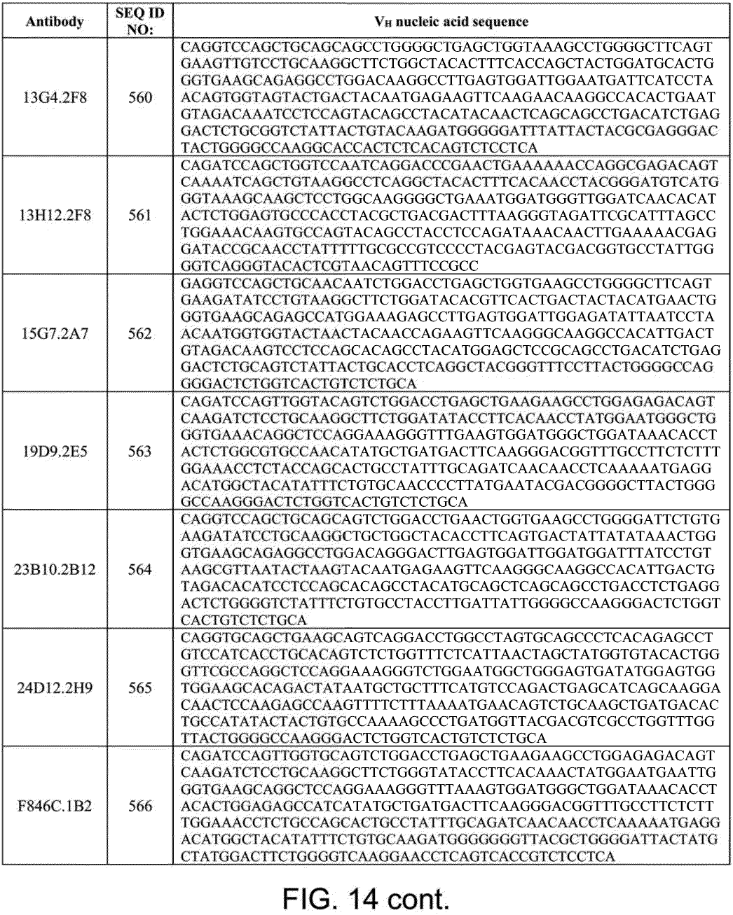

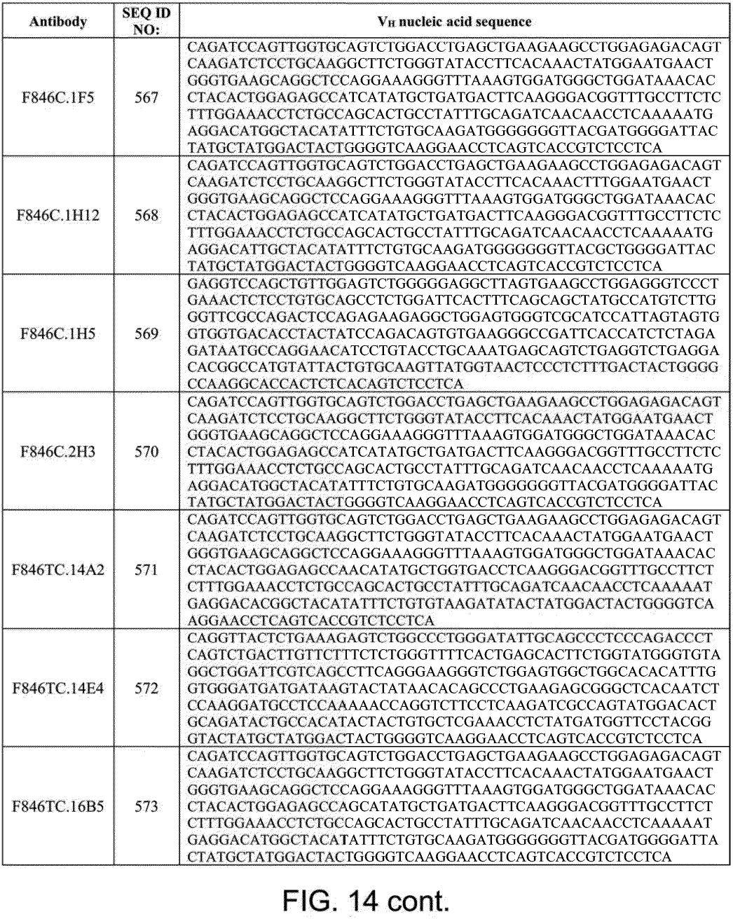

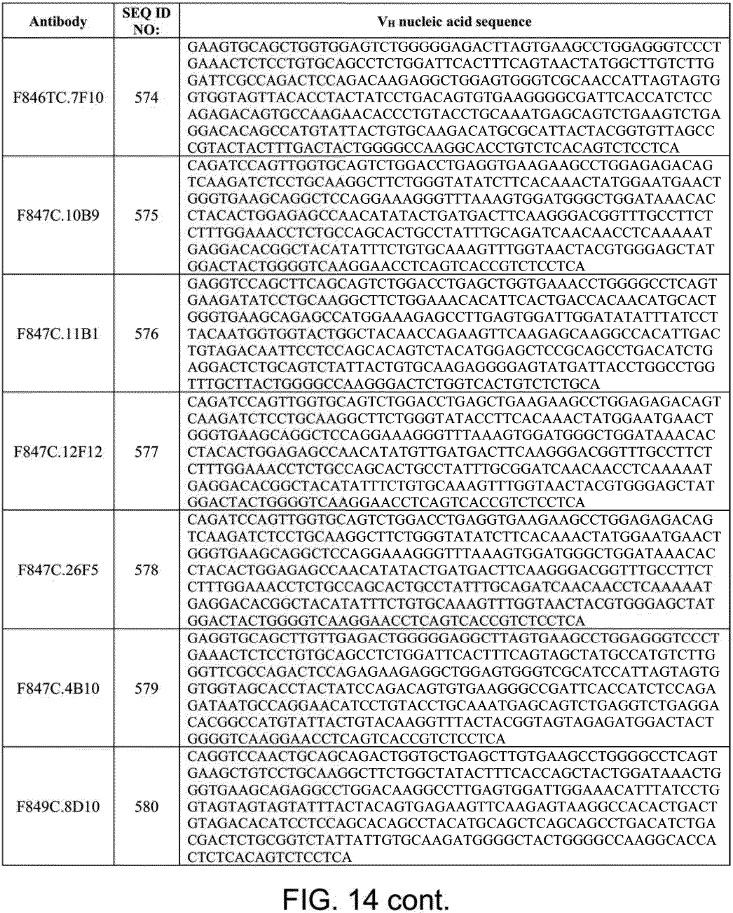

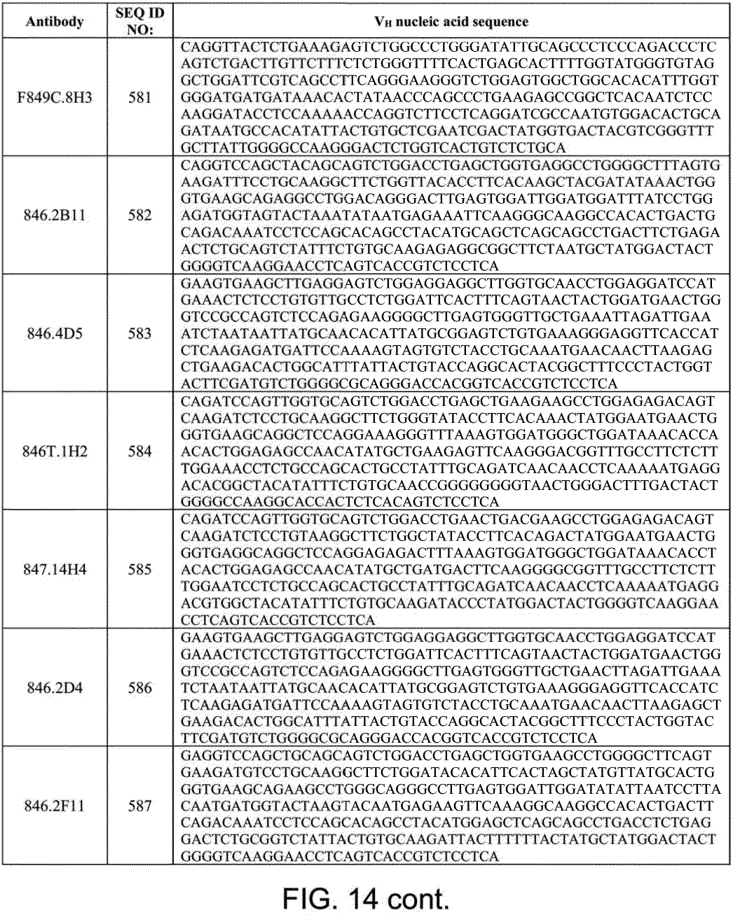

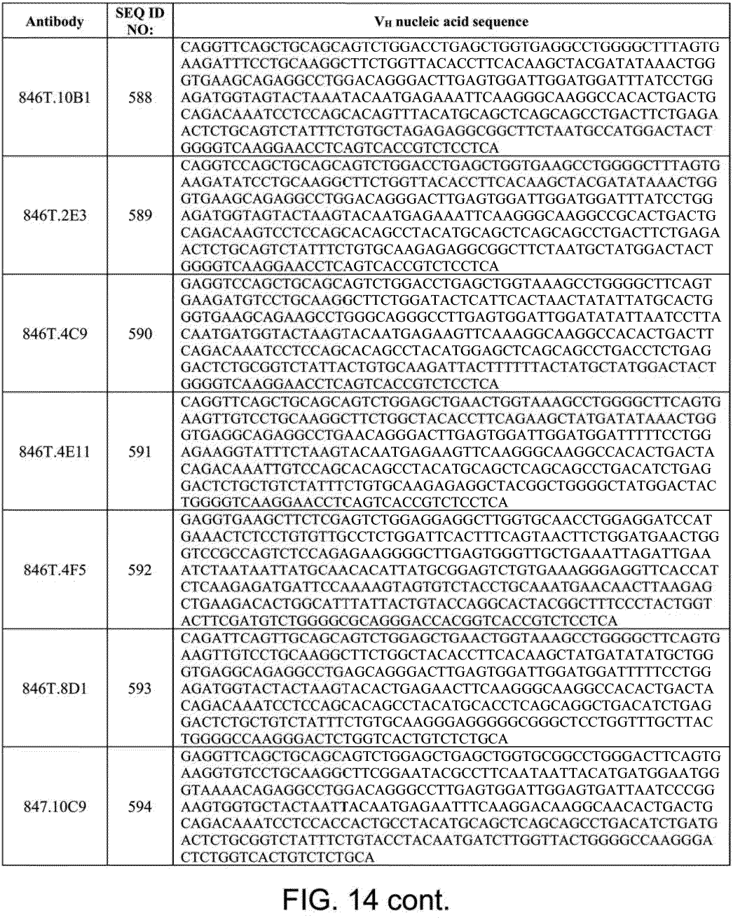

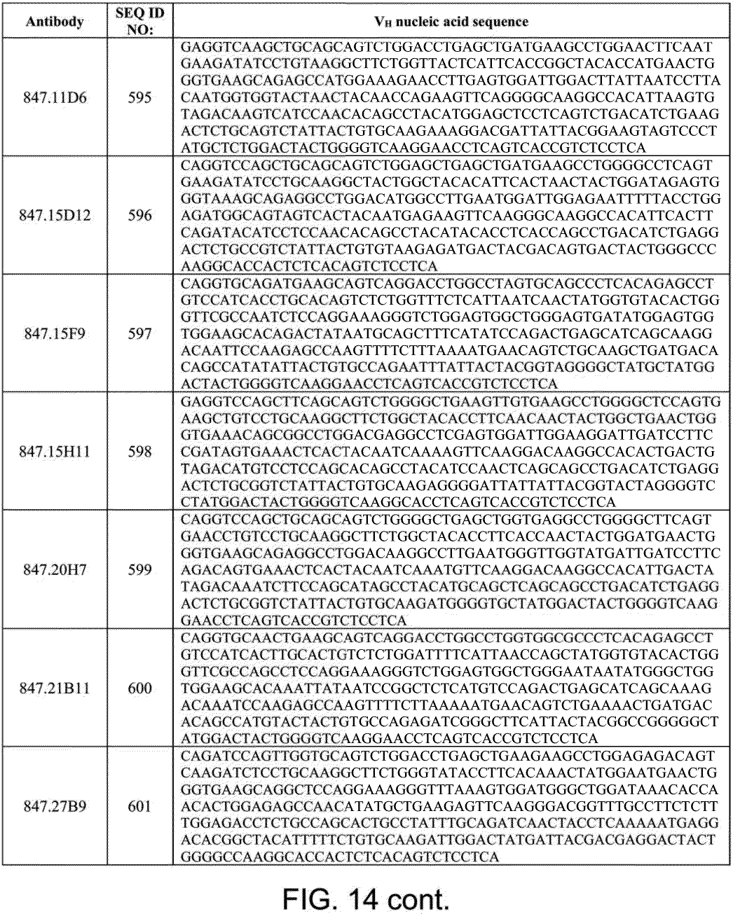

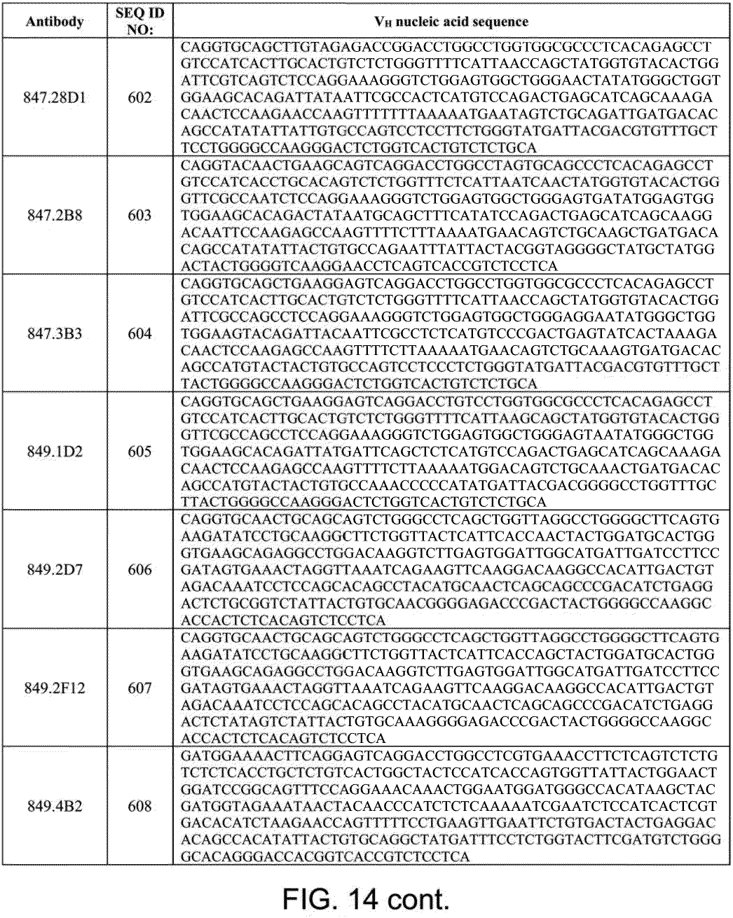

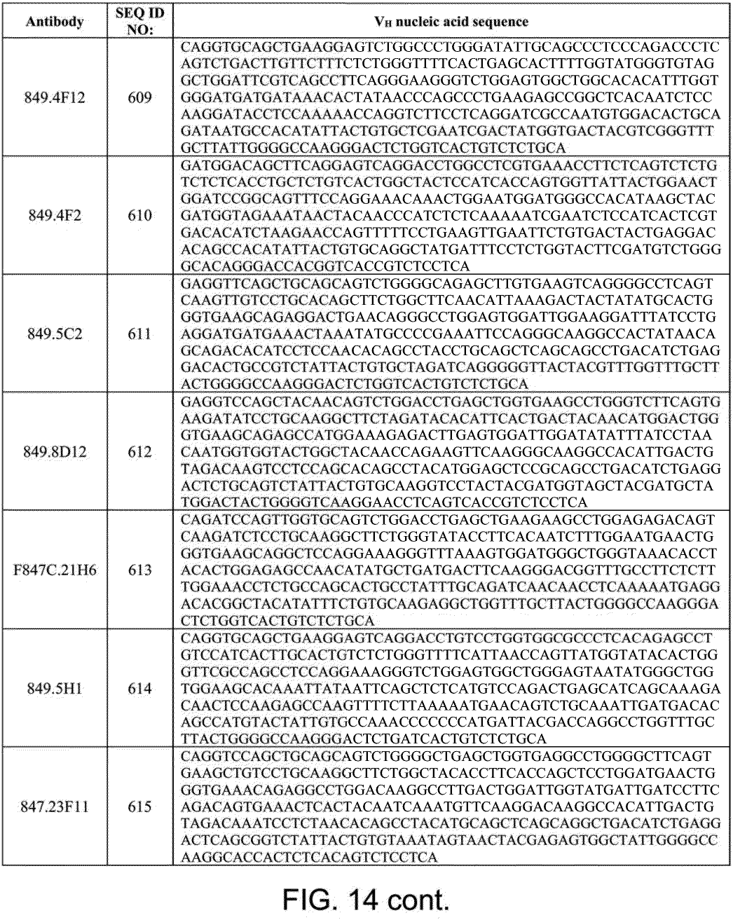

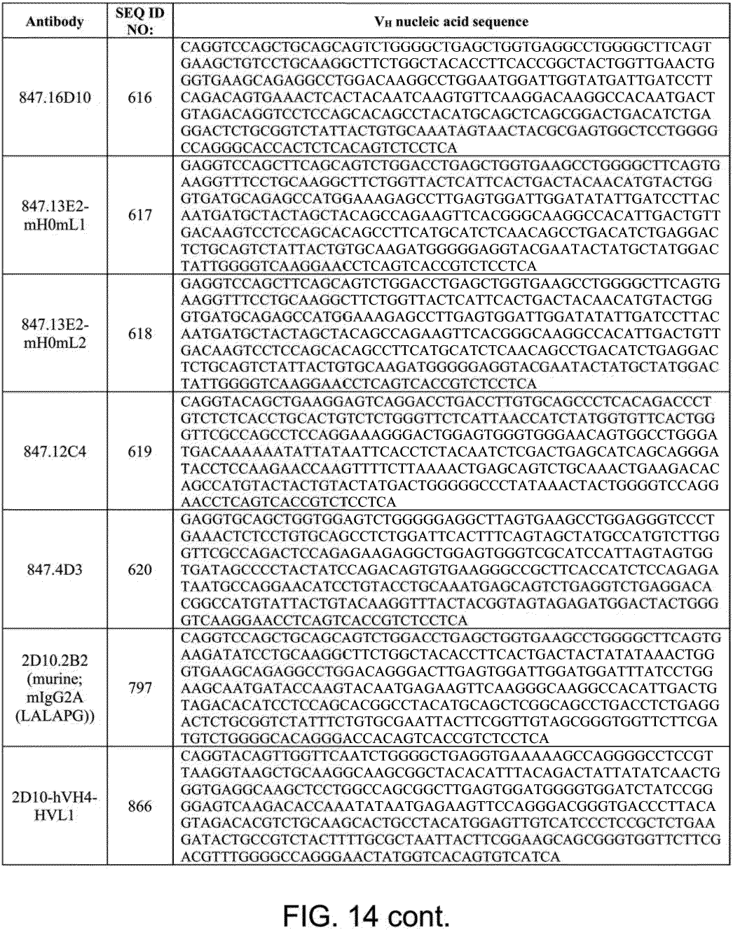

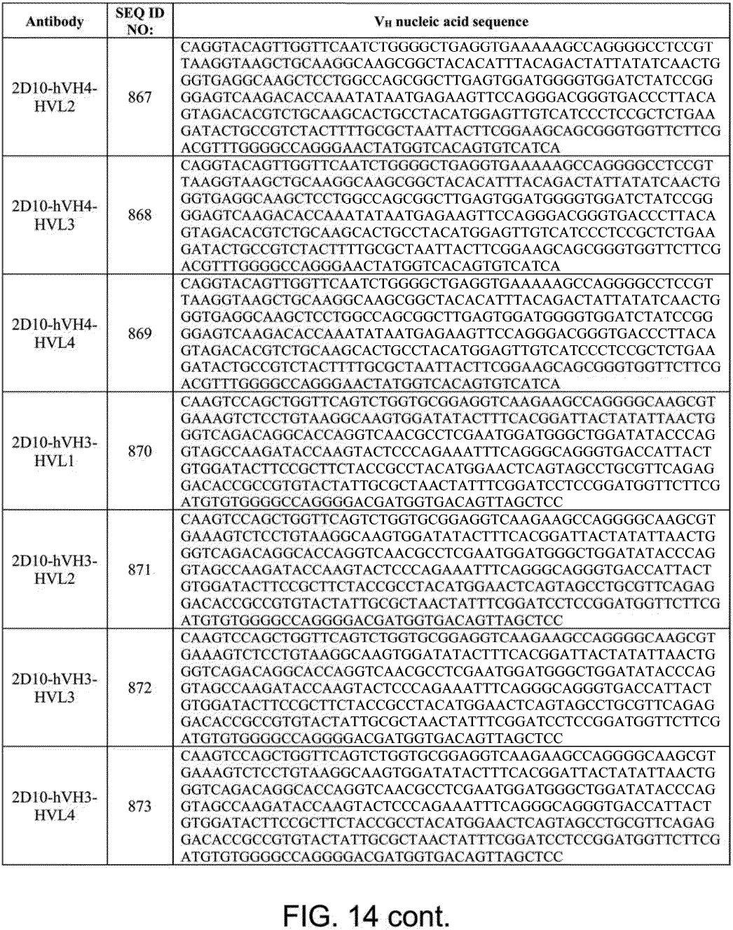

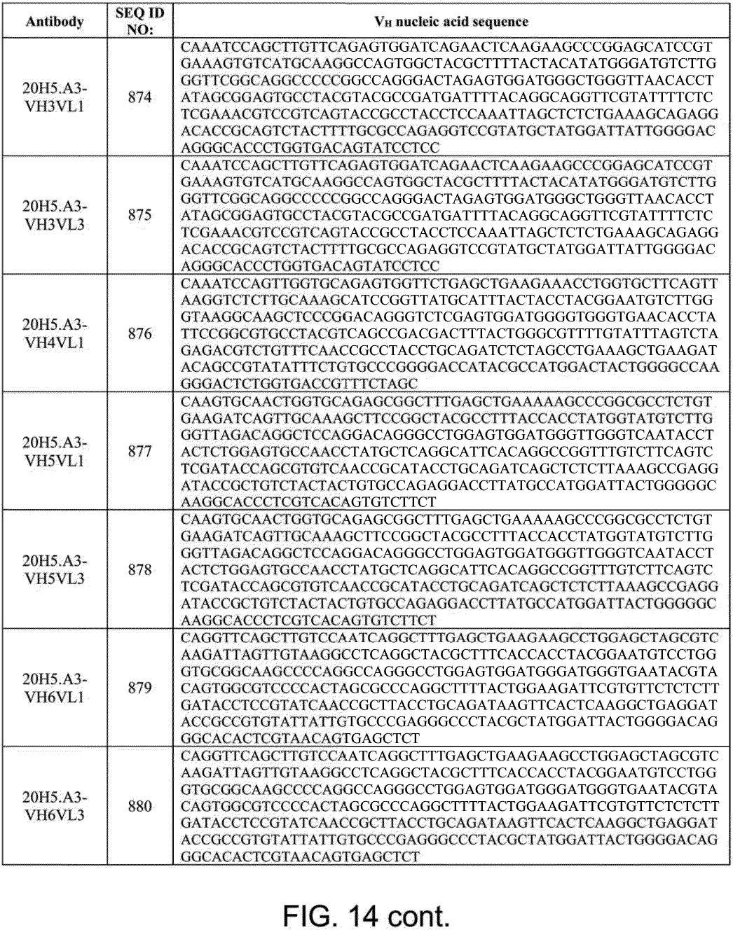

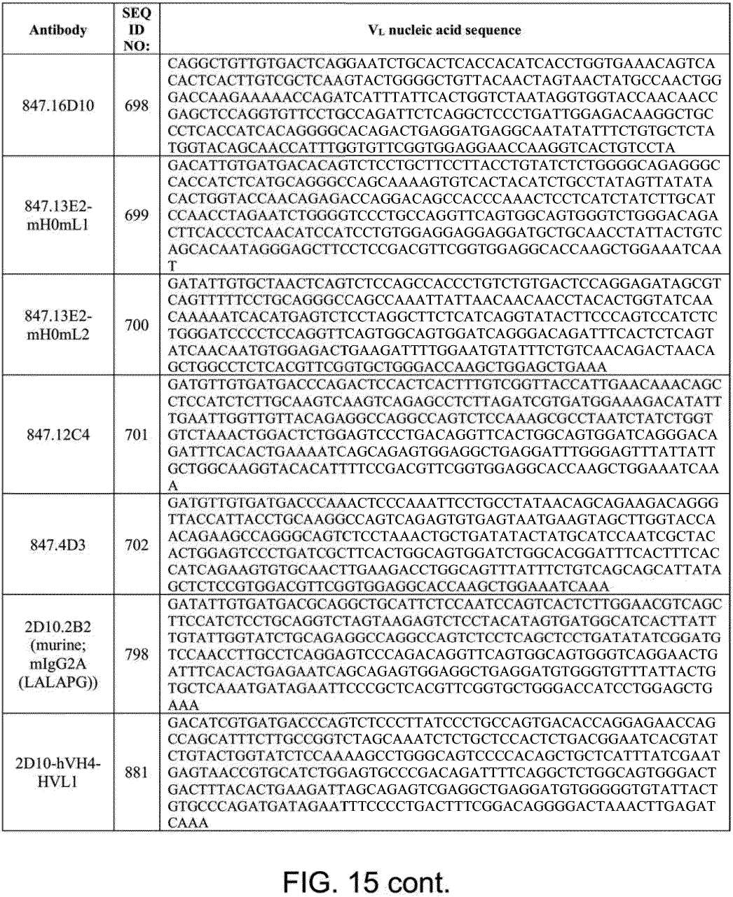

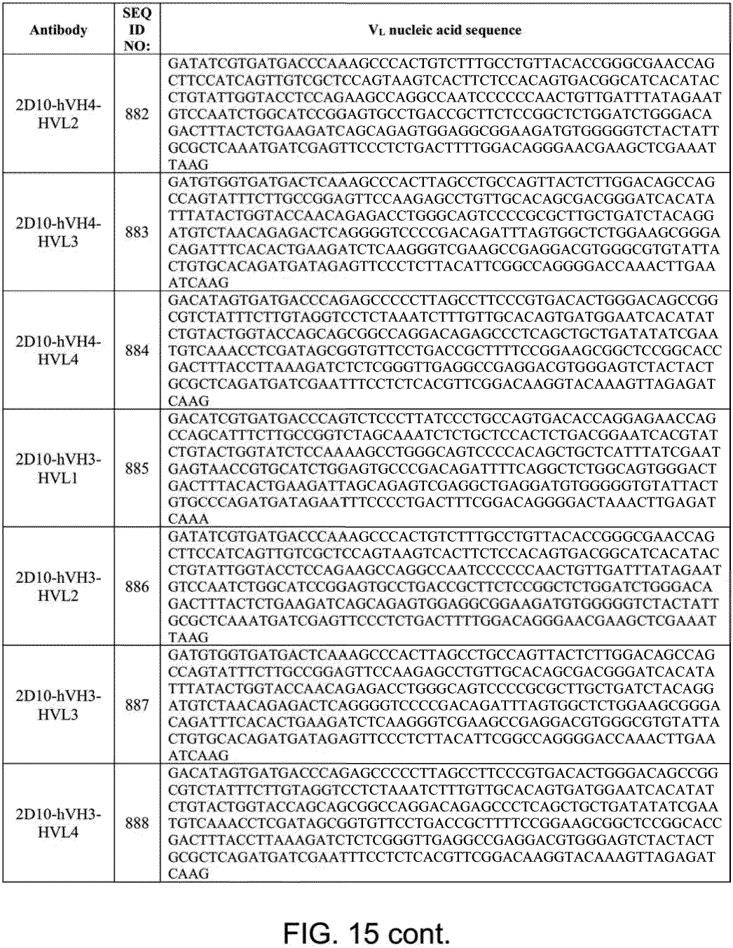

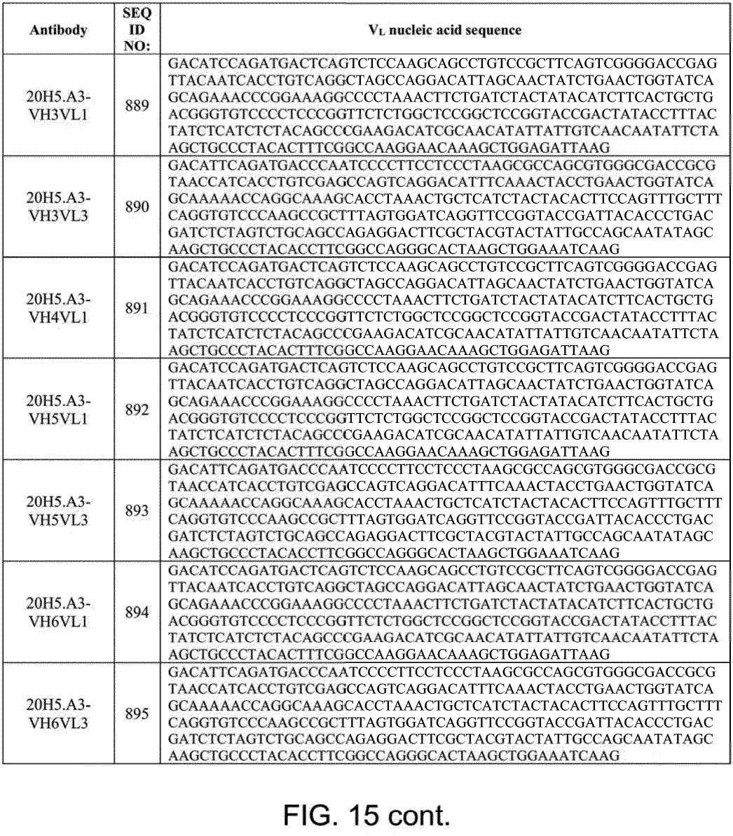

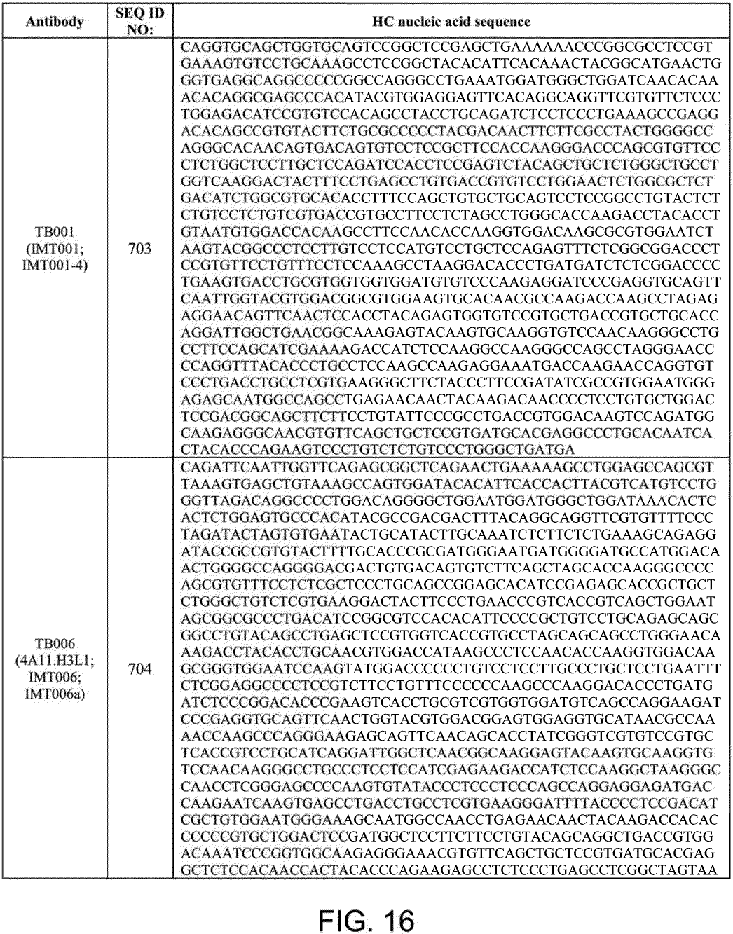

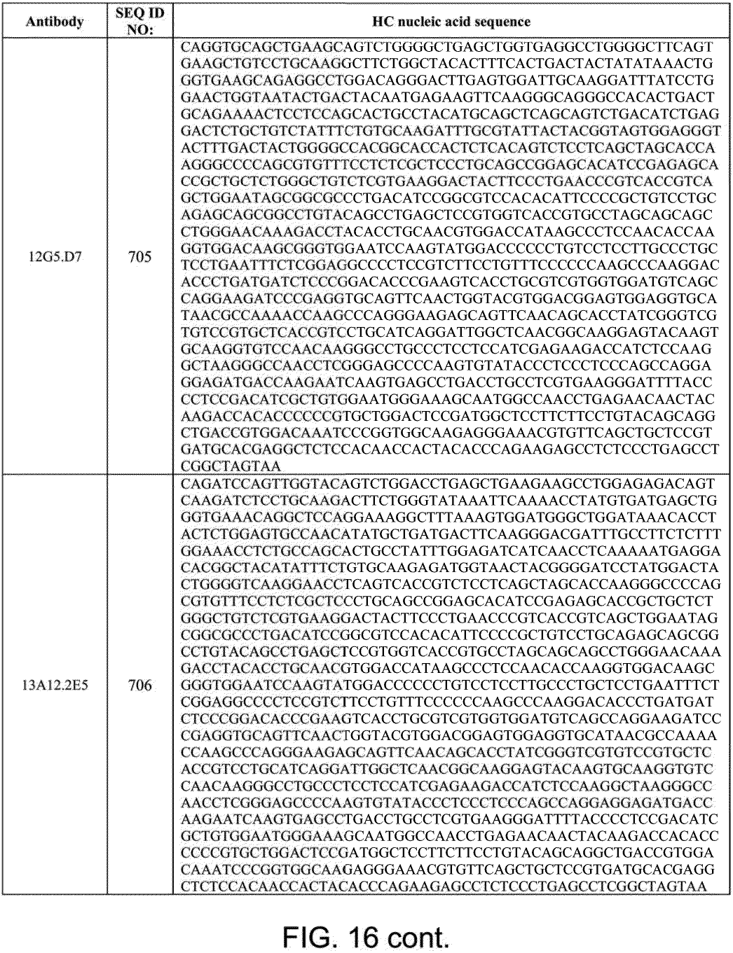

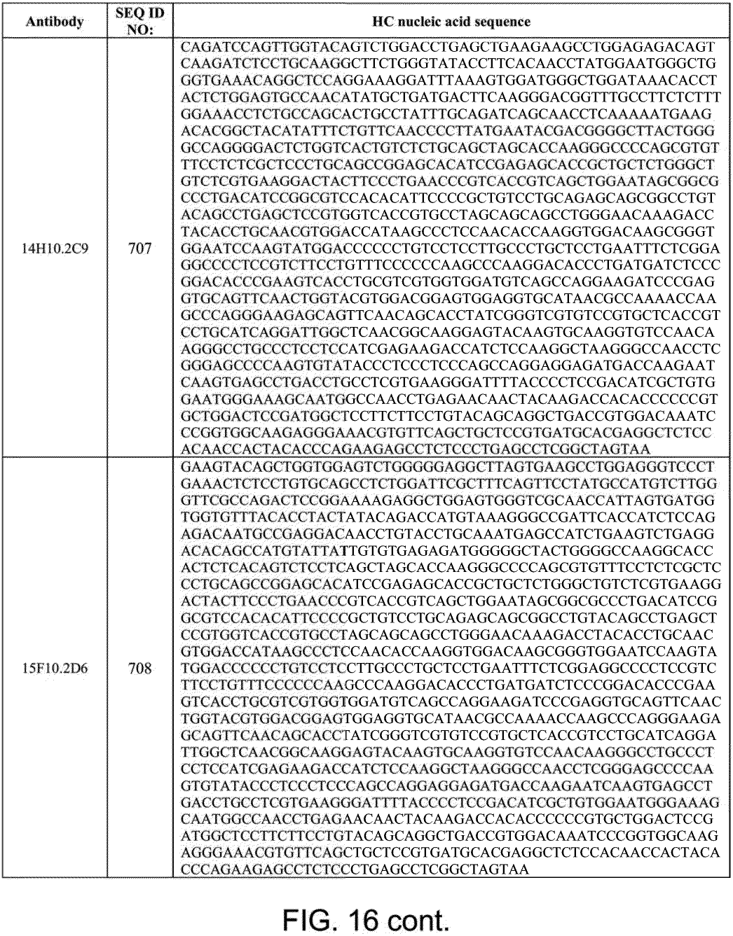

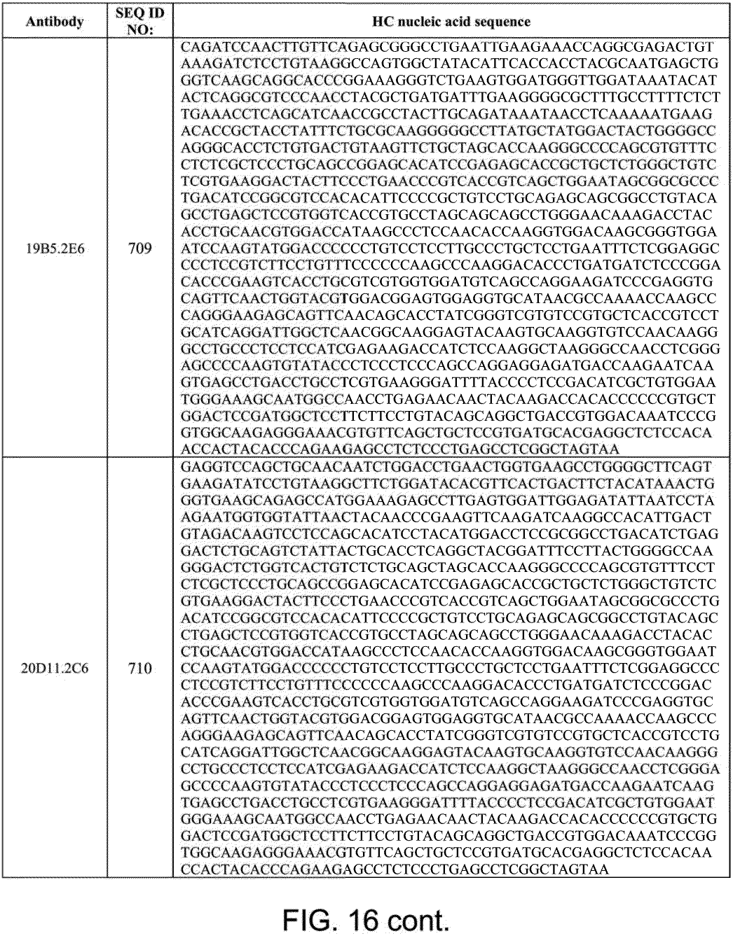

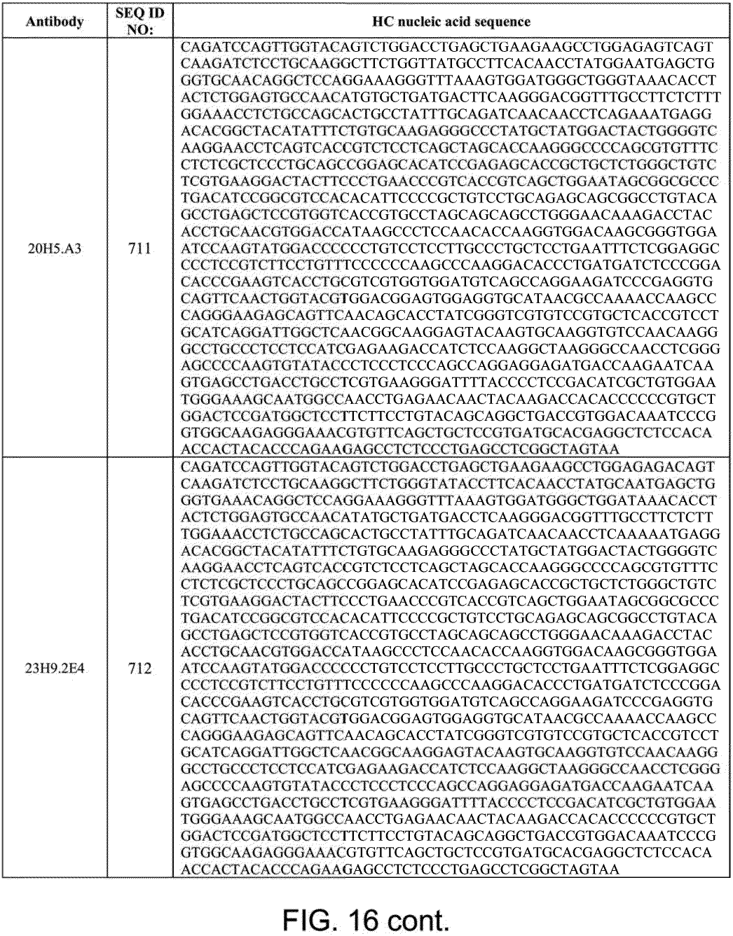

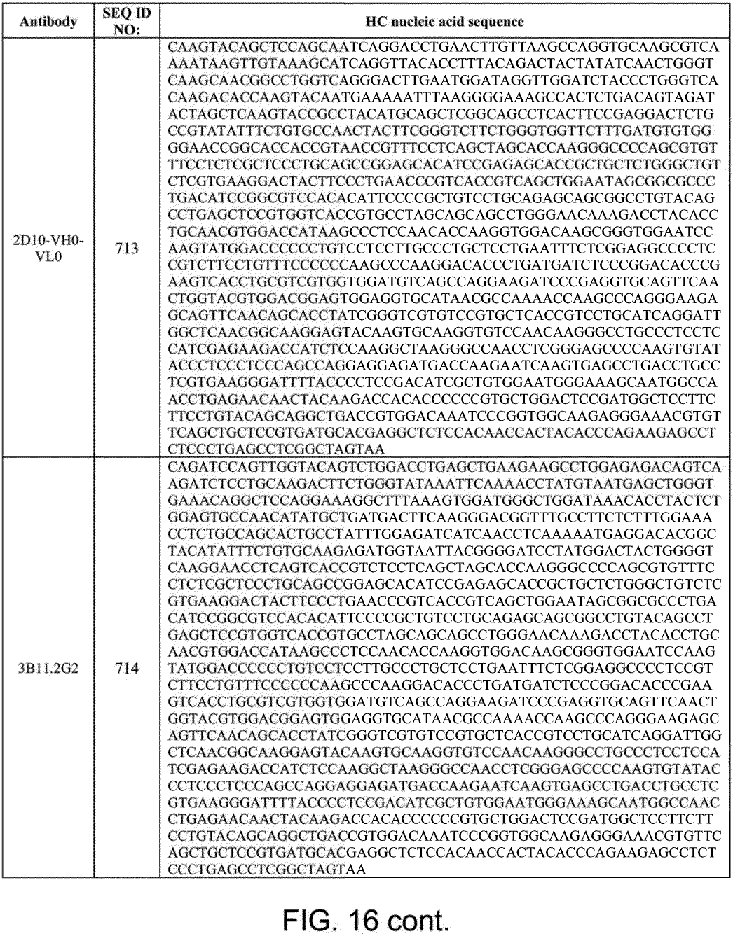

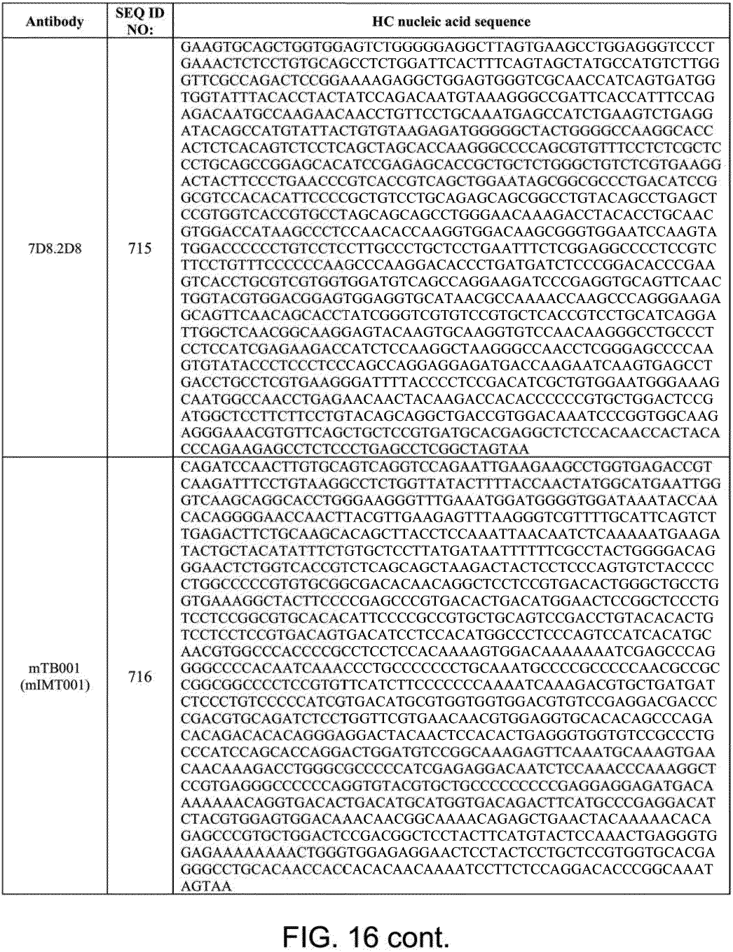

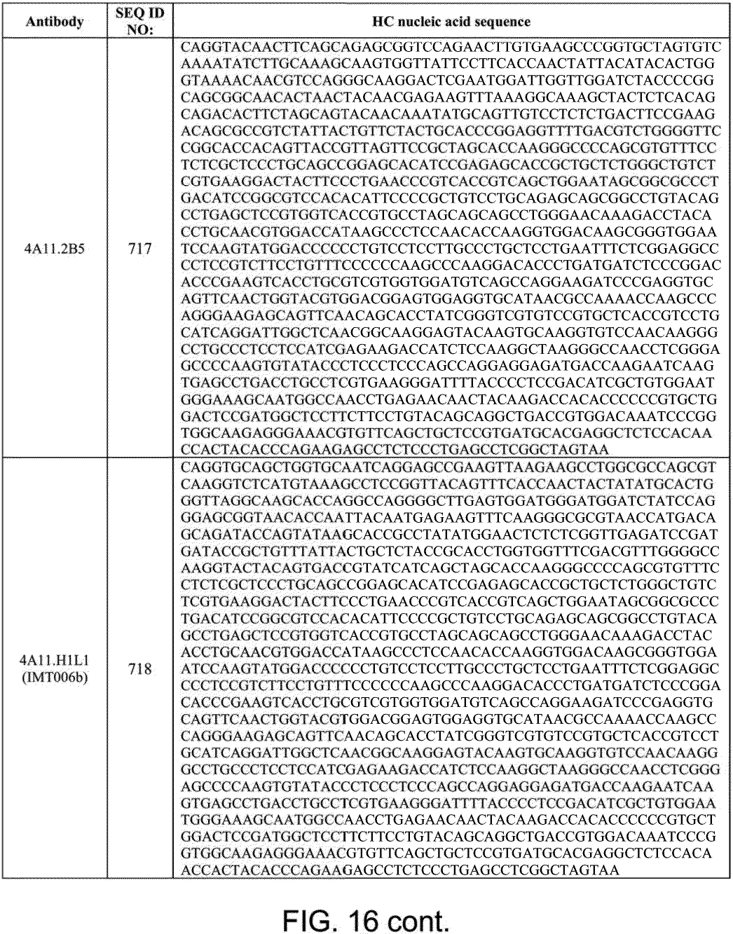

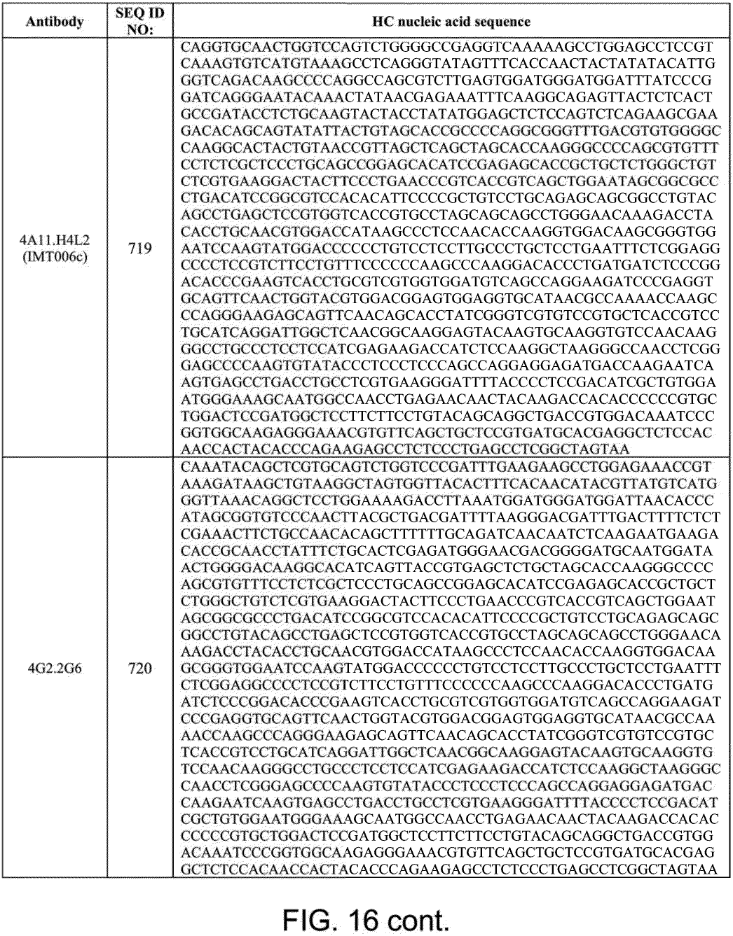

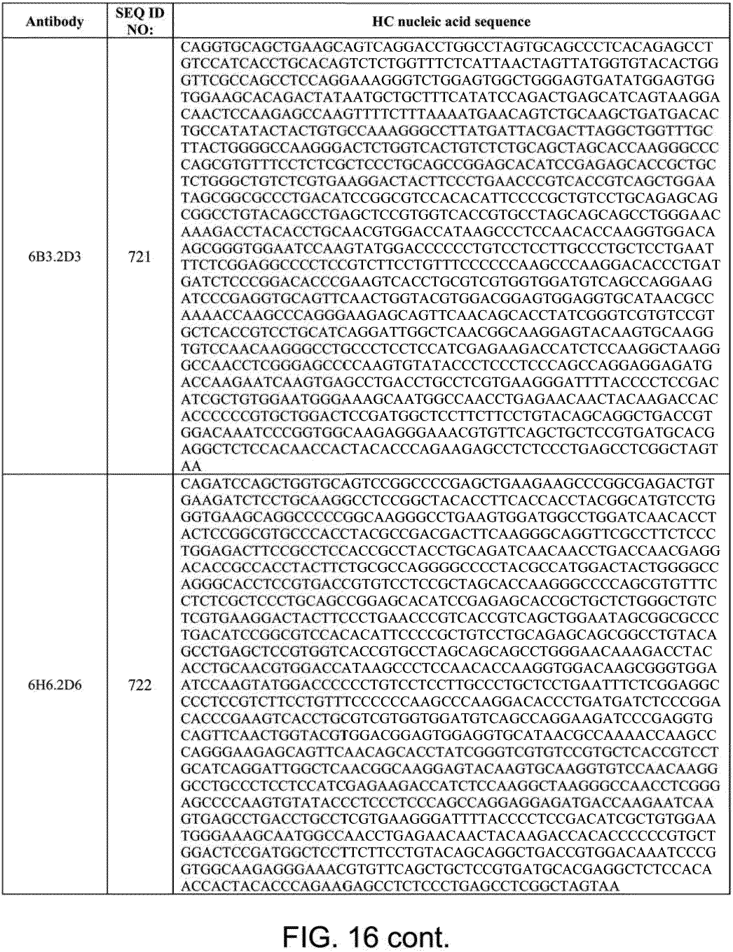

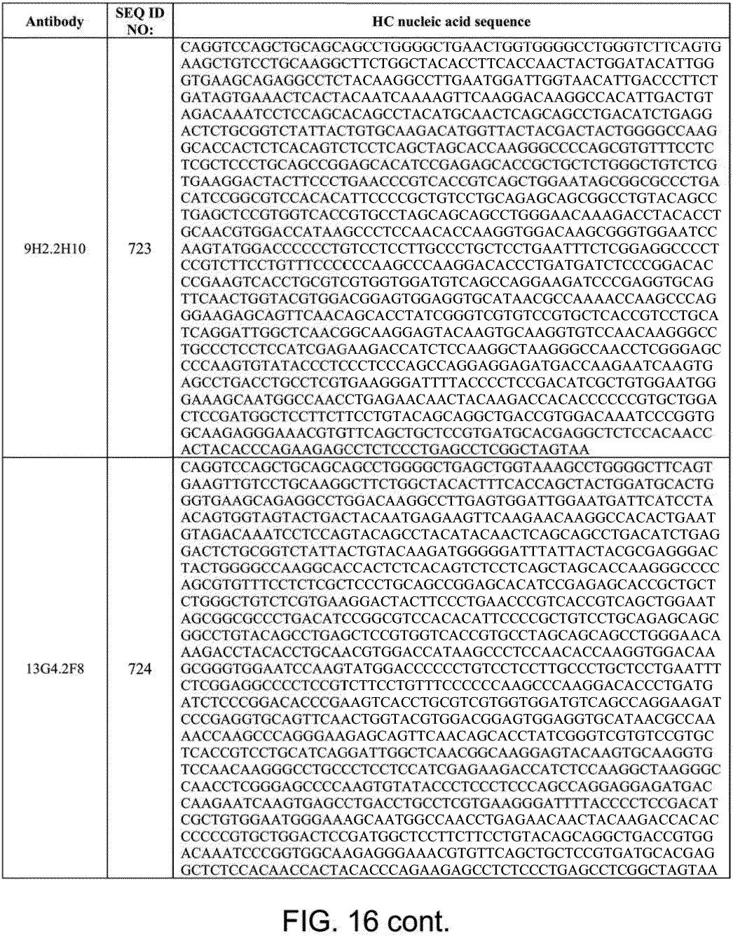

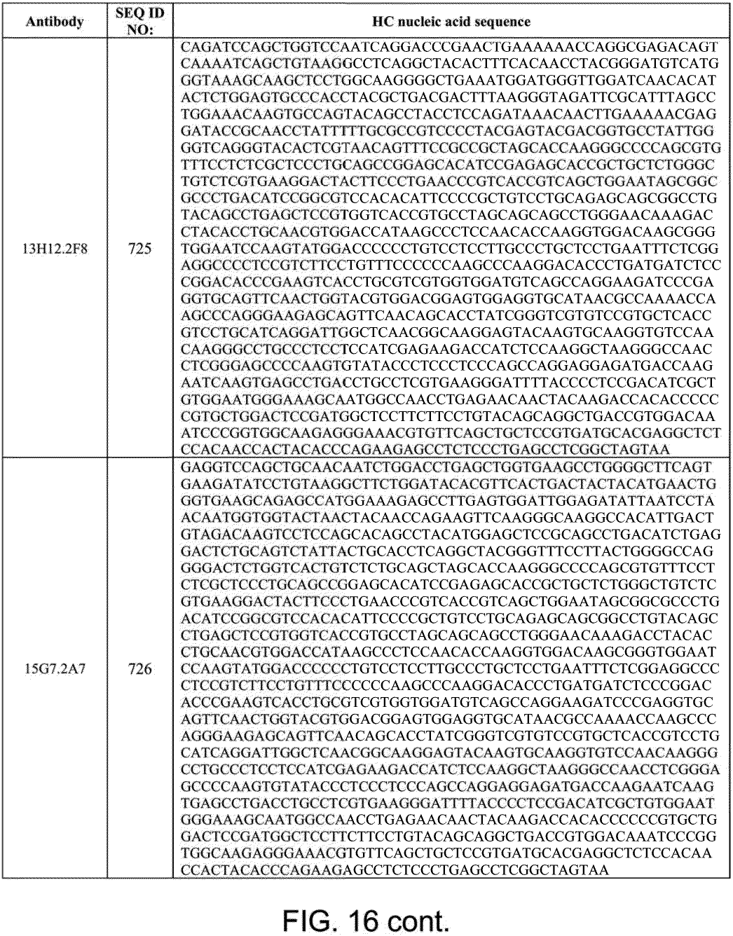

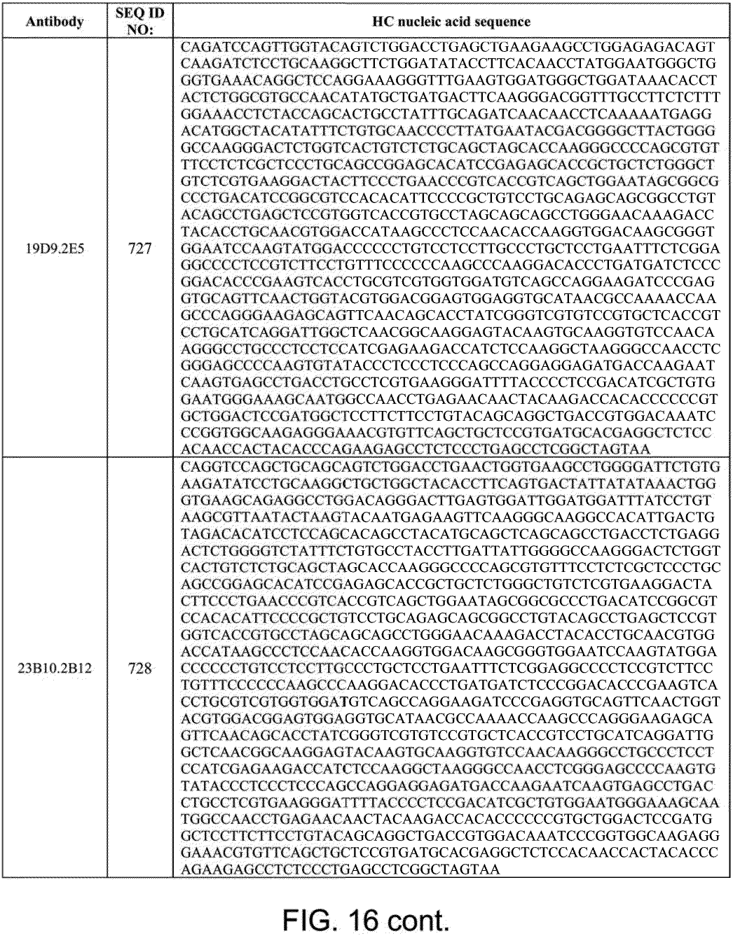

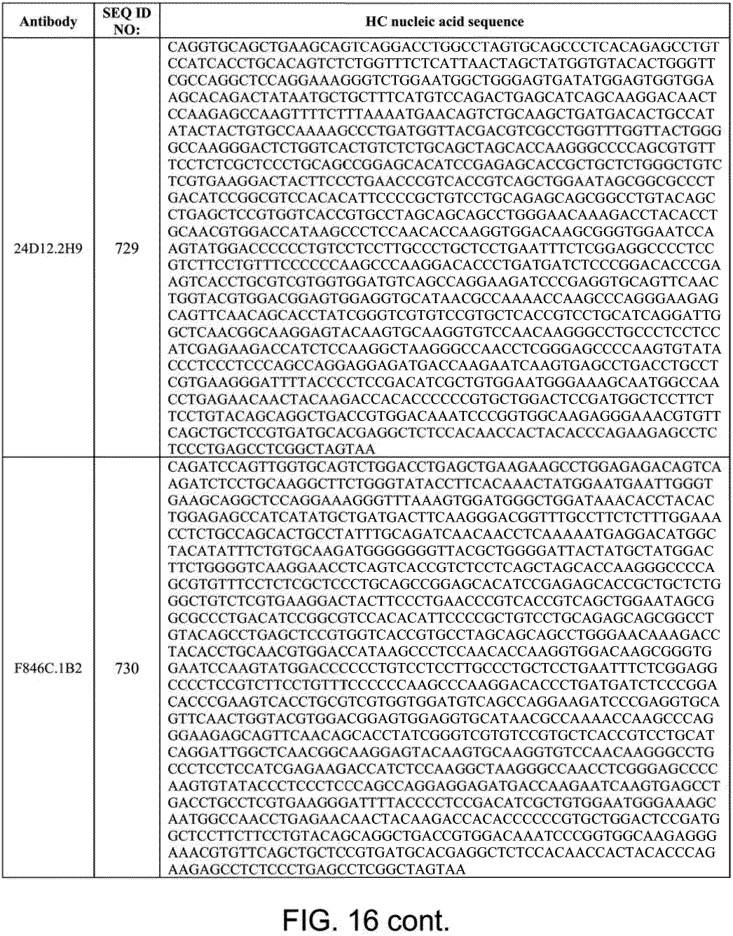

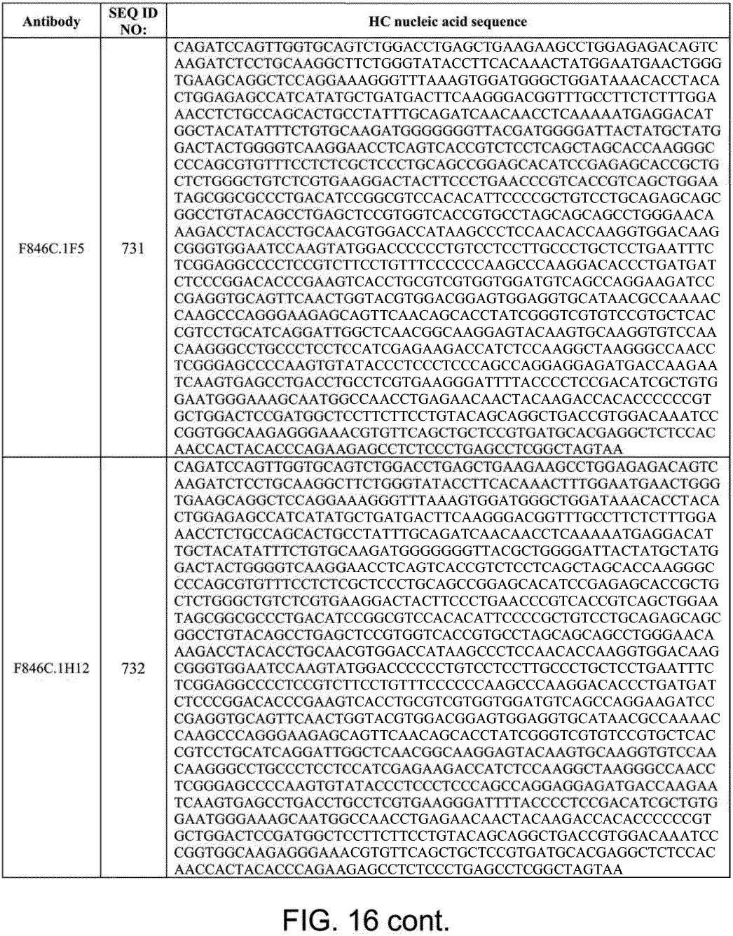

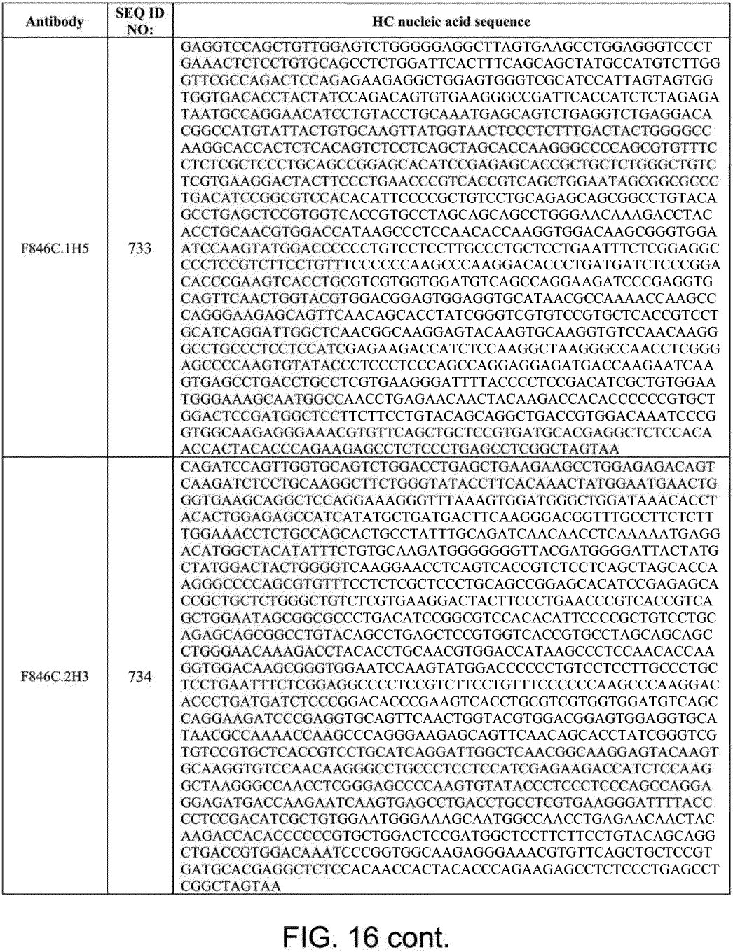

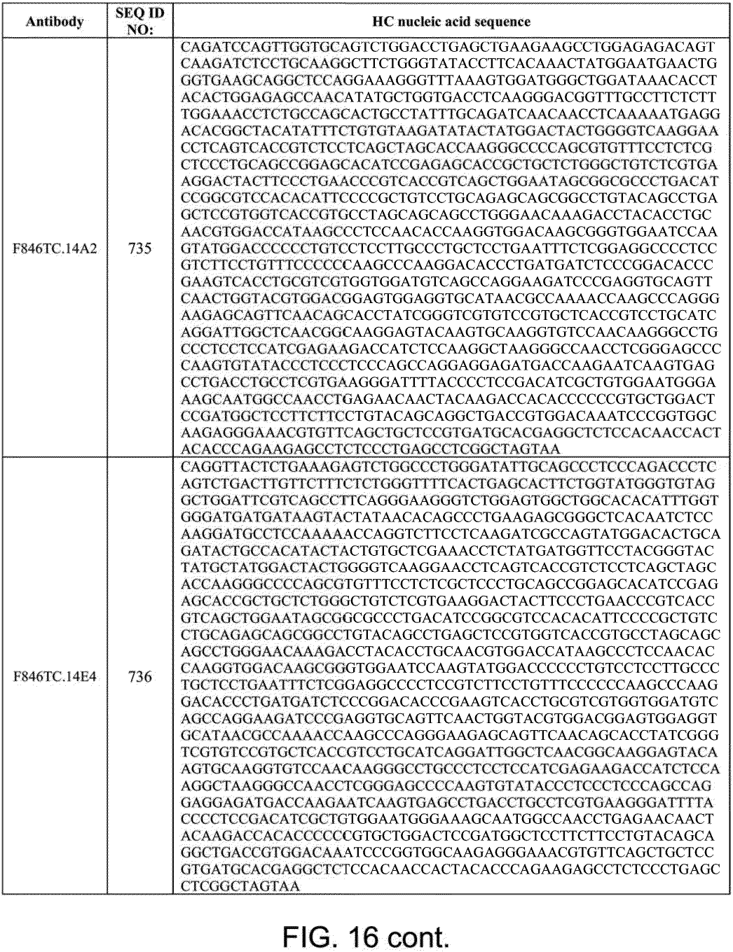

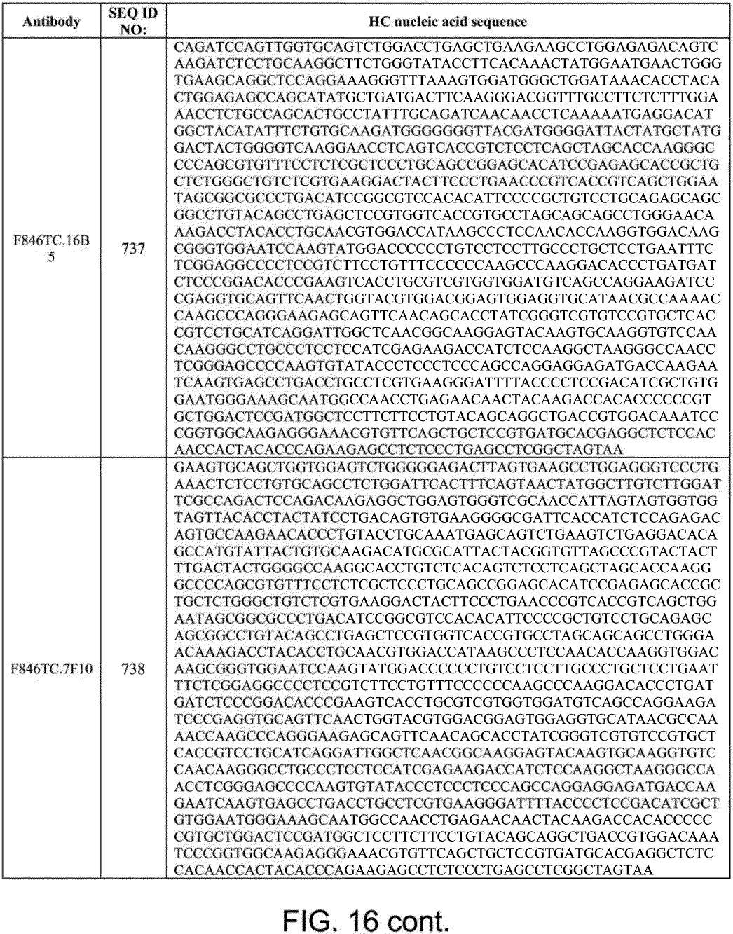

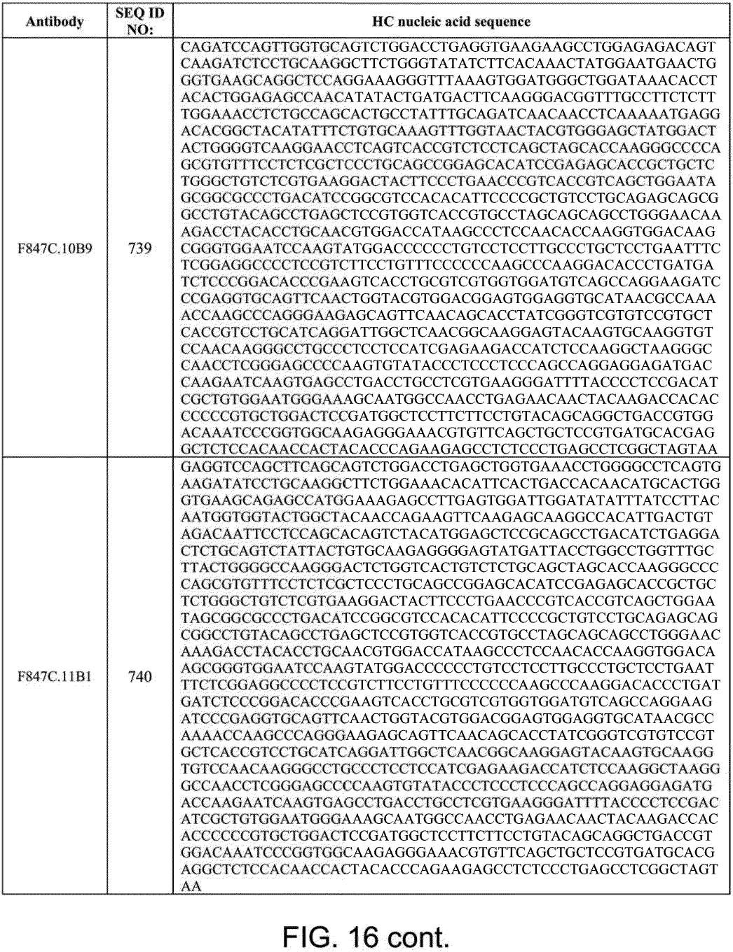

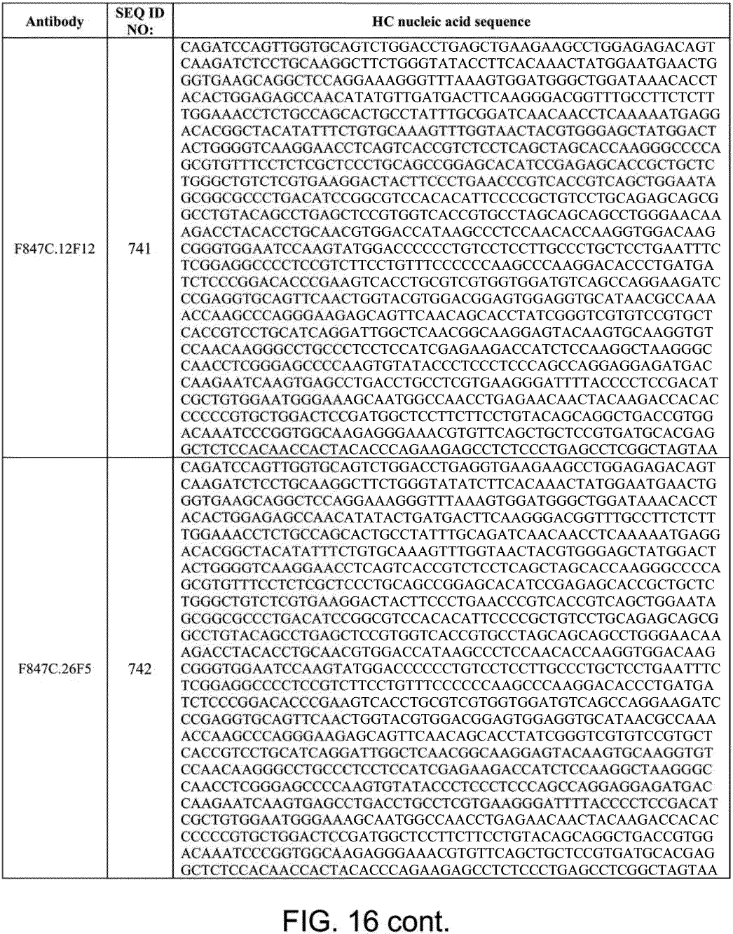

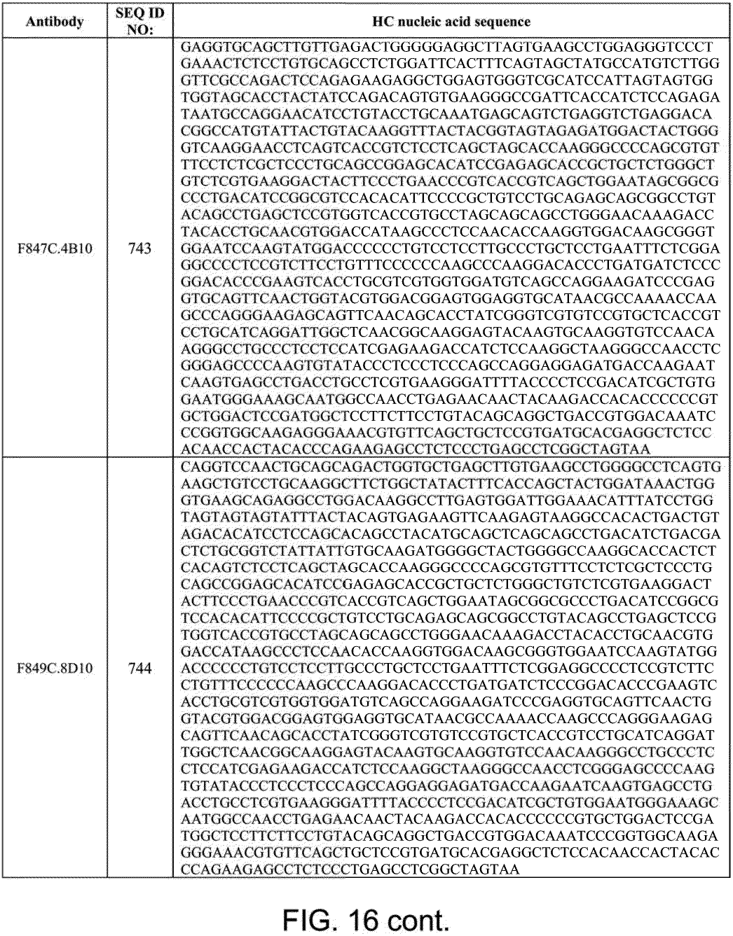

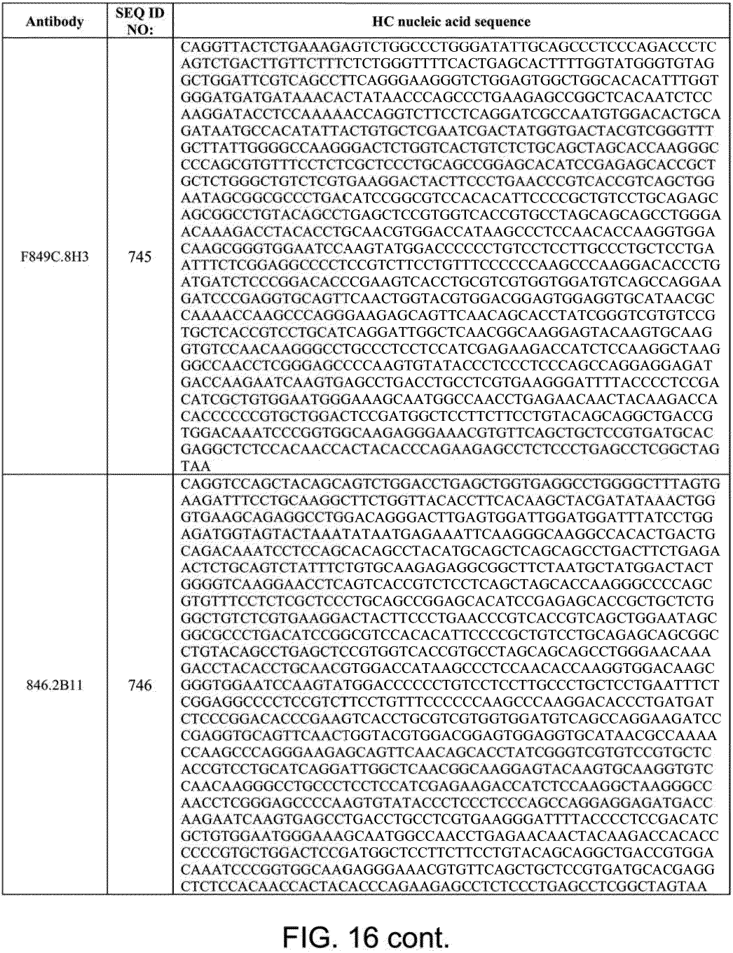

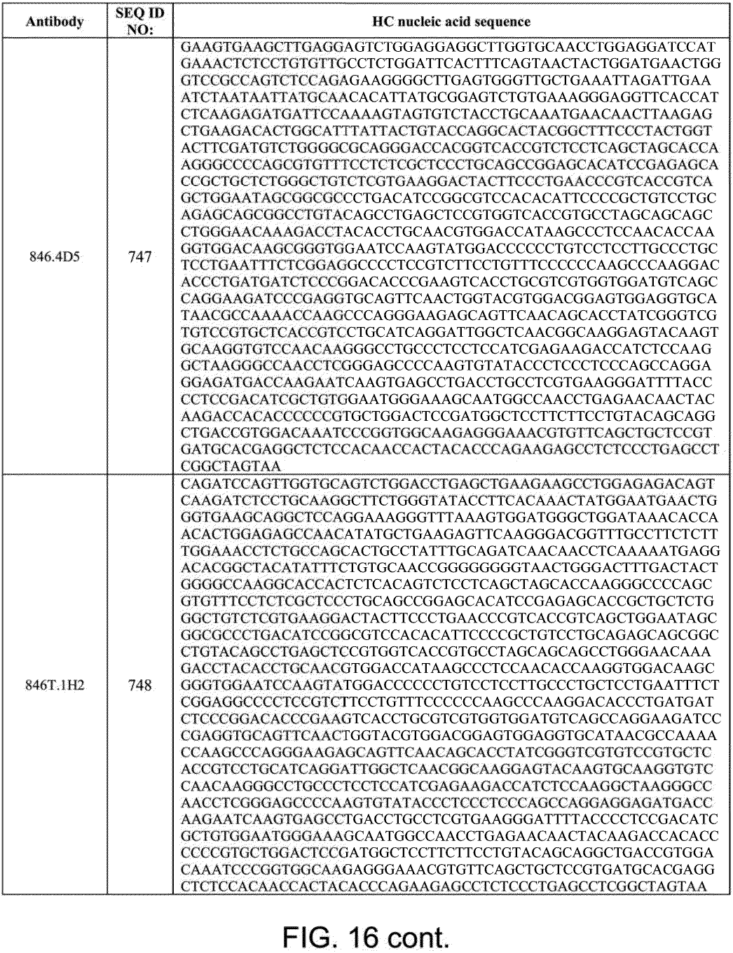

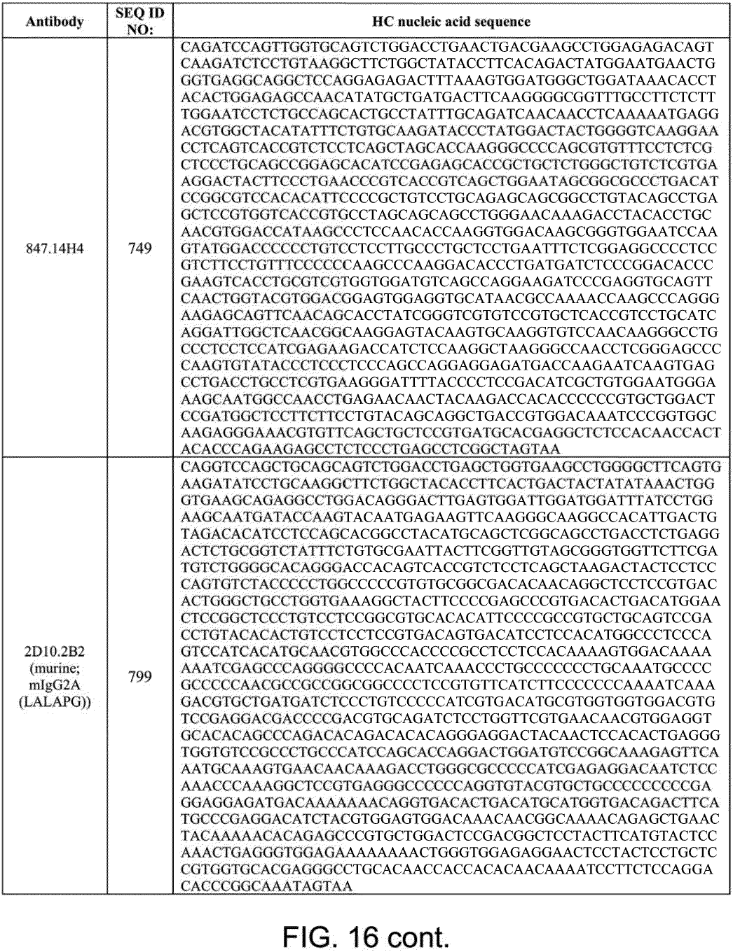

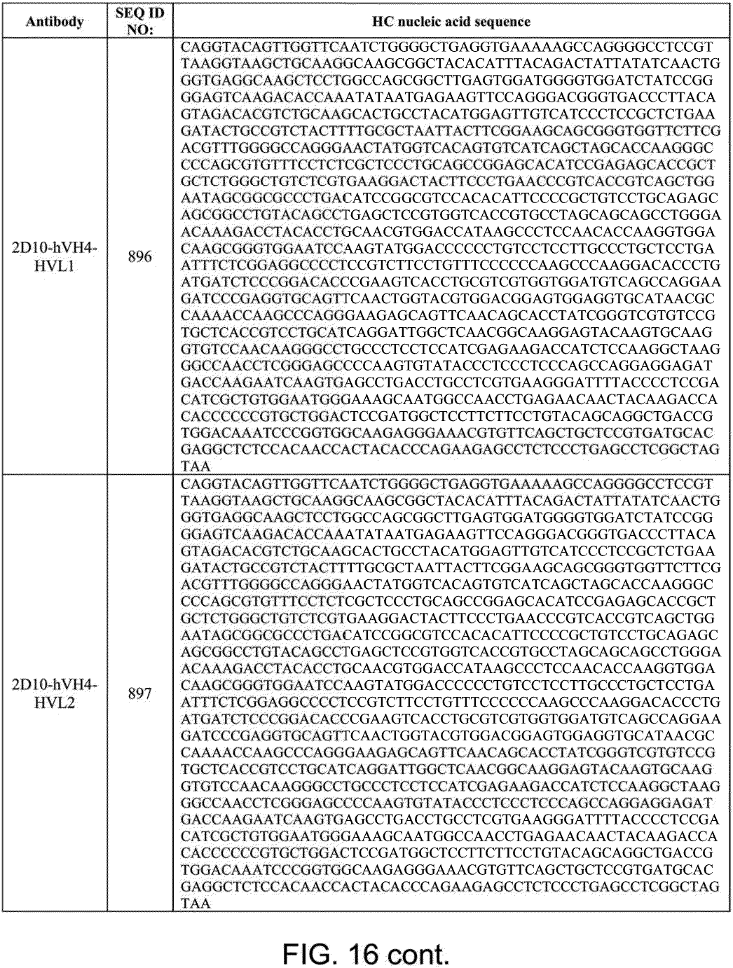

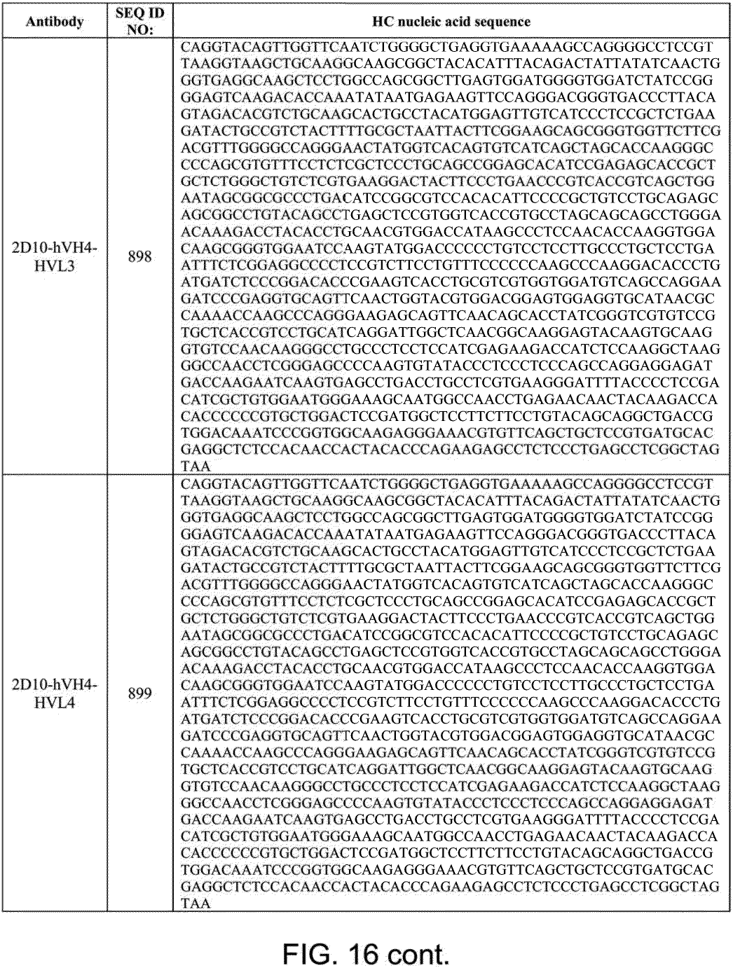

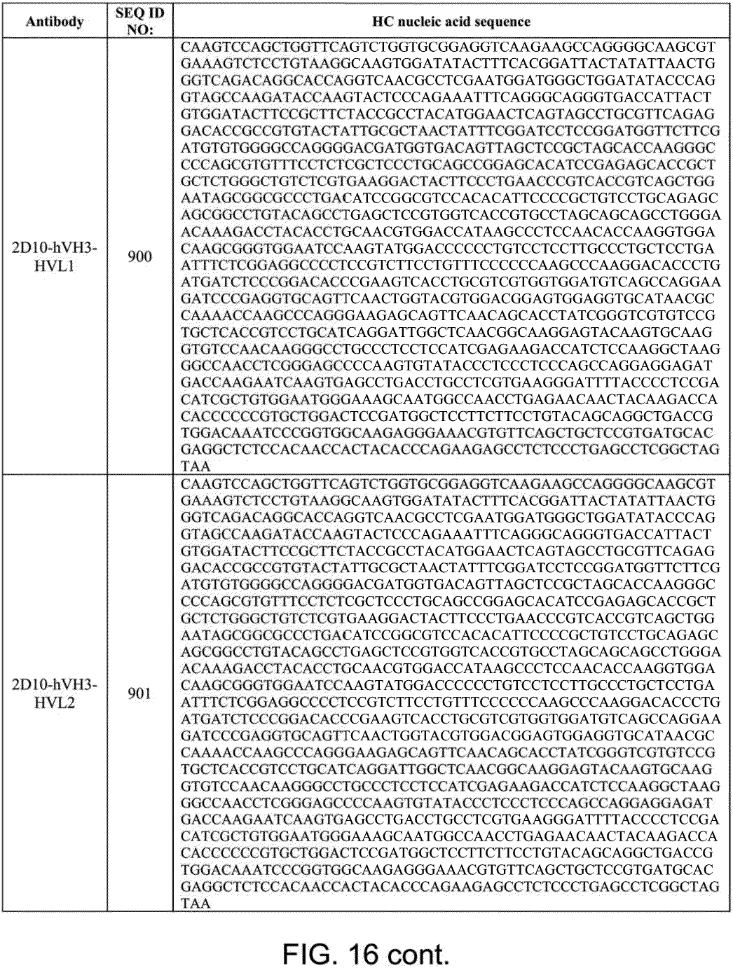

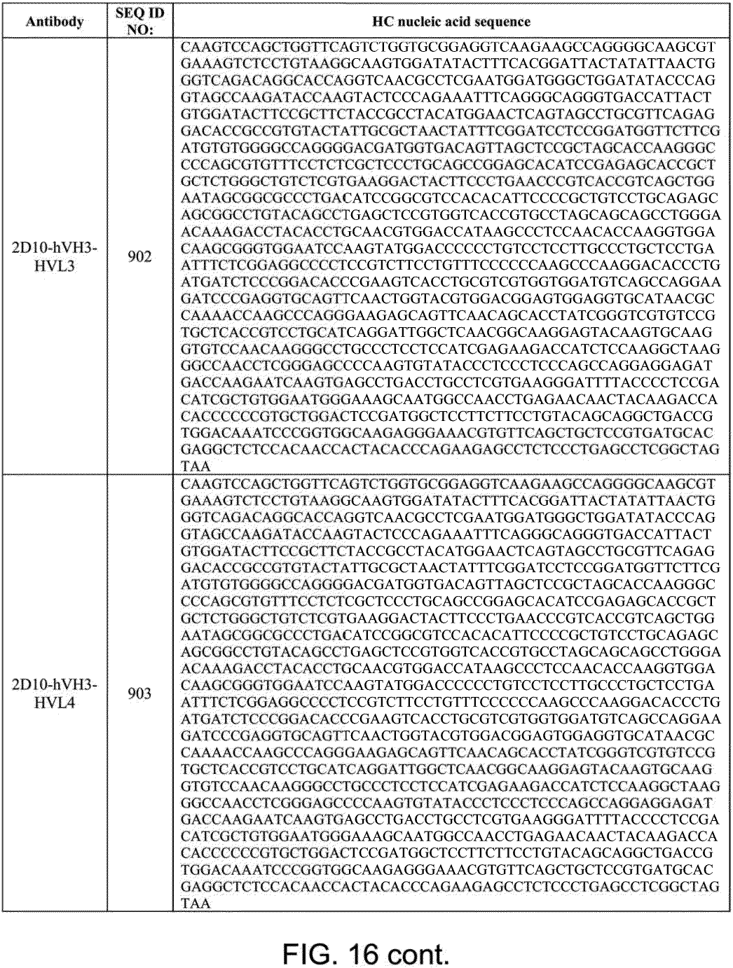

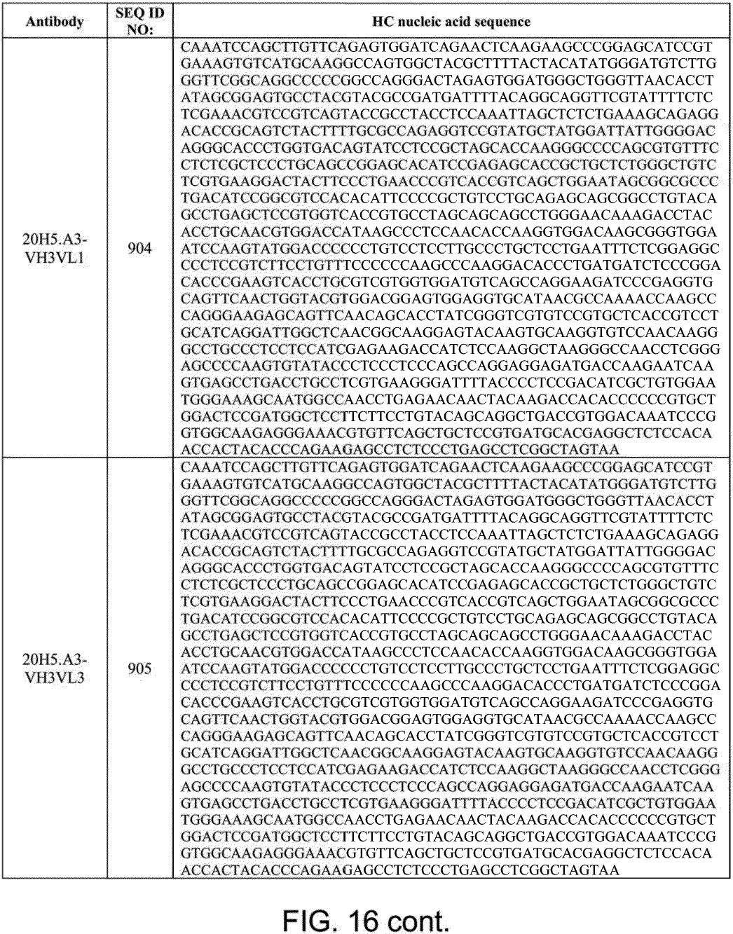

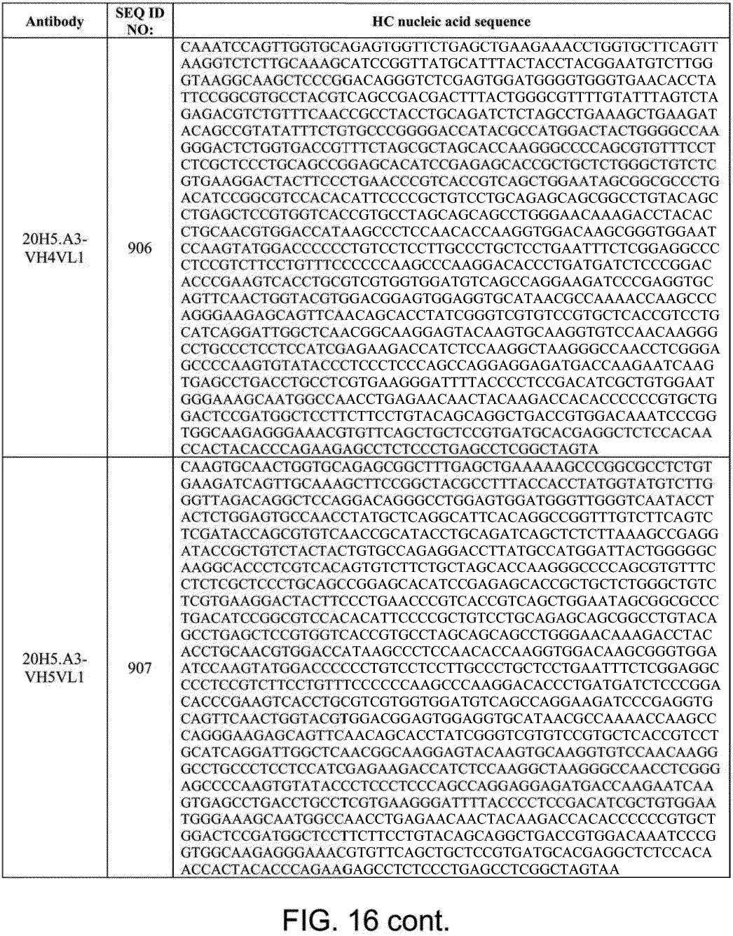

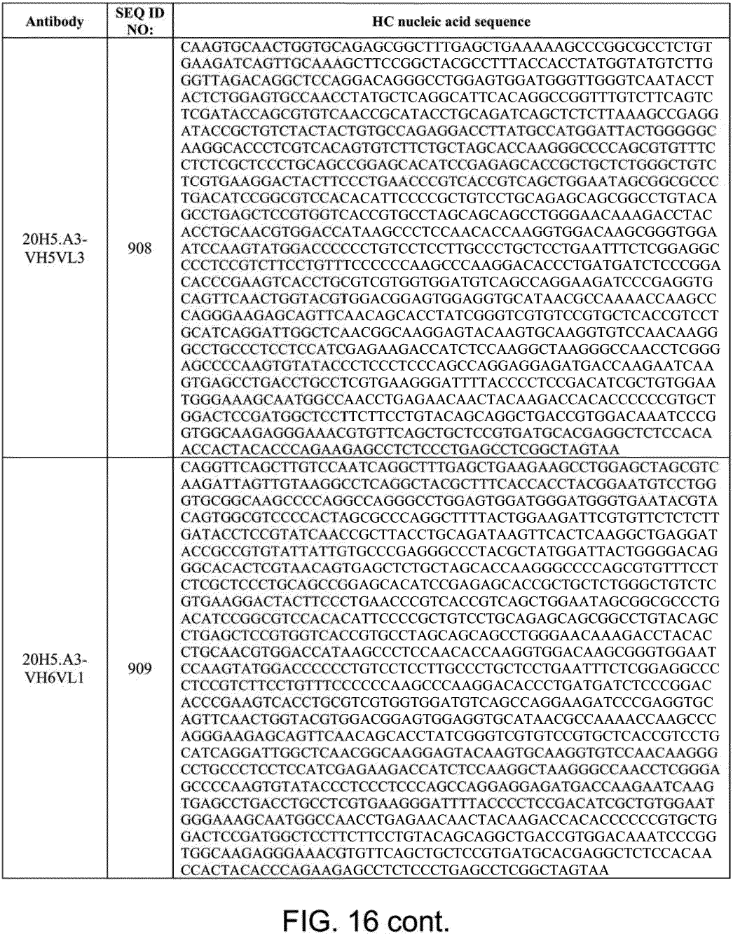

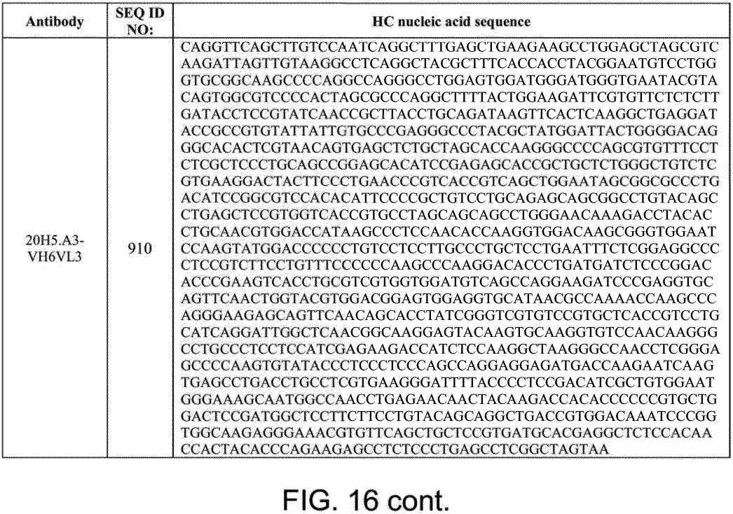

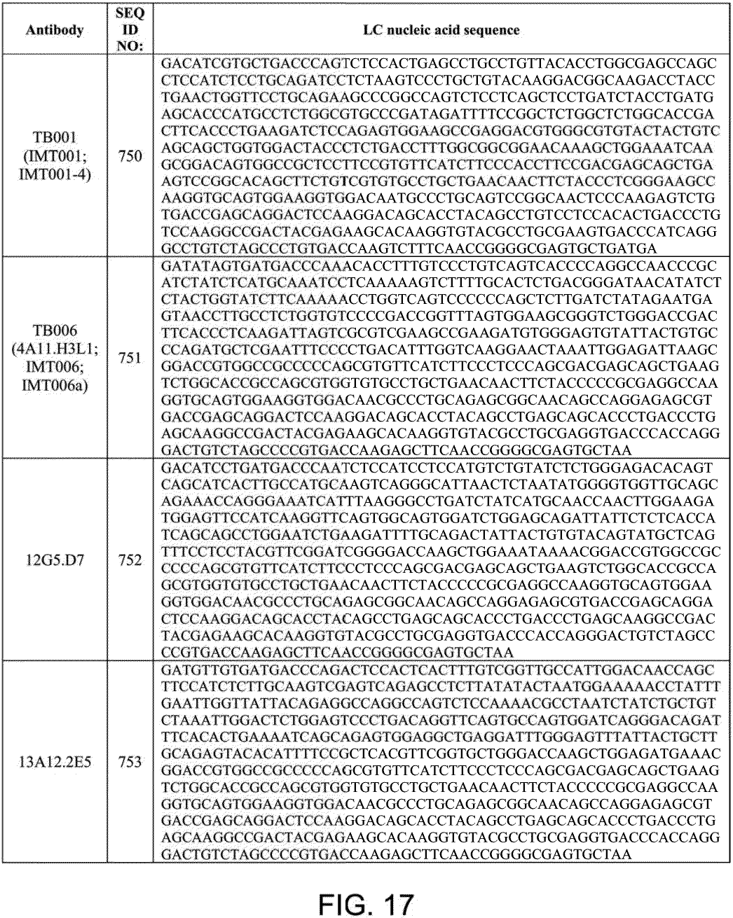

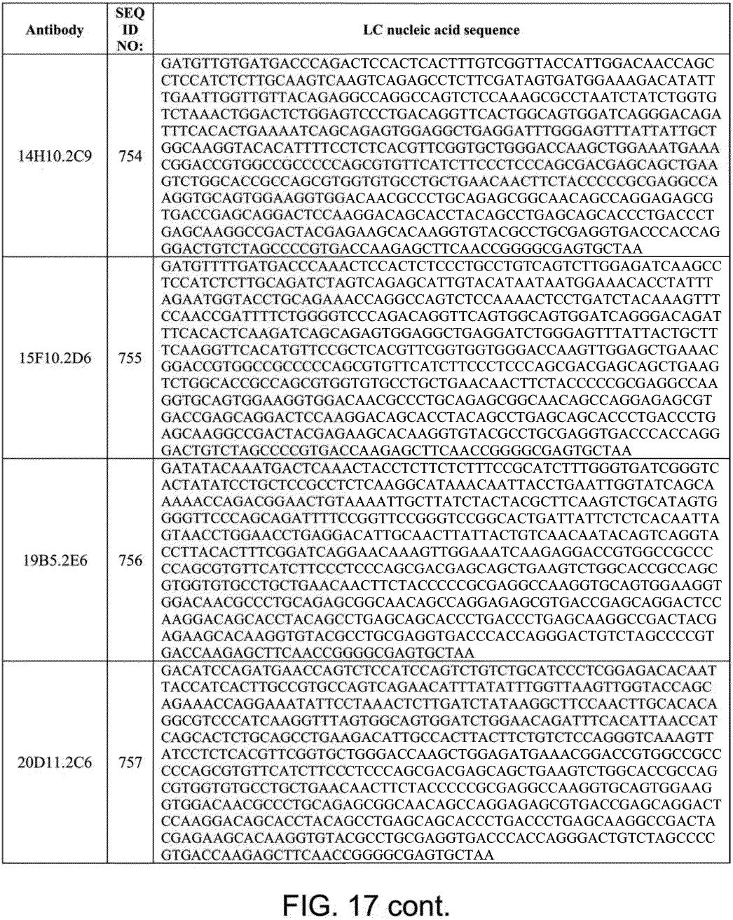

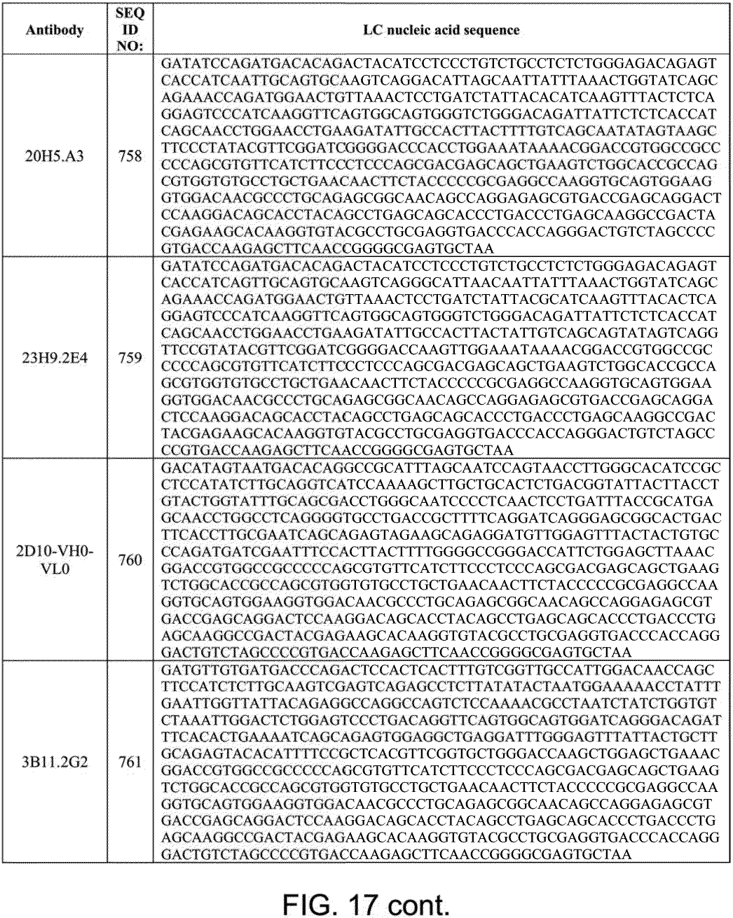

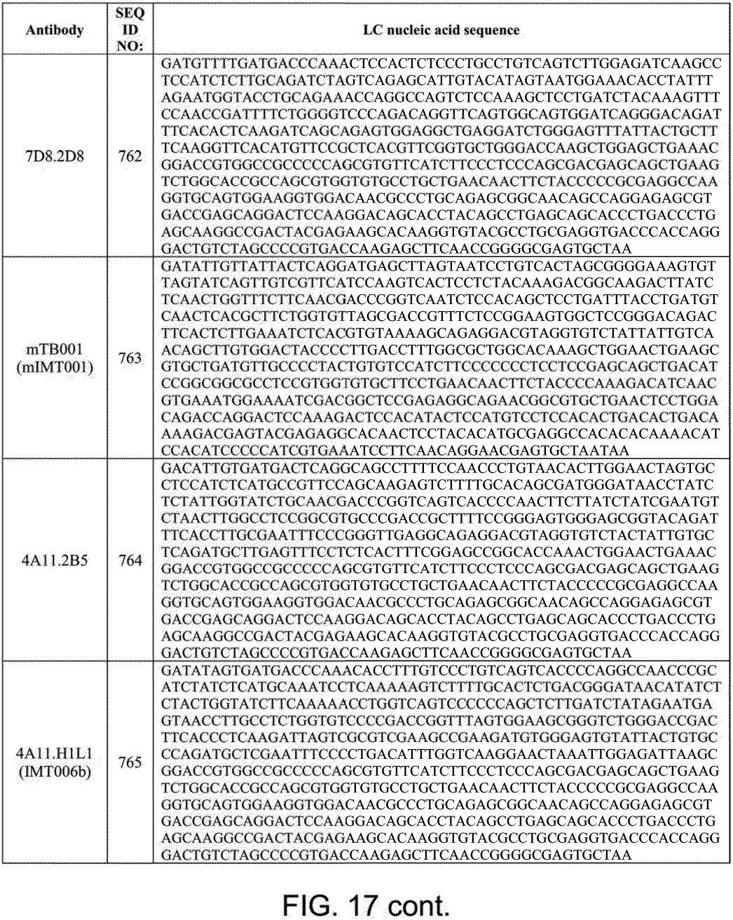

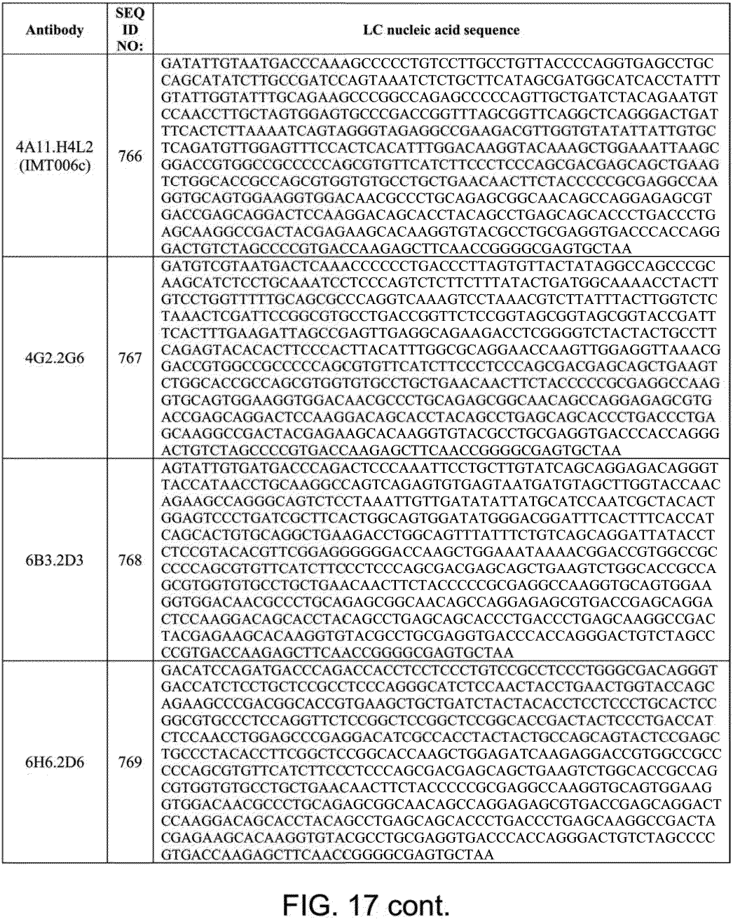

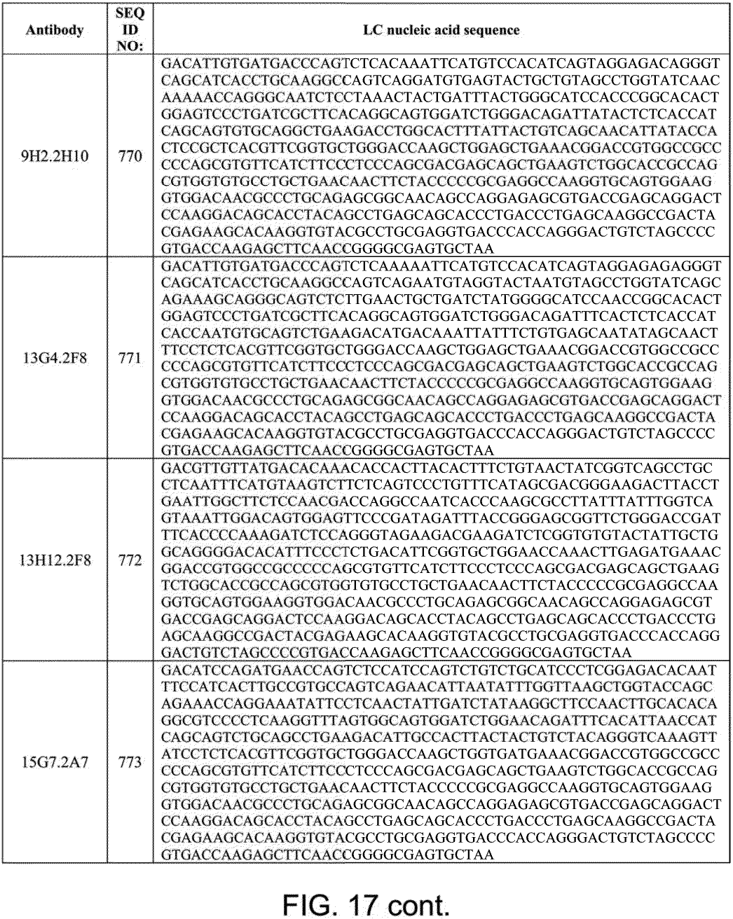

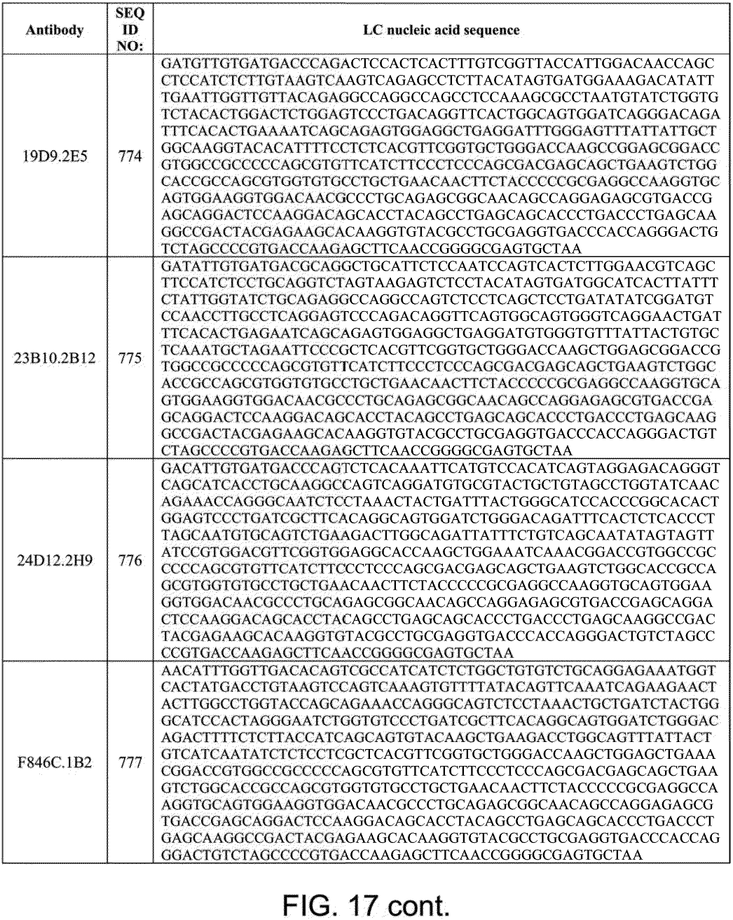

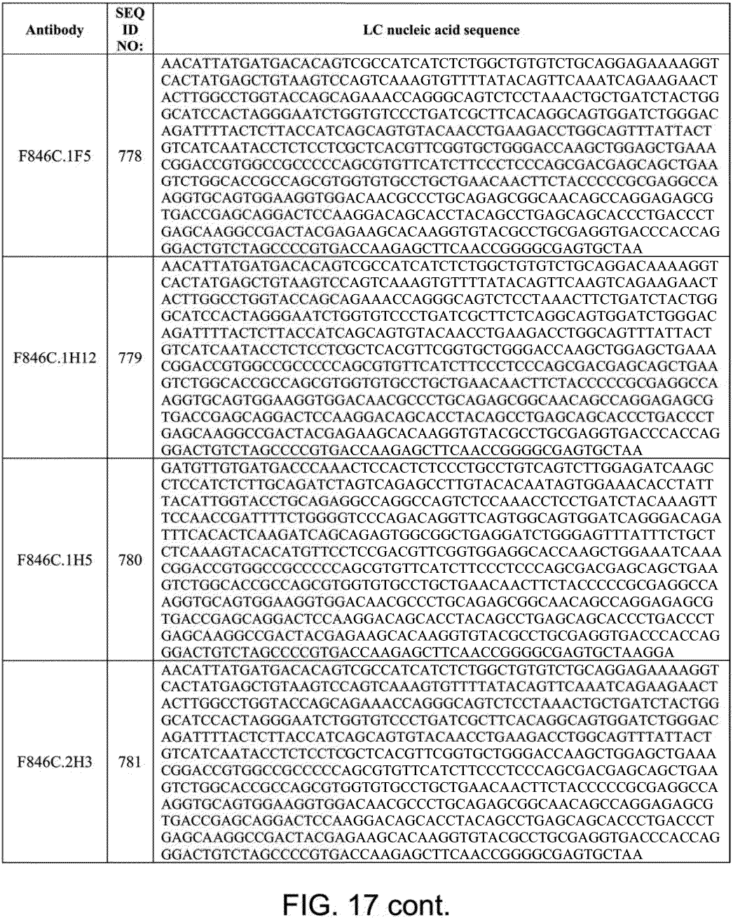

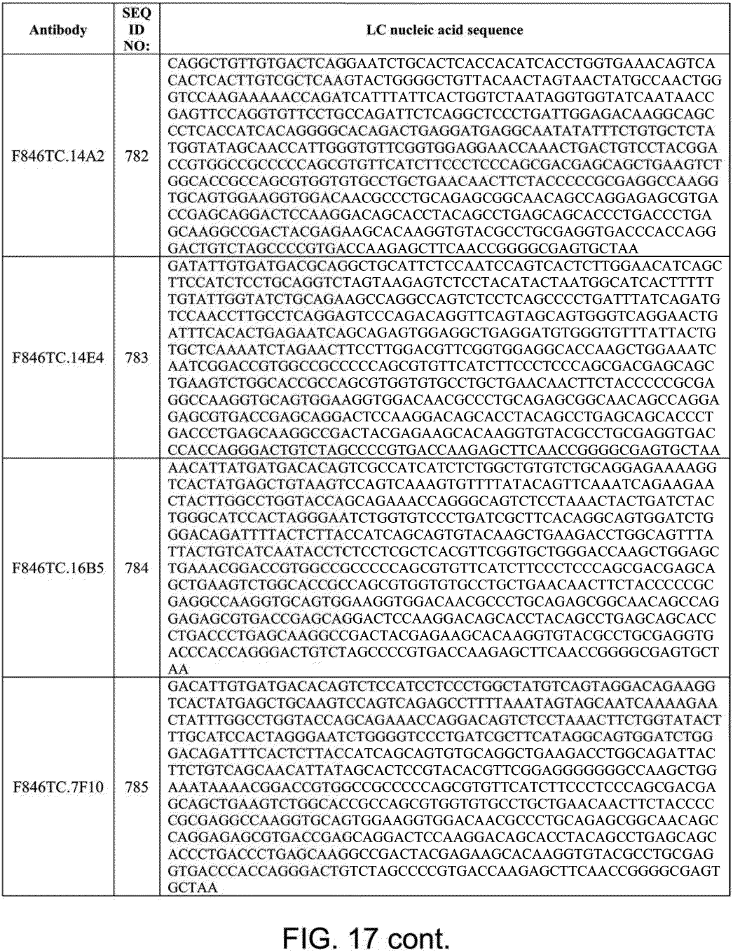

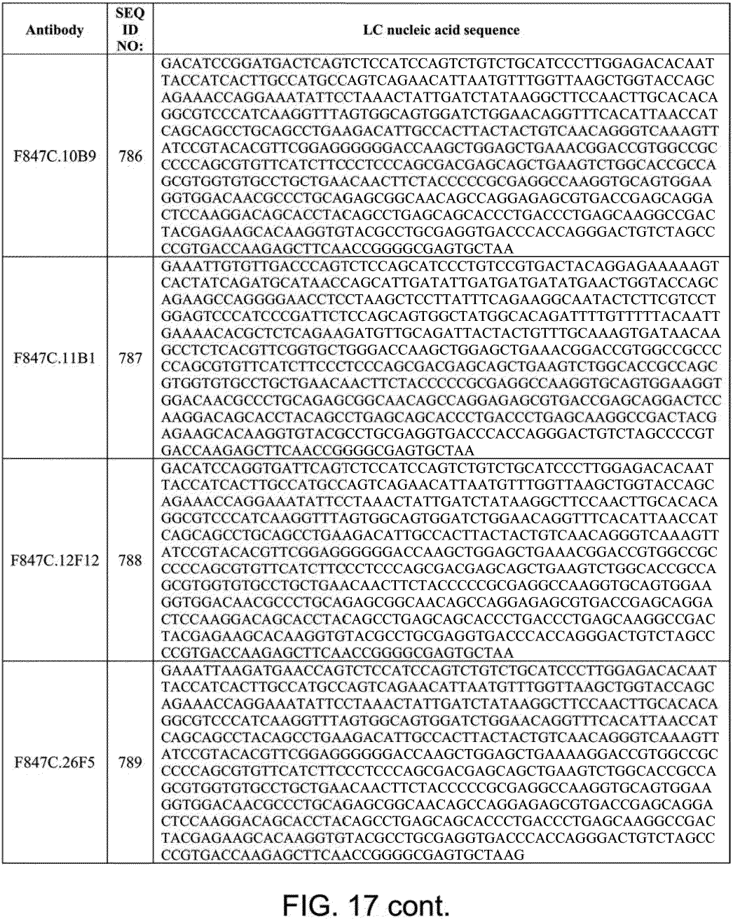

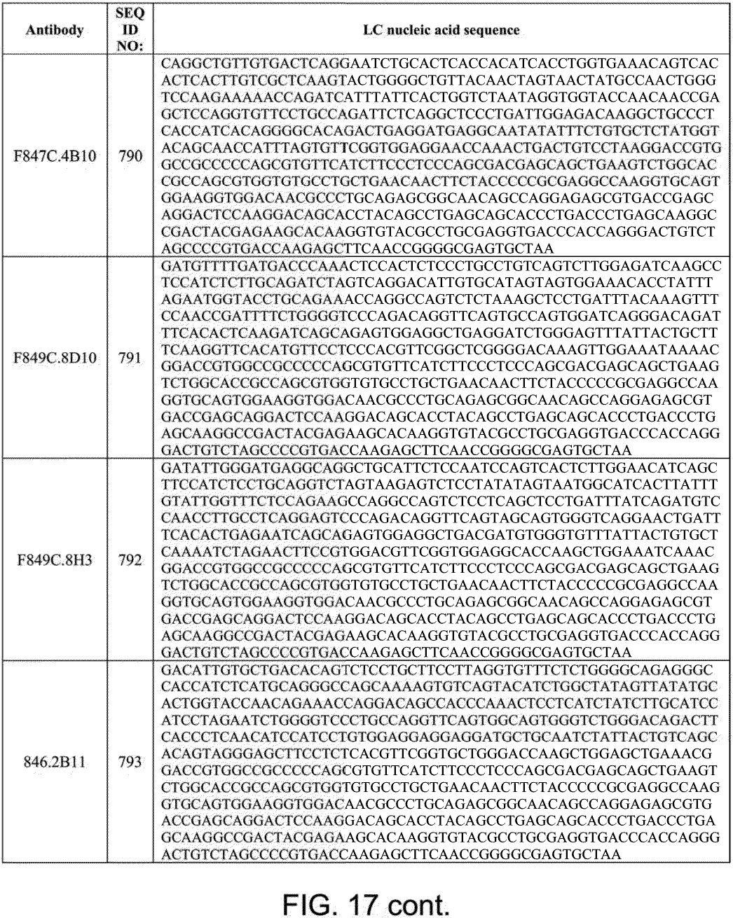

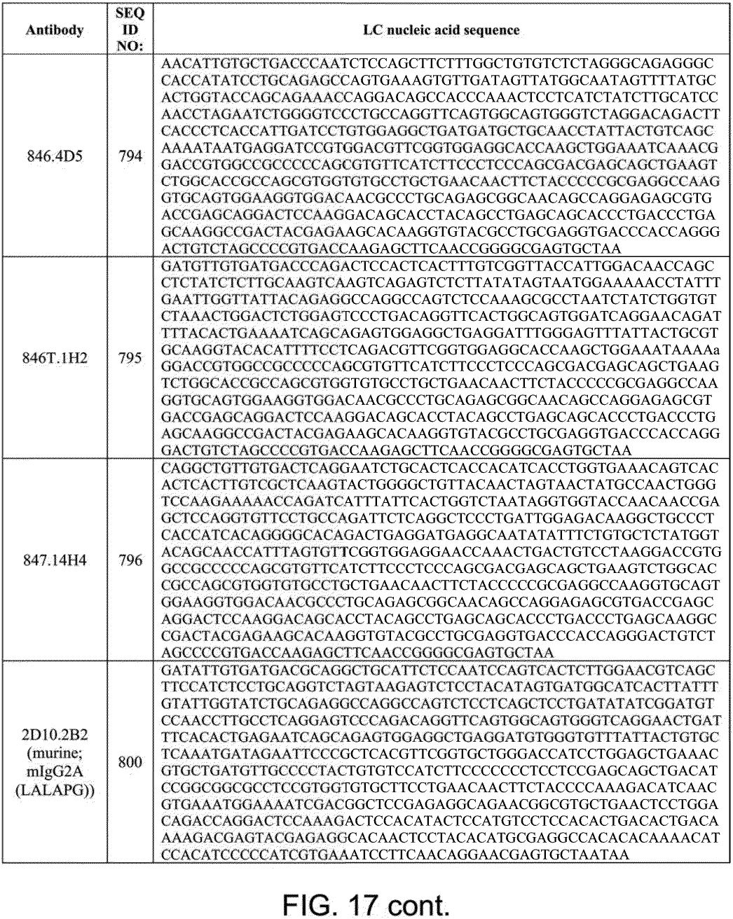

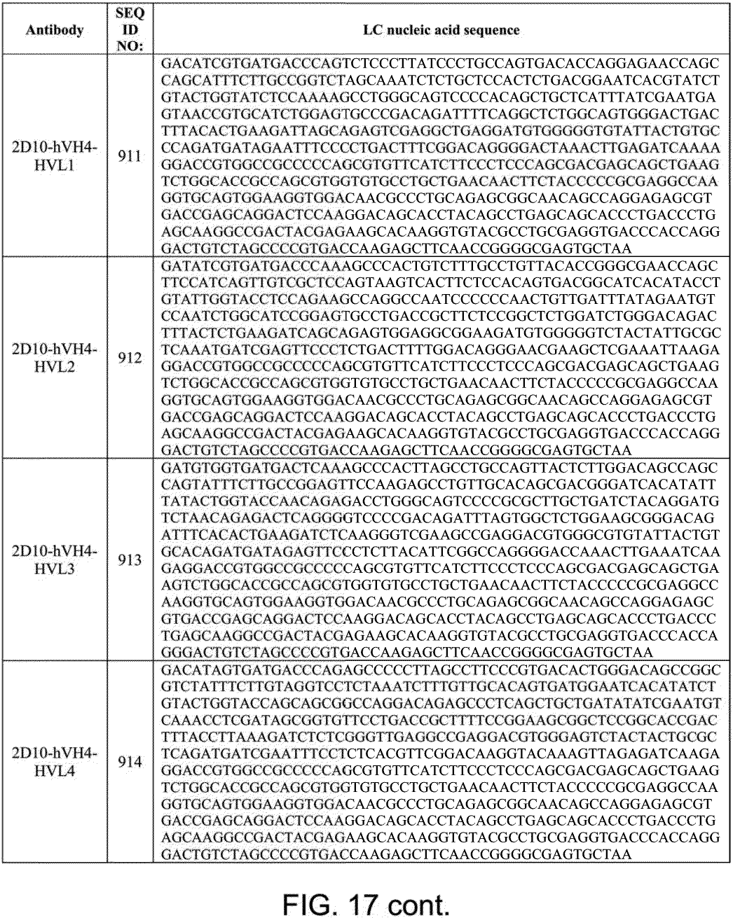

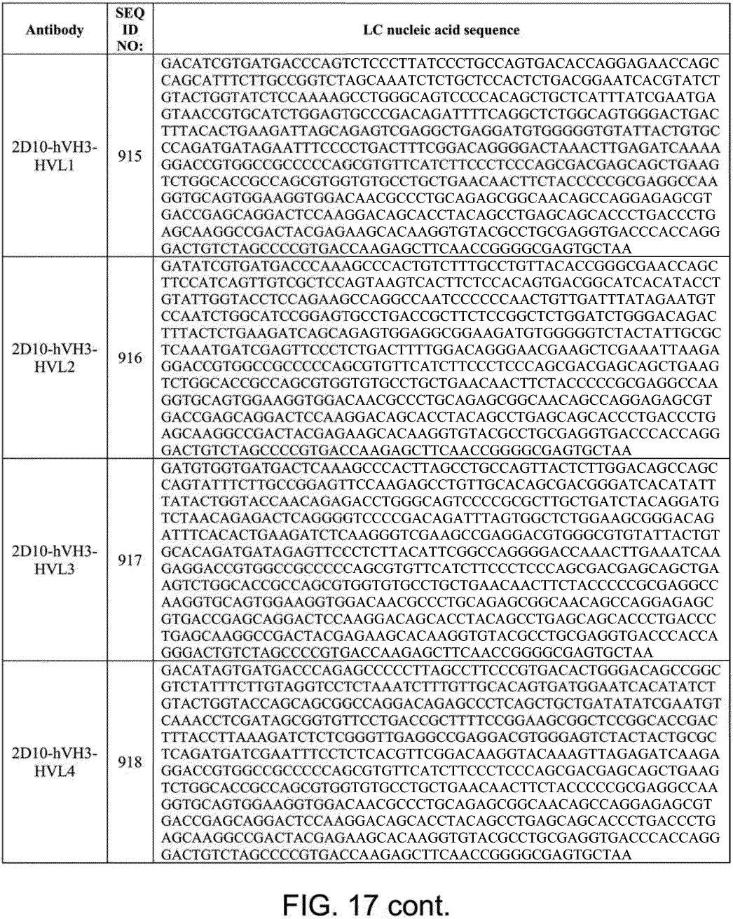

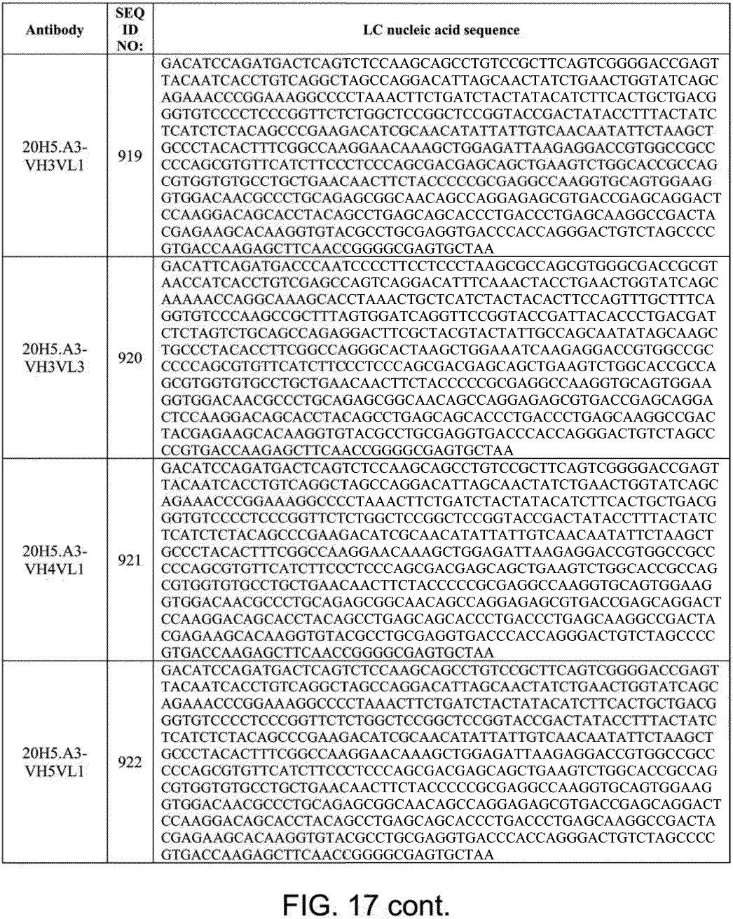

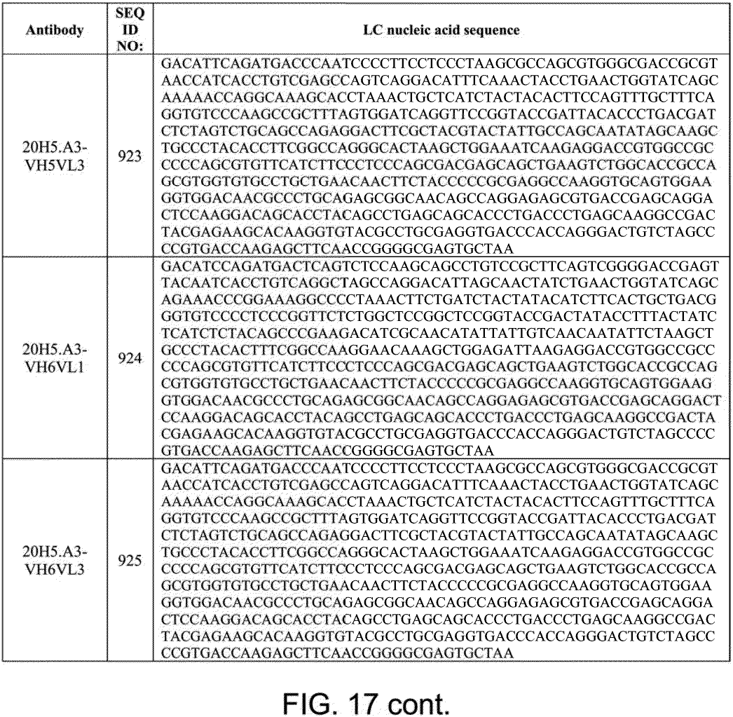

In some embodiments, nucleic acid sequences encoding for heavy chain variable regions are depicted in FIG. 14 (SEQ ID NOs: 539-620, 797, 866-880). In some embodiments, nucleic acid sequences encoding for light chain variable regions are depicted in FIG. 15 (SEQ ID NOs: 621-702, 798, 881-895). In some embodiments, nucleic acid sequences encoding for heavy chains are depicted in FIG. 16 (SEQ ID NO: 703-749, 799, 896-910). In some embodiments, nucleic acid sequences encoding for light chains are depicted in FIG. 17 (SEQ ID NO: 750-796, 800, 911-925).

Any one of the anti-Gal3 antibodies or binding fragments thereof described herein can be prepared by recombinant DNA technology, synthetic chemistry techniques, or a combination thereof. For instance, sequences encoding the desired components of the anti-Gal3 antibodies, including light chain CDRs and heavy chain CDRs are typically assembled cloned into an expression vector using standard molecular techniques known in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector which comprises an anti-Gal3 antibody of interest or binding fragment thereof.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection, which offers a diverse collection of well-characterized hybridoma cell lines. Alternatively, antibody nucleotides can be obtained from immunized or non-immunized rodents or humans, and form organs such as spleen and peripheral blood lymphocytes.

Polynucleotides encoding anti-Gal3 antibodies or binding fragments thereof can also be modified, for example, by substituting the coding sequence for human heavy and light chain constant regions in place of the homologous non-human sequences. In that manner, chimeric antibodies are prepared that retain the binding specificity of the original anti-Gal3 antibody or binding fragment thereof.

Also disclosed herein are methods of producing an anti-Gal3 antibody or binding fragment thereof. In some embodiments, the methods comprise expressing a nucleic acid that encodes for the anti-Gal3 antibody or binding fragment thereof in a cell and isolating the expressed anti-Gal3 antibody or binding fragment thereof from the cell. In some embodiments, the methods further comprise concentrating the anti-Gal3 antibody or binding fragment thereof to a desired concentration. In some embodiments, the cell is a mammalian cell, insect cell, or bacterial cell. In some embodiments, the anti-Gal3 antibody or binding fragment thereof is any one of the anti-Gal3 antibodies or binding fragments disclosed herein. Specific procedures of expressing antibodies in a cell and isolation of the expressed antibodies are conventionally known and can be practiced by one skilled in the art.

### Antibody Production

In some cases, anti-Gal3 antibodies or binding fragments thereof are raised by standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund’s, Freund’s complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal anti-Gal3 antibodies or binding fragments thereof can be produced from animals which have been genetically altered to produce human immunoglobulins. A transgenic animal can be produced by initially producing a “knock-out” animal which does not produce the animal’s natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, each incorporated fully herein by reference in its entirety. Such antibodies can be referred to as human xenogenic antibodies.

Alternatively, anti-Gal3 antibodies or binding fragments thereof can be produced from phage libraries containing human variable regions. See U.S. Pat. No. 6,174,708, incorporated fully herein by reference in its entirety.

In some aspects of any of the embodiments disclosed herein, an anti-Gal3 antibody or binding fragment thereof is produced by a hybridoma.

For monoclonal anti-Gal3 antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the anti-Gal3 antibody or binding fragment thereof may be produced by genetic engineering.

Anti-Gal3 antibodies or binding fragments thereof disclosed herein can have a reduced propensity to induce an undesired immune response in humans, for example, anaphylactic shock, and can also exhibit a reduced propensity for priming an immune response which would prevent repeated dosage with an antibody therapeutic or imaging agent (e.g., the human-anti-murine-antibody “HAMA” response). Such anti-Gal3 antibodies or binding fragments thereof include, but are not limited to, humanized, chimeric, or xenogenic human anti-Gal3 antibodies or binding fragments thereof.

Chimeric anti-Gal3 antibodies or binding fragments thereof can be made, for example, by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains and incorporate only the complementarity-determining regions of the animal-derived antibody. This can be accomplished by carefully examining the sequence of the hyper-variable loops of the variable regions of a monoclonal antigen binding unit or monoclonal antibody and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The disclosure thus encompasses HuAbs with different variable (V) regions. It is within the skill of one in the art to determine suitable V region sequences and to optimize these sequences. Methods for obtaining antibodies with reduced immunogenicity are also described in U.S. Pat. No. 5,270,202 and EP 699,755, each hereby incorporated by reference in its entirety.

Humanized antibodies can be prepared by a process of analysis of the parental sequences and various conceptual humanized products using three dimensional models of the parental and humanized sequences. Three-dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions are selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

As noted above, an anti-Gal3 antibody or binding fragment thereof can be either “monovalent” or “multivalent.” Whereas the former has one binding site per antigen-binding unit, the latter contains multiple binding sites capable of binding to more than one antigen of the same or different kind. Depending on the number of binding sites, antigen binding units may be bivalent (having two antigen-binding sites), trivalent (having three antigen-binding sites), tetravalent (having four antigen-binding sites), and so on.

Multivalent anti-Gal3 antibodies or binding fragments thereof can be further classified on the basis of their binding specificities. A “monospecific” anti-Gal3 antibody or binding fragment thereof is a molecule capable of binding to one or more antigens of the same kind. A “multispecific” anti-Gal3 antibody or binding fragment thereof is a molecule having binding specificities for at least two different antigens. While such molecules normally will only bind two distinct antigens (i.e. bispecific anti-Gal3 antibodies), antibodies with additional specificities such as trispecific antibodies are encompassed by this expression when used herein. This disclosure further provides multispecific anti-Gal3 antibodies. Multispecific anti-Gal3 antibodies or binding fragments thereof are multivalent molecules capable of binding to at least two distinct antigens, e.g., bispecific and trispecific molecules exhibiting binding specificities to two and three distinct antigens, respectively.

In some embodiments, the methods further provide for screening for or identifying antibodies or binding fragments thereof capable of disrupting an interaction between Gal3 and a target protein, such as a target protein associated with stroke, traumatic brain injury, fibrin disorders, atherosclerosis, cardiovascular disease, or any other disease or disorder envisioned herein. In some aspects, the method may comprise: (a) contacting Gal3 protein with an antibody or binding fragment thereof that selectively binds to Gal3, thereby forming a Gal3-antibody complex; (b) contacting the Gal3-antibody complex with the target protein; (c) removing unbound target protein; and (d) detecting the target protein bound to the Gal3-antibody complex, wherein the antibody or binding fragment thereof is capable of disrupting an interaction of Gal3 and the target protein when the target protein is not detected in (d). In some cases, the method comprises an immunoassay. In some cases, the immunoassay is an enzyme-linked immunosorbent assay (ELISA).

### Host Cells

In some embodiments, the present disclosure provides host cells expressing any one of the anti-Gal3 antibodies or binding fragments thereof disclosed herein. A subject host cell typically comprises a nucleic acid encoding any one of the anti-Gal3 antibodies or binding fragments thereof disclosed herein.

The disclosure provides host cells transfected with the polynucleotides, vectors, or a library of the vectors described above. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For most animal cells, any of the above-mentioned methods is suitable for vector delivery. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

Once introduced into a suitable host cell, expression of the anti-Gal3 antibodies or binding fragments thereof can be determined using any nucleic acid or protein assay known in the art. For example, the presence of transcribed mRNA of light chain CDRs or heavy chain CDRs, or the anti-Gal3 antibody or binding fragment thereof can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes the anti-Gal3 antibody or binding fragment thereof.

Expression of the vector can also be determined by examining the expressed anti-Gal3 antibody or binding fragment thereof. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### Payload

In some embodiments, any anti-Gal3 antibody disclosed herein further comprises a payload. In some cases, the payload comprises a small molecule, a protein or functional fragment thereof, a peptide, or a nucleic acid polymer.

In some cases, the number of payloads conjugated to the anti-Gal3 antibody (e.g., the drug-to-antibody ratio or DAR) is about 1:1, one payload to one anti-Gal3 antibody. In some cases, the ratio of the payloads to the anti-Gal3 antibody is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some cases, the ratio of the payloads to the anti-Gal3 antibody is about 2:1. In some cases, the ratio of the payloads to the anti-Gal3 antibody is about 3:1. In some cases, the ratio of the payloads to the anti-Gal3 antibody is about 4:1. In some cases, the ratio of the payloads to the anti-Gal3 antibody is about 6:1. In some cases, the ratio of the payloads to the anti-Gal3 antibody is about 8:1. In some cases, the ratio of the payloads to the anti-Gal3 antibody is about 12:1.

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5208020, 5416064, 7276497, and 6716821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. No. 6884869, 7659241, 7498298, 7964566, 7750116, 8288352, 8703714, and 8871720.

In some embodiments, the payload comprises a DNA modifying agent. In some embodiments, the DNA modifying agent comprises DNA cleavers, DNA intercalators, DNA transcription inhibitors, or DNA cross-linkers. In some instances, the DNA cleaver comprises bleomycine A2, calicheamicin, or derivatives or analogs thereof. In some instances, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some instances, the DNA transcription inhibitor comprises dactinomycin. In some instances, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8404678 and 8163736. In some embodiments, the pyrrolobenzodiazepine is such as described in U.S. Pat. Nos. 8426402, 8802667, 8809320, 6562806, 6608192, 7704924, 7067511, US7612062, 7244724, 7528126, 7049311, 8633185, 8501934, and 8697688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120. In some embodiments, the PBD dimer is an unsymmetrical dimer. Examples of unsymmetrical PBD dimers include, but are not limited to, SJG-136 derivatives such as described in U.S. Pat. Nos. 8697688 and 9242013 and U.S. Publication No. 20140286970.

In some embodiments, the payload comprises an Akt inhibitor. In some cases, the Akt inhibitor comprises ipatasertib (GDC-0068) or derivatives thereof.

In some embodiments, the payload comprises a polymerase inhibitor, including, but not limited to polymerase II inhibitors such as a-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload comprises a detectable moiety. As used herein, a “detectable moiety” may comprise an atom, molecule, or compound that is useful in diagnosing, detecting or visualizing a location and/or quantity of a target molecule, cell, tissue, organ, and the like. Detectable moieties that can be used in accordance with the embodiments herein include, but are not limited to, radioactive substances (e.g. radioisotopes, radionuclides, radiolabels or radiotracers), dyes, contrast agents, fluorescent compounds or molecules, bioluminescent compounds or molecules, enzyme and enhancing agents (e.g. paramagnetic ions), or specific binding moieties such as streptavidin, avidin, or biotin. In addition, some nanoparticles, for example quantum dots or metal nanoparticles can be suitable for use as a detectable moiety.

Exemplary radioactive substances that can be used as detectable moieties in accordance with the embodiments herein include, but are not limited to, ¹⁸F, ¹⁸F-FAC, ³²P, ³³P, ⁴⁵Ti, ⁴⁷Sc ⁵²Fe, ⁵⁹Fe, ⁶²Cu, ⁶⁴Cu, ⁶⁷Cu ⁶⁷Ga, ⁶⁸Ga ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Exemplary paramagnetic ions substances that can be used as detectable markers include, but are not limited to ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the detectable marker is a radioactive metal or paramagnetic ion, in some embodiments, the marker can be reacted with a reagent having a long tail with one or more chelating groups attached to the long tail for binding these ions. The long tail can be a polymer such as a polylysine, polysaccharide, or other derivatized or derivatizable chain having pendant groups to which may be bound to a chelating group for binding the ions. Examples of chelating groups that may be used according to the embodiments herein include, but are not limited to, ethylenediaminetetraacetic acid (EDTA), diethylenetriaminepentaacetic acid (DTPA), DOTA, NOTA, NOGADA, NETA, deferoxamine (DfO), porphyrins, polyamines, crown ethers, bis-thiosemicarbazones, polyoximes, and like groups. The chelate can be linked to the antigen binding construct by a group which allows formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antigen binding constructs and carriers described herein. Macrocyclic chelates such as NOTA, NOGADA, DOTA, and TETA are of use with a variety of metals and radiometals including, but not limited to, radionuclides of gallium, yttrium and copper, respectively. Other ring-type chelates such as macrocyclic polyethers, which are of interest for stably binding radionuclides, such as Radium-223 for RAIT may be used. In certain embodiments, chelating moieties may be used to attach a PET imaging agent, such as an Aluminum-¹⁸F complex, to a targeting molecule for use in PET analysis.

Exemplary contrast agents that can be used as detectable moieties in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof.

Bioluminescent and fluorescent compounds or molecules and dyes that can be used as detectable moieties in accordance with the embodiments of the disclosure include, but are not limited to, fluorescein, fluorescein isothiocyanate (FITC), OREGON GREEN™, rhodamine, Texas red, tetrarhodimine isothiocynate (TRITC), Cy3, Cy5, and the like), fluorescent markers (e.g., green fluorescent protein (GFP), phycoerythrin, and the like), autoquenched fluorescent compounds that are activated by tumor-associated proteases, enzymes (e.g., luciferase, horseradish peroxidase, alkaline phosphatase, and the like), nanoparticles, biotin, digoxigenin or combinations thereof.

Enzymes that can be used as detectable moieties in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

In some embodiments, the payload is a nanoparticle. The term “nanoparticle” refers to a microscopic particle whose size is measured in nanometers, e.g., a particle with at least one dimension less than about 100 nm. Nanoparticles can be used as detectable substances because they are small enough to scatter visible light rather than absorb it. For example, gold nanoparticles possess significant visible light extinction properties and appear deep red to black in solution. As a result, compositions comprising antigen binding constructs conjugated to nanoparticles can be used for the in vivo imaging of T-cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles can be used as payloads to be conjugated to any one of the anti-Gal3 antibodies disclosed herein.

In some embodiments, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and antiandrogens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises an immune modulator. Exemplary immune modulators include, but are not limited to, gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, xanthines, stem cell growth factors, lymphotoxins, hematopoietic factors, tumor necrosis factor (TNF) (e.g., TNF-α), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S1 factor,” erythropoietin and thrombopoietin, or a combination thereof.

In some embodiments, the payload comprises an immunotoxin. Immunotoxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

In some instances, the payload comprises a nucleic acid polymer. In such instances, the nucleic acid polymer comprises short interfering nucleic acid (siNA), short interfering RNA (siRNA), double-stranded RNA (dsRNA), micro-RNA (miRNA), short hairpin RNA (shRNA), an antisense oligonucleotide. In other instances, the nucleic acid polymer comprises an mRNA, encoding, e.g., a cytotoxic protein or peptide or an apoptotic triggering protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL—panton Valentine leukocidin, aerolysin, clostridial Epsilon-toxin, clostridium perfringens enterotoxin), binary toxins (anthrax toxin, edema toxin, C. botulinum C2 toxin, C spirofome toxin, C. perfringens iota toxin, C. difficile cytolethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2). In additional instances, the nucleic acid polymer comprises a nucleic acid decoy. In some instances, the nucleic acid decoy is a mimic of protein-binding nucleic acids such as RNA-based protein-binding mimics. Exemplary nucleic acid decoys include transactivating region (TAR) decoy and Rev response element (RRE) decoy.

In some cases, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some cases, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display enhanced properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modifications include morpholinos, peptide nucleic acids (PNAs), methylphosphonate nucleotides, thiolphosphonate nucleotides, 2′-fluoro N3-P5′-phosphoramidites, 1′, 5′- anhydrohexitol nucleic acids (HNAs), or a combination thereof.

Any of the anti-Gal3 antibodies disclosed herein may be conjugated to one or more (e.g. 1, 2, 3, 4, 5, 6, 7, 8, 9, or 10, or more) payloads described herein.

### Conjugation Chemistry

In some instances, the payload is conjugated to an anti-Gal3 antibody described herein by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910.

In some instances, the payload is conjugated to an anti-Gal3 antibody described herein by a site-directed method utilizing a “traceless” coupling technology (Philochem). In some instances, the “traceless” coupling technology utilizes an N-terminal 1,2-aminothiol group on the binding moiety which is then conjugated with a polynucleic acid molecule containing an aldehyde group.

In some instances, the payload is conjugated to an anti-Gal3 antibody described herein by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond.

In some instances, the payload is conjugated to an anti-Gal3 antibody described herein by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation.

In some instances, the enzyme-catalyzed process comprises microbial transglutaminase (mTG). In some cases, the payload is conjugated to the anti-Gal3 antibody utilizing a microbial transglutaminze catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis.

In some instances, the payload is conjugated to an anti-Gal3 antibody by a method as described in PCT Publication No. WO2014/140317, which utilizes a sequence-specific transpeptidase and is hereby expressly incorporated by reference in its entirety.

In some instances, the payload is conjugated to an anti-Gal3 antibody described herein by a method as described in U.S. Pat. Publication Nos. 2015/0105539 and 2015/0105540.

### Linker

In some instances, a linker described herein comprises a natural or synthetic polymer, consisting of long chains of branched or unbranched monomers, and/or cross-linked network of monomers in two or three dimensions. In some instances, the linker includes a polysaccharide, lignin, rubber, or polyalkylene oxide (e.g., polyethylene glycol).

In some instances, the linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is built up from monomers of another polymer. In some instances, the linker comprises polyalkylene oxide. In some instances, the linker comprises PEG. In some instances, the linker comprises polyethylene imide (PEI) or hydroxy ethyl starch (HES).

In some cases, the polyalkylene oxide (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some embodiments, the linker is poly- or monodispersed polyalkylene oxide (e.g., PEG) and the indicated molecular weight represents an average of the molecular weight of the polyalkylene oxide, e.g., PEG, molecules.

In some embodiments, the linker comprises a polyalkylene oxide (e.g., PEG) and the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some cases, a dPEG is synthesized as a single molecular weight compound from pure (e.g., about 95%, 98%, 99%, or 99.5%) staring material in a stepwise fashion. In some cases, a dPEG has a specific molecular weight, rather than an average molecular weight.

In some instances, the linker is a discrete PEG, optionally comprising from 2 to 60, from 2 to 50, or from 2 to 48 repeating ethylene oxide units. In some cases, the linker comprises a dPEG comprising about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units.

In some embodiments, the linker is a polypeptide linker. In some instances, the polypeptide linker comprises at least 2, 3, 4, 5, 6, 7, 8, 10, 15, 20, 25, 30, 35, 40, 45, 50, 60, 70, 80, 90, 100, or more amino acid residues. In some instances, the polypeptide linker comprises at least 2, 3, 4, 5, 6, 7, 8, or more amino acid residues. In some instances, the polypeptide linker comprises at most 2, 3, 4, 5, 6, 7, 8, or less amino acid residues. In some cases, the polypeptide linker is a cleavable polypeptide linker (e.g., either enzymatically or chemically). In some cases, the polypeptide linker is a non-cleavable polypeptide linker. In some instances, the polypeptide linker comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the polypeptide linker comprises a peptide such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the polypeptide linker comprises L-amino acids, D-amino acids, or a mixture of both L- and D-amino acids.

In some instances, the linker comprises a homobifunctional linker. Exemplary homobifunctional linkers include, but are not limited to, Lomant’s reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (ρNPDP), ρ-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO 2015/038426, each of which is hereby expressly incorporated by reference in its entirety.

In some embodiments, the linker is a dendritic type linker. In some instances, the dendritic type linker comprises a branching, multifunctional linker moiety. In some instances, the dendritic type linker comprises PAMAM dendrimers.

In some embodiments, the linker is a traceless linker or a linker in which after cleavage does not leave behind a linker moiety (e.g., an atom or a linker group) to the antibody or payload. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

### Kit/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-Gal3 antibody as disclosed herein, host cells for producing one or more antibodies described herein, and/or vectors comprising nucleic acid molecules that encode the antibodies described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Numbered Arrangements 1

Some embodiments provided herein are described by way of the following provided numbered arrangements and also provided as possible combinations or overlapping embodiments:

1. A method for inhibiting, reducing, preventing, and/or treating stroke in a subject in need thereof, comprising administering an anti-Gal3 antibody or binding fragment thereof to the subject, thereby inhibiting, reducing, preventing, and/or treating the stroke.

2. The method of arrangement 1, further comprising selecting the subject as having the stroke or at risk of contracting the stroke prior to the administering step.

3. The method of arrangement 2, wherein selecting the subject as having the stroke or at risk of contracting the stroke comprises assessing the subject according to the National Institutes of Health Stroke Scale (NIHSS), modified Rankin Scale (mRS), Fugl-Meyer Assessment (FMA), Montreal Cognitive Assessment (MoCA), neuroimaging, optionally CT or MRI, and/or detecting stroke biomarkers, optionally S100 calcium binding protein B (S100B), glial fibrillary acidic protein (GFAP), neuron-specific enolase (NSE), and/or matrix metalloproteinase-9 (MMP9).

4. The method of any one of arrangements 1-3, further comprising detecting an amelioration of symptoms associated with the stroke after the administering step.

5. The method of arrangement 4, wherein detecting an amelioration of symptoms associated with the stroke after the administering step comprises detecting an improvement by the subject according to the NIHSS, mRS, FMA, or MoCA.

6. The method of arrangement 5, wherein:
a) the improvement by the subject according to the NIHSS comprises at least a 4 point decrease on the NIHSS;
b) the improvement by the subject according to the mRS comprises at least a 1 point decrease on the mRS;
c) the improvement by the subject according to the FMA comprises at least a 1 point increase on the FMA; and/or
d) the improvement by the subject according to the MoCA comprises at least a 1 point increase on the MoCA;
after the administering step, optionally as assessed 7, 35, and/or 75 days after the administering step.

7. The method of any one of arrangements 1-6, wherein the stroke is an ischemic stroke, thrombotic stroke, embolic stroke, transient ischemic attack, hemorrhagic stroke, intracerebral hemorrhage, or subarachnoid hemorrhage.

8. The method of any one of arrangements 1-7, wherein the stroke is intracerebral hemorrhage (ICH), optionally wherein the ICH is associated with deposition of amyloid aggregates and/or cerebral amyloid angiopathy (CAA).

9. The method of any one of arrangements 1-8, wherein administration of the anti-Gal3 antibody or binding fragment thereof to the subject prevents the stroke.

10. The method of any one of arrangements 1-9, wherein administration of the anti-Gal3 antibody or binding fragment thereof prevents loss of locomotor dysfunction, prevents incidence of microhemorrhage, prevents elevation of levels of activated microglia, prevents elevation of levels of activated astrocytes, prevents elevation of circulating proinflammatory immune cells, prevents elevation of circulating proinflammatory cytokines, or any combination thereof, in the subject.

11. The method of any one of arrangements 1-10, wherein administration of the anti-Gal3 antibody or binding fragment thereof to the subject treats the stroke.

12. The method of any one of arrangements 1-11, wherein administration of the anti-Gal3 antibody or binding fragment thereof reduces locomotor dysfunction, reduces microhemorrhage, reduces levels of activated microglia, reduces levels of activated astrocytes, reduces levels of circulating proinflammatory immune cells, reduces levels of circulating proinflammatory cytokines, or any combination thereof, in the subject.

13. The method of arrangement 12, wherein the reduction in locomotor dysfunction, reduction in microhemorrhage, reduction in levels of activated microglia, reduction in levels of activated astrocytes, reduction in levels of circulating proinflammatory cytokines, or any combination thereof, in the subject, is by at least 5%, 10%, 20%, 30%, 40%, 50%, 60%, 70%, 80%, 90%, or 95%, or any percentage within a range defined by any two of the aforementioned reductions.

14. The method of arrangements 12 or 13, wherein the reduction in locomotor dysfunction in the subject is by at least 50%.

15. The method of any one of arrangements 12-14, wherein the reduction in locomotor dysfunction is assessed by the Fugl-Meyer Assessment (FMA), Motor Assessment Scale (MAS), and/or the Chedoke-McMaster Stroke Assessment (CMSA).

16. The method of any one of arrangements 12-15, wherein the reduction in microhemorrhage in the subject is by at least 50%.

17. The method of any one of arrangements 12-16, wherein the reduction in microhemorrhage is assessed by neuroimaging, MRI, and/or Prussian blue staining.

18. The method of any one of arrangements 12-17, wherein the reduction in levels of activated microglia in the subject is by at least 20%.

19. The method of any one of arrangements 12-18, wherein the reduction in levels of activated microglia is assessed by detection of microglia activation markers, optionally ionized calcium-binding adaptor molecule 1 (Iba1), monocyte chemoattractant protein-1 (MCP-1), chitinase-3-like protein 1 (YKL-40), and/or soluble CD14 (sCD14), optionally from the cerebrospinal fluid of the subject.

20. The method of any one of arrangements 12-19, wherein the reduction in levels of activated astrocytes in the subject is by at least 30%.

21. The method of any one of arrangements 12-20, wherein the reduction in levels of activated astrocytes is assessed by detection of astrocyte activation markers, optionally GFAP and/or S100B, optionally from the cerebrospinal fluid of the subject.

22. The method of any one of arrangements 12-21, wherein the proinflammatory immune cells comprise NK cells, monocytes, and/or lymphocytes.

23. The method of any one of arrangements 12-22, wherein the proinflammatory cytokines comprise IL-6, TNF-α, and/or IL1β.

24. The method of any one of arrangements 1-23, wherein administering an anti-Gal3 antibody or binding fragment thereof to the subject comprises administration of one or more unit doses of the anti-Gal3 antibody or binding fragment thereof, optionally 1, 2, 3, 4, 5, 6, 7, 8, 9, or 10 unit doses.

25. The method of arrangement 24, wherein the one or more unit doses comprise 50, 60, 70, 80, 90, 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500 or 5000 mg of the anti-Gal3 antibody or binding fragment thereof per unit dose, or any amount per unit dose within a range defined by any two of the aforementioned amounts per unit dose.

26. The method of arrangements 24 or 25, wherein the one or more unit doses comprise 1, 1.1, 1.2, 1.3, 1.4, 1.5, 1.6, 1.7, 1.8, 1.9, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, or 150 mg/kg weight of the subject, or any amount per unit dose within a range defined by any two of the aforementioned amounts per unit dose.

27. The method of any one of arrangements 24-26, wherein the one or more unit doses are administered every 1, 2, 3, 4, 5, 6, or 7 days, or 1, 2, 3, or 4 weeks, or any interval within a range defined by any two of the aforementioned intervals.

28. The method of any one of arrangements 1-27, wherein the anti-Gal3 antibody or binding fragment thereof is administered enterally, orally, intranasally, parenterally, intracranially, subcutaneously, intramuscularly, intradermally, or intravenously, or any combination thereof, optionally intravenously.

29. The method of any one of arrangements 1-28, wherein the anti-Gal3 antibody or binding fragment thereof comprises one or more of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

30. The method of any one of arrangements 1-29, wherein the anti-Gal3 antibody or binding fragment thereof comprises one or more of: TB001, TB006, F846C.1F5, F846TC.16B5, 14H10.2C9, 15G7.2A7, 20H5.A3, 19D9.2E5, 19B5.2E6, or F847C.21H6.

31. The method of any one of arrangements 1-30, wherein the anti-Gal3 antibody or binding fragment thereof comprises TB006.

32. The method of any one of arrangements 1-31, wherein the anti-Gal3 antibody or binding fragment thereof is administered with one or more additional therapeutic compositions.

33. The method of arrangement 32, wherein the stroke is ischemic stroke, thrombotic stroke, embolic stroke, or transient ischemic attack, and the one or more additional therapeutic compositions comprise thrombolytics, tissue plasminogen activator (tPA), alteplase, reteplase, tenecteplase, desmoteplase, anticoagulants, ACE inhibitors, antihypertensives, nicardipine, or any combination thereof.

34. The method of arrangement 32, wherein the stroke is hemorrhagic stroke, intracerebral hemorrhage, or subarachnoid hemorrhage, and the one or more additional therapeutic compositions comprise antihypertensives, or nicardipine, or any combination thereof.

35. A method for inhibiting, reducing, preventing, and/or treating traumatic brain injury (TBI) in a subject in need thereof, comprising administering an anti-Gal3 antibody or binding fragment thereof to the subject, thereby inhibiting, reducing, preventing, and/or treating the TBI.

36. The method of arrangement 35, further comprising selecting the subject as having the TBI or at risk of contracting the TBI prior to the administering step.

37. The method of arrangement 36, wherein selecting the subject as having the TBI comprises assessing the subject for level of consciousness, memory loss, and/or the Glasgow Coma Scale, neuroimaging, optionally CT or MRI and/or detecting TBI biomarkers, optionally GFAP and/or ubiquitin carboxy-terminal hydrolase L1 (UCH-L1).

38. The method of any one of arrangements 35-37, further comprising detecting an amelioration of symptoms associated with the TBI after the administering step.

39. The method of any one of arrangements 35-38, wherein the TBI is associated with a concussion, edema, diffuse axonal injury, spinal cord injury, coma, neuroinflammation, microhemorrhage, astrocytosis, activated microglia, and/or hematoma.

40. The method of any one of arrangements 35-39, wherein administration of the anti-Gal3 antibody or binding fragment thereof to the subject prevents the TBI, concussion, edema, diffuse axonal injury, spinal cord injury, coma, neuroinflammation, microhemorrhage, astrocytosis, activated microglia, and/or hematoma.

41. The method of any one of arrangements 35-40, wherein administration of the anti-Gal3 antibody or binding fragment thereof prevents elevation of levels of activated microglia, prevents elevation of levels of activated astrocytes, prevents elevation of levels of macrophages, prevents elevation of levels of hyperphosphorylated Tau, prevents incidence of microhemorrhage, prevents neuroinflammation, prevents elevation of levels of circulating proinflammatory cytokines, or any combination thereof, in the subject.

42. The method of any one of arrangements 35-41, wherein administration of the anti-Gal3 antibody or binding fragment thereof to the subject treats the TBI, concussion, edema, diffuse axonal injury, spinal cord injury, coma, neuroinflammation, microhemorrhage, astrocytosis, activated microglia, and/or hematoma.

43. The method of any one of arrangements 35-42, wherein administration of the anti-Gal3 antibody or binding fragment thereof improves the level of consciousness, memory loss, and/or the Glasgow Coma Scale of the patient; and/or reduces levels of activated microglia, reduces levels of activated astrocytes, reduces levels of macrophages, reduces levels of hyperphosphorylated Tau, reduces microhemorrhage, reduces neuroinflammation, reduces levels of circulating proinflammatory cytokines or any combination thereof, in the subject.

44. The method of arrangement 43, wherein the reduction in levels of activated microglia, reduction in levels of activated astrocytes, reduction in levels of macrophages, reduction in levels of hyperphosphorylated Tau, reduction in microhemorrhage, reduction in neuroinflammation, reduction in levels of circulating proinflammatory cytokines, or any combination thereof, in the subject, is by at least 5%, 10%, 20%, 30%, 40%, 50%, 60%, 70%, 80%, 90%, or 95%, or any percentage within a range defined by any two of the aforementioned reductions.

45. The method of arrangements 43 or 44, wherein the reduction in levels of activated microglia in the subject is by at least 70%.

46. The method of any one of arrangements 43-45, wherein the reduction in levels of activated microglia is assessed by detection of microglia activation markers, optionally ionized calcium-binding adaptor molecule 1 (Iba1), monocyte chemoattractant protein-1 (MCP-1), chitinase-3-like protein 1 (YKL-40), and/or soluble CD14 (sCD14), optionally from the cerebrospinal fluid of the subject.

47. The method of any one of arrangements 43-46, wherein the reduction in levels of activated astrocytes in the subject is by at least 90%.

48. The method of any one of arrangements 43-47, wherein the reduction in levels of activated astrocytes is assessed by detection of astrocyte activation markers, optionally GFAP, optionally from the cerebrospinal fluid of the subject.

49. The method of any one of arrangements 43-48, wherein the reduction in levels of macrophages in the subject is by at least 70%.

50. The method of any one of arrangements 43-49, wherein the reduction in levels of macrophages is assessed by detection of macrophage activation markers, optionally CD68, optionally from the cerebrospinal fluid of the subject.

51. The method of any one of arrangements 43-50, wherein the reduction in levels of hyperphosphorylated Tau in the subject is by at least 80%.

52. The method of any one of arrangements 43-51, wherein the reduction in levels of microhemorrhage in the subject is by at least 80%.

53. The method of any one of arrangements 43-52, wherein the reduction in levels of microhemorrhage is assessed by neuroimaging, MRI, and/or Prussian blue staining.

54. The method of any one of arrangements 43-53, wherein the reduction in neuroinflammation in the subject is by at least 70%.

55. The method of any one of arrangements 43-54, wherein the proinflammatory cytokines comprise IL-6, IL-10, and/or TNF-α.

56. The method of any one of arrangements 35-55, wherein the anti-Gal3 antibody or binding fragment thereof is administered enterally, orally, intranasally, parenterally, intracranially, subcutaneously, intramuscularly, intradermally, or intravenously, or any combination thereof, optionally intravenously.

57. The method of any one of arrangements 35-56, wherein administering an anti-Gal3 antibody or binding fragment thereof to the subject comprises administration of one or more unit doses of the anti-Gal3 antibody or binding fragment thereof, optionally 1, 2, 3, 4, 5, 6, 7, 8, 9, or 10 unit doses.

58. The method of arrangement 57, wherein the one or more unit doses comprise 50, 60, 70, 80, 90, 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500 or 5000 mg of the anti-Gal3 antibody or binding fragment thereof per unit dose, or any amount per unit dose within a range defined by any two of the aforementioned amounts per unit dose.

59. The method of arrangements 57 or 58, wherein the one or more unit doses comprise 1, 1.1, 1.2, 1.3, 1.4, 1.5, 1.6, 1.7, 1.8, 1.9, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, or 150 mg/kg weight of the subject, or any amount per unit dose within a range defined by any two of the aforementioned amounts per unit dose.

60. The method of any one of arrangements 57-59, wherein the one or more unit doses are administered every 1, 2, 3, 4, 5, 6, or 7 days, or 1, 2, 3, or 4 weeks, or any interval within a range defined by any two of the aforementioned intervals.

61. The method of any one of arrangements 35-60, wherein the anti-Gal3 antibody or binding fragment thereof comprises one or more of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

62. The method of any one of arrangements 35-61, wherein the anti-Gal3 antibody or binding fragment thereof comprises one or more of: TB001, TB006, F846C.1F5, F846TC.16B5, 14H10.2C9, 15G7.2A7, 20H5.A3, 19D9.2E5, 19B5.2E6, or F847C.21H6.

63. The method of any one of arrangements 35-62, wherein the anti-Gal3 antibody or binding fragment thereof comprises TB006.

64. The method of any one of arrangements 35-63, wherein the anti-Gal3 antibody or binding fragment thereof is administered with one or more additional therapeutic compositions.

65. The method of arrangement 64, wherein the one or more additional therapeutic compositions comprise levodopa, bromocriptine, NMDA receptor antagonists, amantadine, memantine, cholinesterase inhibitors, tacrine, rivastigmine, galantamine, donepezil, or any combination thereof.

66. A method for inhibiting, reducing, preventing, and/or treating a disorder associated with fibrin activity or dysfunction in a subject in need thereof, comprising administering an anti-Gal3 antibody or binding fragment thereof to the subject, thereby inhibiting, reducing, preventing, and/or treating the disorder associated with fibrin activity or dysfunction.

67. The method of arrangement 66, wherein the disorder associated with fibrin activity or dysfunction comprises atherosclerosis, thrombosis, thromboembolism, carotid artery disease, coronary artery disease, peripheral artery disease, myocardial infarction, heart failure, heart attack, hypertension, chronic kidney disease, coagulopathy, or thrombocytopathy.

68. The method of arrangement 67, further comprising selecting the subject as having the disorder associated with fibrin activity or dysfunction or at risk of contracting the disorder associated with fibrin activity or dysfunction prior to the administering step.

69. The method of arrangement 68, wherein selecting the subject as having the disorder associated with fibrin activity or dysfunction comprises detecting thrombotic biomarkers, optionally fibrinogen, C-reactive protein (CRP), and/or plasminogen activator inhibitor-1 (PAI-1).

70. The method of any one of arrangements 66-69, further comprising detecting an amelioration of symptoms associated with the disorder associated with fibrin activity or dysfunction after the administering step.

71. The method of any one of arrangements 66-70, wherein administration of the anti-Gal3 antibody or binding fragment thereof to the subject prevents neuroinflammation and/or fibrin oligomerization, in the subject.

72. The method of any one of arrangements 66-71, wherein administration of the anti-Gal3 antibody or binding fragment thereof to the subject reduces neuroinflammation and/or fibrin oligomerization in the subject.

73. The method of arrangement 72, wherein the reduction in neuroinflammation and/or the reduction in fibrin oligomerization, in the subject, is by at least 5%, 10%, 20%, 30%, 40%, 50%, 60%, 70%, 80%, 90%, or 95%, or any percentage within a range defined by any two of the aforementioned reductions.

74. The method of any one of arrangements 66-73, wherein the anti-Gal3 antibody or binding fragment thereof is administered enterally, orally, intranasally, parenterally, intracranially, subcutaneously, intramuscularly, intradermally, or intravenously, or any combination thereof, optionally intravenously.

75. The method of any one of arrangements 66-74, wherein administering an anti-Gal3 antibody or binding fragment thereof to the subject comprises administration of one or more unit doses of the anti-Gal3 antibody or binding fragment thereof, optionally 1, 2, 3, 4, 5, 6, 7, 8, 9, or 10 unit doses.

76. The method of arrangement 75, wherein the one or more unit doses comprise 50, 60, 70, 80, 90, 100, 200, 300, 400, 500, 600, 700, 800, 900, 1000, 1100, 1200, 1300, 1400, 1500, 1600, 1700, 1800, 1900, 2000, 2500, 3000, 3500, 4000, 4500 or 5000 mg of the anti-Gal3 antibody or binding fragment thereof per unit dose, or any amount per unit dose within a range defined by any two of the aforementioned amounts per unit dose

77. The method of arrangements 75 or 76, wherein the one or more unit doses comprise 1, 1.1, 1.2, 1.3, 1.4, 1.5, 1.6, 1.7, 1.8, 1.9, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, or 150 mg/kg weight of the subject, or any amount per unit dose within a range defined by any two of the aforementioned amounts per unit dose.

78. The method of any one of arrangements 75-77, wherein the one or more unit doses are administered every 1, 2, 3, 4, 5, 6, or 7 days, or 1, 2, 3, or 4 weeks, or any interval within a range defined by any two of the aforementioned intervals.

79. The method of any one of arrangements 66-78, wherein the anti-Gal3 antibody or binding fragment thereof comprises one or more of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof

80. The method of any one of arrangements 66-79, wherein the anti-Gal3 antibody or binding fragment thereof comprises one or more of: TB001, TB006, F846C.1F5, F846TC.16B5, 14H10.2C9, 15G7.2A7, 20H5.A3, 19D9.2E5, 19B5.2E6, or F847C.21H6.

81. The method of any one of arrangements 66-80, wherein the anti-Gal3 antibody or binding fragment thereof comprises TB006.

82. The method of any one of arrangements 66-81, wherein the anti-Gal3 antibody or binding fragment thereof is administered with one or more additional therapeutic compositions.

83. The method of arrangement 82, wherein the one or more additional therapeutic compositions comprises anti-clotting agents, aspirin, statins, antihypertensives, diuretics, calcium channel blockers, ACE inhibitors, angiotensin II receptor antagonists, adrenergic receptor antagonists, vasodilators, renin inhibitors, aldosterone receptor antagonists, endothelium receptor blockers, or any combination thereof.

84. The method of any one of arrangements 1-83, wherein administering an anti-Gal3 antibody or binding fragment thereof to the subject comprises administration of 5 unit doses of the anti-Gal3 antibody or binding fragment thereof, wherein each unit dose comprises 1000 mg or about 1000 mg of the anti-Gal3 antibody or binding fragment thereof, wherein the unit doses are administered every 7 days or about 7 days, and wherein each unit dose is administered over the course of 1 hour or about 1 hour.

85. A method comprising administering 5 unit doses of an anti-Gal3 antibody or binding fragment thereof, wherein each unit dose comprise 1000 mg or about 1000 mg of the anti-Gal3 antibody or binding fragment thereof, wherein the unit doses are administered every 7 days or about 7 days, and wherein each unit dose is administered over the course of 1 hour or about 1 hour.

86. The method of arrangements 84 or 85, wherein the anti-Gal3 antibody or binding fragment thereof is TB006 or a binding fragment thereof.

87. The method of any one of arrangements 84-86, wherein each unit dose is formulated as 1000 mg or about 1000 mg of the anti-Gal3 antibody or binding fragment thereof in 250 mL of sterile saline.

88. The method of any one of arrangements 84-87, wherein each unit dose is prepared from single-use sealed injectable glass vials comprising 8 mL of 20 mg/mL of the anti-Gal3 antibody or binding fragment thereof.

89. The method of any one of arrangements 84-88, wherein the anti-Gal3 antibody or binding fragment thereof is administered intravenously.

90. A single-use sealed injectable glass vial comprising 8 mL of 20 mg/mL of an anti-Gal antibody or binding fragment thereof, optionally wherein the anti-Gal3 antibody or binding fragment thereof is TB006 or a binding fragment thereof.

91. An IV infusion bag comprising 1000 mg or about 1000 mg of an anti-Gal3 antibody or binding fragment thereof dissolved in 250 mL or about 250 mL of saline, optionally wherein the anti-Gal3 antibody or binding fragment thereof is TB006 or a binding fragment thereof.

### Numbered Arrangements 2

Some embodiments provided herein are described by way of the following provided numbered arrangements and also provided as possible combinations or overlapping embodiments:

1. An anti-Gal3 antibody for use in inhibiting, reducing, preventing, and/or treating stroke in a subject in need thereof, wherein
the anti-Gal3 antibody or binding fragment thereof comprises one or more of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, TB001, TB006, F846C.1F5, F846TC.16B5, 14H10.2C9, 15G7.2A7, 20H5.A3, 19D9.2E5, 19B5.2E6, or F847C.21H6, or a binding fragment thereof; and wherein
administration of the anti-Gal3 antibody or binding fragment thereof to the subject, inhibits, reduces, prevents, and/or treats the stroke.

2. An anti-Gal3 antibody for use in inhibiting, reducing, preventing, and/or treating traumatic brain injury (TBI) in a subject in need thereof, wherein
the anti-Gal3 antibody or binding fragment thereof comprises one or more of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, TB001, TB006, F846C.1F5, F846TC.16B5, 14H10.2C9, 15G7.2A7, 20H5.A3, 19D9.2E5, 19B5.2E6, or F847C.21H6, or a binding fragment thereof; and wherein
administration of the anti-Gal3 antibody or binding fragment thereof to the subject, thereby inhibits, reduces, prevents, and/or treats the TBI.

3. An anti-Gal3 antibody for use in inhibiting, reducing, preventing, and/or treating a disorder associated with fibrin activity or dysfunction in a subject in need thereof, An anti-Gal3 antibody for use in inhibiting, reducing, preventing, and/or treating traumatic brain injury (TBI) in a subject in need thereof, wherein
the anti-Gal3 antibody or binding fragment thereof comprises one or more of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, TB001, TB006, F846C.1F5, F846TC.16B5, 14H10.2C9, 15G7.2A7, 20H5.A3, 19D9.2E5, 19B5.2E6, or F847C.21H6, or a binding fragment thereof; and wherein
administration of the anti-Gal3 antibody or binding fragment thereof to the subject, thereby inhibits, reduces, prevents, and/or treats the disorder associated with fibrin activity or dysfunction.

4. A method comprising administering 5 unit doses of an anti-Gal3 antibody or binding fragment thereof, wherein each unit dose comprise 1000 mg or about 1000 mg of the anti-Gal3 antibody or binding fragment thereof, wherein the unit doses are administered every 7 days or about 7 days, and wherein each unit dose is administered over the course of 1 hour or about 1 hour.

5. A single-use sealed injectable glass vial comprising 8 mL of 20 mg/mL of an anti-Gal antibody or binding fragment thereof, optionally wherein the anti-Gal3 antibody or binding fragment thereof is TB006 or a binding fragment thereof.

6. An IV infusion bag comprising 1000 mg or about 1000 mg of an anti-Gal3 antibody or binding fragment thereof dissolved in 250 mL or about 250 mL of saline, optionally wherein the anti-Gal3 antibody or binding fragment thereof is TB006 or a binding fragment thereof.

7. The use of any one of the previous arrangements, wherein
a.) the subject is identified as having the stroke or at risk of contracting the stroke prior to the administering step;
b.) selecting the subject as having the stroke or at risk of contracting the stroke prior to the administering step;
c.) selecting the subject as having the stroke or at risk of contracting the stroke comprises assessing the subject according to the National Institutes of Health Stroke Scale (NIHSS), modified Rankin Scale (mRS), Fugl-Meyer Assessment (FMA), Montreal Cognitive Assessment (MoCA), neuroimaging, optionally CT or MRI, and/or detecting stroke biomarkers, optionally S100 calcium binding protein B (S100B), glial fibrillary acidic protein (GFAP), neuron-specific enolase (NSE), and/or matrix metalloproteinase-9 (MMP9);
d.) an amelioration of symptoms associated with the stroke is detected after the administering step;
e.) detecting an amelioration of symptoms associated with the stroke after the administering step comprises detecting an improvement by the subject according to the NIHSS, mRS, FMA, or MoCA;
f.) the improvement by the subject according to the NIHSS comprises at least a 4 point decrease on the NIHSS;
g.) the improvement by the subject according to the mRS comprises at least a 1 point decrease on the mRS;
h.) the improvement by the subject according to the FMA comprises at least a 1 point increase on the FMA;
i.) the improvement by the subject according to the MoCA comprises at least a 1 point increase on the MoCA;
j.) improvement by the subject according to the NIHSS, mRS, FMA, or MoCA is assessed 7, 35, and/or 75 days after the administering step;
k.) improvement by the subject according to the NIHSS, mRS, FMA, or MoCA is detected 7, 35, and/or 75 days after the administering step;
ischemic attack, hemorrhagic stroke, intracerebral hemorrhage, or subarachnoid hemorrhage;
m.) the stroke is intracerebral hemorrhage (ICH), optionally wherein the ICH is associated with deposition of amyloid aggregates and/or cerebral amyloid angiopathy (CAA);
n.) administration of the anti-Gal3 antibody or binding fragment thereof to the subject prevents the stroke;
o.) administration of the anti-Gal3 antibody or binding fragment thereof prevents loss of locomotor dysfunction, prevents incidence of microhemorrhage, prevents elevation of levels of activated microglia, prevents elevation of levels of activated astrocytes, prevents elevation of circulating proinflammatory immune cells, prevents elevation of circulating proinflammatory cytokines, or any combination thereof, in the subject;
p.) administration of the anti-Gal3 antibody or binding fragment thereof to the subject treats the stroke;
q.) the reduction in locomotor dysfunction, reduction in microhemorrhage, reduction in levels of activated microglia, reduction in levels of activated astrocytes, reduction in levels of circulating proinflammatory cytokines, or any combination thereof, in the subject, is by at least 5%, 10%, 20%, 30%, 40%, 50%, 60%, 70%, 80%, 90%, or 95%, or any percentage within a range defined by any two of the aforementioned reductions;
r.) the reduction in locomotor dysfunction is assessed by the Fugl-Meyer Assessment (FMA), Motor Assessment Scale (MAS), and/or the Chedoke-McMaster Stroke Assessment (CMSA);
s.) the reduction in microhemorrhage is assessed by neuroimaging, MRI, and/or Prussian blue staining;
t.) the reduction in levels of activated microglia is assessed by detection of microglia activation markers, optionally ionized calcium-binding adaptor molecule 1 (Iba1), monocyte chemoattractant protein-1 (MCP-1), chitinase-3-like protein 1 (YKL-40), and/or soluble CD14 (sCD14), optionally from the cerebrospinal fluid of the subject;
u.) the reduction in levels of activated astrocytes is assessed by detection of astrocyte activation markers, optionally GFAP and/or S100B, optionally from the cerebrospinal fluid of the subject;
v.) the proinflammatory immune cells comprise NK cells, monocytes, and/or lymphocytes;
w.) the proinflammatory cytokines comprise IL-6, TNF-α, and/or IL1β; x.) administering an anti-Gal3 antibody or binding fragment thereof to the subject comprises administration of one or more unit doses of the anti-Gal3 antibody or binding fragment thereof, optionally 1, 2, 3, 4, 5, 6, 7, 8, 9, or 10 unit doses;
500, 600, 700, 800, 900, 1000, 1100, 1200, 1300, 1400, 1500, 1600, 1700, 1800, 1900, 2000, 2500, 3000, 3500, 4000, 4500 or 5000 mg of the anti-Gal3 antibody or binding fragment thereof per unit dose, or any amount per unit dose within a range defined by any two of the aforementioned amounts per unit dose;
z.) the one or more unit doses comprise 1, 1.1, 1.2, 1.3, 1.4, 1.5, 1.6, 1.7, 1.8, 80, 85, 90, 95, 100, 105, 110, 115, 120, 125, 130, 135, 140, 145, or 150 mg/kg weight of the subject, or any amount per unit dose within a range defined by any two of the aforementioned amounts per unit dose;
or 1, 2, 3, or 4 weeks, or any interval within a range defined by any two of the aforementioned intervals;
bb.) the anti-Gal3 antibody or binding fragment thereof is administered enterally, orally, intranasally, parenterally, intracranially, subcutaneously, intramuscularly, intradermally, or intravenously, or any combination thereof, optionally intravenously;
cc.)the anti-Gal3 antibody or binding fragment thereof is administered with one or more additional therapeutic compositions;
dd.) the stroke is ischemic stroke, thrombotic stroke, embolic stroke, or transient ischemic attack, and the one or more additional therapeutic compositions comprise thrombolytics, tissue plasminogen activator (tPA), alteplase, reteplase, tenecteplase, desmoteplase, anticoagulants, ACE inhibitors, antihypertensives, nicardipine, or any combination thereof;
ee.)the stroke is hemorrhagic stroke, intracerebral hemorrhage, or subarachnoid hemorrhage, and the one or more additional therapeutic compositions comprise antihypertensives, or nicardipine, or any combination thereof;
ff.) the subject is selected as having the TBI or at risk of contracting the TBI prior to the administering step;
gg.) selecting the subject as having the TBI comprises assessing the subject for level of consciousness, memory loss, and/or the Glasgow Coma Scale, neuroimaging, optionally CT or MRI and/or detecting TBI biomarkers, optionally GFAP and/or ubiquitin carboxy-terminal hydrolase L1 (UCH-L1);
hh.) an amelioration of symptoms associated with the TBI is detected after the administering step;
ii.) the TBI is associated with a concussion, edema, diffuse axonal injury, spinal cord injury, coma, neuroinflammation, microhemorrhage, astrocytosis, activated microglia, and/or hematoma;
jj.) administration of the anti-Gal3 antibody or binding fragment thereof to the subject prevents the TBI, concussion, edema, diffuse axonal injury, spinal cord injury, coma, neuroinflammation, microhemorrhage, astrocytosis, activated microglia, and/or hematoma;
kk.) administration of the anti-Gal3 antibody or binding fragment thereof prevents elevation of levels of activated microglia, prevents elevation of levels of activated astrocytes, prevents elevation of levels of macrophages, prevents elevation of levels of hyperphosphorylated Tau, prevents incidence of microhemorrhage, prevents neuroinflammation, prevents elevation of levels of circulating proinflammatory cytokines, or any combination thereof, in the subject;
11.) administration of the anti-Gal3 antibody or binding fragment thereof to the subject treats the TBI, concussion, edema, diffuse axonal injury, spinal cord injury, coma, neuroinflammation, microhemorrhage, astrocytosis, activated microglia, and/or hematoma;
mm.) administration of the anti-Gal3 antibody or binding fragment thereof improves the level of consciousness, memory loss, and/or the Glasgow Coma Scale of the patient; and/or reduces levels of activated microglia, reduces levels of activated astrocytes, reduces levels of macrophages, reduces levels of hyperphosphorylated Tau, reduces microhemorrhage, reduces neuroinflammation, reduces levels of circulating proinflammatory cytokines or any combination thereof, in the subject;
nn.) reduction in levels of activated microglia is assessed by detection of microglia activation markers, optionally ionized calcium-binding adaptor molecule 1 (Iba1), monocyte chemoattractant protein-1 (MCP-1), chitinase-3-like protein 1 (YKL-40), and/or soluble CD14 (sCD14), optionally from the cerebrospinal fluid of the subject;
oo.) the reduction in levels of activated astrocytes is assessed by detection of astrocyte activation markers;
pp.) the reduction in levels of macrophages is assessed by detection of macrophage activation markers, optionally CD68, optionally from the cerebrospinal fluid of the subject;
qq.) the reduction in levels of microhemorrhage is assessed by neuroimaging, MRI, and/or Prussian blue staining;
rr.) the proinflammatory cytokines comprise IL-6, IL-10, and/or TNF-α;
ss.) the anti-Gal3 antibody or binding fragment thereof is administered enterally, orally, intranasally, parenterally, intracranially, subcutaneously, intramuscularly, intradermally, or intravenously, or any combination thereof, optionally intravenously;
tt.) the disorder associated with fibrin activity or dysfunction comprises atherosclerosis, thrombosis, thromboembolism, carotid artery disease, coronary artery disease, peripheral artery disease, myocardial infarction, heart failure, heart attack, hypertension, chronic kidney disease, coagulopathy, or thrombocytopathy;
uu.) the subject is selected as having a disorder associated with fibrin activity or dysfunction or at risk of contracting a disorder associated with fibrin activity or dysfunction prior to the administering step;
vv.) selecting the subject as having the disorder associated with fibrin activity or dysfunction comprises detecting thrombotic biomarkers, optionally fibrinogen, C-reactive protein (CRP), and/or plasminogen activator inhibitor-1 (PAI-1);
ww.) an amelioration of symptoms associated with a disorder associated with fibrin activity or dysfunction is detected after the administering step;
xx.) administration of the anti-Gal3 antibody or binding fragment thereof to the subject prevents neuroinflammation and/or fibrin oligomerization, in the subject;
yy.) administration of the anti-Gal3 antibody or binding fragment thereof to the subject reduces neuroinflammation and/or fibrin oligomerization in the subject;
zz.) the reduction in neuroinflammation and/or the reduction in fibrin oligomerization, in the subject, is by at least 5%, 10%, 20%, 30%, 40%, 50%, 60%, 70%, 80%, 90%, or 95%, or any percentage within a range defined by any two of the aforementioned reductions;
aaa.) the one or more additional therapeutic compositions comprises anti-clotting agents, aspirin, statins, antihypertensives, diuretics, calcium channel blockers, ACE inhibitors, angiotensin II receptor antagonists, adrenergic receptor antagonists, vasodilators, renin inhibitors, aldosterone receptor antagonists, endothelium receptor blockers, or any combination thereof;
bbb.) administering an anti-Gal3 antibody or binding fragment thereof to the subject comprises administration of 5 unit doses of the anti-Gal3 antibody or binding fragment thereof, wherein each unit dose comprises 1000 mg or about 1000 mg of the anti-Gal3 antibody or binding fragment thereof, wherein the unit doses are administered every 7 days or about 7 days, and wherein each unit dose is administered over the course of 1 hour or about 1 hour;
ccc.) each unit dose is formulated as 1000 mg or about 1000 mg of the anti-Gal3 antibody or binding fragment thereof in 250 mL of sterile saline; or wherein
ddd.) each unit dose is prepared from single-use sealed injectable glass vials comprising 8 mL of 20 mg/mL of the anti-Gal3 antibody or binding fragment thereof.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1. Therapeutic Efficacy of Gal3 Antibody in an Intracerebral Hemorrhagic Stroke Model

Anti-Gal3 antibody treatment (using mTB001 as a representative antibody) was assessed for efficacy and pharmacodynamics in an intracerebral hemorrhage (ICH) model of stroke. Stroke was induced in C57BL/6J mice by stereotactic injection of autologous tail blood. Beginning one week after ICH induction, mice were treated with 8 doses of 10 mg/kg mTB001 or MOPC21 isotype control. Mice that did not receive ICH served as healthy controls. Six weeks after stroke induction, brain pathology was measured with immunohistochemistry (IHC). Anti-Gal3 antibody mTB001 significantly improved locomotor function compared to MOPC21 controls. In the brain, mTB001 reduced levels of microhemorrhages.

Intracerebral hemorrhage is a potentially fatal stroke subtype that accounts for an in-hospital mortality rate and a disability rate of 40% and 80%, respectively. ICH is responsible for 10-15% of all strokes, and the worldwide incidence of ICH is 2 million cases per year with approximately 120,000 cases per year in the United States. However, the incidence is expected to have doubled by 2050 due to aging and the spreading use of anticoagulants. Notably, there is no effective treatment for ICH, and the pathophysiology of the disease is poorly defined. Some pathogenic effects of ICH may be attributed to deposition of amyloid β (Aβ) in the vasculature, as discussed herein.

Gal3 belongs to the family of water-soluble lectins, which are sugar binding proteins. Galectins preferentially bind to β-galactoside derivatives and can crosslink surface glycoproteins by binding galactose residues. Gal3 is the only known member of a family of chimera-type galectins which contains a C-terminal carbohydrate recognition domain (CRD) for sugar binding and an N-terminal non-CRD for self-association. Gal3 has ubiquitous subcellular distribution and functions in the nuclear, cytoplasmic, and extracellular compartments. Increasing body of evidence indicates Gal3 as a crucial player involved in regulation of brain inflammation and AD pathology. However, there exists a knowledge gap in the understanding of their cellular expression and function after ICH.

Induction of intracerebral hemorrhage: Eighteen 10-weeks old C57BL/6J mice had ICH induced by stereotactic injection of autologous tail blood. Animals were anesthetized with isoflurane, placed on a stereotaxic surgery frame, then their shaved heads were sterilized with 70% ethanol and povidone-iodine prep. ICH was induced by the stereotactic injection of autologous tail blood. Hamilton syringe (25 µL) was mounted onto the injection pump, and the needle (26 gauge) stereotactically directed over the bregma. Next, stereotactic manipulator arms were adjusted to position the needle 0.2 mm anterior and 2 mm laterally to the right. At these coordinates, a small cranial burr hole was made using a variable speed drill with a 1 mm drill bit. The tail of mice was disinfected with 70% ethanol. Central tail artery was punctured with a sterile needle (e.g. 26 gauge) and arterial blood was collected into an un-heparinized capillary tube. Blood was quickly transferred from the capillary tube into the glass barrel of the Hamilton syringe, then the plunger was inserted. The Hamilton syringe containing 30 µL or more of arterial blood was mounted onto the injection pump with inserted needle (with its beveled edge facing the sagittal suture) through the burr hole just until its bevel was no longer visible. From this point, the needle was advanced 3 mm ventrally and 5 µL of autologous blood was injected at a rate of 2 µL/min. After completion of the first injection, the needle was advanced to 0.7 mm further in depth. After 5 minutes, 25 µL of blood was injected into the right striatum. Upon completion of the second injection, the needle was left in position for an additional 10 minutes, before withdrawing it at a rate of 1 mm/min. The burr hole was sealed with bone wax, and the skin was sutured.

Locomotor test using Rotarod: The Rotarod test was used to assess motor coordination and balance in rodents. Testing was performed on a modified Rotarod apparatus. On the day of testing, mice were acclimated in their home cages in the testing room for at least 15 minutes (acclimation phase). Testing consisted of three trials on a modified Rotarod separated by 15 minutes inter-trial intervals. The Rotarod apparatus is a rotating rod with a 5 cm diameter made of hard plastic material covered by grey rubber foam divided into lanes that are each 5 cm wide. Mice were placed in individual lanes while the rod was rotated at 4 revolutions per minute (rpm). Once all the mice were able to walk forward for a few seconds, the trial was started with the rod accelerating from 4 rpm to 40 rpm over the course of 300 seconds. The latency (time) at which each mouse falls off the rod, the distance travelled before falling and the reason for trial end (falling, jumping, or passive rotation) were recorded.

Dose groups and drug administration: After one week following ICH induction and based on the pre-dose behavior, animals were randomized into two groups based on the Rotarod locomotor function test. One group (n=9) received 8 doses of mTB001 (804-mIgG2a-LALAPG), another group (n=9) received 8 doses of MOPC21 (MOPC21-mIgG2a-LALAPG) as isotype control antibody, each dose was 10 mg/kg. The antibodies were injected intraperitoneally (i.p.) twice in a week. After treatment, mice again underwent locomotor function test. In this behavior procedure, wild type un-injected C57BL/6J mice were also included.

Sample collection: Mice were anesthetized with isoflurane, blood was collected by cardiac puncture, and mice were perfused transcardially with cold phosphate-buffer saline (PBS). For the comparison, mice dosed eight times either with mTB001 and MOPC21 (isotype control) antibody along with wild type un-injected mice were sacrificed. Brain tissues were fixed for 48 hours with 4% paraformaldehyde in PBS, pH 7.4 at 4° C. and stored in PBS/0.02% sodium azide (NaN₃) at 4° C. until use.

Immunohistochemistry: Fixed brain tissues were sectioned (40 µm) with a vibratome. Sagittal sections were collected in PBS containing 0.02% NaN₃ and stored at 4° C. prior to staining. Determination of microhemorrhages was performed by staining for ferric acid using Prussian Blue (5% potassium hexacyanoferrate trihydrate + 5% hydrochloric acid) for 30 minutes. Sections were then rinsed with water and counterstained with Nuclear Fast Red. Slides were cover-slipped using DPX mounting media. To stain for microglia and astrocytes, endogenous peroxidase in tissue was blocked by treating with 3% H₂O₂ in PBS for 10 min at 25° C. Non-specific background staining was blocked by 1 hour incubation in 2.5% horse serum, 0.3% Triton-X 100 (TX) at 25° C. Tissues were incubated with primary antibodies against ionized calcium binding adaptor molecule 1 (Iba1; which stains for microglia) or glial fibrillary acidic protein (GF AP; which stains for astrocytes) overnight at 4° C., rinsed three times with PBS, 0.1% TX, followed by biotinylated secondary antibody (anti-mouse) detection with an avidin-biotin complex (ABC) peroxidase kit, and visualized with a 3,3′-diaminobenzidine (DAB) substrate kit. After DAB staining, brain tissues were mounted on Superfrost Plus (ThermoFisher) microscope slides and dehydrated using different percentages of alcohols and xylene. Slides were cover-slipped using DPX mounting media. Control experiments omitting either primary or secondary antibody resulted in no staining. Brain tissues were scanned using an Aperio VERSA Brightfield, Fluorescence & FISH Digital Pathology Scanner.

Statistical Analysis: The behavioral data were analyzed using GraphPad Prism-8. All values were reported as mean ± SEM and significance set at p < 0.05. NIH ImageJ software was used to analyze the immunohistochemistry and quantification was done using GraphPad Prism-8.

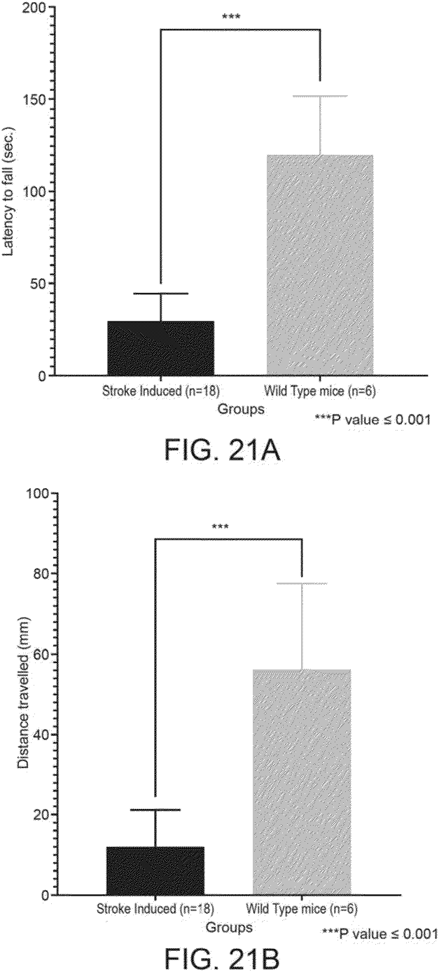

Induction of Intracerebral hemorrhage (ICH) leads to locomotor deficit: C57BL/6J mice induced with ICH demonstrated significant deficit in locomotor activity before mTB001 dosing, as evidenced by the significantly shorter latency to fall (FIG. 21A) and distance travelled (FIG. 21B) in the Rotarod test as compared to age matched healthy control C57BL/6J mice.

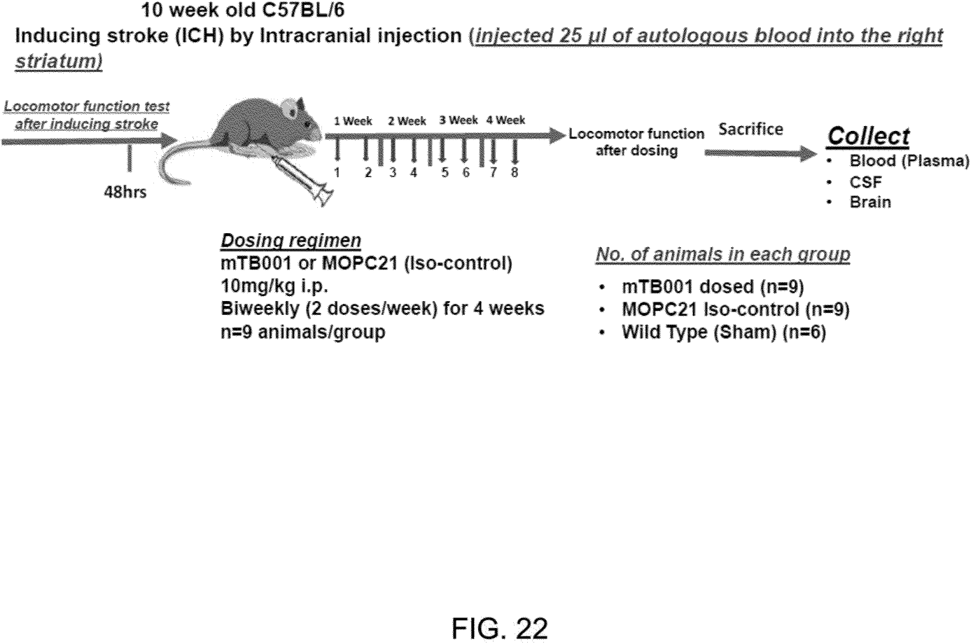

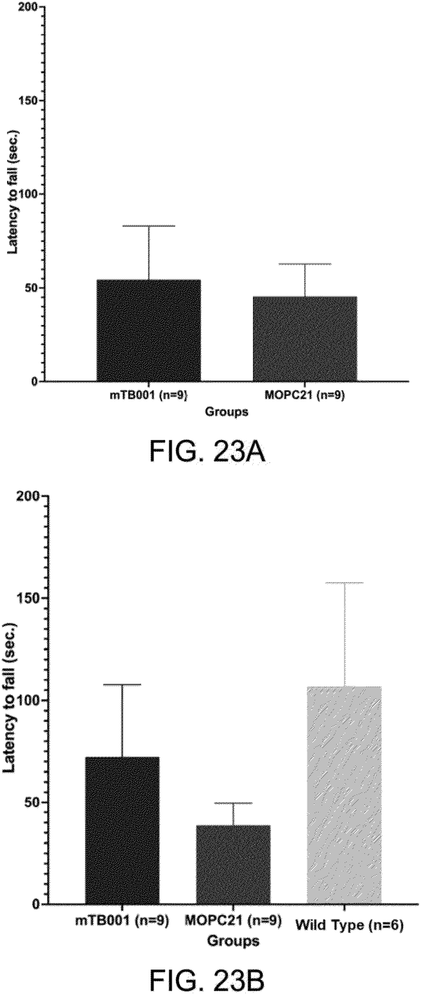

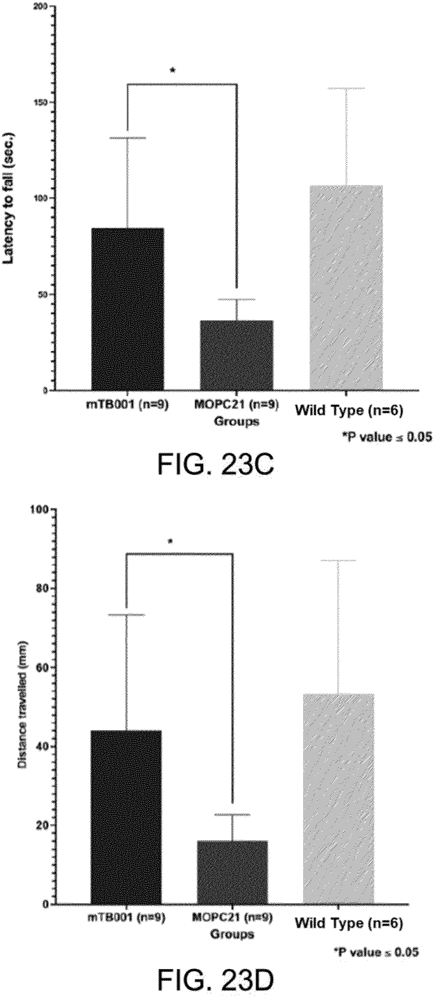

Treatment with mTB001 improves locomotor function test: Based on the locomotor activity test, mice were randomized and divided into two groups (n=9 animals/group) with similar capabilities (“Before Dosing” measurement). mTB001 and MOPC21 antibodies (10 mg/kg) was administered intraperitoneally. Wild-type mice served as controls. FIG. 22 depicts the exemplary dose regimen used after inducing ICH. Animals were tested for locomotor ability after 4 (FIG. 23A), 6 (FIG. 23B), and 8 doses. After 8 doses of mTB001 antibody, animals performed significantly better on the Rotarod test, as seen by increased latency before fall (FIG. 23C) and distance travelled (FIG. 23D). These data demonstrate that treatment with an anti-Gal3 antibody results in significant improvement in locomotor activity in an animal model of stroke.

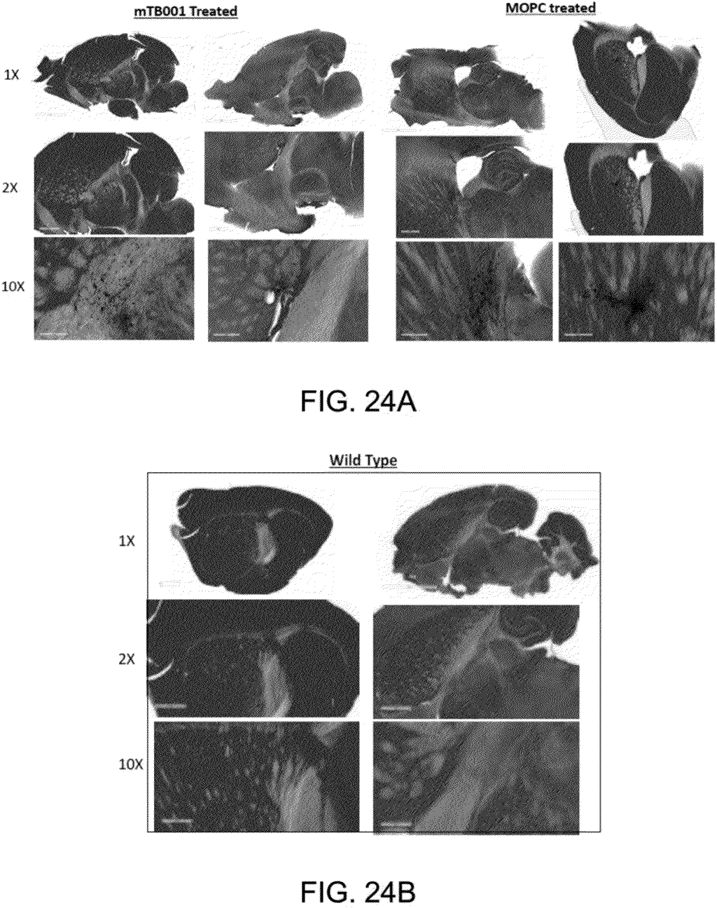

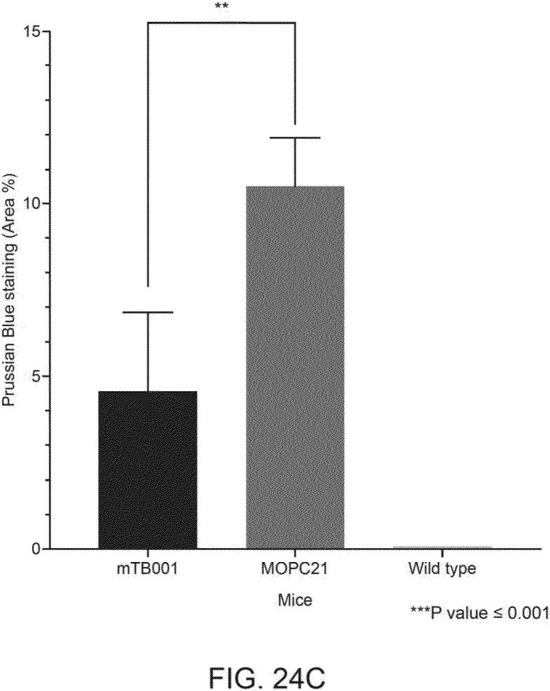

Treatment with mTB001 reduces microhemorrhage: An increased incidence of microhemorrhage has been observed associated with ICH. In addition, the accumulation of Aβ in the blood vessel wall increases the risk of intracranial hemorrhage and stroke in the human brain. The effect of mTB001 on microhemorrhages was measured by Prussian Blue staining. Blood accumulation in the brains of mTB001-treated mice (after 8 doses) was reduced (FIG. 24A), while no microhemorrhage was observed in healthy control sham mice (FIG. 24B). FIG. 24C depicts a quantification of microhemorrhage by brain tissue area seen in FIGS. 24A-B. These data demonstrate that treatment with an anti-Gal3 antibody improves microhemorrhage symptoms in an animal model of stroke.

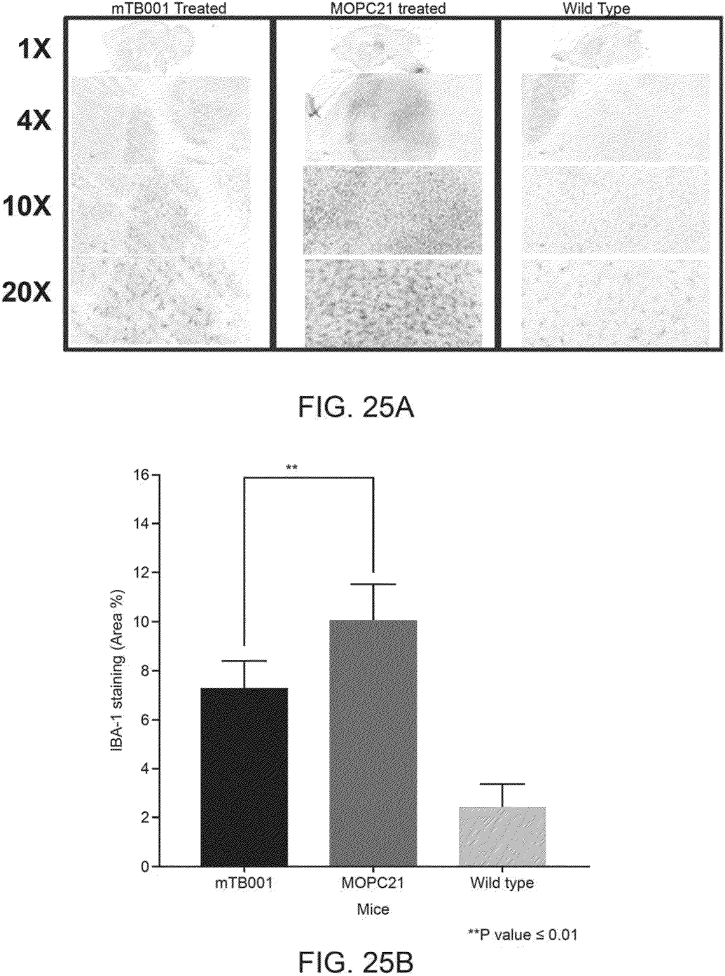

Treatment with mTB001 reduces activated microglia: One of the main characteristics of ICH is an enhanced neuroinflammatory response characterized by activation of microglia. The effect of treatment with mTB001 on microglial reactivity was assessed as measured by microglia marker Iba1. FIG. 25A depicts representative photomicrographs from brain sections of mTB001 and MOPC21 (isotype control) treated mice along with age matched wild type mice stained with Iba1 antibody. Image analysis of sections from multiple animals demonstrated that activated microglia deposits in mTB001 treated mice were significantly reduced (**p ≤ 0.01) as compared to isotype control mice (FIG. 25B). These data demonstrate that treatment with an anti-Gal3 antibody improves inflammatory symptoms related to microglia activity in an animal model of stroke.

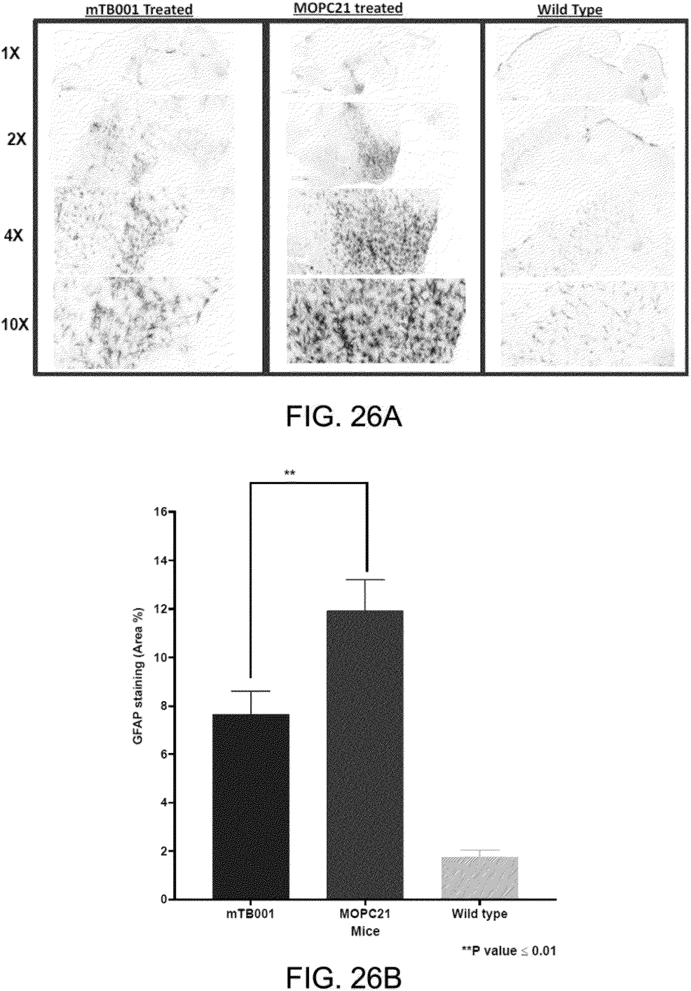

Treatment with mTB001 reduces activated astrocytes: Astrogliosis or activation of astrocytes is another characteristic feature of neuroinflammation due to ICH. The effect of treatment with mTB001 on astrocyte reactivity was assessed as measured by astrocyte marker GFAP. FIG. 26A depicts representative photomicrographs from brain sections of mTB001 and MOPC21 (isotype control) treated mice along with age matched wild type mice stained with GFAP antibody. Image analysis of sections from multiple animals demonstrated that activated astrocyte deposits in mTB001 treated mice were significantly reduced (**p ≤ 0.01) as compared with isotype control mice (FIG. 26B). These data demonstrate that treatment with an anti-Gal3 antibody improves inflammatory symptoms related to astrocyte activity in an animal model of stroke.

In summary, anti-Gal3 antibody treatment is shown to have beneficial effects towards stroke and symptoms thereof. This is particularly striking, as the treatment is able to have efficacy across the blood-brain barrier.

### Example 2. Anti-Gal3 Antibodies Reduce Brain Pathology in a Traumatic Brain Injury Model

Anti-Gal3 antibody treatment (using mTB001 as a representative antibody) was assessed for efficacy and pharmacodynamics in a controlled cortical impact (CCI) model of traumatic brain injury (TBI). TBI was induced in C57BL/6J mice using a controlled cortical impactor. Following 48 hours after TBI induction, mice were treated with 10 mg/kg mTB001 or MOPC21 isotype control. Mice that did not receive a cortical impact served as healthy controls. After a further 48 hours, brain pathology was measured with IHC, and plasma biomarkers were tested by ELISA. The exemplary anti-Gal3 antibody mTB001 significantly improved locomotor function compared to MOPC21 controls. In the brain, mTB001 reduced levels of CD68+ cells, activated microglia and astrocytes, reduced evidence of microhemorrhage, and enhanced levels of neurons. mTB001 also reduced the exploratory biomarker neurofilament-light (NF-L) in the plasma.

TBI, which can be caused by a blow, bump, or jolt to the head, penetrating head injury, or explosive blast is an injury that disrupts normal brain function. TBI can be divided into two stages: 1) the primary injury due to the initial mechanical trauma; and 2) the secondary and potentially reversible damage, characterized by cell loss, axonal damage, and neuroinflammation. Specifically, there is evidence of blood-brain barrier damage with microhemorrhage, astrocytosis, and perivascular clusters of activated microglia. In some cases, focal accumulations of hyperphosphorylated tau as well as macrophage infiltration is detected in close proximity to regions of axonal injury.

Health effects associated with TBI can be broadly categorized into cognitive, behavioral/emotional, motor, and somatic symptoms. Without effective repair, long-term consequences can develop even decades after the original injury, such as chronic traumatic encephalopathy (CTE), dementia, and Parkinson’s disease, each which is associated with the accumulation of hyperphosphorylated tau and destructive neurofibrillary tangles. Therapeutics that can block and reverse disease progression after the primary injury is incurred may have profound, positive life-long effects on patients and their families.

Gal3 was elevated in TBI patients, and was associated with symptom severity (Glasgow Coma Scale scores) and levels of inflammation (plasma C-reactive protein). In preclinical models of TBI using CCI, expression of Gal3 significantly increased in the cortex, hippocampus, and cerebrospinal fluid (CSF) by 24 hours after TBI induction.

Mechanistically, Gal3 mainly co-localized with microglia 24 hours after TBI induction and Gal3 could interact with TLR4, a receptor that senses infection and damage and leads to pro-inflammatory activation when triggered. In vitro stimulation of microglia with Gal3 skewed them towards a pro-inflammatory M1 phenotype and away from an antiinflammatory, pro-repair M2 phenotype. When Gal3 levels were reduced by genetic knockout or neutralizing antibodies, neuroinflammation was reduced in vivo after CCI or after direct TLR4 stimulation with intranigral LPS injection.

Herein, the mouse-reactive anti-Gal3 mIG2a-LALA antibody mTB001 was assessed for efficacy in reducing brain pathology and biomarker levels in plasma of a TBI mouse model.

Study Design: 10-week old C57BL/6J mice had TBI induced with a controlled cortical impactor. After 48 hours, animals were randomized into two groups. One group was dosed intraperitoneally with 10 mg/kg mTB001 and the other group was dosed with MOPC21 isotype control. Three mice that did not receive CCI served as healthy controls. After 48 hours, brains were harvested for IHC and plasma was collected for biomarker detection.

Controlled Cortical Impact Procedure: Animals were anesthetized with isoflurane, placed on a stereotaxic surgery frame, and then their shaved heads were sterilized with 70% ethanol and betadine. A midline incision was made to expose the skull and a 3.0 mm craniotomy was made over the right parietal bone (central point: -2.0 mm bregma; lateral point: 2.5 mm) using a 0.5 mm diameter burr bit connected to a hand-held dental drill. A bone flap of radius 2 mm to 2.5 mm was carved out of the skull. The contusion was next made using a controlled cortical impactor (Leica) with 3.0 m/s velocity, 100 ms dwell time, and 2.2 mm impact depth at 20° angle. The bone flap was affixed to the skull with bone wax and skin sutured over it.

Sample Collection: Mice were anesthetized with isoflurane, blood was collected by cardiac puncture, and mice were perfused transcardially with cold PBS. Brain tissues were fixed for 48 hours with 4% paraformaldehyde in PBS, pH 7.4 at 4° C. and stored in PBS/0.02% NaN₃ at 4° C. until use.

Immunohistochemistry: Fixed brain tissues were sectioned (40 µm) with a vibratome. Sagittal sections were collected in PBS containing 0.02% NaN₃ from 3 different planes of the brain (about 2.5 mm apart) and stored at 4° C. prior to staining. Endogenous peroxidase in tissue was blocked for all stains by treating with 3% H₂O₂ in PBS for 10 minutes at 25° C. Nonspecific background staining was blocked by 1 hour incubation in 2.5% serum, 0.3% Triton-X 100 (TX) at 25° C. Tissues were incubated with primary antibodies against GFAP, Iba1, CD68, and tau protein (AT8 antibody) overnight at 4° C., rinsed three times with PBS + 0.1% TX, followed by biotinylated secondary antibody (anti-mouse) detection with an ABC peroxidase kit, and visualized with a DAB substrate kit. After DAB staining, brain tissues were mounted on Superfrost Plus slides and dehydrated using different percentages of alcohols and xylene. Determination of microhemorrhages was performed by staining for ferric acid using Prussian Blue for 30 minutes. Sections were then rinsed with water and counterstained with Nuclear Fast Red. Slides were cover-slipped using DPX mounting media. Control experiments omitting either primary or secondary antibody resulted in no staining. Brain tissues were scanned using an Aperio VERSA Brightfield, Fluorescence & FISH Digital Pathology Scanner.

Statistical Analysis: The data were analyzed using GraphPad Prism-8 using one-way ANOVA. All values are reported as mean ± standard deviation and significance set at p < 0.05.

mTB001 treatment reduces pathological changes in the brain after traumatic brain injury: To study changes in brain pathology induced by TBI and whether Gal3 antibodies can reduce these changes, animals that did not receive any CCI were compared to animals that received a CCI and treatment with either mTB001 or MOPC21 isotype control. Antibodies were dosed 48 hours after CCI and pathology was assessed by IHC a further 48 hours after antibody treatment. Comparisons first were made between healthy and MOPC21 isotype controls to assess TBI-induced changes. Comparisons were then made between MOPC21 isotype control and mTB001 dosed groups to assess efficacy of anti-Gal3 antibody for TBI.

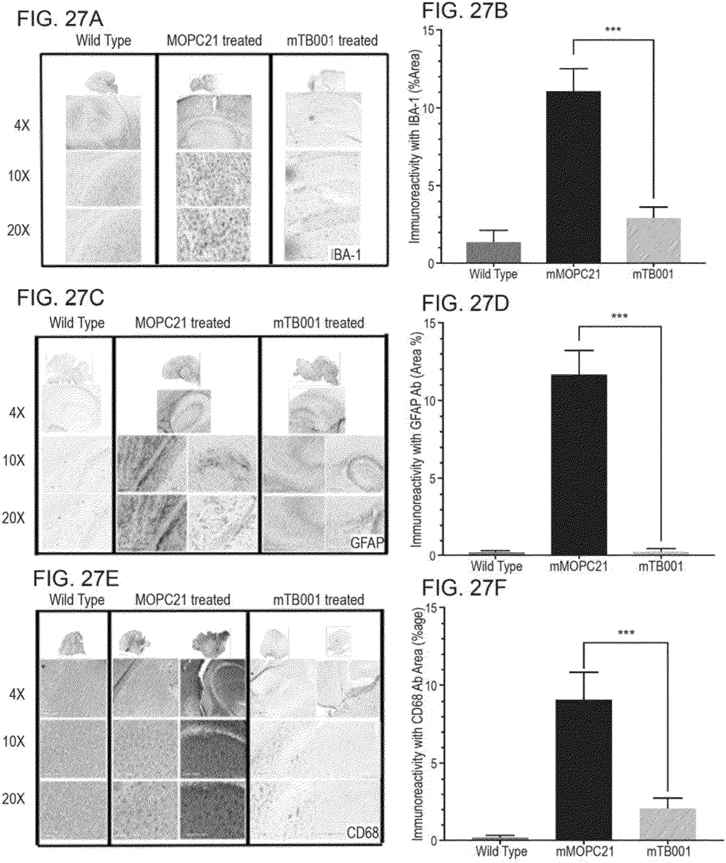

The TBI model exhibited increased levels of activated microglia as detected by Iba1 staining, and treatment with mTB001 significantly reduced this (FIG. 27B). Representative stains are shown in FIG. 27A.

The TBI model exhibited increased levels of activated astrocytes as detected by GFAP staining, and treatment with mTB001 significantly reduced this (FIG. 27D). Representative stains are shown in FIG. 27C.

The TBI model exhibited increased levels of myeloid populations (e.g., macrophages, microglia) that can be detected by CD68 staining in the brain, and treatment with mTB001 significantly reduced this (FIG. 27F). Representative stains are shown in FIG. 27E.

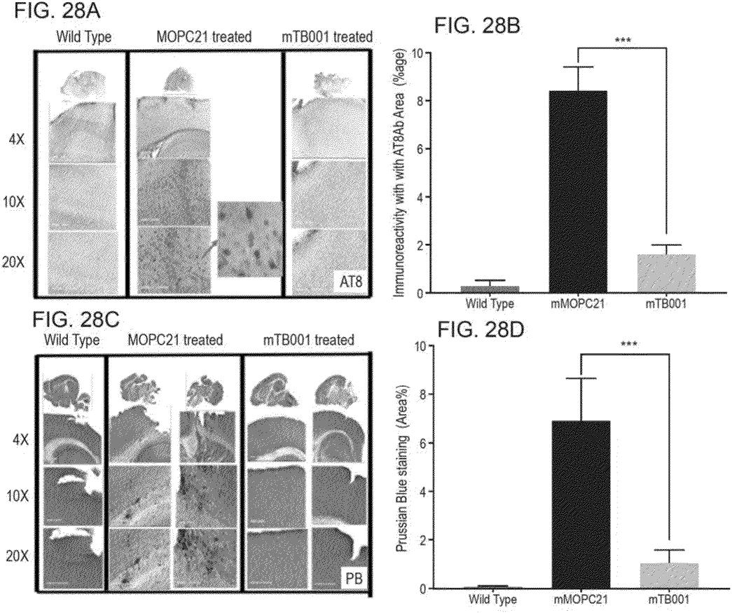

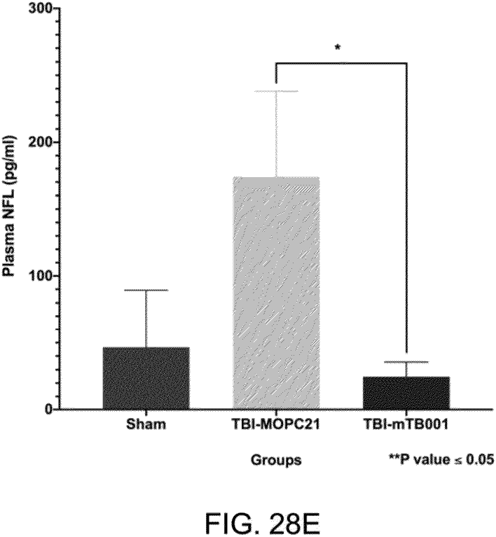

The TBI model exhibited increased levels of hyperphosphorylated tau as detected by staining with antibody AT8, and treatment with mTB001 significantly reduced this (FIG. 28B). Representative stains are shown in FIG. 28A.

The TBI model exhibited increased microhemorrhages as detected by Prussian Blue staining, and treatment with mTB001 significantly reduced this (FIG. 28D). Representative stains are shown in FIG. 28C.

The exploratory neurological disorder biomarker neurofilament-light (NF-L) was detected in plasma of the TBI model. Treatment with mTB001 reduced NF-L in the plasma of TBI-induced mice after 48 hours of treatment (FIG. 28E). Untreated and MOPC21 isotype-treated mice were used as control.

In summary, anti-Gal3 antibody treatment is shown to have beneficial effects towards traumatic brain injury (and related neurological insults) and symptoms thereof. This is particularly striking, as the treatment is able to have efficacy across the blood-brain barrier.

### Example 3. Anti-Gal3 Antibodies Inhibit Fibrin Oligomerization

Thrombosis is a pivotal event in the pathogenesis of diseases such as myocardial infarction, stroke and aneurysms with a 70% to 90% incidence of acute thrombotic occlusion of the artery supplying the infarcted zone in patients with transmural infarctions. In addition, the development of thrombosis overlying the infarcted area may lead to systemic embolization, and deep venous thrombosis develops in 25% of patients as detected by ¹²⁵I-fibrinogen scanning. The fibrin deposition that occurs with these thrombotic events is associated with changes in plasma fibrinogen that may be interpreted either as indicative of a preexisting “hypercoagulable state” or as secondary to the local thrombosis.

Fibrin polymer is an end product of the enzymatic cascade of blood clotting. In vivo formation of the polymeric fibrin network, along with platelet adhesion and aggregation, are the key events in salutary stopping of bleeding at the site of injury (hemostasis) as well as in pathological vascular occlusion (thrombosis). Fibrin polymerization comprises a number of consecutive reactions, each affecting the ultimate structure and properties of the fibrin scaffold. These properties determine the development and outcomes of various diseases, such as heart attack, ischemic stroke, cancers, trauma, surgical and obstetrical complications, hereditary and acquired coagulopathies, and thrombocytopathies. In addition to providing a better understanding of the pathogenesis of such disorders, the knowledge of the molecular mechanisms of fibrin formation provides a foundation for new diagnostic tools and therapeutic approaches.

Rupture of an atherosclerotic plaque causes platelet aggregation and activation of the coagulation cascade with formation of thrombin prompting conversion of fibrinogen to fibrin. As a result, a platelet-rich thrombus entrapped in a cross-linked fibrin network is formed, which may result in arterial occlusion and subsequent organ damage.

Studies have shown that altered structure of fibrin clots is associated with coronary artery disease (CAD) and myocardial infarction (MI). Furthermore, a compact clot structure is associated with cardiovascular risk factors such as diabetes and smoking. Thrombotic and inflammatory markers including fibrinogen and high-sensitive C-reactive protein (hs-CRP) have independently been associated with increased risk of cardiovascular events. Increased plasma levels of fibrinogen directly modulate fibrin clot properties and compromise fibrinolysis, representing one mechanism explaining the association between fibrinogen levels and cardiovascular disease. In addition, markers of inflammation may affect fibrin clot structure and platelet reactivity during antiplatelet therapy. In the brains of Alzheimer’s disease patients, tissue plasminogen activator (tPA)/plasmin fibrinolytic activity is expected to be reduced due to the elevation of plasminogen activator inhibitor-1 (PAI-1) levels in the cerebrospinal fluid of AD patients and the reduction of plasmin activity in AD brains.

Demonstrated herein, Gal3 can accelerate fibrin oligomerization, and anti-Gal3 antibodies can interfere with this oligomerization, which may have therapeutic benefits.

To determine if Gal3 can oligomerize fibrin, fibrin protein was incubated with or without Gal3 and samples taken at various time points. Samples were probed on a dot blot with a conformation-specific antibody that binds oligomers. Inclusion of Gal3 rapidly promotes fibrin oligomer formation. To determine if Gal3 antibodies can block this function, various anti-Gal3 antibody clones (and MOPC21-hIgG4 isotype control) were co-incubated with fibrin and Gal3, and oligomer formation assessed on a dot blot.

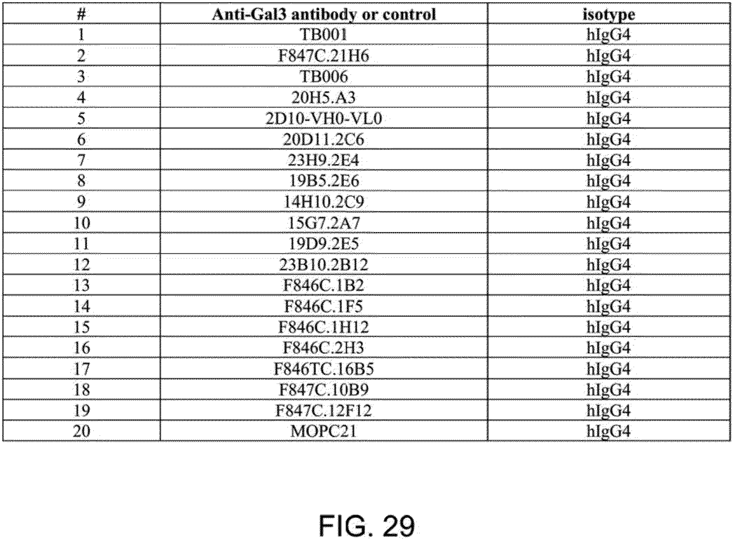

FIG. 29 depicts exemplary anti-Gal3 antibodies tested and assigned numbers (#) used throughout this Example and associated data.

Study Design: To determine the ability of Gal3 to promote oligomerization, 100 µg/mL Gal3 was added to 100 µg/mL fibrin before incubation. For oligomerization of fibrin using Gal3 as an inducer, samples were spotted for 1-5 hours. Alternatively, samples were also taken at 0, 2, 4, 6, and 24 hours after Gal3 addition to fibrin for analysis on a dot blot. During the screening of antibodies, fibrin with and without Gal3 was incubated for 24 hours prior to addition of antibodies. To determine the ability of Gal3 antibodies to degrade fibrin oligomerization, MOPC21-IgG4 isotype control, TB001, TB006, and other anti-Gal3 antibody clones were added to the fibrin/Gal3 mixture and incubated for 24 hours to a final concentration of 100 µg/mL of antibody. Samples were taken at 24 hours for analysis on a dot blot probed with conformation oligomer specific antibody A11.

Oligomerization of fibrin: 1 mg of fibrin peptide was resuspended in 90 µL of 100 mM NaOH and incubated for 10 minutes. This solution was then diluted to a final concentration of 0.1 mg/mL by adding 10 mM sodium phosphate buffer, pH 7.4. Gal3 with or without antibodies were added to test their effects. The solution was continuously stirred using a stir bar while incubating at room temperature. At various time points after mixing, samples were taken and probed on a dot blot to detect formation of oligomers using A11.

Dot blots: 2 µL of sample was pipetted onto a Whatman nitrocellulose membrane at the appropriate time points for dot blot. Dot blot membrane was incubated in 10% non-fat milk in TBS-T for 1 hour at room temperature to block nonspecific binding. The blot was then incubated with primary antibody A11 (rabbit polyclonal oligomer conformation antibody) overnight at 4° C. After three 5-minute washes in TBS-T, the membranes were incubated with the appropriate secondary antibody (goat anti-rabbit IgG H&L (HRP)) for 1 hour at room temperature. Following three 5-minute washes in TBS-T, the membrane was incubated in detection reagents for 1-5 seconds. The dot blot images of the results were obtained using an imager (Azure Biosystems) and quantified using Image Studio software. Local background to each dot was subtracted from image intensity to correct for any differences at different areas of the blots.

Statistical analysis: The quantification was done using Licor Image Studio Lite and results were analyzed in GraphPad Prism-8.

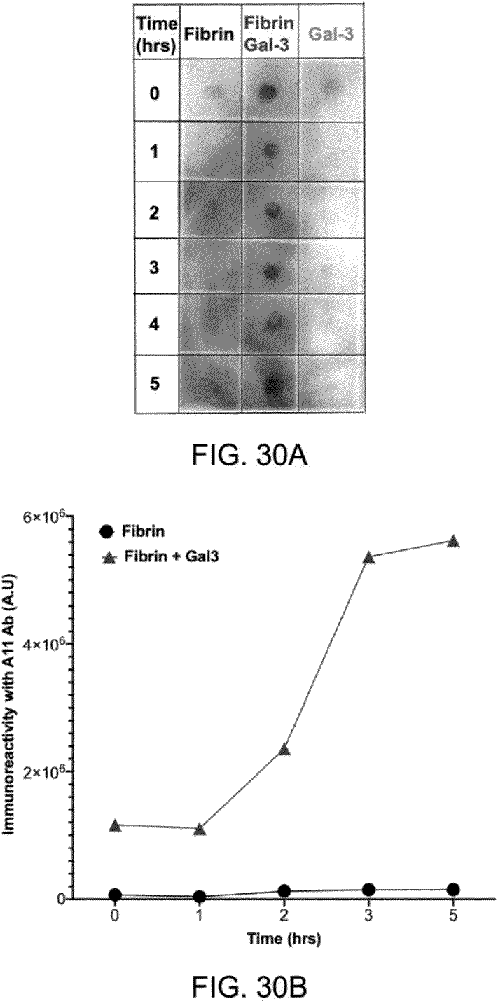

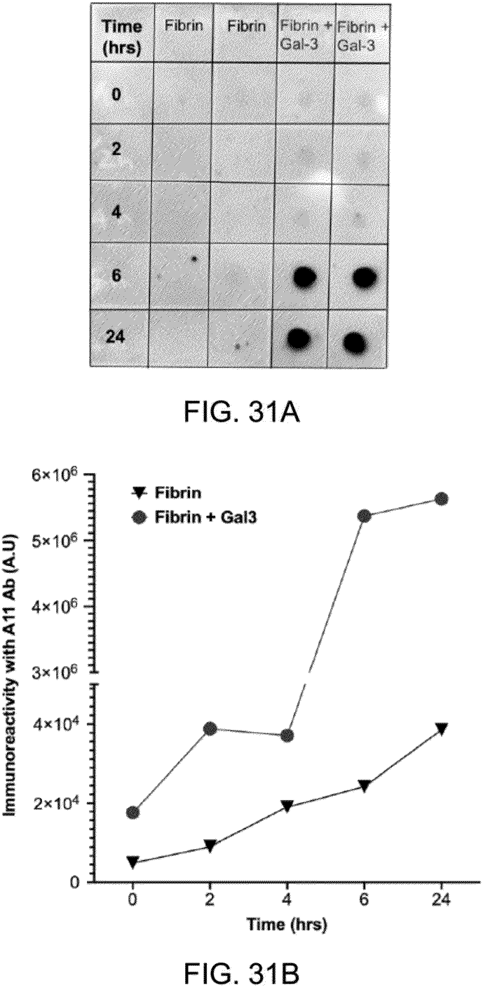

Results: A11 antibody detects conformational oligomeric structures of various amyloid proteins like fibrin. Fibrin oligomerization is promoted by Gal3 incubation compared to controls without Gal3, as evidenced by the increased intensity of the A11-reactive dots. FIGS. 30A-B show a time course of Gal3 and fibrin incubation over 0-5 hours. FIGS. 31A-B show a time course of Gal3 and fibrin incubation over 0-24 hours (which corresponds to the duration used in later antibody incubation experiments).

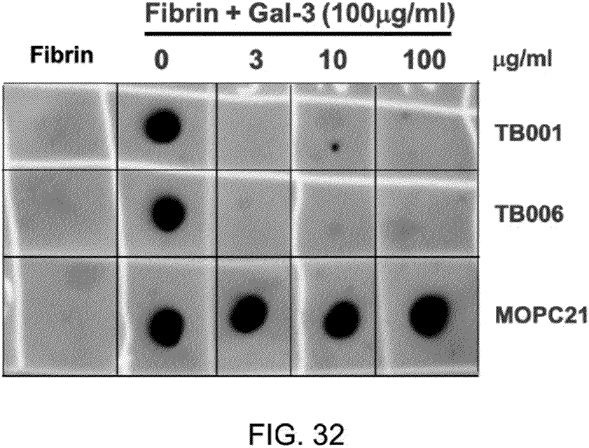

As seen in FIG. 32, Gal3-induced fibrin oligomerization detected by A11 antibody was unaffected by inclusion of MOPC21-hIgG4 isotype control. However, anti-Gal3 antibodies TB001 and TB006 degrade Gal3-induced fibrin oligomerization at tested concentrations of 3, 10, and 100 µg/mL of antibody.

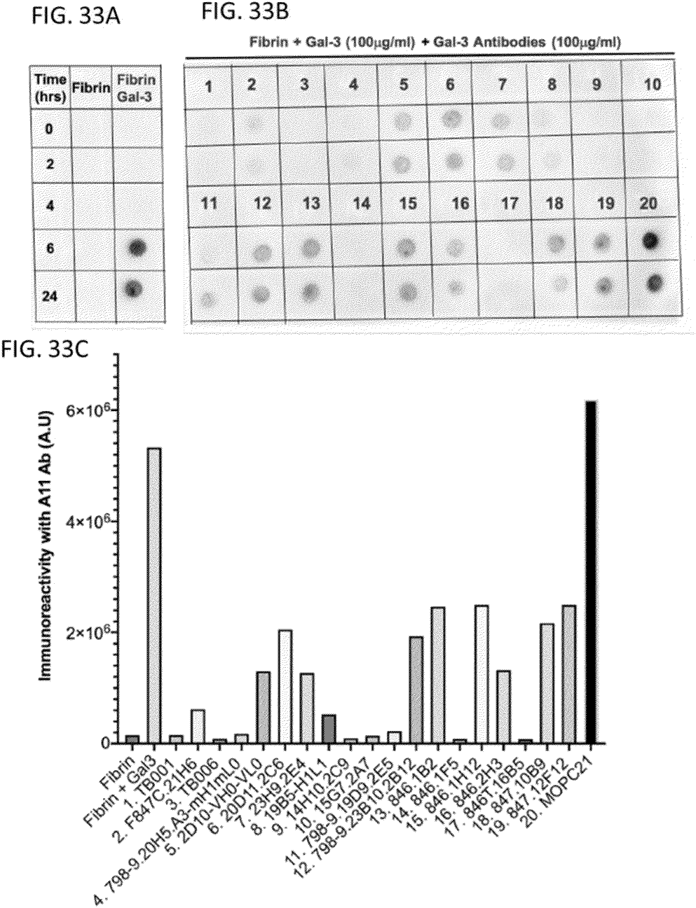

In addition to TB001 and TB006, various other anti-Gal3 antibody clones were screened for ability to degrade Gal3-induced fibrin oligomers. 100 µg/mL each of fibrin and Gal3 were incubated for 24 hours prior to addition of 100 µg/mL anti-Gal3 antibody clones. The samples were continuously stirred using a stir bar at room temperature. After 24 hours, samples were taken and probed on a dot blot with A11 antibody. FIG. 33A shows that Gal3 intrinsically promotes fibrin oligomers. FIG. 33B shows a dot blot of fibrin oligomer degradation by different anti-Gal3 antibodies as listed according to number in FIG. 29, in duplicate. FIG. 33C shows the quantitative profile of degradation of fibrin oligomers by different anti-Gal3 antibodies of FIG. 33B.

As demonstrated here, Gal3 promotes fibrin oligomerization and various anti-Gal3 antibody clones can degrade these Gal3-induced fibrin oligomers, while MOPC21 isotype control had no effect. Accordingly, this suggests that anti-Gal3 antibodies can interfere with molecular mechanisms controlling the development and severity of diseases associated with fibrin activity, such as heart attack, ischemic stroke, cancers, trauma, surgical and obstetrical complications, hereditary and acquired coagulopathies, and thrombocytopathies.

### Example 4. Anti-Gal3 Antibodies as Therapeutics for Cerebral Amyloid Angiopathy

Cerebral amyloid angiopathy (CAA) consists of the deposition of amyloid β (Aβ) peptide in the walls of small to medium-sized arteries, arterioles, and capillaries in the cerebral cortex, leptomeninges, and cerebellum. In as many as 15% of patients with spontaneous ICH, the pathogenesis of ICH is attributed to CAA. However, the deposition of amyloid in cerebral vessels is common, and the extent of amyloid deposition is age related. CAA may be confirmed from the autopsy of 10% to 50% of the elderly population, in 80% of patients with Alzheimer’s disease and in 50% of patients with ICH. Stroke and Alzheimer’s disease are cerebral pathologies with high socioeconomic impact that can occur together and mutually interact. Vascular factors predisposing to cerebrovascular disease have also been specifically associated with development of AD, and acute stroke is known to increase the risk to develop dementia. Despite the apparent association, it remains unknown how acute cerebrovascular disease and development of AD are precisely linked and act on each other. It has been suggested that this interaction is strongly related to vascular deposition of Aβ (i.e., CAA). Furthermore, the blood-brain barrier (BBB), perivascular space, and the glymphatic system, the latter proposedly responsible for the drainage of solutes from the brain parenchyma, may represent key pathophysiological pathways linking stroke, Aβ deposition, and dementia.

There are many links between hemostasis and cerebrovascular pathology in AD. Aβ can activate platelets, induce microhemorrhages in the brain, and interact with several coagulation factors. Aggregates of Aβ can activate coagulation factor XII (FXII) to initiate blood clotting, and can increase fibrin density and resistance to fibrinolysis. CAA deposits contain coagulation factors, and antiplatelet therapy reduces accumulation of CAA deposits and improves cognitive function in mice. Aβ forms stable complexes with FXIIIa and colocalizes with FXIIIa and fibrin in CAA deposits of AD patients.

Transgenic mice with human APP751 bearing the E693Q mutation under the murine Thy1 promoter is a model for CAA. This transgenic mouse line bears a mutation that was determined to cause hereditary cerebral hemorrhage with amyloidosis-Dutch type, a rare autosomal dominant disorder characterized by CAA, strokes, and dementia (APPDutch). These mice have an increased Aβ40/Aβ42 ratio, but parenchymal amyloid plaques are not observed. Instead, mice develop extensive vascular Aβ deposition starting at 22 to 24 months of age, and appearing first in leptomeningeal vessels followed by cortical vessels. This leads to smooth muscle cell degeneration, hemorrhages, and neuroinflammation. The mice develop robust microgliosis immediately adjacent to amyloid-laden vessels, and widespread activation of astrocytes in neocortical regions affected by CAA. Female mice have earlier onset of amyloid deposition.

APPDutch mice are used for in vivo study. Mice will undergo behavior tests and compared to age matched control mice. Pre-dose plasma/serum is collected for different biomarker assays. Dose range therapeutic efficacy of the anti-Gal3 antibodies disclosed herein are conducted. Briefly, different doses of anti-Gal3 antibodies are administered i.p. twice in a week, with a total of 8 doses administered. After dosing, mice will undergo behavior tests. Mice are sacrificed, and blood, CSF, brain, and other organs are collected for quantifying various biomarkers, histology, and biochemical characterization.

In AD and CAA, Gal3 expression levels are increased. Gal3 intrinsically promotes aggregation of various amyloid proteins like Aβ and non-amyloid proteins like fibrin during the disease stage. Anti-Gal3 antibodies degrade these oligomers in a dose dependent manner in the case of Aβ oligomers, and also degrades fibrin oligomers. Four doses of exemplary anti-Gal3 antibody mTB001 not only significantly reduces the pathological hallmarks of AD but also reduces inflammatory markers such as activated microglia and astrocytes. Improvement of CAA may also be assessed through biomarkers such as ApoE, clusterin, and sushi repeat-containing protein X-linked 1 (SRPX1).

### Example 5: Anti-Gal3 Antibodies as Therapeutics for Atherosclerosis

Atherosclerosis is marked by macrophage accumulation, activation and differentiation into cholesterol-laden “foam cells” within atherosclerotic vascular walls, indicating that atherosclerosis can be considered a chronic inflammatory disease. Gal3 levels are elevated in human atherosclerosis plaques and in preclinical animal models of hypercholesterolemia and stenosis (ApoE-/- mice). Moreover, Gal3 is increased in the unstable parts of patient plaques, and human monocytes treated with Gal3 show increased chemotaxis and activation. Within the plaque, Gal3 is mainly distributed in macrophages and foam cells (rarely in vascular smooth muscle cells), and its expression increases with plaque severity (i.e., extent and inflammation) in human patients and ApoE-/- mice. Atherosclerosis is also associated with and/or may be a cause of thrombosis and thromboembolism, carotid artery disease, coronary artery disease, peripheral artery disease, atherosclerosis-associated heart failure and myocardial infarction, chronic kidney disease, and stroke.

Gal3 activates secretion of proinflammatory cytokines in myeloid cell lines, and anti-Gal3 antibodies can prevent and/or reduce such effect. The Gal3 inhibitor MCP (modified citrus pectin) inhibits the adhesion of leukocytes to endothelial cells and reduces the size of atherosclerotic lesion areas in ApoE-/- mice.

### Example 6: Anti-Gal3 Antibodies Prevent Gal3-induced Inflammatory Cytokine Expression in Monocyte and Neutrophil Cell Lines

The effect of Gal3 on enhancing cytokine expression in neutrophils and the ability to block this effect by anti-Gal3 antibodies were assessed. HL60 promyeloblast cells (ATCC) were maintained in IMDM with 20% heat-inactivated FBS (Gibco) and 1x Penicillin-Streptomycin (Gibco). HL60 cells were differentiated to Neutrophil-like cells with 1.3% DMSO for 96 hours. Differentiated HL60 cells were washed with FBS-free IMDM media to be used for stimulation at a later time. In 96-well U bottom plates, 50 µL of 4 µM Gal3 (4X, which will result in a 1X (1 µM) final concentration after other component volumes are added) was added to the plates as the highest concentration, followed by a 1:2 serial dilution in FBS-free IMDM media to establish a dose response curve (0, 0.015625, 0.03125, 0.0625, 0.125, t0.25, 0.5, or 1 µM final concentration of Gal3). 50 µL of each Gal3 dilution was then incubated with 50 µL of exemplary anti-Gal3 antibodies or MOPC21 isotype control (at concentrations of 0, 0.0625, 0.125, 0.25, 0.5, 1, 2, or 4 µM final concentration of antibody) for 30 min at 37° C. After that, 0.4 ng/mL of LPS (4X, which will result in a 1X (0.1 ng/mL) final concentration with the other component volumes) was added to every well in the plates and incubated for 30 min at 37° C. Finally, 50 µL of differentiated HL-60 cells (a total of 100k cells) were added to every well in the plates and incubated for 4 hours at 37° C. At the end of the incubation, plates were centrifuged for 5 min at 1000 rpm and supernatant was removed to measure TNF-α, IL-6, and IL-8 by ELISA.

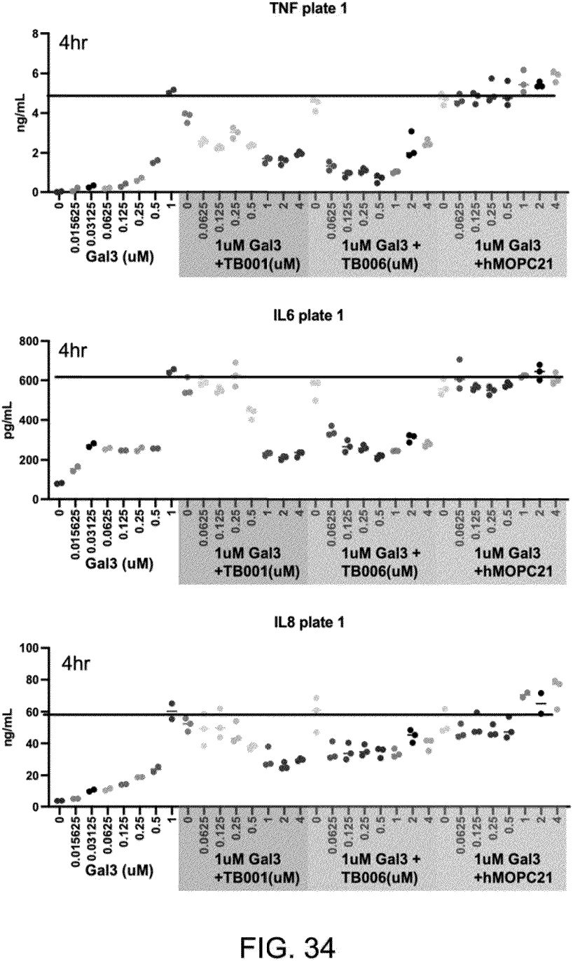

Tested anti-Gal3 antibodies TB001 and TB006 were shown to have a dose-dependent effect in reducing TNF-α, IL-6, and IL-8 secretion of the HL60 cells during LPS stimulation (FIG. 34). MOPC21 isotype control had no effect on cytokine secretion. Some nonspecific cytokine stimulation may be observed due to Fc domains on the antibodies.

The effect of Gal3 on enhancing cytokine expression in monocytes and the ability to block this effect by anti-Gal3 antibodies was also assessed. THP-1 Monocyte-like cells (ATCC) were maintained in RPMI-1640 media with 10% heat-inactivated FBS and 1X Penicillin-Streptomycin. THP-1 cells were washed once with FBS-free RPMI-1640 media to be used for stimulation at a later time. In 96-well U bottom plates, 50 µL of 4 µM of Gal3 (4X 1 µM) was added to the plates as the highest concentration, followed with a 1:2 serial dilution in FBS-free RPMI-1640 media to establish a dose response curve. 50 µL of 4 µM Gal3 was then incubated with 50 µL of exemplary anti-Gal3 antibodies or MOPC21 isotype control (at concentrations of 0, 0.0625, 0.125, 0.25, 0.5, 1, 2, or 4 µM final concentration of antibody) for 30 min at 37° C. After that, 0.4 ng/mL of LPS (4× 0.1 ng/mL) was added to every well in the plates and incubated for 30 min at 37° C. Finally, 50 µL of THP-1 cells (a total of 100k cells) were added to every well and incubated for 6 or 24 hours at 37° C. At the end of the incubation, plates were centrifuged for 5 min at 1000 rpm and supernatant was removed to measure TNF-α, IL-6, and IL-8 by ELISA.

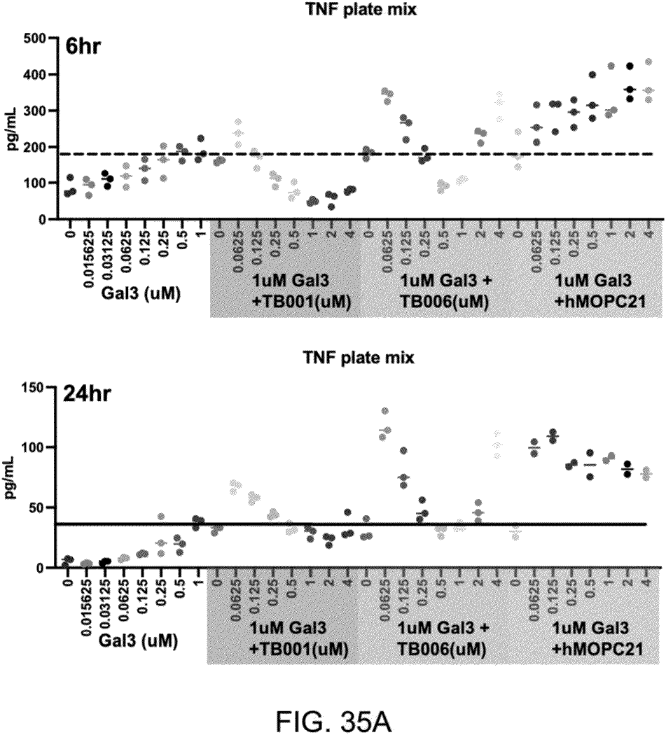

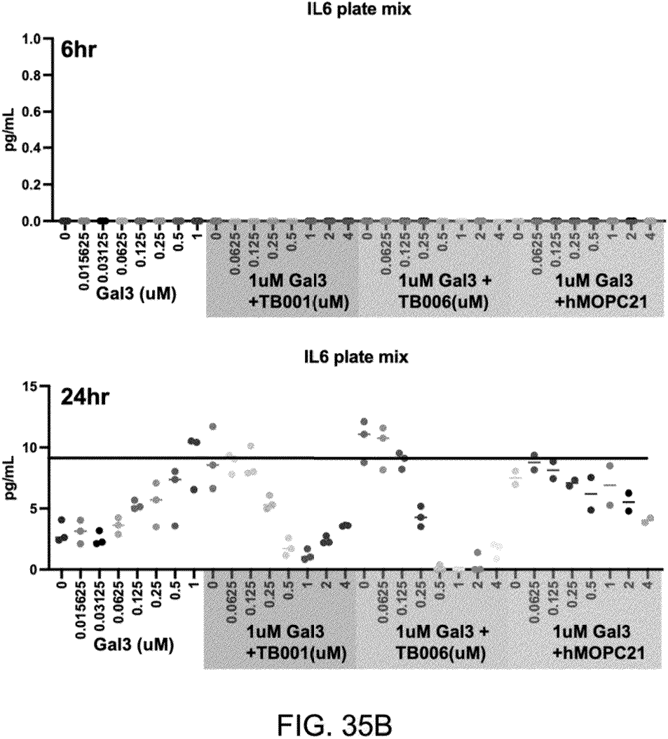

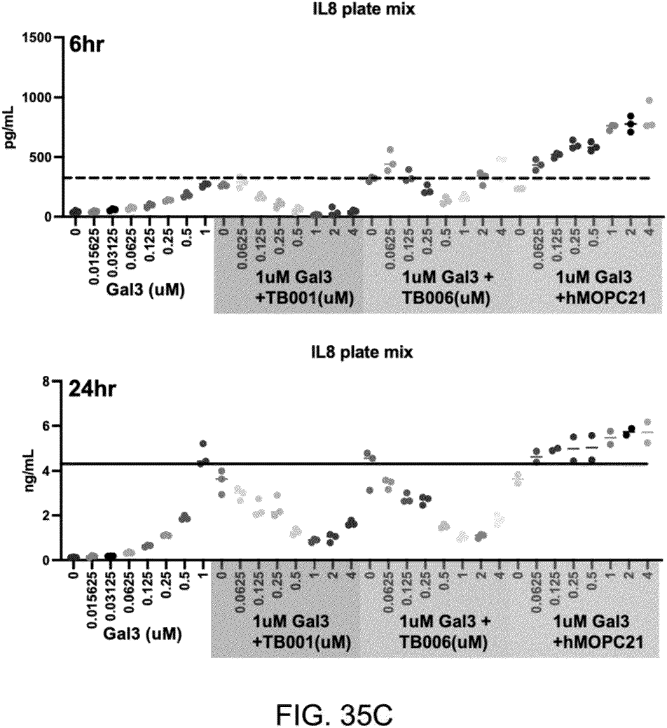

Tested anti-Gal3 antibodies TB001 and TB006 were shown to have a dose-dependent effect in reducing TNF-α, IL-6, and IL-8 secretion of the THP-1 cells during LPS stimulation (FIGS. 35A-C). MOPC21 isotype control had no effect on cytokine secretion. Some nonspecific cytokine stimulation may be observed due to Fc domains on the antibodies.

In summary, anti-Gal3 antibody treatment is able to inhibit proinflammatory activity of immune cells such as neutrophils and monocytes.

### Example 7: Binning and Peptide Binding Assay of Additional Exemplary Anti-Gal3 Antibodies

A large-scale antibody binning assay was performed on exemplary anti-Gal3 antibodies.

The epitope binning assay was done in a sandwich format on the high-throughput SPR-based Carterra LSA unit (CarterraBio, Salt Lake City, UT). First, the purified antibodies were diluted to 10 µg/ml concentration in 10 mM NaOAc (pH 5.0) and then were covalently coupled via amine group to HC200M chip activated by EDC and S-NHS to immobilize antibodies to different positions of a 384-spot array. One hundred thirty-eight binning cycles were run on the array of immobilized antibodies. In each cycle, first, human Gal3 (AcroBio GA3-H5129) was injected over the entire array to bind to different antibodies (primary antibody), followed by one antibody (secondary antibody) selected among the panel of 150 anti-GAL3 antibodies tested. At the end of each cycle, the array was regenerated by 10 mM Glycine (pH 2.0) to remove bound antigen and secondary antibody from the array. The data analysis was done using the Epitope software by CarterraBio.

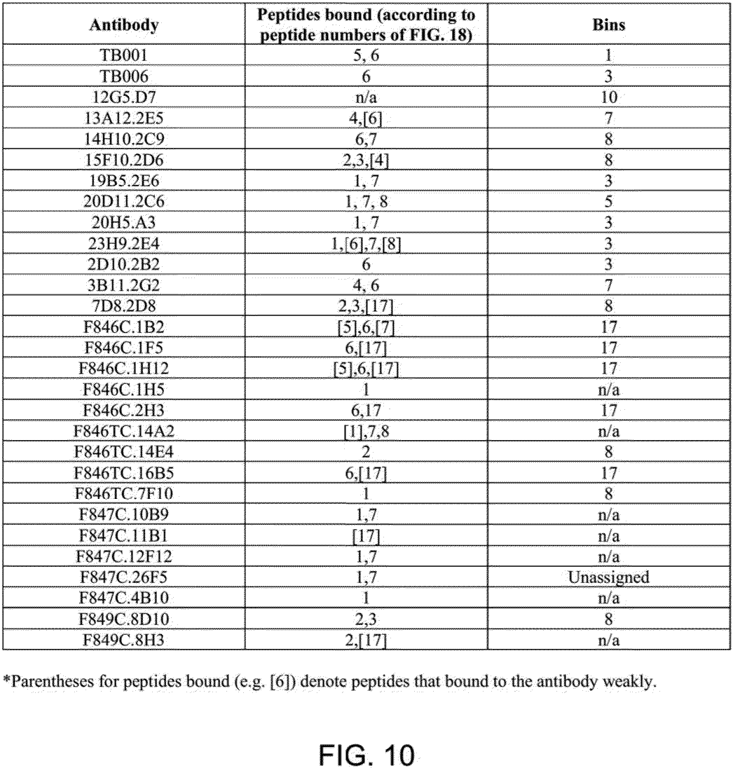

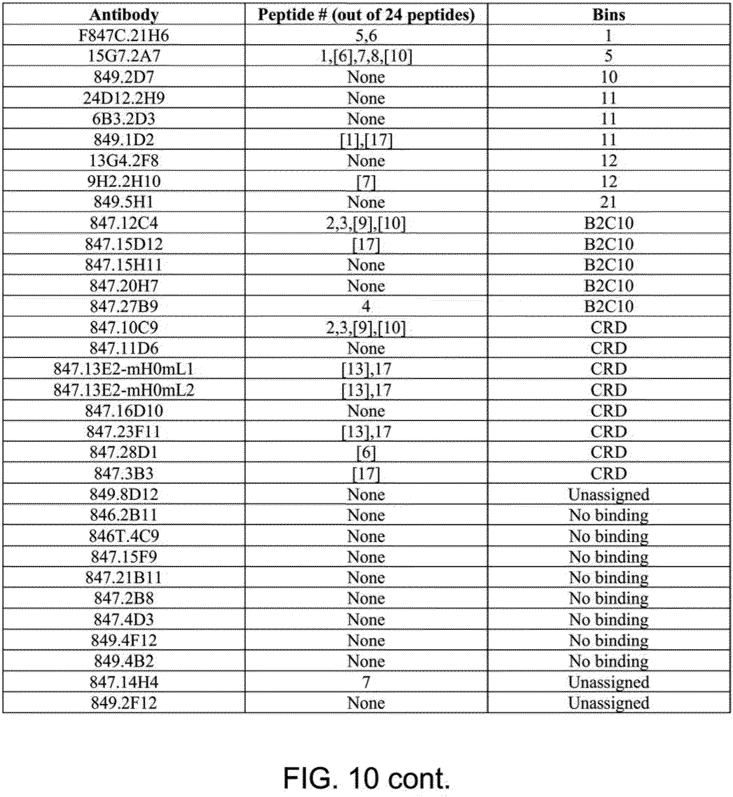

Binning results are shown in FIG. 10. In total, 49 distinct bins were identified for 120 anti-GAL3 antibodies that demonstrate binding to hGAL3 (30 antibodies out of 150 tested did not bind hGAL3 when immobilized on HC200M chip and were thus excluded from further analysis). Antibodies that strongly blocked the association of GAL3 and APP695 belonged to a number of distinct bins defined below. This highlights the utility of these bins as predictors of GAL3 binding activity.

Clones IMT001 (TB001) and F847C.21H6 exhibited mutual competitive binding for Gal3, but did not prevent binding of the rest of the clones, thus defining bin 1. Clones IMT006 (TB006), 19B5.2E6, 20H5.A3, 23H9.2E4, 2D10.2B2 exhibited mutual competitive binding for Gal3, but did not prevent binding of the rest of the clones, thus defining bin 3. Clones 20D11.2C6 and 15G7.2A7 exhibited mutual competitive binding for Gal3, but did not prevent binding of the rest of the clones, thus defining bin 5. Clones 3B11.2G2, 13A12.2E5 exhibited mutual competitive binding for Gal3, but did not prevent binding of the rest of the clones, thus defining bin 7. Clones 14H10.2C9, 15F10.2D6, 7D8.2D8, F846TC.14E4, F846TC.7F10, F849C.8D10 exhibited mutual competitive binding for Gal3, but did not prevent binding of the rest of the clones, thus defining bin 8. Clones 12G5.D7 and 849.2D7 exhibited mutual competitive binding for Gal3, but did not prevent binding of the rest of the clones, thus defining bin 10. Clones 24D12.2H9, 6B3.2D3, 849.1D2 exhibited mutual competitive binding for Gal3, but did not prevent binding of the rest of the clones, thus defining bin 11. Clones 13G4.2F8 and 9H2.2H10 exhibited mutual competitive binding for Gal3, but did not prevent binding of the rest of the clones, thus defining bin 12. Clones F846C.1B2, F846C.1F5, F846C.1H12, F846C.2H3, F846TC.16B5 exhibited mutual competitive binding for Gal3, but did not prevent binding of the rest of the clones, thus defining bin 17. In addition, clone 849.5H1 did not compete for binding to Gal3 with other antibodies tested, therefore defining bin 21.

Antibodies 847.12C4, 847.15D12, 847.15H11, 847.20H7, and 847.27B9 exhibited mutual competitive binding and competed with binding with the commercially available anti-Gal3 antibody B2C10 for hGAL3, but did not prevent binding of the rest of the clones, thus defining bin “B2C10”. B2C10 has been epitope mapped to bind to the first 18 amino acids of Gal3. Antibodies 847.10C9, 847.11D6, 847.13E2-mH0mL1, 847.13E2-mH0mL2, 847.16D10, 847.23F11, 847.28D1, and 847.3B3 exhibited mutual competitive binding to the C-terminal carbohydrate-recognition domain (CRD), but did not prevent binding of the rest of the clones, thus defining bin “CRD”. Antibodies F846TC.14A2, F847C.10B9, F847C.11B1, F847C.12F12, F847C.4B10, F849C.8H3, 849.8D12, F846C.1H5, 847.14H4, F847C.26F5, 849.2F12, were either not tested (“n/a”) or not assigned (“Unassigned”) to a bin. Antibodies 846.2B11, 846T.4C9, 847.15F9, 847.21B11, 847.2B8, 847.4D3, 849.4F12, and 849.4B2 were determined to not bind to hGAL3 under the conditions tested.

Gal3 peptide binding activity of additional exemplary anti-Gal3 antibodies was assessed with the same peptides as described in FIG. 2 (SEQ ID NOs: 3-26). Human Gal3 peptides (LifeTein, custom order) and human Gal3 proteins (R&D Systems, 8259-GA; TrueBinding, QCB200349) were diluted in PBS (Corning, 21-030-CM) to concentrations of at least 100 µg/mL (peptides) or 1 µg/mL (proteins), and added to the wells of several 96-well ELISA plates (Thermo Fisher, 44-2404-21). After incubating the plates at 4° C. overnight, the plates were washed three times with PBST (PBS with 0.05% Tween 20 [VWR, 0777]). The plates were then blocked for an hour with 2% BSA (EMD Millipore, 126609) in PBST at room temperature with gentle rocking. Thereafter, the 2% BSA in PBST was discarded and Gal3-binding antibodies (reformatted hIgG4 [S228P]) were diluted in 2% BSA in PBST to concentrations of 5 µg/mL and added to the wells. The plates were incubated for an hour at room temperature with gentle rocking and then washed three times with PBST. Afterwards, Peroxidase AffiniPure F(ab′)₂ Fragment Goat Anti-Human IgG (H+L) polyclonal antibody (Jackson ImmunoResearch, 109-036-003), was diluted in 2% BSA in PBST (1:4000) and added to the wells. The plates were incubated for 1 hour at room temperature with gentle rocking and then washed three times with PBST. TMB substrate (Thermo Scientific, 34029) was then added to each well. The reaction was stopped with 1 M HCl (JT Baker, 5620-02) and read using a plate reader (Molecular Devices) at absorbance of 450 nm.

Peptide binding results are depicted in FIG. 10. Peptide numbering is based on FIG. 2. Binding of Gal3-binding antibodies to the peptide array was observed at multiple locations, with the majority of binding observed in peptides 1-8 and some binding to peptides 13 and 17.

13 separate Gal3-binding antibodies (19B5.2E6, 20D11.2C6, 20H5.A3, 23H9.2E4, F846C.1H5, F846TC.14A2, F846TC.7F10, F847C.10B9, F847C.12F12, F847C.26F5, F847C.4B10, 15FG7.2A7, 849.1D2) all bound peptide 1 of Gal3, corresponding to amino acids ADNFSLHDALSGSGNPNPQG (SEQ ID NO: 3) of Gal3.

7 separate Gal3-binding antibodies (15F10.2D6, 7D8.2D8, F846TC.14E4, F849C.8D10, F849C.8H3, 847.12C4, 847.10C9) bound peptide 2 of Gal3, corresponding to amino acids SGSGNPNPQGWPGAWGNQPA (SEQ ID NO: 4) of Gal3.

5 separate Gal3-binding antibodies (15F10.2D6, 7D8.2D8, F849C.8D10, 847.12C4, 847.10C9) bound peptide 3 of Gal3, corresponding to amino acids WPGAWGNQPAGAGGYPGASY (SEQ ID NO: 5) of Gal3.

4 separate Gal3-binding antibodies (13A12.2E5, 15F10.2D6, 3B11.2G2, 847.27B9) bound peptide 4 of Gal3, corresponding to amino acids GAGGYPGASYPGAYPGQAPP (SEQ ID NO: 6) of Gal3.

4 separate Gal3-binding antibodies (TB001 (IMT001), F846C.1B2, F846C.1H12, F847C.21H6) bound peptide 5 of Gal3, corresponding to amino acids PGAYPGQAPPGAYPGQAPPG (SEQ ID NO: 7) of Gal3.

15 separate Gal3-binding antibodies (TB001 (IMT001), TB006 (IMT006), 13A12.2E5, 14H10.2C9, 23H9.2E4, 2D10.2B2, 3B11.2G2, F846C.1B2, F846C.1F5, F846C.1H12, F846C.2H3, F846TC.16B5, F847C.21H6, 15FG7.2A7, 847.28D1) bound peptide 6 of Gal3, corresponding to amino acids GAYPGQAPPGAYPGAPGAYP (SEQ ID NO: 8) of Gal3.

13 separate Gal3-binding antibodies (14H10.2C9, 19B5.2E6, 20D11.2C6, 20H5.A3, 23H9.2E4, F846C.1B2, F846TC.14A2, F847C.10B9, F847C.12F12, F847C.26F5, 15FG7.2A7, 9H2.2H10, 847.14H4) all bound peptide 7 of Gal3, corresponding to amino acids AYPGAPGAYPGAPAPGVYPG (SEQ ID NO: 9) of Gal3.

4 separate Gal3-binding antibodies (20D11.2C6, 23H9.2E4, F846TC.14A2, 15G7.2A7) all bound peptide 8 of Gal3, corresponding to amino acids GAPAPGVYPGPPSGPGAYPS (SEQ ID NO: 10) of Gal3.

2 separate Gal3-binding antibodies (847.12C4, 847.10C9) bound peptide 9 of Gal3, corresponding to amino acids PPSGPGAYPSSGQPSATGAY (SEQ ID NO: 11) of Gal3.

3 separate Gal3-binding antibodies (15G7.2A7, 847.12C4, 847.10C9) all bound peptide 10 of Gal3, corresponding to amino acids SGQPSATGAYPATGPYGAPA (SEQ ID NO: 12) of Gal3.

3 separate Gal3-binding antibodies (847.13E2-mH0mL1, 847.13E2-mH0mL2, 847.23F11) all bound peptide 13 of Gal3, corresponding to amino acids LPGGVVPRMLITILGTVKPN (SEQ ID NO: 15) of Gal3.

13 separate Gal3-binding antibodies (7D8.2D8, F846C.1F5, F846C.1H12, F846C.2H3, F846TC.16B5, F847C.11B1, F849C.8H3, 849.1D2, 847.15D12, 847.13E2-mH0mL1, 847.13E2-mH0mL2, 847.23F11, 847.3B3) all bound peptide 17, corresponding to amino acids RFNENNRRVIVCNTKLDNNW (SEQ ID NO: 19) of Gal3.

19 separate Gal-3 binding antibodies (849.2D7, 24D12.2H9, 6B3.2D3, 13G4.2F8, 849.5H1, 847.15H11, 847.20H7, 847.11D6, 847.16D10, 849.8D12, 846.2B11, 846T.4C9, 847.15F9, 847.21B11, 847.2B8, 847.4D3, 849.4F12, 849.4B2, 849.2F12) were not found to bind to any of the tested Gal3 peptides under the conditions used in this Example.

As illustrated in FIG. 2, peptides 4, 5, 6, and 7 share repeated amino acid sequences comprised of proline-glycine (PG) and tyrosine-proline-glycine (YPG), indicating a common feature that may explain the ability of Gal3-targeted antibodies to bind to multiple Gal3 peptides. Further, the amino acid sequence glycine-x-tyrosine-proline-glycine (GxYPG), where x may be the amino acids alanine (A), glycine (G), or valine (V), is shared in peptides 4, 6, and 7, each of which possess two such sequences separated by 3 amino acids. Accordingly, the presence of two GxYPG sequences in close apposition is likely predictive of the ability to bind Gal3-targeted antibodies. Additionally, the Grantham distance of alanine, glycine, and valine is Ala-Val: 64, Ala-Gly: 60, Val-Gly: 109, thereby predicting that amino acids with similarly low Grantham distances may similarly be able to substitute at the variable region, including proline and threonine.

### Example 8. Humanized Anti-Gal3 Antibodies Have High Affinity for Gal3 of Different Species

Cross reactivity of exemplary anti-Gal3 antibodies TB001 and TB006 to human or mouse Gal3 were tested. Kinetics experiments were performed on a Carterra LSA at 25° C. An HC30M chip was immobilized with recombinant Protein A/G. TB001 (IMT001; IMT001-4), TB006 (IMT006; IMT006-5), and a negative control antibody Synagis (10 µg/mL each diluted in HBSTE buffer (HEPES based saline with Tween-20 and EDTA) with 0.5 mg/mL BSA) were captured onto different spots in the 384-spot array. Then, a dilution series of Gal3 (2, 4, 8, 16, 32, 64, and 128 nM) in HBSTE with 0.5 mg/mL BSA was injected to the whole array for 300 seconds to allow association followed by dissociation for 240 seconds. The kinetics constants were calculated by the software NextGenKIT.

Separately, the affinity kinetics of TB001 and TB006 to cynomolgus Gal3 (cynoGal3) was measured on a Biacore T200 at 25° C. A CM5 chip coated with anti-mouse Fc/anti-human Fc mixture was used to load purified antibody TB001 or TB006 at 10 µg/mL in HBS-EP+ buffer (HEPES based saline with polysorbate 20 and EDTA) with 0.5 mg/mL BSA for 180 seconds, and then a dilution series of His-enterokinase cleavage site (EK)-cynoGal3 (TrueBinding in-house antigen) in HBS-EP+ with 0.5 mg/mL BSA starting from 100 nM, 1:2 dilution for 7 points for 200 seconds, followed by dissociation for 300 seconds. In each cycle, duplicates were made by loading the same antibody to flow cells FC2 and FC4, and then analyte was flowed to all 4 flow cells. The detection was done pairwise, FC2-FC1 and FC4-FC3, to subtract non-specific binding of analyte to the surface. A cycle of 0 nM analyte was run prior to a dilution series of analyte. The 0 nM raw data were subtracted from each non-zero concentration data to minimize machine drifts. The data were analyzed by Biacore Evaluation software version 2.0. The binding affinities were characterized by fitting the kinetic sensograms to a monovalent binding model (1:1 binding).

Separately, the affinity kinetics of TB001 and TB006 to rat Gal3 was measured on a Biacore T200 at 25° C. A CM5 chip was coated with goat anti-human Fc antibody on all 4 flow cells, and was used to load purified antibodies TB001, TB006, and Synagis (negative control), which all use an hIgG4 backbone. In each sample cycle, duplicates of the same antibody in HBS-EP+ with 0.5 mg/mL BSA at 10 µg/mL were captured to FC2 and FC4 by flowing at 10.0 µL/min for 120 seconds, and then diluted His-EK-ratGal3 in HBS-EP+ with 0.5 mg/mL BSA was injected to all 4 flow cells at 30.0 µL/min for 180 seconds, followed by dissociation for 240 seconds. A dilution series of His-EK-ratGal3 started from 300 nM, and was 1:3 diluted for 6 points. FC1 and FC3 were used as reference flow cells, and their sensograms were subtracted from FC2 and FC4 respectively to reduce non-specific binding background. Also, an additional sample cycle at 0 nM analyte was run, and its sensogram was subtracted from those of non-zero nM cycles to address machine drifts and bulk shifts. The data were analyzed by Biacore Evaluation software version 2.0. The binding affinities were characterized by fitting the kinetic sensograms to a monovalent binding model (1:1 binding).

The affinity of Gal3 binding to antibody was determined by acquiring real time Ligand:Analyte binding kinetics data and fitting the data with a 1:1 monovalent binding model. The kinetic evaluation procedure determines association and dissociation constants by fitting the experimental data to a 1:1 interaction model between analyte A and ligand B:

Kₐ: A+B → AB; and K_{d}: A+B ← AB, where Kₐ is the association rate constant (M⁻¹s⁻¹) and the K_{d} is the dissociation rate constant (s⁻¹).

The net rate of complex formation during injection is given by:

$$\text{d} \left[\text{AB}\right] / \text{dt} \text{=} {\text{K}}_{\text{a}} \left[\text{A}\right] \left[\text{B}\right] - {\text{K}}_{\text{d}} \left[\text{AB}\right]$$

and the rate of dissociation after the end of the injection is:

$$\text{d} \left[\text{AB}\right] / \text{dt} \text{=} - {\text{K}}_{\text{d}} \left[\text{AB}\right] .$$

The affinity KD is the result of K_{d} divided by Kₐ.

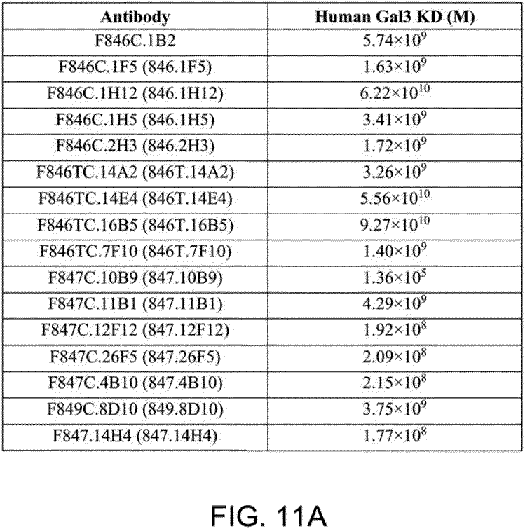

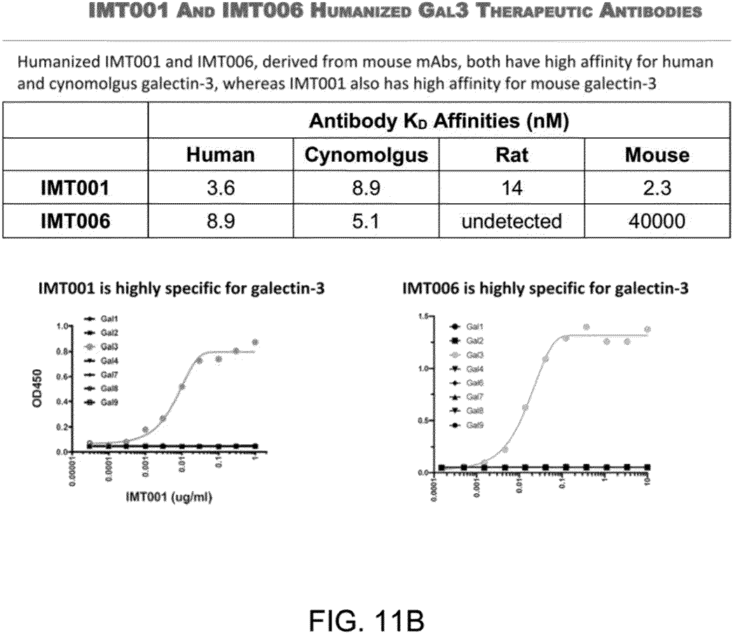

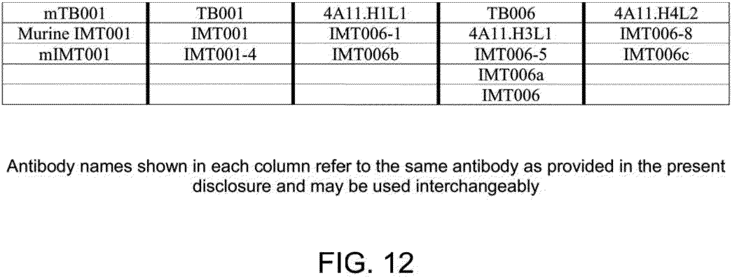

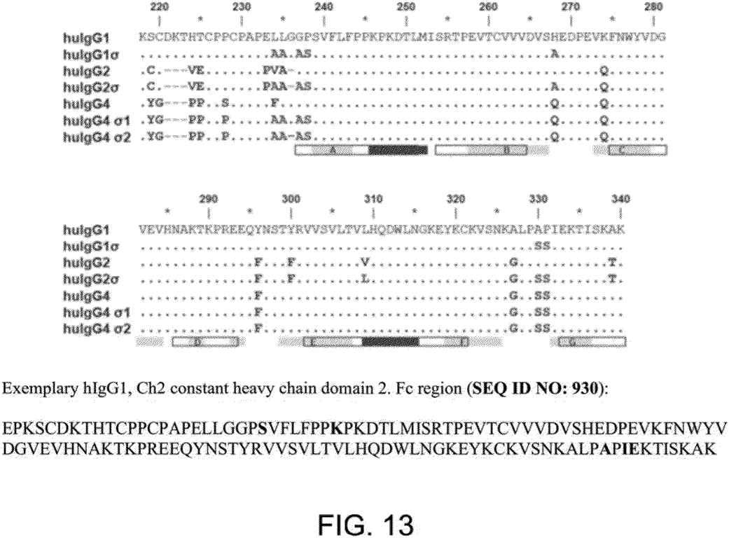

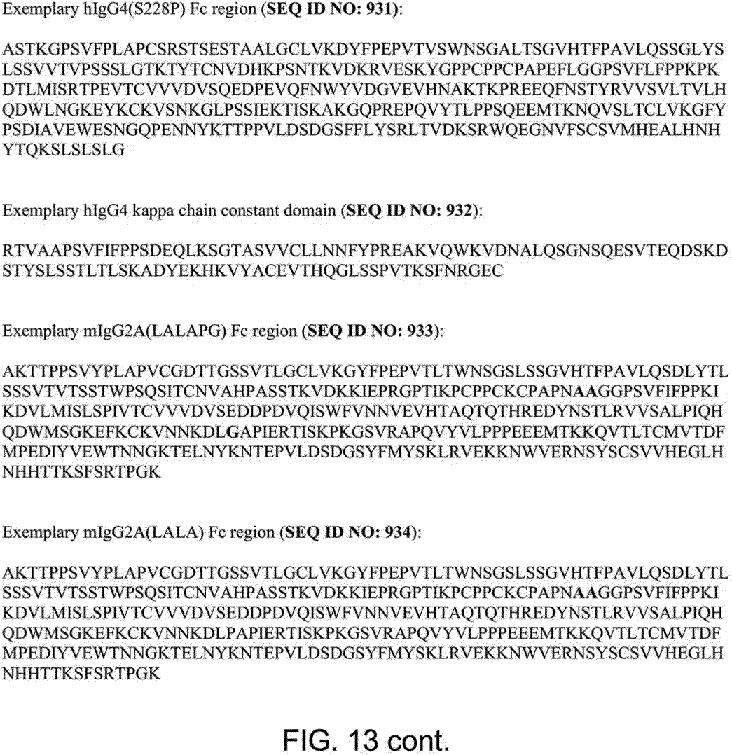

Humanized TB001 (IMT001) and TB006 (IMT006a), which were derived from mouse mAbs, both have high affinity for human (IMT001: 3.6 nM, IMT006a: 8.9 nM) and cynomolgus (IMT001: 8.9 nM, IMT006a: 5.1 nM) Gal3 (FIG. 11B). IMT001 also has high affinity for mouse Gal3 (IMT001: 2.3 nM, IMT006a: 40000 nM) and rat Gal3 (IMT001: 14 nM, IMT006a: undetected).

### Example 9: Anti-Gal3 Antibodies for Use in the Treatment of Stroke or Cerebral Amyloid Angiopathy

Patients present with stroke, cerebral amyloid angiopathy or symptoms thereof. The stroke may be an ischemic stroke, thrombotic stroke, embolic stroke, transient ischemic attack, hemorrhagic stroke, intracerebral hemorrhage, or subarachnoid hemorrhage. The cerebral amyloid angiopathy may be associated with the stroke. One or more anti-Gal3 antibodies or binding fragments thereof disclosed herein are administered to the patients enterally, orally, intranasally, parenterally, intracranially, subcutaneously, intramuscularly, intradermally, or intravenously.

The anti-Gal3 antibodies or binding fragments thereof are administered as doses in at an amount of 1000 mg (or in the alternative: 0.1, 1, 10, 100, 1000 ng, or 1, 10, 50, 100, 200, 300, 400, 500, 600, 700, 800, 900, 1000 µg, or 1, 10, 100, 200, 300, 400, 500, 600, 700, 800, 900, 1100, 1200, 1300, 1400, 1500, 1600, 1700, 1800, 1900, 2000 mg, or any amount within a range defined by any two of the aforementioned amounts, or any other amount appropriate for optimal efficacy in humans). Multiple doses may be administered as part of a treatment schedule. For example, 5 doses (or in the alternative: 2, 3, 4, 6, 7, 9, 8, 10, 11, 12, 13, 14, 15, or 16 doses) may be administered. The doses are administered every 1 day (or in the alternative: every 2, 3, 4, 5, 6, 7, 8, 9, 10, 11, 12, 24, 36, or 48 days or any time within a range defined by any two of the aforementioned times). The doses may be administered for a combined total of 5000 mg (or in the alternative: 1000, 2000, 3000, 4000, 6000, 7000, 8000, 9000, or 10000 mg) to each patient.

A treatment of the stroke, cerebral amyloid angiopathy, or symptoms thereof is observed in the patients following administration of the anti-Gal3 antibodies or binding fragments thereof. Administration of the anti-Gal3 antibodies or binding fragments may be performed in conjunction with another therapy for stroke or cerebral amyloid angiopathy, for example, thrombolytics, tissue plasminogen activator (tPA), alteplase, reteplase, tenecteplase, desmoteplase, anticoagulants, ACE inhibitors, antihypertensives, nicardipine, or surgery, or any combination thereof.

### Example 10: Anti-Gal3 Antibodies for Use in the Treatment of Traumatic Brain Injury

Patients present with traumatic brain injury or symptoms thereof. One or more anti-Gal3 antibodies or binding fragments thereof disclosed herein are administered to the patients enterally, orally, intranasally, parenterally, intracranially, subcutaneously, intramuscularly, intradermally, or intravenously.

The anti-Gal3 antibodies or binding fragments thereof are administered as doses in at an amount of 1000 mg (or in the alternative: 0.1, 1, 10, 100, 1000 ng, or 1, 10, 50, 100, 200, 300, 400, 500, 600, 700, 800, 900, 1000 µg, or 1, 10, 100, 200, 300, 400, 500, 600, 700, 800, 900, 1100, 1200, 1300, 1400, 1500, 1600, 1700, 1800, 1900, 2000 mg, or any amount within a range defined by any two of the aforementioned amounts, or any other amount appropriate for optimal efficacy in humans). Multiple doses may be administered as part of a treatment schedule. For example, 5 doses (or in the alternative: 2, 3, 4, 6, 7, 9, 8, 10, 11, 12, 13, 14, 15, or 16 doses) may be administered. The doses are administered every 1 day (or in the alternative: every 2, 3, 4, 5, 6, 7, 8, 9, 10, 11, 12, 24, 36, or 48 days or any time within a range defined by any two of the aforementioned times). The doses may be administered for a combined total of 5000 mg (or in the alternative: 1000, 2000, 3000, 4000, 6000, 7000, 8000, 9000, or 10000 mg) to each patient.

A treatment of the traumatic brain injury or symptoms thereof is observed in the patients following administration of the anti-Gal3 antibodies or binding fragments thereof. Administration of the anti-Gal3 antibodies or binding fragments may be performed in conjunction with another therapy for TBI, for example, levodopa, bromocriptine, NMDA receptor antagonists, amantadine, memantine, cholinesterase inhibitors, tacrine, rivastigmine, galantamine, donepezil, surgery, or any combination thereof.

### Example 11: Anti-Gal3 Antibodies for Use in the Treatment of Fibrin Disorders

Patients present with a disorder associated with fibrin oligomerization, or symptoms thereof. For example, the patient may present with or be at risk for one or more of atherosclerosis, thrombosis, thromboembolism, carotid artery disease, coronary artery disease, peripheral artery disease, myocardial infarction, heart failure, heart attack, hypertension, chronic kidney disease, stroke, or other cardiovascular diseases. In some embodiments, the patients may present with atherosclerosis, which may be associated with one or more of these other diseases. One or more anti-Gal3 antibodies or binding fragments thereof disclosed herein are administered to the patients enterally, orally, intranasally, parenterally, intracranially, subcutaneously, intramuscularly, intradermally, or intravenously.

The anti-Gal3 antibodies or binding fragments thereof are administered as doses in at an amount of 1000 mg (or in the alternative: 0.1, 1, 10, 100, 1000 ng, or 1, 10, 50, 100, 200, 300, 400, 500, 600, 700, 800, 900, 1000 µg, or 1, 10, 100, 200, 300, 400, 500, 600, 700, 800, 900, 1100, 1200, 1300, 1400, 1500, 1600, 1700, 1800, 1900, 2000 mg, or any amount within a range defined by any two of the aforementioned amounts, or any other amount appropriate for optimal efficacy in humans). Multiple doses may be administered as part of a treatment schedule. For example, 5 doses (or in the alternative: 2, 3, 4, 6, 7, 8, 9, 10, 11, 12, 13, 14, 15, or 16 doses) may be administered. The doses are administered every 1 day (or in the alternative: every 2, 3, 4, 5, 6, 7, 8, 9, 10, 11, 12, 24, 36, or 48 days or any time within a range defined by any two of the aforementioned times). The doses may be administered for a combined total of 5000 mg (or in the alternative: 1000, 2000, 3000, 4000, 6000, 7000, 8000, 9000, or 10000 mg) to each patient.

A treatment of the disorder associated with fibrin oligomerization or symptoms thereof is observed in the patients following administration of the anti-Gal3 antibodies or binding fragments thereof. Administration of the anti-Gal3 antibodies or binding fragments may be performed in conjunction with another therapy for disorders associated with fibrin oligomerization or cardiovascular diseases, for example, anti-clotting agents (e.g. aspirin), statins, antihypertensives, diuretics, calcium channel blockers, ACE inhibitors, angiotensin II receptor antagonists, adrenergic receptor antagonists, vasodilators, renin inhibitors, aldosterone receptor antagonists, endothelium receptor blockers, or surgery, such as vascular bypass surgery, percutaneous coronary intervention, coronary artery bypass graft, carotid endarterectomy, or angioplasty.

### Example 12: Clinical Study of Efficacy Safety, and Pharmacokinetics of TB006 in Patients With Acute Ischemic Stroke

Stroke is a leading cause of death and disability. In 2018, more than 795,000 people in the US experienced a stroke event and stroke related costs exceed $46 billion. Stroke reduces mobility in more than half of stroke survivors aged 65 and over. About 87% of all strokes are ischemic strokes in which blood flow to the brain is blocked. Nonetheless, IV thrombolysis with tPA and thrombectomy are still the only approved treatments for AIS. Due to limitations in tPA dosing, especially a narrow therapeutic window, only < 5% of hospitalized patients for stroke receive tPA. Researchers have been desperately trying to identify novel treatment strategies beyond recanalization, i.e., neuroprotective and neurorestorative treatments. In the last few decades, more than 1,000 acute stroke treatments have been tested successfully in the preclinical lab, with nearly 100% failure in large clinical trials. Therefore, there is a huge need to develop therapies using different approaches that would be accessible to a greater number of patients for the treatment of acute stroke.

Activity in stroke research peaked in the mid-1990s to the mid-2000s. The vast majority of experimental drugs developed to treat AIS have been classified as either thrombolytics or neuroprotectants. The former group encompasses compounds that restore blood flow, while the latter term covers drugs that act to preserve neurons. One of the limitations with these approaches, even if patients gain access to them and they are successful, is that neuronal damage is done. After the acute period, stroke survivors face a myriad of challenges, including, but not limited to, hemiparesis and aphasia. While evidence supports the utility of rehabilitation efforts after acute stroke, complete neurological and physical recovery is rarely complete. The profound vacuum in this field is particularly disappointing because evidence suggests that functional recovery is possible. Innovative approaches to enhance the body’s endogenous regenerative abilities remain an opportunity for improvement.

Galectin-3 levels are elevated for up to 2 months following brain ischemia in animals. The primary source of Gal-3 in the acute ischemic brain tissue is the microglia. Galectin-3 expression leads to infiltration of inflammatory cells, and then the production of reactive oxygen species. Reduction or inhibition of Gal-3 contributes to the revascularization of previously ischemic tissue and improvement of post stroke angiogenesis and plays an important role in remyelination and recovery from neuronal loss. Therefore, compounds that inhibit Gal-3 may contribute to neuro-restoration. In addition, the benefits of blocking Gal-3 are evident if the treatment is started 8 days after the stroke event.

Anti-Gal3 antibodies can be used as therapeutic agents for acute ischemic stroke (AIS) by neutralizing Gal3, which regulates activation of astrocytes and microglia. Activation of these cells, which occurs in stroke, leads to neuronal damage and infiltration of other inflammatory factors compounding the cascade of functional decline. TB006 has been shown in in vitro models to reduce activated astrocytes and microglia, and has demonstrated improvement in in vivo models of stroke in both locomotor and cognitive functioning. This evidence supports clinical testing in an AIS setting. The preclinical safety profile of TB006 further supports the clinical investigation of TB006.

This example represents a Phase 2 study in patients with a mild to moderate AIS event that will evaluate the efficacy, safety and tolerability, and pharmacokinetic (PK) profile of TB006, administered once weekly (QW) as an intravenous (IV) infusion over 1 hour. This example will commence after the safety, tolerability, and PK of single TB006 doses up to 5,000 mg have been established in healthy volunteers. The primary efficacy endpoint will be the National Institutes of Health Stroke Scale (NIHSS), and the primary efficacy assessment will be the change from Baseline in the active treatment group versus placebo. Other efficacy measures include the modified Rankin Scale (mRS), Fugl-Meyer Assessment (FMA), and Montreal Cognitive Assessment (MoCA). A variety of assessments, including physical exams, vital signs, electrocardiograms (ECGs), and clinical labs, will be used to assess safety.

Patients with mild to moderate ischemic stroke are eligible to participate if all eligibility criteria are met and they are able to receive their first dose of treatment within 7 days of the documented stroke event. Patients may be enrolled into the example trial while either in an inpatient facility or following discharge; likewise, patients may receive their 5 weekly doses of drug while in an inpatient facility or following discharge. All patients will be followed up for safety, efficacy, and PK assessments through Day 104 of the example.

This example will evaluate the efficacy and safety of TB006 in patients with an AIS event with 1 month of treatment. The total example study duration of each patient will be up to approximately 15 weeks. This example has a parallel-group design with 2 treatment groups: TB006 and placebo. 112 patients are randomized in a 1:1 ratio (56 patients per group) to receive either treatment. Doses will be administered QW for a total of 5 doses (Days 1, 8, 15, 22, 29). The dose of TB006 is 1,000 mg QW (total of 5,000 mg over 1 month). This example will commence after the safety and tolerability of a single 5,000 mg dose has been established in a single ascending dose (SAD) study.

Patients with an ischemic stroke event, as documented with a computed tomography (CT) scan or magnetic resonance imaging (MRI), with a Baseline NIHSS total score of 7 to 21 (inclusive), are eligible to participate. Patients must be randomized and dosed within 7 days of their known documented AIS event. The last known awake time will be used as the stroke onset time for patients whose stroke occurred during sleep. They may be either hospitalized or have been discharged at Baseline. Despite the 7-day window from time of stroke onset to first dose of administration, every effort should be made to administer the first dose as early as possible. Hospitalized patients may be dosed, monitored and evaluated initially as inpatients; following discharge, patients will be followed on an outpatient basis for dose administration (if any remain), and all follow-up assessments through Day 104. Patients may receive standard of care treatments for ischemic stroke, including the administration of tissue plasminogen activator if they are eligible, throughout the trial.

All randomized patients will follow the dosing schedule, and efficacy and safety procedures through Day 104, and at early discontinuation (E/D) if necessary. Patients will return to the clinic on dosing days and on follow-up visit days following hospital discharge. If a patient reports adverse events (AEs), they may be required to return to the clinic (if they are outpatients) at discretion for additional assessments. All AEs must be followed to adequate resolution. Efficacy assessments include the NIHSS, mRS, FMA, and MoCA, and are conducted by trained and certified staff. TB006 infusions are administered IV over 1 hour. Doses may be prepared by either hospital or research site pharmacy staff.

### Inclusion Criteria

1) Male and/or female 18 to 85 (inclusive) years of age at the time of signing the informed consent.

2) Body mass index (BMI) between 18 and 40 kg/m², inclusive.

3) Clinical diagnosis of AIS in anterior circulation, supported by acute brain CT or MRI consistent with the clinical diagnosis.

4) Able to be randomized and dosed within 7 days of index stroke event. The last known awake time will be used for patients whose stroke occurred during sleep.

5) National Institute of Health Stroke Scale total score of 7 to 21, inclusive.

6) Participants or their legally authorized representative confirms that prior to index stroke event, no significant impairment in participant’s ability to perform Activities of Daily Living (ADL) without assistance.

7) Patients may have received standard of care therapy including tPA, thrombectomy, mannitol, hypertonic saline, and other treatments per local guidelines.

8) Contraceptive use by men or women should be consistent with local regulations regarding the methods of contraception for those participating in clinical studies.

a) Male subjects are eligible to participate if they agree to the following from the first day of dosing until at least 90 days after dosing: refrain from donating sperm, PLUS, either: be abstinent from heterosexual intercourse as their preferred and usual lifestyle (abstinent on a long term and persistent basis) and agree to remain abstinent unless partner is not a Woman of Childbearing Potential (WOCBP) OR must agree to use contraception/barrier.

b) Female subjects are eligible to participate if they are not pregnant or breastfeeding, and at least one of the following condition applies: is not a WOCBP, OR be abstinent from heterosexual intercourse as their preferred and usual lifestyle (abstinent on a long term and persistent basis) and agree to remain abstinent throughout the example study, OR is a WOCBP and using a contraceptive method that is highly effective (with a failure rate of < 1% per year), preferably with low user dependency from Baseline until completion of the example study, and agrees not to donate eggs (ova, oocytes) for the purpose of reproduction during this period. A WOCBP must have a negative highly sensitive pregnancy test (serum) prior to randomization.

9) Patients or caregiver or legally authorized representative has the ability to understand the purpose and risks of the example study and provide signed and dated informed consent which includes compliance with the requirements and restrictions. Patients whose caregiver signs the informed consent must provide their assent.

### Exclusion Criteria

1) Evidence of severe stroke on imaging (e.g., sulcal effacement or blurring of gray-white junction in greater than ⅓ of middle cerebral artery [MCA] territory, Alberta Stroke Program Early CT [ASPECT] score of 0 to 4 based on head CT, acute infarct volume on MRI diffusion weighted imaging ≥ 70 mL) based on acute imaging studies performed under the standard of care.

2) Lacunar or isolated brainstem or cerebellar stroke based on clinical assessment and available acute imaging studies performed under the standard of care.

3) Evidence of seizure at the onset of index stroke.

4) Evidence of acute myocardial infarction (MI) at Baseline, including any of the following:
a) acute ST elevation MI,
b) acute decompensated heart failure, or New York Heart Association Class III/IV heart failure,
c) admission for an acute coronary syndrome, MI, cardiac arrest, or non-voluntary coronary intervention (percutaneous coronary intervention or coronary artery surgery) within the past 3 months,
d) QT interval corrected using Bazett’s formula (QTcB) > 520 msec.

5) Evidence of acute intracranial or subarachnoid hemorrhage or evidence of active bleeding based on acute brain CT or MRI performed under the standard of care. However, petechial hemorrhages of ≤ 1 cm are not exclusionary.

6) Symptoms are considered likely to resolve within the subsequent few hours (e.g. transient ischemic attack [TIA]).

7) Known history prior to randomization of clinically significant medical conditions (other than current AIS) or any other reason, including any physical, psychological, or psychiatric condition that in the investigator’s opinion would compromise the safety or interfere with the participant’s participation in this example.

8) Known medical history of repeated episodes of complex migraine. Participants of complex migraine, but with imaging conclusively demonstrating an AIS are still allowed.

9) Subjects with, in the opinion of the investigator, a life expectancy of < 3 months not related to current event, or those unlikely to be compliant with example study procedures or follow-up visits.

10) Any substances detected in the drug screen dipstick test at Baseline that, in the opinion of the investigator, cannot be adequately explained.

11) Female who is pregnant, breastfeeding, or considering becoming pregnant during the example study or within 10 weeks after the last dose of drug.

12) Active coronavirus disease (COVID-19) infection.

13) Known recipient of any investigational product within 30 days or 5 half-lives of the drug (whichever is longer) prior to the first dose of drug. No current enrollment in another interventional clinical study, including behavioral interventional studies.

Summary of statistical considerations: Summary statistics will be reported by treatment group for efficacy endpoints (NIHSS total score, MoCA total score) and change from Baseline in each of these efficacy endpoints at each applicable visit. Unadjusted and adjusted mean treatment differences, along with the 95% CI of the change from Baseline in each efficacy endpoint at each applicable visit will also be reported. Adjusted mean treatment difference will be derived by using a mixed-effect model for repeated measures (MMRM) that includes the fixed-effects of treatment, visit (categorical covariate), treatment-by-visit interaction and Baseline score. An unstructured variance-covariance matrix might be used, where appropriate. Appropriate linear contrast will be used to compare the 2 treatment groups.

Baseline values will be taken as the most recent assessment prior to dosing, reported during screening and up to and including Day 1.

Primary Endpoint: The primary efficacy measure is the change in neurological function from Baseline in the NIHSS total score through Day 104. The primary analysis will determine whether there is a difference between the active treatment group and the placebo treatment group in mean change from Baseline up to Day 104 in the NIHSS total score, according to the methods noted above. Effect size will also be determined. The NIHSS is performed at Baseline, then at Days 15, 36, 64, and 104 following initial intervention (intervention or safety follow-up period).

For testing as the change from Baseline on the NIHSS, the adjusted mean treatment differences will be derived by using a MMRM that includes the fixed effects of treatment, visit (categorical covariate), treatment-by-visit interaction, and Baseline score. An unstructured variance-covariance matrix might be used, where appropriate. Appropriate linear contrast will be used to compare the 2 treatment groups.

Secondary Endpoint: For testing the proportion of patients with clinically significant improvement on the mRS (defined as a 1-point decrease) and NIHSS (defined as a 4-point decrease) on Day 36 (and sustained through Day 104) will be compared using a Mantel-Haenszel test.

Primary and secondary objectives and endpoints are described in Table 1. ADA = anti-drug antibody; AEs = adverse events; AIS = acute ischemic stroke; Cₘₐₓ = maximum concentration; C-SSRS = Columbia Suicide Severity Rating Score; C_{trough} = minimum concentration; ECG = electrocardiogram; FMA = Fugl-Meyer Assessment; MoCA = Montreal Cognitive Assessment; mRS = modified Rankin Scale; NIHSS = National Institutes of Health Stroke Scale; PK = pharmacokinetic(s); SAE = serious adverse event; t_{½} = time required to reduce by half the plasma concentration of the drug.

TABLE 1

Objectives and Endpoints for AIS Treatment

| | |
|---|---|
| Objectives | Endpoints |
| Primary Objective: To determine the clinical efficacy of TB006 in patients with AIS | • Change in neurological function from Baseline through Day 104 on the NIHSS |
| Secondary Objective: To determine the clinical efficacy of TB006 in patients with AIS | • The proportion of patients who demonstrate clinically significant improvement on the NIHSS (defined as a 4-point decrease) on Day 36 (and sustained through Day 104) |
| | • Change in neurological function from Baseline through Day 36 on the NIHSS |
| | • The proportion of patients who demonstrate clinically significant improvement on the mRS (defined as a 1-point decrease) on Day 36 (and sustained through Day 104) |
| | • Change in the total score on the FMA on Day 36 and Day 104 |
| | • Change in the total score on the MoCA on Day 36 and Day 104 |
| Secondary Objective: To determine the safety and tolerability of TB006 | Safety endpoints, including the incidence of AEs and SAEs, clinical laboratory parameters, vital signs, ECGs, C-SSRS, physical and neurological examinations, and ADA until Day 104 after the first TB006 dosing |
| Secondary Objective: To determine the PK following multiple doses of TB006 | PK parameters including, but not limited to, C_{trough}, Cₘₐₓ, and t_{½}. |

For testing the change from Baseline on the FMA total score and the MoCA total score, the adjusted mean treatment differences will be derived by using a MMRM that includes the fixed effects of treatment, visit (categorical covariate), treatment-by-visit interaction, and Baseline score. An unstructured variance-covariance matrix might be used, where appropriate. Appropriate linear contrast will be used to compare the 2 treatment groups. For the immunogenicity assessment, ADA results will be descriptively summarized.

Safety, PK and Other Data: Safety data will be summarized descriptively, including tables, listings and figures, as appropriate. Unless otherwise stated, descriptive summary statistics for continuous variables will include number (n), mean (i.e., arithmetic mean), standard deviation (SD), minimum, median and maximum. Descriptive statistics for categorical data will include frequency and percentages.

All AEs will be coded using the most recent version of Medical Dictionary for Regulatory Activities (MedDRA). The incidence of AEs will be summarized by system organ class (SOC) and preferred term (PT). Similarly, summaries will be produced for AEs by severity, serious AEs (SAEs), treatment related AEs, and AEs leading to discontinuation. AEs are collected from the first dose of drug; thus all AEs are considered treatment-emergent AEs.

The ECG results will be presented by visit, summarizing the continuous measurements based on the average of triplicate values, including QT, QTc, QRS, RR, PR intervals, as well as change from Baseline values, and clinically significant changes in heart rate and rhythm.

Reported values and change from Baseline values of clinical laboratory data for safety (and the determinations relevant to the normal ranges and appropriate clinically significant or Common Terminology Criteria for Adverse Events (CTCAE) toxicity gradings) will be summarized by laboratory test for each assessment day, using descriptive statistics.

Vital signs (blood pressure, pulse rate, respiratory rate, and oral temperature) will be summarized by visit showing absolute values, change from Baseline values using descriptive statistics.

Physical and neurological examinations will be summarized by assessment day showing shift from Baseline.

The Columbia Suicide Severity Rating Scale (C-SSRS) assessment will be presented in a by-subject listing.

For continuous PK variables, descriptive summary statistics will, in addition, include geometric mean, geometric SD (GSD), arithmetic coefficients of variation (CV%), and geometric coefficients of variation (GCV%). Plasma TB006 concentrations over time will be presented, showing both individual and mean concentrations. Plasma PK parameters will also be calculated and presented descriptively. Plasma PK parameters of interest will include maximum concentration (Cₘₐₓ), minimum concentration (C_{trough}) time at which maximum plasma concentration occurs (Tₘₐₓ), area under the concentration-time profile over the dosing interval (tau) at steady state (AUC₀₋ₜₐᵤ), time required to reduce by half the plasma concentration of the drug (t½), clearance at steady state (CLₛₛ), and volume in steady state (Vₛₛ), as data permit. More details will be outlined in the statistical analysis plan (SAP).

The subject disposition showing numbers randomized, treated, completed, and discontinued from the example study will be summarized. For those subjects who discontinued the example study prematurely, the reason for discontinuation will be summarized.

All subject data will be reviewed for the occurrence of protocol deviations. Summaries will be presented showing the numbers of subjects with each class and type of deviation.

Demographic variables to be summarized will include age, gender, race, ethnicity, height, weight, and BMI.

Baseline characteristics to be summarized will include medical history.

Prior and concomitant medications will be summarized showing the number and percentage of subjects taking each medication. Medications will be coded using the World Health Organization Drug Dictionary (WHO DD) preferred name.

Determination of Sample Size: The sample size estimation is based on the change in neurological function from Baseline through Day 104 in the active treatment group compared with placebo group on the NIHSS total score. A 2.0 point absolute difference between placebo and the active treatment group in the change from Baseline is assumed.

The example is powered to assure 90% likelihood of identifying an absolute difference of 2.0 points between the active and placebo groups in the change from Baseline in the NIHSS total score. Sample size estimation is based on a repeated measures when the SD is 4.2 and a within correlation between visits is 0.5 for comparing the 2 means at Day 104. The maximum sample size required for randomization is 112 patients (56 per treatment group).

Benefit Assessment: TB006 is a humanized IgG4 (S228P) type monoclonal antibody that is highly specific and has high affinity to human Gal3. It has demonstrated efficacy in preclinical models of neurodegenerative disorders including stroke, AD, and TBI, and results are consistent with or superior to other compounds that have advanced into late-stage clinical studies. Preclinical data in the acute neurodegenerative models of stroke and TBI indicate a wide treatment window. Thus, there is potential for TB006 to be effective in these disorders. The preclinical safety profile of TB006 supports the further investigation of TB006 in clinical studies.

Patients will undergo clinical evaluations/assessments associated with example procedures (e.g., safety laboratory tests, vital signs, and ECG measurements, and physical and neurological examinations), which would be a potential benefit for their own health awareness.

The preclinical data on TB006 to date suggests a wide margin of safety and minimal risk of adverse effects. The risks associated with example procedures such as ECGs and blood draws are also very low. Any potential adverse effects associated with the example study drug and procedures are further minimized by intense safety monitoring and medical oversight by the investigator and staff. Therefore, the potential risks identified in association with TB006 are justified by the anticipated benefits that may be afforded to patients with AIS if TB006 is successfully developed.

Justification for Dose: A SAD study established the safety, tolerability, and PK of TB006 doses ranging from 70 to 5,000 mg in healthy volunteers. Exposures (Cₘₐₓ and AUC) at the highest dose afforded adequate safety margins from the exposures observed in the definitive GLP 1-month toxicology study. In the ongoing Phase 2 study in patients with Alzheimer’s Disease (AD), the same dose level of 1,000 mg QW × 5 doses is being assessed. The exposures (both Cₘₐₓ and AUC) from this dosing regimen will be lower than a single 5,000 mg dose, given that the same total dose is administered in divided doses over a month. Therefore, the exposures in the present example confer no additional risk than in previous studies.

The preclinical efficacy program established the potential of TB006 in several models of acute and chronic neurodegenerative disorders. Although plasma therapeutic concentration ranges have not been established, the data indicate a positive dose-response relationship across these models. Therefore, the highest safe dose that in the range of doses that are being tested in the Phase 1 program will likely afford the greatest probability of success. For these reasons, a dose regimen of 1,000 mg QW, which again is similar to the dose regimen used in the ongoing AD study, is selected as the dose level for this example.

### Example Study Intervention Administered

The example study intervention administered is depicted in Table 2. AxMP = alternative medicinal product; Gal3 = galectin-3; IB = Investigator’s Brochure; IMP = investigational medical product; NA = not applicable; NIMP = non investigational medicinal product; IV = intravenous(ly); QW = once per week.

TABLE 2

Example study intervention administered

| | | |
|---|---|---|
| Intervention label | TB006 | Placebo |
| Intervention Name | Anti-Gal3 antibody (TB006) | Placebo |
| Intervention description | IV administration of 1,000 mg, QW | IV administration of placebo, QW |
| Type | Drug | NA |
| Dose Formulation | Sterile, white, off-white, or light-yellow solution for injection | Placebo will be normal saline, 250 mL |
| Unit Dose Strength(s) | 20 mg/mL in 8 mL vials (160 mg total) | NA |
| Dosage Level(s) | Prior to administration, the drug product, at a dose of 1,000 mg, is | NA |
| | diluted in 250 mL of sterile normal saline | |
| Route of Administration | IV infusion over 1 hour | IV infusion over 1 hour |
| IMP and NIMP/AxMP | IMP | NIMP |
| Sourcing | Provided centrally by the sponsor | Provided centrally by the sponsor |
| Packaging and Labeling | TB006 will be provided as single-use injectable glass vials sealed with rubber stopper and aluminum plain flip top for sterile vial. Each vial will be labeled as required per country requirement. TB006 can be stored at \2-8° C. (36-46° F.) | An unblinded pharmacist will prepare the example drug. The placebo is plain saline bag, 250 mL, and will be identical in appearance to the active drug. |

TB006 sterilized drug product is supplied in 8 mL glass vials, sealed with a rubber stopper and an aluminum flip top, with strength of 160 mg (20 mg/mL, 8 mL) TB006 drug product per vial. TB006 drug product can be stored at 2-8° C. (36-46° F.).

TB006 drug product should be administered via IV infusion over 60 minutes, after dilution in 0.9% Sodium Chloride Injection, USP (normal saline) to a final volume of 250 mL. The dose formulation should be a sterile, white, off-white, or light-yellow solution.

For control, a placebo of 250 mL of normal saline will be administered IV QW over 1 hour. An unblinded pharmacist will prepare the example drug. The placebo is a plain saline bag of 250 mL and will be identical in appearance to the active drug.

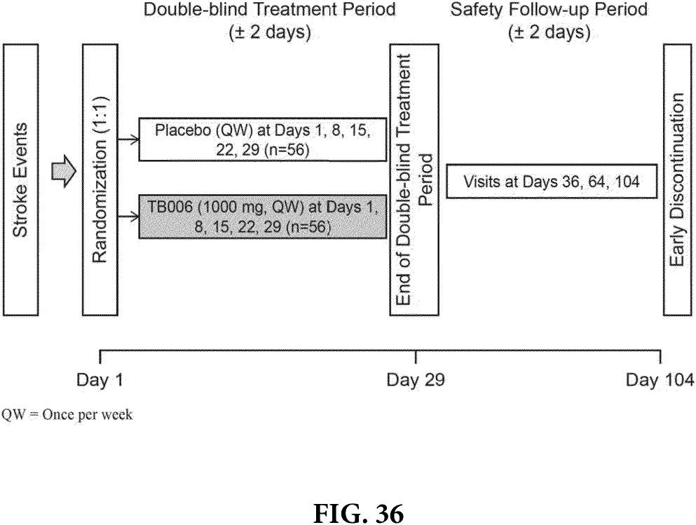

An exemplary dose administration and follow up schema for the present example is provided in FIG. 36.

Example Study Intervention Compliance: Patients may be initially dosed as inpatients or at the investigator’s clinic. In either case, following discharge, patients will return to the clinic for dose administration (if any remain).

When participants are dosed, they will receive example study intervention directly from the investigator or designee, under medical supervision. TB006 infusions are administered IV over 1 hour. The date and time of each dose administered will be recorded in the source documents. The dose of example study intervention and participant identification will be confirmed at the time of dosing by a member of the site staff other than the person administering the example study intervention.

A record of the quantity of TB006 dispensed to and administered by each participant must be maintained and reconciled with study intervention and compliance records. Intervention start and stop dates, including dates for intervention delays and/or dose reductions will also be recorded.

### Efficacy Assessments

The primary efficacy assessment scale(s) selected for this example are the list of scale for the primary efficacy endpoints. Efficacy assessments will be performed at planned timepoints. Every effort should be made to ensure that the protocol required tests and procedures are completed as described. The primary efficacy assessment scales can include the National Institutes of Health Stroke Scale, Modified Rankin Scale, Fugl-Meyer Assessment, and/or the Montreal Cognitive Assessment, as described herein as well as generally known in the art. Other approaches of assessing qualities of stroke patients, including locomotor activity, and behavioral and cognitive ability, may also be employed.

### Safety Assessments

Physical examinations: A complete physical examination will include, at a minimum, assessments of the cardiovascular, respiratory, gastrointestinal, and neurological systems. Height (at screening only) and weight will also be measured and recorded. Investigators should pay special attention to clinical signs related to previous serious illnesses.

Vital signs: Oral temperature, pulse rate, respiratory rate, and blood pressure will be assessed. Blood pressure and pulse measurements will be assessed with a completely automated device. Manual techniques will be used only if an automated device is not available. Blood pressure and pulse measurements should be preceded by at least 5 minutes of rest for the participant in a quiet setting without distractions (e.g., television, cell phones). Vital signs will be taken before blood collection for laboratory tests, if that is scheduled for the same time point.

Electrocardiograms: 12-lead ECG(s) will be obtained using an ECG machine that automatically calculates the heart rate and measures PR, RR, QRS, QT, and QTc intervals. On non-dosing days, effort should be made to perform 12-lead ECGs time-matched with the pre-dose time point on dosing days. The investigator will review and assess all ECGs as Normal, Abnormal, or Not Evaluable.

The following clinical safety laboratory samples will be evaluated during the example:

Hematology: Hemoglobin, hematocrit, RBC count, WBC, platelet count, WBC count with differential (neutrophils, eosinophils, basophils, lymphocytes, and monocytes), % reticulocytes, MCV, MCH, and MCHC.

Clinical chemistry: albumin, total protein, blood glucose, sodium, potassium, creatinine, AST, ALT, ALP, GGT, total bilirubin, triglyceride, total cholesterol, CPK, LDH, calcium, uric acid, SGOT, total and direct bilirubin, BUN, SGPT, TSH, HbA1c, PT/INR.

Urinalysis: urine specific gravity, pH, protein, glucose, ketones, urobilinogen, bilirubin, leukocyte esterase, nitrite by dipstick and blood. Microscopic examination (if blood or protein is abnormal).

### Urine Alcohol and Drug Screen

Pharmacokinetics: Whole blood samples of approximately 5 mL will be collected for measurement of plasma concentrations of TB006. The timing of sampling may be altered during the course of the example based on newly available data (e.g., to obtain data closer to the time of peak plasma concentrations) to ensure appropriate monitoring. Samples will be used to evaluate the PK of TB006. Each plasma will be divided into 2 aliquots (1 each for PK and a backup). Samples collected for analyses of TB006 (plasma) concentration may also be used to evaluate safety or efficacy aspects related to concerns arising during or after the example study.

Immunogenicity Assessments: Antibodies to TB006 will be evaluated in plasma samples collected from all participants. Additionally, plasma samples should also be collected at the final visit from participants who discontinued example study intervention or were withdrawn from the example study. Plasma samples will be screened for antibodies binding to TB006 and the titer of confirmed positive samples will be reported. Other analyses may be performed to verify the stability of antibodies to TB006 and/or further characterize the immunogenicity of TB006.

The detection and characterization of antibodies to TB006 will be performed using a validated assay method by or under the supervision of the sponsor. ADA assessment is performed on blood sample collected for PK assessments; Day 1 assessment is performed on the pre dose sample. All samples collected for detection of antibodies to example study intervention will also be evaluated for TB006 plasma concentration to enable interpretation of the antibody data. Antibodies may be further characterized and/or evaluated for their ability to neutralize the activity of the example study intervention(s). Samples may be stored for a maximum of 15 years (or according to local regulations) following the last participant’s last visit for the example study at a facility selected by the sponsor to enable further analysis of immune responses to TB006.

### Statistical Analyses

General considerations: Summary statistics will be reported by treatment group for efficacy endpoints (NIHSS total score, mRS, FMA, MoCA total score) and change from Baseline in each of these efficacy endpoints at each applicable visit. Unadjusted and adjusted mean treatment differences, along with the 95% CI of the change from Baseline in each efficacy endpoint at each applicable visit will also be reported. Adjusted mean treatment difference will be derived by using a MMRM that includes the fixed-effects of treatment, visit (categorical covariate), treatment-by-visit interaction, and Baseline score. An unstructured variance covariance matrix might be used, where appropriate. Appropriate linear contrast will be used to compare the 2 treatment groups.

Baseline values will be taken as the most recent assessment prior to dosing, reported during screening and up to and including Day 1.

Primary Endpoint(s): The primary efficacy measure is the change in neurological function from Baseline in the NIHSS total score through Day 104. The primary analysis will determine whether there is a difference between the active treatment group and the placebo treatment group in mean change from Baseline at Day 104 in the NIHSS total score, according to the methods noted above. Effect size will also be determined.

For testing as the change from Baseline on the NIHSS, the adjusted mean treatment differences will be derived by using a MMRM that includes the fixed effects of treatment, visit (categorical covariate), treatment-by-visit interaction, and Baseline score. An unstructured variance-covariance matrix might be used, where appropriate. Appropriate linear contrast will be used to compare the 2 treatment groups.

Secondary Endpoint(s): For testing the proportion of patients with clinically significant improvement on the mRS (defined as a 1-point decrease) and NIHSS (defined as a 4-point decrease) on Day 36 (and sustained through Day 104) will be compared using a Mantel-Haenszel test.

For testing the change from Baseline on the FMA total score and the MoCA total score, the adjusted mean treatment differences will be derived by using a MMRM that includes the fixed-effects of treatment, visit (categorical covariate), treatment-by-visit interaction, and Baseline score. An unstructured variance-covariance matrix might be used, where appropriate. Appropriate linear contrast will be used to compare the 2 treatment groups. For the immunogenicity assessment, ADA results will be descriptively summarized.

### Example 13: Treatment of Acute Ischemic Stroke With an Anti-Gal3 Antibody

A patient presents with acute ischemic stroke (AIS). An anti-Gal3 antibody or binding fragment thereof as described herein, such as TB006, is administered to the patient, ideally within 7 days of the incidence of the stroke event. The patient is administered with 5 unit doses of 1000 mg or about 1000 mg of the anti-Gal3 antibody or binding fragment thereof per each unit dose. The unit doses are administered to the patient every 7 days or about 7 days (i.e. administering the full course of 5 unit doses will take approximately 30 days from the time of first dose). The unit doses are each prepared as 250 mL solutions of saline containing the 1000 mg or about 1000 mg of antibody or binding fragment thereof, and are administered over the course of 1 hour or about 1 hour.

Following the full course of administration, the patient is monitored for improvements related to the stroke or symptoms thereof, such as locomotor activity, and behavioral and cognitive ability. The patient can be assessed starting 7 days following the last dose administration, and can also be assessed subsequently approximately every month if needed (e.g. after 7, 35, and 75 days after the last dose administration). The patient is assessed by standardized approaches used for diagnosing stroke, such as the NIHSS, mRS, FMA, or MoCA, or any combination thereof. If the NIHSS is used, the patient is considered to have an amelioration of the stroke if the patient exhibits at least a 4 point decrease on the NIHSS. If the mRS is used, the patient is considered to have an amelioration of the stroke if the patient exhibits at least a 1 point decrease on the mRS. If the FMA is used, the patient is considered to have an amelioration of the stroke if the patient exhibits at least a 1 point increase on the FMA. If the MoCA is used, the patient is considered to have an amelioration of the stroke if the patient exhibits at least a 1 point increase on the MoCA. Additional and/or alternative approaches may also be used, such as neuroimaging, CT, MRI, and/or detecting stroke biomarkers such as S100B, GFAP, NSE, and/or MMP9.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

A Sequence Listing in electronic format is submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Ajjan R, Grant PJ (2006) Coagulation and atherothrombotic disease. Atherosclerosis 186: 240-59.

Andrade RJ, Robles M, Lucena MI. Rechallenge in drug-induced liver injury: the attractive hazard. Expert Opin Drug Saf. 2009;8:709-714.

Ashraf GM et. al. “Investigation of Gal-3 Expression Pattern in Serum and Cerebrospinal Fluid of Patients Suffering From Neurodegenerative Disorders.” Front. Neurosci. 2018, 12:430. PMCID: PMC6033997

Banks JL, Marotta CA. Outcomes Validity and Reliability of the Modified Rankin Scale: Implications for Stroke Clinical Trials. Stroke 2007;38(3):1091-1096.

Barua RS, Sy F, Srikanth S, Huang G, Javed U, et al. (2010) Effects of cigarette smoke exposure on clot dynamics and fibrin structure: an ex vivo investigation. Arterioscler Thromb Vasc Biol 30: 75-9.

Benjamin EJ, Blaha MJ, Chiuve SE, et al. (2017). Heart disease and stroke statistics-2017 update: a report from the American Heart Association. Circulation, 135, e146-e603.

Boza-Serrano A, et. al. “Galectin-3, a novel endogenous TREM2 ligand, detrimentally regulates inflammatory response in Alzheimer’s disease.” Acta Neuropathol. 2019, 138(2):251-273. PMCID: PMC6660511

Brott T, Adams HP, Olinger CP, et al. Measurements of acute cerebral infarction-a clinical examination scale. Stroke 1989;20 (7):864-70.

Burguillos MA, et. al. “Microglia-Secreted Galectin-3 Acts as a Toll-like Receptor 4 Ligand and Contributes to Microglial Activation.” Cell Rep. 2015, 10:1626-1638.

Carter AM, Cymbalista CM, Spector TD, Grant PJ (2007) Heritability of clot formation, morphology, and lysis: the EuroCLOT study. Arterioscler Thromb Vasc Biol 27: 2783-9.

Centers for Disease Control and Prevention. (2015). Report to Congress on Traumatic Brain Injury in the United States: Epidemiology and Rehabilitation. National Center for Injury Prevention and Control; Division of Unintentional Injury Prevention. Atlanta, GA.

Chistiakov DA et al. The role of monocytosis and neutrophilia in atherosclerosis. J. Cell Mol. Med. (2018) 22(3):1366-1382.

Collet JP, Allali Y, Lesty C, Tanguy ML, Silvain J, et al. (2006) Altered fibrin architecture is associated with hypofibrinolysis and premature coronary atherothrombosis. Arterioscler Thromb Vasc Biol 26: 2567-73

Corrado E, Rizzo M, Coppola G, Fattouch K, Novo G, et al. (2010) An update on the role of markers of inflammation in atherosclerosis. J Atheroscler Thromb 17: 1-11.

Dunn EJ, Ariens RA, Grant PJ (2005) The influence of type 2 diabetes on fibrin structure and function. Diabetologia 48: 1198-206.

Dunn EJ, Philippou H, Ariens RA, Grant PJ (2006) Molecular mechanisms involved in the resistance of fibrin to clot lysis by plasmin in subjects with type 2 diabetes mellitus. Diabetologia 49: 1071-80.

Eric S Donkor Stroke in the 21st Century: A Snapshot of the Burden, Epidemiology, and Quality of Life Stroke Res Treat. 2018; 2018: 3238165.

Fang MC, Cutler DM, Rosen AB. (2010). Trends in Thrombolytic Use for Ischemic Stroke in the United States. J Hosp Med, 5(7), 406-409.

Fatkhullina AR et al. The Role of Cytokines in the Development of Atherosclerosis. Biochemistry (Mosc) (2016) 81(11):1358-1370.

Fugl-Meyer, AR et al. The post-stroke hemiplegic patient. I. a method for evaluation of physical performance”. Scandinavian Journal of Rehabilitation Medicine 1975;7(1):13-31.

Freynhofer MK, Bruno V, Wojta J, Huber K (2012) The role of platelets in athero-thrombotic events. Curr Pharm Des 18: 5197-214.

Go AS, Mozaffarian D, Roger VL, et al. (2014). Executive summary: heart disease and stroke statistics—2014 update: a report from the American Heart Association. Circulation, 129, 399-410.

Goldstein LB, Bartels C, Davis JN. Interrater reliability of the NIH Stroke Scale. Arch Neurology 1989;46 (6):660-662.

Green AR, Ashwood T. (2005). Free radical trapping as a therapeutic approach to neuroprotection in stroke: experimental and clinical studies with NXY-059 and free radical scavengers. Curr Drug Targets-CNS & Neurol Dis, 4, 109-118.

Hachinski V, Iadecola C, Petersen RC, et al. National Institute of Neurological Disorders and Stroke-Canadian Stroke Network vascular cognitive impairment harmonization standards. Stroke 2006;37:2220-2241.

Hunt, CM. Mitochondrial and immunoallergic injury increase risk of positive drug rechallenge after drug-induced liver injury: A systematic review. Hepatol. 2010;52:2216-2222.

Inoue Y et al. Current Management and Therapeutic Strategies for Cerebral Amyloid Angiopathy. Int. J. Mol. Sci. (2021) 22, 3869 p. 1-15.

Leander K, Blomback M, Wallen H, He S (2012) Impaired fibrinolytic capacity and increased fibrin formation associate with myocardial infarction. Thromb Haemost 07: 1092-9.

Lee Y-J et al. Spatial and temporal expression, and statin responsiveness of galectin-1 and galectin-3 in murine atherosclerosis. Korean Circ. J. (2013) 43(4):223-30.

Liu Y et al. Association of the Total White Blood Cell, Neutrophils, and Monocytes Count with the Presence, Severity, and Types of Carotid Atherosclerotic Plaque. Front. Med. (2020) 7:313.

Lu Y et al. Modified citrus pectin inhibits galectin-3 function to reduce atherosclerotic lesions in apoE-deficient mice. Mol. Med. Rep. (2017) 16(1):647-653.

Madrigal-Matute J, Lindholt JS, Fernandez-Garcia CE, et al. (2014). Galectin-3, a biomarker linking oxidative stress and inflammation with the clinical outcomes of patients with atherothrombosis. J Am Heart Assoc, 3(4), pii:e00078.

McKee AC, Robinson ME. “Military-related traumatic brain injury and neurodegeneration.” Alzheimers Dement. 2014, 10(30): S242-S253.

Mehndiratta P. et al. “Cerebral amyloid angiopathy-associated intracerebral hemorrhage: pathology and management” Neurosurg Focus 2012 Apr;32(4).

Mills JD, Ariens RA, Mansfield MW, Grant PJ (2002) Altered fibrin clot structure in the healthy relatives of patients with premature coronary artery disease. Circulation 106: 1938-42.

Murphy TH, Corbett D. (2009). Plasticity during stroke recovery: from synapse to behaviour. Nat Rev Neurosci, 10, 861-872.

Nachtigal M et al. Galectin-3 expression in human atherosclerotic lesions. Am. J. Pathol. (1998) 152(5):1199-1208.

Nasreddine ZS, Phillips NA, Bédirian V, et al. The Montreal Cognitive Assessment, MoCA: a brief screening tool for mild cognitive impairment. J Am Geriatr Soc.2005;53:695-699.

Nishikawa H, Suzuki H. “Possible Role of Inflammation and Galectin-3 in Brain Injury after Subarachnoid Hemorrhage.” Brain Sci 2018, 8(2):30.

O’Collins VE, Macleod MR, Donnan GA, et al. (2006). 1,026 experimental treatments in acute stroke. Ann Neurol, 59, 467-477.

Osmancik P, Paulu P, Tousek P, Kocka V, Widimsky P (2012) High leukocyte count and interleukin-10 predict high on-treatment-platelet-reactivity in patients treated with clopidogrel. J Thromb Thrombolysis 33: 349-54.

Page S, Fulk G, Boyne P. Clinically Important Differences for the Upper-Extremity Fugl-Meyer Scale in People With Minimal to Moderate Impairment Due to Chronic Stroke. Journal of the American Physical Therapy Association 2012;92 (6):791-798.

Papaspyridonos M et al. Galectin-3 Is an Amplifier of Inflammation in Atherosclerotic Plaque Progression Through Macrophage Activation and Monocyte Chemoattraction. Arteriosclerosis, Thrombosis, and Vascular Biology. (2008);28:433-440.

Papay JI, Clines D, Rafi R, Yuen N, Britt SD, Walsh JS, Hunt CM. Drug-induced liver injury following positive drug rechallenge. Regul Tox Pharm. 2009;54:84-90.

Paul J. et al. Fibrin deposition accelerates neurovascular damage and neuroinflammation in mouse models of Alzheimer’s disease. J Exp Med. 2007 Aug 6;204(8):1999-2008.

Powers WJ, Rabinstein AA, Ackerson T, et al. (2018). 2018 guidelines for the early management of patients with acute ischemic stroke: a guideline for healthcare professionals from the American Heart Association/American Stroke Association. Stroke, 49, e46-e110.

Puigdellivol M. et al. “Sialylation and Galectin-3 in Microglia-Mediated Neuroinflammation and Neurodegeneration.” Front. Cell. Neurosci. 2020, 14:162. PMCID: PMC7296093

Reijmer YD. et al. “Ischemic brain injury in cerebral amyloid angiopathy” J Cereb Blood Flow Metab. 2016 Jan; 36(1): 40-54.

Rodrigues MA et al. The Edinburgh CT and genetic diagnostic criteria for lobal intracerebral haemorrhage associated with cerebral amyloid angiopathy: model development and diagnostic test accuracy study. Lancet Neurol. (2018) 17(3):232-240.

Romain Goulay et al. “From Stroke to Dementia: a Comprehensive Review Exposing Tight Interactions Between Stroke and Amyloid-β Formation” Transl Stroke Res. 2020; 11(4): 601-614.

Sanford J, Moreland J. Reliability of the Fugl-Meyer Assessment for Testing Motor Performance in Patients Following Stroke. Rehabilitation Measures Database1993;73 (7):447-54.

Satoh K, Niwa M, Goda W, et al. (2011). Galectin-3 expression in delayed neuronal death of hippocampal CA1 following transient forebrain ischemia, and its inhibition by hypothermia. Brain Res, 1382, 266-274.

Scott EM, Ariens RA, Grant PJ (2004) Genetic and environmental determinants of fibrin structure and function: relevance to clinical disease. Arterioscler Thromb Vasc Biol 24: 1558-66.

Shan R et. al. “A New Panel of Blood Biomarkers for the Diagnosis of Mild Traumatic Brain Injury/Concussion in Adults.” J Neurotrauma 2016, 33(1):49-57.

Shen, YF et. al. “The change of plasma galectin-3 concentrations after traumatic brain injury.” Clin. Chim. Acta 2016, 456:75-80.

Toglia J, Fitzgerald KA, O’Dell MW, Mastrogiovanni AR, Lin CD. The Mini-Mental State Examination and Montreal Cognitive Assessment in persons with mild subacute stroke: relationship to functional outcome. Arch Phys Med Rehabil. 2011;92:792-798.

Undas A, Kolarz M, Kopec G, Tracz W (2008) Altered fibrin clot properties in patients on long-term haemodialysis: relation to cardiovascular mortality. Nephrol Dial Transplant 23: 2010-5.

Undas A, Plicner D, Stepien E, Drwila R, Sadowski J (2007) Altered fibrin clot structure in patients with advanced coronary artery disease: a role of C-reactive protein, lipoprotein(a) and homocysteine. J Thromb Haemost 5: 1988-90.

Undas A, Szuldrzynski K, Stepien E, Zalewski J, Godlewski J, et al. (2008) Reduced clot permeability and susceptibility to lysis in patients with acute coronary syndrome: effects of inflammation and oxidative stress. Atherosclerosis 196: 551-7.

van Swieten JC, Koudstaal PJ, Visser MC, Schouten HJ, van GJ. Interobserver agreement for the assessment of handicap in stroke patients. Stroke 1988;19:604-607.

Veerbeek JM, van Wegen E, van Peppen R, et al. (2014). What is the evidence for physical therapy poststroke? A systematic review and meta-analysis. PLoS One, 9,e87987.

Virani SS, Alonso A, Benjamin EJ, et al. (2020). Heart disease and stroke statistics-2020 update: a report from the American Heart Association. Circulation, 141(9), e139-e596.

Viswanathan A & Greenberg SM. Cerebral amyloid angiopathy in the elderly. Ann. Neurol. (2011) 70(6):871-80.

Wang X. et. al “Elevated Galectin-3 Levels in the Serum of Patients With Alzheimer’s Disease.” Am J Alzheimers Dis Other Demen 2015, 30(8):729-32. PMID: 23823143.

Wang Y, Hel S, Liu X, et al. (2021). Galectin-3 Mediated Inflammatory Response Contributes to Neurological Recovery by QiShenYiQi in Subacute Stroke Model. Front.Pharmacol, https://doi.org/10.3389/fphar.2021.588587.

Weisel JW & Litvinov RI. Mechanisms of fibrin polymerization and clinical implications. Blood (2013) 121(10):1712-1719.

Yan YP, Lang BT, Vemuganti R, Dempsey RJ. (2009). Galectin-3 mediates post-ischemic tissue remodeling. Brain Res, 1288, 116-124.

Yip PK, et. al. “Galectin-3 released in response to traumatic brain injury acts as an alarmin orchestrating brain immune response and promoting neurodegeneration.” Sci. Rep. 2017, 7:41689.

Yoo SH, Kim JS, Kwon SU, Yun SC, Koh JY, Kang DW. Undernutrition as a predictor of poor clinical outcomes in acute ischemic stroke patients. Archives of neurology. 2008Jan 1;65(1):39-43.

What is claimed is:

1. A method for inhibiting, reducing, preventing, and/or treating stroke in a subject in need thereof, comprising administering an anti-Gal3 antibody or binding fragment thereof to the subject, thereby inhibiting, reducing, preventing, and/or treating the stroke.

2. The method of claim 1, further comprising selecting the subject as having the stroke or at risk of contracting the stroke prior to the administering step, wherein selecting the subject as having the stroke or at risk of contracting the stroke comprises assessing the subject according to the National Institutes of Health Stroke Scale (NIHSS), modified Rankin Scale (mRS), Fugl-Meyer Assessment (FMA), Montreal Cognitive Assessment (MoCA), neuroimaging, optionally CT or MRI, and/or detecting stroke biomarkers, optionally S100 calcium binding protein B (S100B), glial fibrillary acidic protein (GFAP), neuron-specific enolase (NSE), and/or matrix metalloproteinase-9 (MMP9).

3-4. (canceled)

5. The method of claim 14, further comprising detecting an improvement by the subject according to the NIHSS, mRS, FMA, or MoCA, wherein:
a) the improvement by the subject according to the NIHSS comprises at least a 4 point decrease on the NIHSS;
b) the improvement by the subject according to the mRS comprises at least a 1 point decrease on the mRS;
c) the improvement by the subject according to the FMA comprises at least a 1 point increase on the FMA; and/or
d) the improvement by the subject according to the MoCA comprises at least a 1 point increase on the MoCA;
after the administering step, optionally as assessed 7, 35, and/or 75 days after the administering step.

6. The method of claim 5, wherein:
a) the improvement by the subject according to the NIHSS comprises at least a 4 point decrease on the NIHSS;
b) the improvement by the subject according to the mRS comprises at least a 1 point decrease on the mRS;
c) the improvement by the subject according to the FMA comprises at least a 1 point increase on the FMA; and/or
d) the improvement by the subject according to the MoCA comprises at least a 1 point increase on the MoCA;
after the administering step, optionally as assessed 7, 35, and/or 75 days after the administering step.

7. The method of claim 1, wherein the stroke is an ischemic stroke, thrombotic stroke, embolic stroke, transient ischemic attack, hemorrhagic stroke, intracerebral hemorrhage, or subarachnoid hemorrhage.

8-9. (canceled)

10. The method of claim 1, wherein administration of the anti-Gal3 antibody or binding fragment thereof prevents or reduces loss of locomotor dysfunction, prevents incidence of microhemorrhage, prevents elevation of levels of activated microglia, prevents elevation of levels of activated astrocytes, prevents elevation of circulating proinflammatory immune cells, prevents elevation of circulating proinflammatory cytokines, or any combination thereof, in the subject.

11-12. (canceled)

13. The method of claim 10, wherein the reduction in locomotor dysfunction, reduction in microhemorrhage, reduction in levels of activated microglia, reduction in levels of activated astrocytes, reduction in levels of circulating proinflammatory cytokines, or any combination thereof, in the subject, is by at least 5%, 10%, 20%, 30%, 40%, 50%, 60%, 70%, 80%, 90%, or 95%, or any percentage within a range defined by any two of the aforementioned reductions.

14-18. (canceled)

19. The method of claim 10, wherein the reduction in levels of activated microglia is assessed by detection of microglia activation markers, optionally ionized calcium-binding adaptor molecule 1 (Iba1), monocyte chemoattractant protein-1 (MCP-1), chitinase-3-like protein 1 (YKL-40), and/or soluble CD14 (sCD14), optionally from the cerebrospinal fluid of the subject.

20. (canceled)

21. The method of claim 10, wherein the reduction in levels of activated astrocytes is assessed by detection of astrocyte activation markers, optionally GFAP and/or S100B, optionally from the cerebrospinal fluid of the subject.

22. The method of claim 10, wherein the proinflammatory immune cells comprise NK cells, monocytes, and/or lymphocytes and/or the proinflammatory cytokines comprise IL-6, TNF-α, and/or IL1β.

23-28. (canceled)

29. The method of claim 1, wherein the anti-Gal3 antibody or binding fragment thereof comprises one or more of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B 11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

30-33. (canceled)

34. The method of claim 1, wherein the stroke is hemorrhagic stroke, intracerebral hemorrhage, or subarachnoid hemorrhage, and the one or more additional therapeutic compositions comprise antihypertensives, or nicardipine, or any combination thereof.

35. A method for inhibiting, reducing, preventing, and/or treating traumatic brain injury (TBI) in a subject in need thereof, comprising administering an anti-Gal3 antibody or binding fragment thereof to the subject, thereby inhibiting, reducing, preventing, and/or treating the TBI.

36. The method of claim 35, further comprising selecting the subject as having the TBI or at risk of contracting the TBI prior to the administering step, wherein selecting the subject as having the TBI comprises assessing the subject for level of consciousness, memory loss, and/or the Glasgow Coma Scale, neuroimaging, optionally CT or MRI and/or detecting TBI biomarkers, optionally GFAP and/or ubiquitin carboxy-terminal hydrolase L1 (UCH-L1).

37-38. (canceled)

39. The method of claim 35, wherein the TBI is associated with a concussion, edema, diffuse axonal injury, spinal cord injury, coma, neuroinflammation, microhemorrhage, astrocytosis, activated microglia, and/or hematoma.

40. The method of claim 35, wherein administration of the anti-Gal3 antibody or binding fragment thereof to the subject prevents or treats the TBI, concussion, edema, diffuse axonal injury, spinal cord injury, coma, neuroinflammation, microhemorrhage, astrocytosis, activated microglia, and/or hematoma.

41. The method of claim 35, wherein administration of the anti-Gal3 antibody or binding fragment thereof prevents or reduces elevation of levels of activated microglia, prevents elevation of levels of activated astrocytes, prevents elevation of levels of macrophages, prevents elevation of levels of hyperphosphorylated Tau, prevents incidence of microhemorrhage, prevents neuroinflammation, prevents elevation of levels of circulating proinflammatory cytokines, or any combination thereof, in the subject.

42. (canceled)

43. The method of claim 35, wherein administration of the anti-Gal3 antibody or binding fragment thereof improves the level of consciousness, memory loss, and/or the Glasgow Coma Scale of the patient; and/or reduces levels of activated microglia, reduces levels of activated astrocytes, reduces levels of macrophages, reduces levels of hyperphosphorylated Tau, reduces microhemorrhage, reduces neuroinflammation, reduces levels of circulating proinflammatory cytokines or any combination thereof, in the subject.

44. The method of claim 43, wherein the reduction in levels of activated microglia, reduction in levels of activated astrocytes, reduction in levels of macrophages, reduction in levels of hyperphosphorylated Tau, reduction in microhemorrhage, reduction in neuroinflammation, reduction in levels of circulating proinflammatory cytokines, or any combination thereof, in the subject, is by at least 5%, 10%, 20%, 30%, 40%, 50%, 60%, 70%, 80%, 90%, or 95%, or any percentage within a range defined by any two of the aforementioned reductions.

45-60. (canceled)

61. The method of claim 35, wherein the anti-Gal3 antibody or binding fragment thereof comprises one or more of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B 11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

62-65. (canceled)

66. A method for inhibiting, reducing, preventing, and/or treating a disorder associated with fibrin activity or dysfunction in a subject in need thereof, comprising administering an anti-Gal3 antibody or binding fragment thereof to the subject, thereby inhibiting, reducing, preventing, and/or treating the disorder associated with fibrin activity or dysfunction.

67. The method of claim 66, wherein the disorder associated with fibrin activity or dysfunction comprises atherosclerosis, thrombosis, thromboembolism, carotid artery disease, coronary artery disease, peripheral artery disease, myocardial infarction, heart failure, heart attack, hypertension, chronic kidney disease, coagulopathy, or thrombocytopathy.

68. The method of claim 67, further comprising selecting the subject as having the disorder associated with fibrin activity or dysfunction or at risk of contracting the disorder associated with fibrin activity or dysfunction prior to the administering step, wherein selecting the subject as having or being at risk of having the disorder associated with fibrin activity or dysfunction comprises detecting thrombotic biomarkers, optionally fibrinogen, C-reactive protein (CRP), and/or plasminogen activator inhibitor-1 (PAI-1).

69-71. (canceled)

72. The method of claim 66, wherein administration of the anti-Gal3 antibody or binding fragment thereof to the subject reduces neuroinflammation and/or fibrin oligomerization in the subject by at least 5%, 10%, 20%, 30%, 40%, 50%, 60%, 70%, 80%, 90%, or 95%, or any percentage within a range defined by any two of the aforementioned reductions.

73-78. (canceled)

79. The method of claim 66, wherein the anti-Gal3 antibody or binding fragment thereof comprises one or more of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B 11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

80-84. (canceled)

85. A method comprising administering 5 unit doses of an anti-Gal3 antibody or binding fragment thereof, wherein each unit dose comprise 1000 mg or about 1000 mg of the anti-Gal3 antibody or binding fragment thereof, wherein the unit doses are administered every 7 days or about 7 days, and wherein each unit dose is administered over the course of 1 hour or about 1 hour.

86. The method of claim 85, wherein the anti-Gal3 antibody or binding fragment thereof is TB006 or a binding fragment thereof.

87-89. (canceled)

90. A single-use sealed injectable glass vial comprising 8 mL of 20 mg/mL of an anti-Gal antibody or binding fragment thereof, optionally wherein the anti-Gal3 antibody or binding fragment thereof is TB006 or a binding fragment thereof.

91. An IV infusion bag comprising 1000 mg or about 1000 mg of an anti-Gal3 antibody or binding fragment thereof dissolved in 250 mL or about 250 mL of saline, optionally wherein the anti-Gal3 antibody or binding fragment thereof is TB006 or a binding fragment thereof.
